(19) United States
(12) Patent Application Publication
(10) Pub. No.: US 2024/0182870 A1
(43) Pub. Date: Jun. 6, 2024

(54) PRODUCTION BACTERIAL CELLS AND USE THEREOF IN PRODUCTION METHODS

(71) Applicant: Eligo Bioscience, Paris (FR)

(72) Inventors: Jesus FERNANDEZ RODRIGUEZ, Paris (FR); Antoine DECRULLE, Paris (FR); Aymeric LEVEAU, Paris (FR); Ines CANADAS BLASCO, Paris (FR); Aurélie MATHIEU, Paris (FR); Thibault CARLIER, Paris (FR)

(21) Appl. No.: 18/424,461

(22) Filed: Jan. 26, 2024

Related U.S. Application Data
(63) Continuation of application No. 18/319,918, filed on May 18, 2023, now Pat. No. 11,939,598.
Application No. 18/319,918 is a division of application No. 17/742,671, filed on May 12, 2022, now Pat. No. 11,697,802.
(60) Provisional application No. 63/187,531, filed on May 12, 2021.
(60) Provisional application No. 63/187,532, filed on May 12, 2021.

(51) Int. Cl. C12N 7/00; C12N 1/20; C12N 15/70; C12N 15/74

(52) U.S. Cl. CPC C12N 7/00; C12N 1/20; C12N 15/70; C12N 15/74; C12N 2795/10322; C12N 2795/10352; C12N 2800/101; C12N 2800/40

(57) ABSTRACT

The present invention concerns a production bacterial cell for producing phage particles or phage-derived delivery vehicles, said production bacterial cell stably comprising at least one phage structural gene(s) and at least one phage DNA packaging gene(s), said phage structural gene(s) and phage DNA packaging gene(s) being derived from a first type of bacteriophage,
wherein the expression of at least one of said phage structural gene(s) and/or at least one of said phage DNA packaging gene(s) in said production bacterial cell is controlled by at least one induction mechanism, and
wherein said production bacterial cell is from a bacterial species or strain different from the bacterial species or strain from which said first type of bacteriophage comes and/or that said first type of bacteriophage targets.

### REFERENCE TO SEQUENCE LISTING SUBMITTED ELECTRONICALLY

The instant application contains a Sequence Listing which has been filed electronically in ST.26 format and is hereby incorporated by reference in its entirety. Said ST.26 copy, created on May 16, 2023, is named EB2021-04a_USDiv.xml and is 172,032 bytes bytes in size.

### CROSS REFERENCE TO RELATED APPLICATIONS

This application is a continuation of U.S. application Ser. No. 18/319,918, which is a divisional of U.S. application Ser. No. 17/742,671 filed on May 12, 2022, which claims the benefit of U.S. application 63/187,531 filed May 12, 2021, and U.S. application 63/187,532 filed May 12, 2021, which are incorporated herein by reference in their entirety.

### FIELD OF THE INVENTION

The present invention concerns bacterial cells for producing phage particles and methods using such bacterial cells.

### BACKGROUND

Most current phage or phage-derived delivery vehicle production methods imply the use, as production cell, of the bacterial species or strain which is the natural host of said phages. Such methods can turn out to be dangerous when such bacterial cells are pathogenic, for example when they produce toxins. Moreover, many bacterial species cannot be easily manipulated, for instance because of their growth conditions or because there is no efficient genetic tool for those bacteria. It can also be difficult to identify a bacterial strain that only contains a prophage, since in many cases, a same bacterial strain contains several prophages, which can give rise to unwanted particles being produced or unwanted recombination events, etc., and/or to induce and/or stably maintain a prophage/phage in a given species or strain, for example.

There is thus a need for a method enabling the safe, easier and efficient production of any phage or phage-derived particle.

The present invention meets this need.

The present inventors considered that phages can be viewed as more or less large genetic circuits, the final output of which is the generation of more phage particles. To do this, no matter if the phage is lytic, temperate or chronic (for instance filamentous phages such as M13), the present inventors considered that the information encoded in their genomes can be roughly categorized depending on the function it performs:
Genes devoted to insertion/excision (for temperate phages).
Genes devoted to DNA replication, RNA transcription, etc. Indeed, some lytic phages encode their own RNA or DNA polymerases, for instance. Some genes modify the host's RNA polymerases to be able to work past terminators, and some other genes are involved in the segregation of the prophage sequence if it exists in a plasmid or linear plasmid form.
Genes devoted to packaging of the newly synthesized phage genome into the newly synthesized phage capsids: terminases and accessory proteins, ligases, etc.
Structural genes devoted to building a protein capsid for the DNA: apart from strictly structural genes, such as capsid genes, tape measure, fibers, baseplate etc, many other genes are needed to assemble the components (chaperones, proteases) as well as proteins that can be packaged inside the capsid, be it as scaffold or as pilot proteins injected into the cell (for instance, the RNA polymerase of phage N4 or some minor pilot proteins in other phages).
Genes related to defense from host's anti-phage mechanisms, degradation/modification of host's elements to complete the lytic cycle, super-exclusion mechanisms or genes that are advantageous for the host.

The DNA packaging and structural genes categories are deeply connected, since the packaging machinery recognizes the pre-assembled capsid heads and the DNA to be packaged in these heads, initiates and terminates DNA packaging.

The present inventors hypothesized that by abstracting and differentiating all the modules defined above, a system could be built that contains all excision/insertion, replication and regulation elements from one phage and encodes the packaging/structural elements for another one, since, as considered by the inventors, they could be viewed as independent genetic modules.

Treating them as independent genetic modules could also allow for the construction of a system that contains only the desired structural and/or regulatory elements of the phage to be produced under the control of a master regulatory element (an inducible repressor, for example) that may not be derived from a phage, as opposed to wild-type phages where gene expression is tightly regulated by phage elements. For instance, only the structural operon and the DNA packaging machinery of a phage could be placed under the control of a repressor that responds to a small molecule or a physical/chemical signal (LacI, AraC, PhIF, Lambda cl, etc.), triggering the production of all the elements necessary to generate pure mature phage delivery particles (phages or packaged phagemids). This “trimmed down” version of a phage genome could be stably maintained in a bacterial production strain.

### SUMMARY OF THE INVENTION

The present invention arises from the unexpected finding by the inventors that it is possible, by exchanging the structural operon of an Escherichia coli production strain encoding a system to generate pure Lambda packaged phagemids with the structural operon of a prophage coming from a different species (such as Klebsiella pneumoniae), to drive the assembly and packaging of pure heterologous phagemid particles when supplemented with a plasmid containing the correct packaging signals (cos site for the Klebsiella pneumoniae phage). The present inventors thus here showed that phagemids can be produced structurally based on a K. pneumoniae prophage, but regulated and maintained in the lysogenic state by the Lambda prophage machinery in an Escherichia coli production strain.

The inventors also showed that the structural operon of a P. freudenreichii prophage can be exchanged with the structural operon of a phage of a C. acnes strain. With this approach, the inventors showed that it is possible, by exchanging the structural operon of a P. freudenreichii prophage with the structural operon of a phage of a C. acnes strain, to drive the assembly and packaging of pure C. acnes phagemids.

This approach represents a novel avenue for easier and/or safer generation of phage particles and/or phage-derived delivery vehicles, targeting bacterial cells known to be pathogenic and/or difficult to manipulate and/or inefficient to use in phage particles and/or phage-derived delivery vehicles production for any reason.

The present invention thus concerns a production bacterial cell for producing phage particles or phage-derived delivery vehicles, said production bacterial cell stably comprising at least one phage structural gene(s) and at least one phage DNA packaging gene(s), said phage structural gene(s) and phage DNA packaging gene(s) being derived from a first type of bacteriophage,
wherein the expression of at least one of said phage structural gene(s) and/or at least one of said phage DNA packaging gene(s) in said production bacterial cell is controlled by at least one induction mechanism, and
wherein said production bacterial cell is from a bacterial species or strain different from the bacterial species or strain from which said first type of bacteriophage comes and/or that said first type of bacteriophage targets.

The present invention also concerns a method for producing phage particles or phage-derived delivery vehicles, comprising:
(a) providing the production bacterial cell of the invention, and
(b) inducing, in said production bacterial cell, expression of said at least one of said phage structural gene(s) and phage DNA packaging gene(s), and assembly of the products expressed by said at least one phage structural gene(s) and said at least one phage DNA packaging gene(s), thereby producing phage particles or phage-derived delivery vehicles.

Another object of the invention concerns a hybrid helper phage system comprising:
(i) at least one phage DNA packaging gene(s) derived from a first type of bacteriophage,
(i′) at least one phage structural gene(s) derived from said first type of bacteriophage, and
(ii) at least one gene, derived from a second type of bacteriophage, involved in phage excision/insertion, phage DNA replication, and/or phage regulation,
wherein said genes (i), (i′) and (ii) are comprised in a unique nucleic acid molecule or in separate nucleic acid molecules,
wherein said first type of bacteriophage comes from and/or target bacterial species or strain different from the bacterial species or strain from which said second type of bacteriophage comes and/or that said second type of bacteriophage targets, and
wherein said hybrid helper phage system does not comprise any expressed phage structural gene derived from said second type of bacteriophage.

### DETAILED DESCRIPTION OF THE INVENTION

### Production Bacterial Cell

The present invention concerns a production bacterial cell for producing phage particles or phage-derived delivery vehicles, said production bacterial cell stably comprising at least one phage structural gene(s) and at least one phage DNA packaging gene(s) derived from a first type of bacteriophage,
wherein the expression of at least one of said phage structural gene(s) and at least one of said phage DNA packaging gene(s) in said production bacterial cell is controlled by an induction mechanism, and
wherein said production bacterial cell is from a bacterial species or strain different from the bacterial species or strain from which said first type of bacteriophage comes and/or that said first type of bacteriophage targets.

As used herein, the term “phage particle” refers to a functional or non-functional (for example non-reproductive and/or replicative) virion.

As used herein, the term “phage-derived delivery vehicle” refers to any means that allows the transfer of a payload into a bacterium and which is derived from a bacteriophage. In the context of the invention, the term “phage-derived delivery vehicle” further encompasses bacteriophage-derived particles which do not comprise any payload but are able to target bacterial cells.

The phage-derived delivery vehicle can refer to a bacteriophage derived scaffold and can be obtained from a natural, evolved or engineered bacteriophage.

### Bacterial Cell

The production bacterial cell of the invention may be of any bacterial species or strain, in particular defined below under the section “Targeted bacteria”, provided that said bacterial species or strain is different from the bacterial species or strain from which said first type of bacteriophage comes and/or that said first type of bacteriophage targets.

However, the production bacterial cell is preferably a non-pathogenic bacterial cell. Still preferably, the production bacterial cell is a bacterial cell which can be easily manipulated.

By “easily manipulated” is meant herein that the bacterial cell can be cultured and/or modified using well-known techniques.

In a particular preferred embodiment, said production bacterial cell is an E. coli bacterial cell. Alternatively, said production bacterial cell may be a Bacteroides bacterial cell, more particularly a Bacteroides thetaiotaomicron bacterial cell, a P. freudenreichii bacterial cell, a Fusobacterium bacterial cell, or a Streptococcus bacterial cell. In a particular embodiment, said production bacterial cell is a P. freudenreichii bacterial cell.

The production bacterial cell of the invention can be obtained by any technique well-known from the skilled person, in particular by introducing into a bacterial cell, said phage structural gene(s) and phage DNA packaging gene(s) derived from a first type of bacteriophage, by any technique well-known in the art.

The production bacterial cell of the invention can typically be obtained by homologous recombination or recombineering including for example MAGE (Wannier et al. Recombineering and MAGE. Nat Rev Methods Primers 1, 7 (2021)), using CRISPR, TALEN, meganucleases and/or Zn-finger technologies for instance or using site specific recombination with phage integrase, PASTE (Ioannidi et al. Drag-and-drop genome insertion without DNA cleavage with CRISPR-directed integrases. Biorxiv 2021.11.01.466786 (2021) doi:10.1101/2021.11.01.466786) or Transposon-Associated CRISPR-Cas System (Ma et al. Trends Microbiol 29, 565-568 (2021)).

### Phage DNA Packaging Genes and Phage Structural Genes

The production bacterial cell of the invention stably comprises at least one phage structural gene(s) and at least one phage DNA packaging gene(s) derived from a first type of bacteriophage.

By “stably comprise” or “stably comprising” is meant herein that the production bacterial cell retains said phage structural gene(s) and phage DNA packaging gene(s) either incorporated into its chromosome, or on an episome that is maintained in the cell typically through selection (e.g., with a nutritional, auxotrophic, or drug resistance marker). Each gene stably comprised by the production bacterial cell can independently be on a plasmid, on a helper phage, or is integrated into the production bacterial cell chromosome.

In a particular embodiment, said production bacterial cell stably comprises at least two, 3, 4, or all phage structural genes derived from said first type of bacteriophage, and at least one phage DNA packaging gene(s) derived from said first type of bacteriophage.

In a particular embodiment, said production bacterial cell stably comprises at least one phage structural gene(s) derived from said first type of bacteriophage, and at least two or all phage DNA packaging genes derived from said first type of bacteriophage.

In a particular embodiment, said production bacterial cell stably comprises at least two, 3, 4, or all phage structural genes derived from said first type of bacteriophage, and at least two or all phage DNA packaging genes derived from said first type of bacteriophage.

In a particular embodiment, said production bacterial cell stably comprises all phage structural genes derived from said first type of bacteriophage, and all phage DNA packaging genes derived from said first type of bacteriophage.

By “phage structural genes” is meant herein genes from a bacteriophage which are involved in the building of the bacteriophage protein capsid. Phage structural genes include genes encoding phage structural elements; genes encoding phage proteins involved in the assembly of the phage structural elements; and genes encoding phage proteins packaged inside the capsid as scaffold or as pilot proteins to be injected into a targeted bacterial cell.

Phage structural elements are well-known from the skilled person and depend on the type of bacteriophage from which they are derived. Phage structural elements can be proteins but also RNAs (for example some phages like phi29 from Bacillus subtilis encode a structural scaffold made of RNA). Phage structural elements typically include capsid proteins, tape measure proteins, fibers, baseplate proteins, tail sheath proteins, whisker proteins, decoration proteins, etc. . . .

Phage proteins involved in the assembly of the structural elements are well-known from the skilled person and depend on the type of bacteriophage from which they are derived, and optionally on the structural elements encoded by the other phage structural genes. Phage proteins involved in the assembly of the structural elements typically include phage chaperone proteins and phage proteases.

Phage proteins packaged inside the capsid as scaffold or as pilot proteins to be injected into a target host cell are well-known from the skilled person and depend on the type of bacteriophage from which they are derived. Examples of such phage proteins are RNA polymerase from phage N4 or minor pilot proteins.

As will be understood by the skilled person, the presence of a particular phage structural gene in the production bacterial cell of the invention will depend on the bacteriophage from which said phage structural genes are derived.

By “phage DNA packaging genes” is meant herein genes from a bacteriophage which are involved in the packaging of the bacteriophage genome into the bacteriophage capsid. Phage DNA packaging genes are well-known from the skilled person and include genes encoding phage terminases, genes encoding phage accessory proteins, genes encoding phage ligases, genes encoding phage exonucleases involved in DNA packaging and genes encoding phage endonucleases involved in DNA packaging.

In a particular embodiment, said production bacterial cell further stably comprises at least one gene involved in phage regulation derived from said first type of bacteriophage.

By “gene involved in phage regulation” is meant herein phage genes involved in the interaction of the phage with the host. Examples of genes involved in phage regulation include phage genes encoding master repressors, phage genes encoding anti-termination proteins, phage genes involved in super-exclusion mechanisms, phage genes involved in defense against host's anti-phage mechanisms, phage genes involved in degradation and/or modification of host's elements for example to complete the lytic cycle, and phage genes advantageous for the host.

In a particular embodiment, said production bacterial cell stably comprises phage gene(s) involved in defense against host's anti-phage mechanisms derived from said first type of bacteriophage.

In a particular embodiment, said phage structural gene(s) and phage DNA packaging gene(s) derived from said first type of bacteriophage, and optionally said gene(s) involved in phage regulation derived from said first type of bacteriophage are comprised in at least one plasmid, chromosome and/or helper phage. In a particular embodiment, said phage structural gene(s) and phage DNA packaging gene(s) derived from said first type of bacteriophage, and optionally said gene(s) involved in phage regulation derived from said first type of bacteriophage are comprised in at least two separate nucleic acid molecules, in particular at least two plasmids, chromosomes, helper phages or combinations thereof.

In a particular embodiment, said phage structural gene(s) and phage DNA packaging gene(s) derived from said first type of bacteriophage, and optionally said gene(s) involved in phage regulation derived from said first type of bacteriophage are comprised in a hybrid helper phage system as defined below.

In a particular embodiment, said phage structural gene(s) and phage DNA packaging gene(s) derived from said first type of bacteriophage, and optionally said gene(s) involved in phage regulation derived from said first type of bacteriophage are comprised in a helper phage.

### Induction Mechanism

In the context of the invention, the expression of at least one of said phage structural gene(s) and/or at least one of said phage DNA packaging genes, as defined in the section “Phage DNA packaging genes, and phage structural genes” above, in said production bacterial cell is controlled by at least one induction mechanism.

In a particular embodiment, the expression of at least one of said phage structural gene(s), in particular at least two, at least three, or all said phage structural genes, in said production bacterial cell is(are) controlled by at least one induction mechanism, in particular by one induction mechanism.

In a particular embodiment, the expression of at least one of said phage DNA packaging gene(s), in particular at least two, at least three, or all said phage DNA packaging genes, in said production bacterial cell is(are) controlled by at least one induction mechanism, in particular by one induction mechanism.

In a particular embodiment, the same induction mechanism controls the expression of the at least one of said phage structural gene(s) and the at least one of said phage DNA packaging gene(s).

In an alternative embodiment, the expression of the at least one of said phage structural gene(s) and the expression of the at least one of said phage DNA packaging gene(s) are controlled by different induction mechanisms.

By “induction mechanism” is meant herein a mechanism, encoded by a gene or group of genes comprised, in particular stably comprised, in said production bacterial cell, able to induce the expression of the genes they control, in response to a given trigger.

In a particular embodiment, said induction mechanism further controls the copy number of said at least one of said phage structural gene(s) and/or said at least one of said phage DNA packaging gene(s). In other words, in a particular embodiment, said induction mechanism further controls the replication of said at least one of said phage structural gene(s) and/or of said at least one of said phage DNA packaging gene(s), in particular the replication of the nucleic acid molecule(s) carrying said at least one of said phage structural gene(s) and/or said at least one of said phage DNA packaging gene(s).

In a particular embodiment, said induction mechanism further controls the assembly of the products expressed by said at least one of said phage structural gene(s) and said at least one of said phage DNA packaging gene(s).

Examples of such induction mechanism include:
Protein repressor or activator-based induction systems responding to small molecules (for example sugars, quorum-sensing molecules, gases, synthetic molecules, peptides, amino acids, metabolites, etc), physical signals (temperature, pressure, etc.), chemical signals (osmolarity, pH, etc.), biological signals (cell density, DNA damage, etc.); these systems may be activated by a secondary protein such as an orthogonal RNA polymerase or sigma factor.
Protein degradation systems to activate or repress transcription from a promoter.
RNA-based induction systems such as aptamers responding to the signals stated above, such as RNAi, CRISPRi, toehold systems, riboswitches, etc.
One or more nucleic acids comprising at least one gene, derived from a second type of bacteriophage, involved in phage excision/insertion, phage DNA replication, and/or phage regulation.

In a particular embodiment, said induction mechanism comprises at least one gene, derived from a second type of bacteriophage, involved in phage excision/insertion, phage DNA replication, and/or phage regulation.

Therefore, in particular embodiment, said production bacterial cell further comprises at least one gene, derived from a second type of bacteriophage, involved in phage excision/insertion, phage DNA replication, and/or phage regulation.

Genes Involved in Phage Excision/Insertion, Phage DNA Replication, and/or Phage Regulation

By “gene involved in phage excision/insertion” is meant herein genes from lysogenic phages involved in the excision of the phage, present as a prophage, from the genome or episome of a bacterial cell and/or the insertion of the phage, as a prophage, in the genome or episome of a bacterial cell.

By “gene involved in phage DNA replication” is meant herein genes from lysogenic or lytic phages, involved in the mechanism of replication of the phage DNA. Examples of genes involved in phage DNA replication include genes encoding DNA polymerase and genes involved in the segregation of the prophage sequence if it exists in a plasmid or linear plasmid form.

By “gene involved in phage regulation” is meant herein phage genes involved in the interaction of the phage with the host. Examples of genes involved in phage regulation include phage genes encoding master repressors, phage genes encoding anti-termination proteins, phage genes involved in super-exclusion mechanisms, phage genes involved in defense against host's anti-phage mechanisms, phage genes involved in degradation and/or modification of host's elements for example to complete the lytic cycle, and phage genes advantageous for the host.

In the context of the invention, said gene(s) involved in phage excision/insertion, phage DNA replication, and/or phage regulation, is(are) not DNA packaging gene(s) nor structural gene(s), as defined above.

In a preferred embodiment, the production bacterial cell of the invention comprises at least one gene, preferably all the genes, involved in phage excision/insertion derived from a second type of bacteriophage; at least one gene, preferably all the genes, involved in phage DNA replication derived from a second type of bacteriophage; and/or at least one gene, preferably all the genes, involved in phage regulation derived from a second bacteriophage.

In the context of the invention, said production bacterial cell does not comprise genes derived from the first type of bacteriophage which are involved in phage excision/insertion and/or phage DNA replication.

In a particular embodiment, said gene(s) involved in phage excision/insertion, phage DNA replication, and/or phage regulation derived from said second type of bacteriophage, are comprised in at least one plasmid, chromosome and/or helper phage. In a particular embodiment, said gene(s) involved in phage excision/insertion, phage DNA replication, and/or phage regulation derived from said second type of bacteriophage are comprised in at least two separate nucleic acid molecules, in particular at least two plasmids, chromosomes, helper phages or combinations thereof.

In a particular embodiment, said gene(s) involved in phage excision/insertion, phage DNA replication, and/or phage regulation derived from said second type of bacteriophage are comprised in a hybrid helper phage system as defined below.

In a particular embodiment, said gene(s) involved in phage excision/insertion, phage DNA replication, and/or phage regulation derived from said second type of bacteriophage, are comprised in a helper phage system, more particularly on the same helper phage system as said phage structural gene(s) and phage DNA packaging gene(s) derived from said first type of bacteriophage, and optionally said gene(s) involved in phage regulation derived from said first type of bacteriophage.

In the context of the invention, said second type of bacteriophage comes from and/or targets bacterial species or strain different from the bacterial species or strain from which said first type of bacteriophage comes and/or that said first type of bacteriophage targets.

In a particular embodiment, said production bacterial cell is from the same bacterial species or strain as the bacterial species or strain from which said second type of bacteriophage comes and/or that said second type of bacteriophage targets.

In a more particular embodiment, said production bacterial cell is an E. coli bacterial cell. In another particular embodiment, said production bacterial cell is a P. freudenreichii bacterial cell.

### Other Elements

In a particular embodiment, the production bacterial cell of the invention further comprises at least one gene involved in phage RNA transcription.

By “gene involved in phage RNA transcription” is meant genes from temperate or lytic phages, involved in the mechanism of transcription of the phage RNA. Examples of such genes include genes encoding phage RNA polymerase and phage genes encoding proteins modifying the host's RNA polymerases, typically to be able to work past terminators.

### Bacteriophage and Gene Derived from a Bacteriophage

By “gene derived from a bacteriophage” is meant herein that the sequence of the gene is obtained from a bacteriophage, said sequence being optionally modified, recoded and/or optimized compared to the sequence initially present in the bacteriophage. For example, said sequence may be recoded for codon exchange or optimization (for example some proteins of the Kappa prophage contain an amber TAG stop codon, which is not recognized by E. coli, and which is preferably changed to TAA or TGA) or preventing recombination.

Bacteriophages are obligate intracellular parasites that multiply inside bacteria by co-opting some or all of the host biosynthetic machinery. Bacteriophage can be found inside bacteria as a prophage whose genome is integrated in the bacterial chromosome or as a phage-plasmid whose genome is part of an extrachromosomal plasmid (such phage-plasmids are for example disclosed in Ravin et al. (1999) Molecular Microbiology 34(5):980-994). Examples of bacteriophage which can be in the form of a phage-plasmid include phages P1, N15, SSU5, P7, D6, pMCR-1-P3, IEBH, phiGILI6c, Bam35c, pBClin15, VP882, KS-14, P88, pLP39, F116, D3, phiSG1. Phage genomes come in a variety of sizes and shapes (e.g., linear or circular). Most phages range in size from 24-200 nm in diameter. Phages contain nucleic acid (i.e., genome) and proteins, and may be enveloped by a lipid membrane. Depending upon the phage, the nucleic acid genome can be either DNA or RNA, and can exist in either circular or linear forms. The size of the phage genome varies depending upon the phage. The simplest phages have genomes that are only a few thousand nucleotides in size, while the more complex phages may contain more than 100,000 nucleotides in their genome, and in rare instances more than 1,000,000. The number and amount of individual types of protein in phage particles will vary depending upon the phage.

In a particular embodiment, the first type of bacteriophage is selected from the Order Caudovirales consisting of, based on the taxonomy of Krupovic et al. (Krupovic et al. Arch Virol. 2016 January; 161(1):233-47):
family Myoviridae (such as, without limitation, genus Cp220virus, Cp8virus, Ea214virus, Felixo1virus, Mooglevirus, Suspvirus, Hp1virus, P2virus, Kayvirus, P100virus, Silviavirus, Spo1virus, Tsarbombavirus, Twortvirus, Cc31virus, Jd18virus, Js98virus, Kp15virus, Moonvirus, Rb49virus, Rb69virus, S16virus, Schizot4virus, Sp18virus, T4virus, Cr3virus, Se1virus, V5virus, Abouovirus, Agatevirus, Agrican357virus, Ap22virus, Arv1virus, B4virus, Bastillevirus, Bc431virus, Bcep78virus, Bcepmuvirus, Biquartavirus, Bxz1virus, Cd119virus, Cp51virus, Cvm10virus, Eah2virus, E1virus, Hapunavirus, Jimmervirus, Kpp10virus, M12virus, Machinavirus, Marthavirus, Msw3virus, Muvirus, Myohalovirus, Nit1virus, P1virus, Pakpunavirus, Pbunavirus, Phikzvirus, Rheph4virus, Rsl2virus, Rslunavirus, Secunda5virus, Sep1virus, Spn3virus, Svunavirus, Tg1virus, Vhm1virus and Wphvirus)
family Podoviridae (such as, without limitation, genus Fri1virus, Kp32virus, Kp34virus, Phikmvvirus, Pradovirus, Sp6virus, T7virus, Cp1virus, P68virus, Phi29virus, Nona33virus, Pocjvirus, T12011virus, Bcep22virus, Bpp1virus, Cba41virus, Dfl12virus, Ea92virus, Epsilon 15virus, F116virus, G7cvirus, Jwalphavirus, Kf1virus, Kpp25virus, Lit1virus, Luz24virus, Luz7virus, N4virus, Nonanavirus, P22virus, Pagevirus, Phieco32virus, Prtbvirus, Sp58virus, Una961virus and Vp5virus) family Siphoviridae (such as, without limitation, genus Camvirus, Likavirus, R4virus, Acadianvirus, Coopervirus, Pg1virus, Pipefishvirus, Rosebushvirus, Brujitavirus, Che9cvirus, Hawkeyevirus, Plotvirus, Jerseyvirus, K1gvirus, Sp31virus, Lmd1virus, Una4virus, Bongovirus, Reyvirus, Buttersvirus, Charlievirus, Redivirus, Baxtervirus, Nymphadoravirus, Bignuzvirus, Fishburnevirus, Phayoncevirus, Kp36virus, Rogue1virus, Rtpvirus, T1virus, Tlsvirus, Ab18virus, Amigovirus, Anatolevirus, Andromedavirus, Attisvirus, Barnyardvirus, Bernal13virus, Biseptimavirus, Bronvirus, C2virus, C5virus, Cba181virus, Cbastvirus, Cecivirus, Che8virus, Chivirus, Cjw1virus, Corndogvirus, Cronusvirus, D3112virus, D3virus, Decurrovirus, Demosthenesvirus, Doucettevirus, E125virus, Eiauvirus, Ff47virus, Gaiavirus, Gilesvirus, Gordonvirus, Gordtnkvirus, Harrisonvirus, Hk578virus, Hk97virus, Jenstvirus, Jwxvirus, Kelleziovirus, Korravirus, L5virus, Lambdavirus, Laroyevirus, Liefievirus, Marvinvirus, Mudcatvirus, N15virus, Nonagvirus, Np1virus, Omegavirus, P12002virus, P12024virus, P23virus, P70virus, Pa6virus, Pamx74virus, Patiencevirus, Pbi1virus, Pepy6virus, Pfr1virus, Phic31virus, Phicbkvirus, Phietavirus, Phifelvirus, Phijl1virus, Pis4avirus, Psavirus, Psimunavirus, Rdjlvirus, Rer2virus, Sap6virus, Send513virus, Septima3virus, Seuratvirus, Sextaecvirus, Sfi11virus, Sfi21dtivirus, Sitaravirus, Sk1virus, Slashvirus, Smoothievirus, Soupsvirus, Spbetavirus, Ssp2virus, T5virus, Tankvirus, Tin2virus, Titanvirus, Tm4virus, Tp21virus, Tp84virus, Triavirus, Trigintaduovirus, Vegasvirus, Vendettavirus, Wbetavirus, Wildcatvirus, Wizardvirus, Woesvirus, Xp10virus, Ydn12virus and Yuavirus) family Ackermannviridae (such as, without limitation, genus Ag3virus, Limestonevirus, Cba120virus and Vi1virus)

In a particular embodiment, the first type of bacteriophage is not part of the Order Caudovirales but from families with Unassigned order such as, without limitation, family Tectiviridae (such as genus Alphatectivirus, Betatectivirus), family Corticoviridae (such as genus Corticovirus), family Inoviridae (such as genus Fibrovirus, Habenivirus, Inovirus, Lineavirus, Plectrovirus, Saetivirus, Vespertiliovirus), family Cystoviridae(such as genus Cystovirus), family Leviviridae(such as genus Allolevivirus, Levivirus), family Microviridae (such as genus Alpha3microvirus, G4microvirus, Phix174microvirus, Bdellomicrovirus, Chlamydiamicrovirus, Spiromicrovirus) and family Plasmaviridae (such as genus Plasmavirus).

In a particular embodiment, the first type of bacteriophage is targeting Archea not part of the Order Caudovirales but from families with Unassigned order such as, without limitation, Ampullaviridae, FuselloViridae, Globuloviridae, Guttaviridae, Lipothrixviridae, Pleolipoviridae, Rudiviridae, Salterprovirus and Bicaudaviridae.

In a particular embodiment, the second type of bacteriophage is selected from the bacteriophages defined above, provided that said second type of bacteriophage is different from said first type of bacteriophage.

In a particular embodiment, said first type of bacteriophage comes from a first bacterial species or strain, and said second type of bacteriophage comes from a second bacterial species or strain, wherein said first and second bacterial species or strains are different.

By “bacteriophage coming from a particular bacterial species or strain” is meant herein a bacteriophage specifically targeting a particular bacterial species or strain and/or a bacteriophage hosted by a particular bacterial species or strain.

A non-exhaustive listing of bacterial genera and their known host-specific bacteria viruses is presented in the following paragraphs. Synonyms and spelling variants are indicated in parentheses. Homonyms are repeated as often as they occur (e.g., D, D, d). Unnamed phages are indicated by “NN” beside their genus and their numbers are given in parentheses.

Bacteria of the genus Actinomyces can be infected by the following phages: Av-I, Av-2, Av-3, BF307, CTI, CT2, CT3, CT4, CT6, CT7, CT8 and 1281.

Bacteria of the genus Aeromonas can be infected by the following phages: AA-I, Aeh2, N, PMI, TP446, 3, 4, 11, 13, 29, 31, 32, 37, 43, 43-1OT, 51, 54, 55R.1, 56, 56RR2, 57, 58, 59.1, 60, 63, Aehl, F, PM2, 1, 25, 31, 40RR2.8t, (syn=44R), (syn=44RR2.8t), 65, PM3, PM4, PM5 and PM6.

Bacteria of the genus Bacillus can be infected by the following phages: A, aizI, AI-K-I, B, BCJAI, BCI, BC2, BLLI, BLI, BP142, BSLI, BSL2, BSI, BS3, BS8, BS15, BS18, BS22, BS26, BS28, BS31, BS104, BS105, BS106, BTB, B1715V1, C, OK-I, Coll, Corl, CP-53, CS-I, CSi, D, D, D, D5, entl, FP8, FP9, FSi, FS2, FS3, FS5, FS8, FS9, G, GH8, GT8, GV-I, GV-2, GT-4, g3, gl2, gl3, gl4, gl6, gl7, g21, g23, g24, g29, H2, kenl, KK-88, Kuml, Kyul, J7W-1, LP52, (syn=LP-52), L7, Mexl, MJ-I, mor2, MP-7, MPIO, MP12, MP14, MP15, Neol, N°2, N5, N6P, PBCI, PBLA, PBPI, P2, S-a, SF2, SF6, Shal, Sill, SP02, (syn=φSPP1), SPβ, STI, STi, SU-II, t, Tbl, Tb2, Tb5, TbIO, Tb26, Tb51, Tb53, Tb55, Tb77, Tb97, Tb99, Tb560, Tb595, Td8, Td6, Tdl5, TgI, Tg4, Tg6, Tg7, Tg9, TgIO, TgII, TgI3, TgI5, Tg21, Tinl, Tin7, Tin8, Tinl3, Tm3, Tocl, Togl, toll, TP-I, TP-10vir, TP-15c, TP-16c, TP-17c, TP-19, TP35, TP51, TP-84, Tt4, Tt6, type A, type B, type C, type D, type E, Tφ3, VA-9, W, wx23, wx26, Yunl, α, γ, pl I, φmed-2, φT, φμ-4, φ3T, φ75, φIO5, (syn=φIO5), IA, IB, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), alel, ARI, AR2, AR3, AR7, AR9, Bace-11, (syn=11), Bastille, BLI, BL2, BL3, BL4, BL5, BL6, BL8, BL9, BP124, BS28, BS80, Ch, CP-51, CP-54, D-5, dart, dent, DP-7, entl, FoSi, FoS2, FS4, FS6, FS7, G, gall, gamma, GEI, GF-2, GSi, GT-I, GT-2, GT-3, GT-4, GT-5, GT-6, GT-7, GV-6, gl5, 19, 110, ISi, K, MP9, MP13, MP21, MP23, MP24, MP28, MP29, MP30, MP32, MP34, MP36, MP37, MP39, MP40, MP41, MP43, MP44, MP45, MP47, MP50, NLP-I, No.I, N17, N19, PBSI, PKI, PMBI, PMB12, PMJI, S, SPOI, SP3, SP5, SP6, SP7, SP8, SP9, SPIO, SP-15, SP50, (syn=SP-50), SP82, SST, subl, SW, Tg8, Tgl2, Tgl3, Tgl4, thul, thu, thuS, Tin4, Tin23, TP-13, TP33, TP50, TSP-I, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, 4 (B. megaterium), 4 (B. sphaericus), AR13, BPP-IO, BS32, BS107, BI, B2, GA-I, GP-10, GV-3, GV-5, g8, MP20, MP27, MP49, Nf, PPS, PP6, SF5, Tgl8, TP-I, Versailles, φI5, φ29, 1-97, 837/IV, mï-Bacillus (1), BatIO, BSLIO, BSLI I, BS6, BSI I, BS16, BS23, BSIOI, BS102, g18, morl, PBLI, SN45, thu2, thu3, Tml, Tm2, TP-20, TP21, TP52, type F, type G, type IV, HN-BacMus (3), BLE, (syn=8c), BS2, BS4, BS5, BS7, BIO, B12, BS20, BS21, F, MJ-4, PBA12, AP50, AP50-04, AP50-11, AP50-23, AP50-26, AP50-27 and Bam35. The following Bacillus-specific phages are defective: DLP10716, DLP-11946, DPB5, DPB12, DPB21, DPB22, DPB23, GA-2, M, No. IM, PBLB, PBSH, PBSV, PBSW, PBSX, PBSY, PBSZ, phi, SPa, type 1 and p.

Bacteria of the genus Bacteroides can be infected by the following phages: crAss-phage, ad 12, Baf-44, Baf-48B, Baf-64, Bf-I, Bf-52, B40-8, FI, βI, φAI, φBrOI, φBrO2, 11, 67.1, 67.3, 68.1, mt-Bacteroides (3), Bf42, Bf71, HN-Bdellovibrio (1) and BF-41.

Bacteria of the genus Bordetella can be infected by the following phages: 134 and NN-Bordetella (3).

Bacteria of the genus Borrelia can be infected by the following phages: NN-Borrelia (1) and NN-Borrelia (2).

Bacteria of the genus Brucella can be infected by the following phages: A422, Bk, (syn=Berkeley), BM29, FOi, (syn=FOI), (syn=FQI), D, FP2, (syn=FP2), (syn=FD2), Fz, (syn=Fz75/13), (syn=Firenze 75/13), (syn=Fi), Fi, (syn=FI), Fim, (syn=Flm), (syn=Fim), FiU, (syn=FIU), (syn=FiU), F2, (syn=F2), F3, (syn=F3), F4, (syn=F4), F5, (syn=F5), F6, F7, (syn=F7), F25, (syn=F25), (syn=25), F25U, (syn=F25u), (syn=F25U), (syn=F25V), F44, (syn-F44), F45, (syn=F45), F48, (syn=F48), I, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F8), (syn=F8), 12m, 24/11, (syn=24), (syn=F9), (syn=F9), 45/111, (syn=45), 75, 84, 212/XV, (syn=212), (syn=Fi0), (syn=FIO), 371/XXIX, (syn=371), (syn=Fn), (syn=FI I) and 513.

Bacteria of the genus Burkholderia can be infected by the following phages: CP75, NN-Burkholderia (1) and 42.

Bacteria of the genus Campylobacter can be infected by the following phages: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, NN-Campylobacter (2), Vfi-6, (syn=V19), VfV-3, V2, V3, V8, V16, (syn=Vfi-1), V19, V20(V45), V45, (syn=V-45) and NN-Campylobacter (1).

Bacteria of the genus Chlamydia can be infected by the following phage: Chpl.

Bacteria of the genus Clostridium can be infected by the following phages: CAKI, CAS, Ca7, CEβ, (syn=1C), CEγ, Cldl, c-n71, c-203 Tox-, DEβ, (syn=ID), (syn=IDt0X+), HM3, KMI, KT, Ms, NAI, (syn=Naltox+), PA135Oe, Pfó, PL73, PL78, PL81, PI, P50, P5771, P19402, ICt0X+, 2Ct0X\2D3 (syn=2Dt0X+), 3C, (syn=3Ctox+), 4C, (syn=4Ct0X+), 56, III-I, NN-Clostridium (61), NBIt0X+, al, CAI, HMT, HM2, PF15 P-23, P-46, Q-05, Q-oe, Q-16, Q-21, Q-26, Q-40, Q-46, S111, SA02, WA01, WA03, Wm, W523, 80, C, CA2, CA3, CPTI, CPT4, cl, c4, c5, HM7, H11/A1, H18/Ax, FWS23, Hi58ZA1, K2ZA1, K21ZS23, ML, NA2t0X; Pf2, Pf3, Pf4, S9ZS3, S41 ZA1, S44ZS23, α2, 41, 112ZS23, 214/S23, 233/Ai, 234/S23, 235/S23, II-I, 11-2, 11-3, NN-Clostridium (12), CAI, FI, K, S2, 1, 5 and NN-Clostridium (8).

Bacteria of the genus Corynebacterium can be infected by the following phages: CGKI (defective), A, A2, A3, AIOI, A128, A133, A137, A139, A155, A182, B, BF, B17, B18, B51, B271, B275, B276, B277, B279, B282, C, capi, CCI, CGI, CG2, CG33, CL31, Cog, (syn=CG5), D, E, F, H, H-I, hqi, hq2, 11ZH33, li/31, J, K, K, (syn=Ktox″), L, L, (syn=Ltox+), M, MC-I, MC-2, MC-3, MC-4, MLMa, N, O, ovi, ov2, ov3, P, P, R, RP6, RS29, S, T, U, UB1, ub2, UH1, UH3, uh3, uh5, uh6, β, (syn=βtox+), βhv64, βvir, γ, (syn=γtoχ−), γI9, δ, (syn=δ′ox+), p, (syn=ptoχ−), φ9, φ984, ω, IA, 1/1180, 2, 2/1180, 5/1180, 5ad/9717, 7/4465, 8/4465, 8ad/10269, 10/9253, 13Z9253, 15/3148, 21/9253, 28, 29, 55, 2747, 2893, 4498 and 5848.

Bacteria of the genus Enterococcus are infected by the following phage: DF78, FI, F2, 1, 2, 4, 14, 41, 867, DI, SB24, 2BV, 182, 225, C2, C2F, E3, E62, DS96, H24, M35, P3, P9, SBIOI, S2, 2B11, 5, 182a, 705, 873, 881, 940, 1051, 1057, 21096C, NN-Enterococcus (1), PEI, FI, F3, F4, VD13, 1,200,235 and 341.

Bacteria of the genus Erysipelothrix can be infected by the following phage: NN-Eiysipelothrix (1).

Bacteria of the genus Escherichia can be infected by the following phages: BW73, B278, D6, D108, E, EI, E24, E41, FI-2, FI-4, FI-5, H18A, Ffl8B, i, MM, Mu, (syn=mu), (syn=Mul), (syn=Mu-I), (syn=MU-I), (syn=Mul), (syn=μ), 025, Phl-5, Pk, PSP3, PI, PID, P2, P4 (defective), SI, Wφ, φK13, φR73 (defective), φI, φ2, φ7, φ92, ψ (defective), 7 A, 8φ, 9φ, 15 (defective), 18, 28 1, 186, 299, HH-Escherichia (2), AB48, CM, 04, C16, DD-VI, (syn=Dd-Vi), (syn=DDVI), (syn=DDVi), E4, E7, E28, FII, FI3, H, HI, H3, H8, K3, M, N, ND-2, ND-3, ND4, ND-5, ND6, ND-7, Ox-I (syn=OXI), (syn=HF), Ox-2 (syn=0x2), (syn=0X2), Ox-3, Ox-4, Ox-5, (syn=0X5), Ox-6, (syn=66F), (syn=φ66t), (syn=φ66t-)5 0111, Phl-I, RB42, RB43, RB49, RB69, S, Sal-I, Sal-2, Sal-3, Sal-4, Sal-5, Sal-6, TC23, TC45, Tull*-6, (syn=Tull*), TuIP-24, Tull*46, TuIP-60, T2, (syn=ganuTia), (syn=γ), (syn=PC), (syn=P.C.), (syn=T-2), (syn=T2), (syn=P4), T4, (syn=T-4), (syn=T4), T6, T35, al, 1, IA, 3, (syn=Ac3), 3A, 3T+, (syn=3), (syn=MI), 5φ, (syn=φ5), 9266Q, CFO103, HK620, J, K, KIF, m59, no. A, no. E, no. 3, no. 9, N4, sd, (syn=Sd), (syn=SD), (syn=Sa)3 (syn=sd), (syn=SD), (syn=CD), T3, (syn=T-3), (syn=T3), T7, (syn=T-7), (syn=T7), WPK, W31, ΔH, φC3888, φK3, φK7, φK12, φV-1, φ04-CF, φ05, φ06, φ07, φI, φI.2, φ20, φ95, φ263, φIO92, φI, φII, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, CI, DDUP, ECI, EC2, E21, E29, FI, F26S, F27S, Hi, HK022, HK97, (syn=φHK97), HK139, HK253, HK256, K7, ND-I, no.D, PA-2, q, S2, TI, (syn=a), (syn=P28), (syn=T-I), (syn=Tx), T3C, T5, (syn=T-5), (syn=T5), UC-I, w, β4, γ2, λ (syn=lambda), (syn=φλ), φD326, φγ, φ06, φ7, φ10, φ80, X, (syn=χi), (syn=φχ), (syn=φχi), 2, 4, 4A, 6, 8A, 102, 150, 168, 174, 3000, AC6, AC7, AC28, AC43, AC50, AC57, AC81, AC95, HK243, KIO, ZG/3A, 5, 5A, 21EL, H19-J, 933H, 0157 typing phages 1 to 16, JES-2013, 121Q, 172-1, 1720a-02, ADB-2, AKFV33, av-05, bV_EcoS_AHP42, bV_EcoS_AHP24, bC_EcoS_AHS24, bV_EcoS_AKS96, CBA120.

Bacteria of the genus Fusobacterium are infected by the following phage: NN-Fusobacterium (2), fv83-554/3, fv88-531/2, 227, fv2377, fv2527 and fv8501.

Bacteria of the genus Haemophilus are infected by the following phage: HPI, S2 and N3.

Bacteria of the genus Helicobacter are infected by the following phage: HPI and \hat{ }\hat{ }-Helicobacter (1).

Bacteria of the genus Klebsiella are infected by the following phage: AIO-2, KI4B, KI6B, KI9, (syn=KI9), KI14, KI15, KI21, KI28, KI29, KI32, KI33, KI35, KI106B, KI171B, KI181B, KI832B, AIO-I, AO-I, AO-2, AO-3, FC3-10, K, KI1, (syn=KII), KI2, (syn=KI2), KI3, (syn=KI3), (syn=KI 70/11), KI4, (syn=KI4), KI5, (syn=KI5), KI6, (syn=KI6), KI7, (syn=KI7), KI8, (syn=KI8), KI19, (syn=KI9), KI27, (syn=KI27), KI31, (syn=KI31), KI35, KI171B, II, VI, IX, CI-I, KI4B, KI8, KI11, KI12, KI13, KI16, KI17, KI18, KI20, KI22, KI23, KI24, KI26, KI30, KI34, KI106B, KIi65B, KI328B, KLXI, K328, P5046, 11, 380, III, IV, VII, VIII, FC3-11, KI2B, (syn=KI2B), KI25, (syn=KI25), KI42B, (syn=KI42), (syn=KI42B), KI181B, (syn=KII 81), (syn=KI181B), KI765/, (syn=KI765/1), KI842B, (syn=KI832B), KI937B, (syn=KI937B), LI, φ28, 7, 231, 483, 490, 632 and 864/100.

Bacteria of the genus Lepitospira are infected by the following phage: LEI, LE3, LE4 and -NN-Leptospira (1).

Bacteria of the genus Listeria are infected by the following phage: A511, 01761, 4211, 4286, (syn=B054), A005, A006, A020, A500, A502, A511, AI 18, A620, A640, B012, B021, B024, B025, B035, B051, B053, B054, B055, B056, BIOI, BI 10, B545, B604, B653, 0707, D441, HSO47, HIOG, H8/73, H19, H21, H43, H46, H107, H108, HI 10, H163/84, H312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Listeria (15).

Bacteria of the genus Morganella are infected by the following phage: 47.

Bacteria of the genus Mycobacterium are infected by the following phage: 13, AGI, ALi, ATCC 11759, A2, B.C3, BG2, BKI, BKS, butyricum, B-I, B5, B7, B30, B35, Clark, CI, C2, DNAIII, DSP1, D4, D29, GS4E, (syn=GS4E), GS7, (syn=GS-7), (syn=GS7), IPa, lacticola, Legendre, Leo, L5, (syn=φL-5), MC-I, MC-3, MC-4, minetti, MTPHI I, Mx4, MyF3P/59a, phlei, (syn=phlei 1), phlei 4, Polonus II, rabinovitschi, smegmatis, TM4, TM9, TMIO, TM20, Y7, YIO, φ630, IB, IF, IH, 1/1, 67, 106, 1430, BI, (syn=Bol), B24, D, D29, F-K, F-S, HP, Polonus I, Roy, RI, (syn=RI-Myb), (syn=Ri), 11, 31, 40, 50, 103a, 103b, 128, 3111-D, 3215-D and NN-Mycobacterium (1).

Bacteria of the genus Neisseria are infected by the following phage: Group I, group II and NPI.

Bacteria of the genus Nocardia are infected by the following phage: MNP8, NJ-L, NS-8, N5 and TtiN-Nocardia.

Bacteria of the genus Proteus are infected by the following phage: Pm5, 13vir, 2/44, 4/545, 6/1004, 13/807, 20/826, 57, 67b, 78, 107/69, 121, 9/0, 22/608, 30/680, PmI, Pm3, Pm4, Pm6, Pm7, Pm9, PmIO, PmI I, Pv2, πI, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, π2600, φχ7, 1/1004, 5/742, 9, 12, 14, 22, 24/860, 2600/D52, Pm8 and 24/2514.

Bacteria of the genus Providencia are infected by the following phage: PL25, PL26, PL37, 9211/9295, 9213/921 I b, 9248, 7/R49, 7476/322, 7478/325, 7479, 7480, 9000/9402 and 9213/921 Ia.

Bacteria of the genus Pseudomonas are infected by the following phage: PfI, (syn=Pf-I), Pf2, Pf3, PP7, PRRI, 7s, im-Pseudomonas (1), AI-I, AI-2, B 17, B89, CB3, Col 2, Col 11, Col 18, Col 21, 0154, 0163, 0167, 02121, E79, F8, ga, gb, H22, K1, M4, N2, Nu, PB-I, (syn=PBI), pf16, PMN17, PPI, PP8, PsaI, PsPI, PsP2, PsP3, PsP4, PsP5, PS3, PS17, PTB80, PX4, PX7, PYOI, PYO2, PYO5, PYO6, PYO9, PYOIO, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, PIK, SLPI, SL2, S2, UNL-I, wy, Yai, Ya4, Yan, (PBE, φCTX, φC17, φKZ, (syn=ϵβKZ), φ-LT, φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, HN-Pseudomonas (23), A856, B26, CI-I, CI-2, C5, D, gh-1, FI 16, HF, H90, K5, K6, KI 04, K109, K166, K267, N4, N5, 06N-25P, PE69, Pf, PPN25, PPN35, PPN89, PPN91, PP2, PP3, PP4, PP6, PP7, PP8, PP56, PP87, PPI 14, PP206, PP207, PP306, PP651, Psp231a, Pssy401, Pssy9220, psi, PTB2, PTB20, PTB42, PXI, PX3, PXIO, PX12, PX14, PYO70, PYO71, R, SH6, SH133, tf, Ya5, Ya7, pBS, OKf77, φ-MC, OmnF82, φβLS27, cpPLS743, pS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-I, 022, D3, D37, D40, D62, D3112, F7, FIO, g, gd, ge, gξ Hwl2, Jb 19, KFI, L°, OXN-32P, 06N-52P, PCH-I, P013-1, P035-1, PH2, PH51, PH93, PH 132, PMW, PM 13, PM57, PM61, PM62, PM63, PM69, PM 105, PMI 13, PM681, PM682, PO4, PPI, PP4, PPS, PP64, PP65, PP66, PP71, PP86, PP88, PP92, PP401, PP711, PP891, Pssy41, Pssy42, Pssy403, Pssy404, Pssy420, Pssy923, PS4, PS-IO, Pz, SDI, SLI, SL3, SLS, SM, (PC5, φCI I, φCI I-1, φC13, φC15, φMO, φX, φO4, φI I, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, HN-Pseudomonas (6), GIOI, M6, M6a, LI, PB2, Pssy15, Pssy4210, Pssy4220, PYO12, PYO34, PYO49, PYO50, PYO51, PYO52, PYO53, PYO57, PYO59, PYO200, PX2, PX5, SL4, φO3, φO6 and 1214.

Bacteria of the genus Rickettsia are infected by the following phage: NN-Rickettsia.

Bacteria of the genus Salmonella are infected by the following phage: b, Beccles, CT, d, Dundee, f, Fels 2, GI, GUI, GVI, GVIII, k, K, i, j, L, 01, (syn=0-1), (syn=O1), (syn=O-I), (syn=7), 02, 03, P3, P9a, PIO, Sab3, Sab5, SanIS, San17, SI, Taunton, Vil, (syn=Vil), 9, imSalmonella (1), N-I, N-5, N-10, N-17, N-22, 11, 12, 16-19, 20.2, 36, 449C/C178, 966A/C259, a, B.A.O.R., e, G4, GUI, L, LP7, M, MG40, N-18, PSA68, P4, P9c, P22, (syn=P22), (syn=PLT22), (syn=PLT22), P22al, P22-4, P22-7, P22-11, SNT-I, SNT-2, SP6, Villi, ViIV, ViV, ViVI, ViVII, Worksop, Sj5, c34, 1,37, 1(40), (syn=(pl[40]), 1,422, 2, 2.5, 3b, 4, 5, 6,14(18), 8, 14(6,7), 10, 27, 28B, 30, 31, 32, 33, 34, 36, 37, 39, 1412, SNT-3, 7-11, 40.3, c, 0236, 0557, 0625, C966N, g, GV, G5, GI 73, h, IRA, Jersey, MB78, P22-1, P22-3, P22-12, Sab1, Sab2, Sab2, Sab4, San1, San2, San3, San4, San6, San7, San8, San9, San13, San14, San16, San18, San19, San20, San21, San22, San23, San24, San25, San26, SasL1, SasL2, SasL3, SasL4, SasL5, SIBL, SII, Vill, φI, 1, 2, 3a, 3al, 1010, Ym-Salmonella (1), N-4, SasL6 and 27.

Bacteria of the genus Serratia are infected by the following phage: A2P, PS20, SMB3, SMP, SMP5, SM2, V40, V56, ic, OCP-3, cPCP-6, 3M, 10/la, 20A, 34CC, 34H, 38T, 345G, 345P, 501B, SMB2, SMP2, BC, BT, CW2, CW3, CW4, CW5, Lt232, L2232, L34, L.228, SLP, SMPA, V.43, β, φCWI, φCP6-1, φCP6-2, φCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/1Oa, L.359 and SMBI.

Bacteria of the genus Shigella are infected by the following phage: Fsa, (syn=a), FSD2d, (syn=D2d), (syn=W2d), FSD2E, (syn=W2e), fv, F6, f7.8, H-Sh, PE5, P90, SflI, Sh, SHm, SHrv, (syn=HIV), SHvi, (syn=HVI), SHVvm, (syn=HVIII), SKy66, (syn=gamma 66), (syn=γββ), (syn=γ66b), SKm, (syn=SIIIb)5 (syn=UI), SKw, (syn=Siva), (syn=IV), SIC™, (syn=SIVA.), (syn=IVA), SKvi, (syn=KVI), (syn=Svi), (syn=VI), SKvm, (syn=Svm), (syn=VIII), SKVΠIA, (syn=SvmA), (syn=VIIIA), STvi, STK, STx1, STxn, S66, W2, (syn=D2c), (syn=D20), φI, φIVb 3-SO-R, 8368-SO-R, F7, (syn=FS7), (syn=K29), FIO, (syn=FSIO), (syn=K31), 11, (syn=alfa), (syn=FSa), (syn=KI 8), (syn=a), 12, (syn=a), (syn=K19), SG33, (syn=G35), (syn=SO-35/G), SG35, (syn=SO-55/G), SG3201, (syn=SO-3201/G), SHn, (syn=HII), SHv, (syn=SHV), SHx, SHX, SKn, (syn=K2), (syn=KII), (syn=Sn), (syn=Ssll), (syn=II), SKrv, (syn=Sm), (syn=SsIV), (syn=IV), SK1Va, (syn=Swab), (syn=SsIVa), (syn=IVa), SKV, (syn=K4), (syn=KV), (syn=SV), (syn=SsV), (syn=V), SKx, (syn=K9), (syn=KX), (syn=SX), (syn=SsX), (syn=X), STV, (syn=T35), (syn=35-50-R), STvm, (syn=T8345), (syn=8345-SO—S-R), W1, (syn=D8), (syn=FSD8), W2a, (syn=D2A), (syn=FS2a), DD-2, Sf6, FSi, (syn=FI), SF6, (syn=F6), SG42, (syn=SO-42/G), SG3203, (syn=SO-3203/G), SKF12, (syn=SsF12), (syn=F12), (syn=F12), STn, (syn=1881-SO-R), γ66, (syn=gamma 66a), (syn=Ssγ66), φ2, BII, DDVII, (syn=DD7), FSD2b, (syn=W2B), FS2, (syn=F2), (syn=F2), FS4, (syn=F4), (syn=F4), FS5, (syn=F5), (syn=F5), FS9, (syn=F9), (syn=F9), FI I, P2-SO-S, SG36, (syn=SO-36/G), (syn=G36), SG3204, (syn=SO-3204/G), SG3244, (syn=SO-3244/G), SHi, (syn=HI), SHvπ, (syn=HVII), SHK, (syn=HIX), SHx1, SHxπ, (syn=HXn), SKI, KI, (syn=Si), (syn=Ssl), SKVII, (syn=KVII), (syn=Svπ), (syn=SsVII), SKIX, (syn=KIX), (syn=Six), (syn=SsIX), SKXII, (syn=KXII), (syn=Sxn), (syn=SsXII), STi, STffl, STrv, STVi, STvπ, S70, S206, U2-S0-S, 3210-SO-S, 3859-SO-S, 4020-SO-S, φ3, φ5, φ7, φ8, φ9, φIO, φI I, φI3, φI4, φI8, SHm, (syn=Hπi), SHχi, (syn=HXt) and SKxl, (syn=KXI), (syn=Sχi), (syn=SsXI), (syn=XI).

Bacteria of the genus Staphylococcus are infected by the following phage: A, EW, K, Ph5, Ph9, PhIO, PhI3, PI, P2, P3, P4, P8, P9, PIO, RG, SB-i, (syn=Sb-I), S3K, Twort, φSK311, φ812, 06, 40, 58, 119, 130, 131, 200, 1623, STCI, (syn=stcl), STC2, (syn=stc2), 44AHJD, 68, ACI, AC2, A6“C”, A9“C”, b581, CA-I, CA-2, CA-3, CA-4, CA-5, DI I, L39x35, L54a, M42, NI, N2, N3, N4, N5, N7, N8, NIO, Ni I, N12, N13, N14, N16, Ph6, Phl2, Phl4, UC-18, U4, U15, SI, S2, S3, S4, S5, X2, Z1, φB5-2, φD, ω, 11, (syn=φI I), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83 A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, AIO, A13, b594n, D, HK2, N9, N15, P52, P87, SI, S6, Z4, pRE, 3A, 3B, 3C, 6, 7, 16, 21, 42B, 42C, 42E, 44, 47, 47A5 47C, 51, 54, 54x1, 70, 73, 75, 78, 81, 82, 88, 93, 94, 101, 105, 110, 115, 129/16, 174, 594n, 1363/14, 2460 and mS-Staphylococcus (1).

Bacteria of the genus Streptococcus are infected by the following phage: EJ-I, NN-Streptococais (1), a, Cl, FL0Ths, H39, Op-I, Cp-5, Cp-7, Cp-9, Cp-IO, AT298, A5, aIO/JI, aIO/J2, aIO/J5, aIO/J9, A25, BTI I, b6, CAI, c20-I, c20-2, DP-I, Dp-4, DTI, ET42, eIO, FA101, FEThs, F_{K}, FKKIOI, FKLIO, FKP74, FKH, FLOThs, Fy101, fl, F10, F20140/76, g, GT-234, HB3, (syn=HB-3), HB-623, HB-746, M102, 01205, φO1205, PST, PO, PI, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, Sfll 1, (syn=SFiI I), (syn=φSFill), (syn=ϵPSfiI I), (syn=φSfil I), sfil9, (syn=SFil9), (syn=φSFil9), (syn=φSfil9), Sfi21, (syn=SFi21), (syn=φSFi21), (syn=φSfi21), STO, STX, st2, ST2, ST4, S3, (syn=φS3), s265, P17, φ42, φ57, φ80, φ81, φ82, φ83, φ84, φ85, φ86, φ87, φ88, φ89, φ90, φ91, φ92, φ93, φ94, φ95, φ96, φ97, φ98, φ99, φIOO, φ101, φIO2, φ227, ϵP7201, ω1, ω2, ω3, ω4, ω5, ω6, ω8, ωIO, 1, 6, 9, 1OF, 12/12, 14, 17SR, 19S, 24, 50/33, 50/34, 55/14, 55/15, 70/35, 70/36, 71/ST15, 71/45, 71/46, 74F, 79/37, 79/38, 80/J4, 80/J9, 80/5T16, 80/15, 80/47, 80/48, 101, 103/39, 103/40, 121/41, 121/42, 123/43, 123/44, 124/44, 337/ST17 and mStreptococcus (34).

Bacteria of the genus Treponema are infected by the following phage: NN-Treponema (1).

Bacteria of the genus Vibrio are infected by the following phage: CTXcP, fs, (syn=si), fs2, lvpfs, Vfl2, Vf33, VPIφ, VSK, v6, 493, OP-TI, ET25, kappa, K139, Labol, XN-69P, OXN-86, O6N-21P, PB-I, P147, rp-1, 5E3, VA-I, (syn=VcA-I), VcA-2, VPI, VP2, VP4, VP7, VP8, VP9, VPIO, VP17, VP18, VP19, X29, (syn=29 d'Herelle), t, PHAWI-1, PHAWI-2, PHAWI-3, PHAWI-4, PHAWI-5, PHAWI-6, PHAWI-7, XHAWI-8, PHAWI-9, PHAWI-10, PHCI-1, PHC1-2, PHC1-3, PHC1-4, PHC2-1, >HC2-2, PHC2-3, PHC2-4, OHC3-1, OHC3-2, OHC3-3, PHD1S-1, PHD1S-2, PHD2S-1, PHD2S-2, PHD2S-3, PHD2S-4, PHD2S-5, OHDO-1, OHDO-2, OHDO-3, PHDO-4, OHDO-5, OHDO-6, OKL-33, OKL-34, OKL-35, OKL-36, PKWH-2, PKWH-3, PKWH-4, PMARQ-1, PMARQ-2, PMARQ-3, PMOAT-1, P0139, OPEL1A-1, OPEL1A-2, OPEL8A-1, OPEL8A-2, OPEL8A-3, PPEL8C-1, PPEL8C-2, PPEL13A-1, OPEL13B-1, OPEL13B-2, OPEL13B-3, OPEL13B-4, OPEL13B-5, OPEL13B-6, OPEL13B-7, OPEL13B-8, OPEL13B-9, OPEL13B-10, φVP143, φVP253, φ16, φ138, 1-II, 5, 13, 14, 16, 24, 32, 493, 6214, 7050, 7227, II, (syn=group II), (syn==φ2), V, VIII, ˜m-Vibrio (13), KVP20, KVP40, nt-1, 06N-22P, P68, e1, e2, e3, e4, e5, FK, G, I, K, nt-6, NI, N2, N3, N4, N5, 06N-34P, OXN-72P, OXN-85P, OXN-100P, P, Ph-I, PL163/10, Q, S, T, φ92, 1-9, 37, 51, 57, 70A-8, 72A-4, 72A-10, 11OA-4, 333, 4996, I (syn=group I), III (syn=group III), VI, (syn=A-Saratov), VII, IX, X, HN-Vibrio (6), pAI, 7, 7-8, 70A 2, 71A-6, 72A-5, 72A-8, 108A-10, 109A-6, 109A-8, I IOA-1, 11OA-5, 11OA-7, by-1, OXN-52P, P13, P38, P53, P65, P108, Pill, TP13 VP3, VP6, VP12, VP13, 70A-3, 70A-4, 70A-10, 72A-1, 108A-3, 109-B1, 11OA-2, 149, (syn=φ149), IV, (syn=group IV), NN-Vibrio (22), VP5, VPII, VP15, VP16, α1, α2, α3a, α3b, 353B and HN-Vibrio (7).

Bacteria of the genus Yersinia are infected by the following phage: H, H-1, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D′Herelle, EV, H, Kotljarova, PTB, R, Y, YerA41, φYerO3-12, 3, 4/C1324-76, 7/F783-76, 903, 1/M6176 and Yer2AT.

In a particular embodiment, the first type of bacteriophage is selected from the group consisting of the bacteriophages listed above, and the second type of bacteriophage is selected from the group consisting of the bacteriophages listed above, said second type of bacteriophage being a type of bacteriophage different from the first type of bacteriophage.

In a particular embodiment, the first and/or the second type of bacteriophage is selected in the group consisting of Salmonella virus SKML39, Shigella virus AG3, Dickeya virus Limestone, Dickeya virus RC2014, Escherichia virus CBA120, Escherichia virus Phaxl, Salmonella virus 38, Salmonella virus Det7, Salmonella virus GG32, Salmonella virus PM10, Salmonella virus SFP10, Salmonella virus SH19, Salmonella virus SJ3, Escherichia virus ECML4, Salmonella virus Marshall, Salmonella virus Maynard, Salmonella virus SJ2, Salmonella virus STML131, Salmonella virus Vil, Erwinia virus Ea2809, Klebsiella virus 0507KN21, Serratia virus IME250, Serratia virus MAM1, Campylobacter virus CP21, Campylobacter virus CP220, Campylobacter virus CPt10, Campylobacter virus IBB35, Campylobacter virus CP81, Campylobacter virus CP30A, Campylobacter virus CPX, Campylobacter virus NCTC12673, Erwinia virus Ea214, Erwinia virus M7, Escherichia virus AYO145A, Escherichia virus EC6, Escherichia virus HY02, Escherichia virus JH2, Escherichia virus TP1, Escherichia virus VpaE1, Escherichia virus wV8, Salmonella virus FelixO1, Salmonella virus HB2014, Salmonella virus Mushroom, Salmonella virus UAB87, Citrobacter virus Moogle, Citrobacter virus Mordin, Escherichia virus SUSP1, Escherichia virus SUSP2, Aeromonas virus phiO18P, Haemophilus virus HP1, Haemophilus virus HP2, Pasteurella virus F108, Vibrio virus K139, Vibrio virus Kappa, Burkholderia virus phi52237, Burkholderia virus phiE122, Burkholderia virus phiE202, Escherichia virus 186, Escherichia virus P4, Escherichia virus P2, Escherichia virus Wphi, Mannheimia virus PHL101, Pseudomonas virus phiCTX, Ralstonia virus RSA1, Salmonella virus Fels2, Salmonella virus PsP3, Salmonella virus SopEphi, Yersinia virus L413C, Staphylococcus virus G1, Staphylococcus virus G15, Staphylococcus virus JD7, Staphylococcus virus K, Staphylococcus virus MCE2014, Staphylococcus virus P108, Staphylococcus virus Rodi, Staphylococcus virus S253, Staphylococcus virus S25-4, Staphylococcus virus SA12, Listeria virus A511, Listeria virus P100, Staphylococcus virus Remus, Staphylococcus virus SA11, Staphylococcus virus Stau2, Bacillus virus Camphawk, Bacillus virus SPO1, Bacillus virus BCP78, Bacillus virus TsarBomba, Staphylococcus virus Twort, Enterococcus virus phiEC24C, Lactobacillus virus Lb338-1, Lactobacillus virus LP65, Enterobacter virus PG7, Escherichia virus CC31, Klebsiella virus JD18, Klebsiella virus PKO111, Escherichia virus Bp7, Escherichia virus IME08, Escherichia virus JS10, Escherichia virus JS98, Escherichia virus QL01, Escherichia virus VR5, Enterobacter virus Eap3, Klebsiella virus KP15, Klebsiella virus KP27, Klebsiella virus Matisse, Klebsiella virus Miro, Citrobacter virus Merlin, Citrobacter virus Moon, Escherichia virus JSE, Escherichia virus phil, Escherichia virus RB49, Escherichia virus HX01, Escherichia virus JS09, Escherichia virus RB69, Shigella virus UTAM, Salmonella virus S16, Salmonella virus STML198, Vibrio virus KVP40, Vibrio virus nt1, Vibrio virus VaIKK3, Escherichia virus VR7, Escherichia virus VR20, Escherichia virus VR25, Escherichia virus VR26, Shigella virus SP18, Escherichia virus AR1, Escherichia virus 040, Escherichia virus E112, Escherichia virus ECML134, Escherichia virus HY01, Escherichia virus Ime09, Escherichia virus RB3, Escherichia virus RB14, Escherichia virus T4, Shigella virus Pss1, Shigella virus Shf12, Yersinia virus D1, Yersinia virus PST, Acinetobacter virus 133, Aeromonas virus 65, Aeromonas virus Aeh1, Escherichia virus RB16, Escherichia virus RB32, Escherichia virus RB43, Pseudomonas virus 42, Cronobacter virus CR3, Cronobacter virus CR8, Cronobacter virus CR9, Cronobacter virus PBES02, Pectobacterium virus phiTE, Cronobacter virus GAP31, Escherichia virus 4MG, Salmonella virus SE1, Salmonella virus 55E121, Escherichia virus FFH2, Escherichia virus FV3, Escherichia virus JES2013, Escherichia virus V5, Brevibacillus virus Abouo, Brevibacillus virus Davies, Bacillus virus Agate, Bacillus virus Bobb, Bacillus virus Bp8pC, Erwinia virus Deimos, Erwinia virus Ea35-70, Erwinia virus RAY, Erwinia virus Simmy50, Erwinia virus SpecialG, Acinetobacter virus AB1, Acinetobacter virus AB2, Acinetobacter virus AbC62, Acinetobacter virus AP22, Arthrobacter virus ArV1, Arthrobacter virus Trina, Bacillus virus AvesoBmore, Bacillus virus B4, Bacillus virus Bigbertha, Bacillus virus Riley, Bacillus virus Spock, Bacillus virus Troll, Bacillus virus Bastille, Bacillus virus CAM003, Bacillus virus Bc431, Bacillus virus Bcp1, Bacillus virus BCP82, Bacillus virus BM15, Bacillus virus Deepblue, Bacillus virus JBP901, Burkholderia virus Bcep1, Burkholderia virus Bcep43, Burkholderia virus Bcep781, Burkholderia virus BcepNY3, Xanthomonas virus OP2, Burkholderia virus BcepMu, Burkholderia virus phiE255, Aeromonas virus 44RR2, Mycobacterium virus Alice, Mycobacterium virus Bxz1, Mycobacterium virus Dandelion, Mycobacterium virus HyRo, Mycobacterium virus 13, Mycobacterium virus Nappy, Mycobacterium virus Sebata, Clostridium virus phiC2, Clostridium virus phiCD27, Clostridium virus phiCD119, Bacillus virus CP51, Bacillus virus JL, Bacillus virus Shanette, Escherichia virus CVM10, Escherichia virus ep3, Erwinia virus Asesino, Erwinia virus EaH2, Pseudomonas virus EL, Halomonas virus HAP1, Vibrio virus VP882, Brevibacillus virus Jimmer, Brevibacillus virus Osiris, Pseudomonas virus Ab03, Pseudomonas virus KPP10, Pseudomonas virus PAKP3, Sinorhizobium virus M7, Sinorhizobium virus M12, Sinorhizobium virus N3, Erwinia virus Machina, Arthrobacter virus Brent, Arthrobacter virus Jawnski, Arthrobacter virus Martha, Arthrobacter virus Sonny, Edwardsiella virus MSW3, Edwardsiella virus PEi21, Escherichia virus Mu, Shigella virus SfMu, Halobacterium virus phiH, Bacillus virus Grass, Bacillus virus NIT1, Bacillus virus SPG24, Aeromonas virus 43, Escherichia virus P1, Pseudomonas virus CAb1, Pseudomonas virus CAb02, Pseudomonas virus JG004, Pseudomonas virus PAKP1, Pseudomonas virus PAKP4, Pseudomonas virus PaP1, Burkholderia virus BcepF1, Pseudomonas virus 141, Pseudomonas virus Ab28, Pseudomonas virus DL60, Pseudomonas virus DL68, Pseudomonas virus F8, Pseudomonas virus JG024, Pseudomonas virus KPP12, Pseudomonas virus LBL3, Pseudomonas virus LMA2, Pseudomonas virus PB1, Pseudomonas virus SN, Pseudomonas virus PA7, Pseudomonas virus phiKZ, Rhizobium virus RHEph4, Ralstonia virus RSF1, Ralstonia virus RSL2, Ralstonia virus RSL1, Aeromonas virus 25, Aeromonas virus 31, Aeromonas virus Aes12, Aeromonas virus Aes508, Aeromonas virus AS4, Stenotrophomonas virus IME13, Staphylococcus virus IPLAC1C, Staphylococcus virus SEP1, Salmonella virus SPN3US, Bacillus virus 1, Geobacillus virus GBSV1, Yersinia virus R1RT, Yersinia virus TG1, Bacillus virus G, Bacillus virus PBS1, Microcystis virus Ma-LMM01, Vibrio virus MAR, Vibrio virus VHML, Vibrio virus VP585, Bacillus virus BPS13, Bacillus virus Hakuna, Bacillus virus Megatron, Bacillus virus WPh, Acinetobacter virus AB3, Acinetobacter virus Abp1, Acinetobacter virus Fri1, Acinetobacter virus IME200, Acinetobacter virus PD6A3, Acinetobacter virus PDAB9, Acinetobacter virus phiAB1, Escherichia virus K30, Klebsiella virus K5, Klebsiella virus K11, Klebsiella virus Kp1, Klebsiella virus KP32, Klebsiella virus KpV289, Klebsiella virus F19, Klebsiella virus K244, Klebsiella virus Kp2, Klebsiella virus KP34, Klebsiella virus KpV41, Klebsiella virus KpV71, Klebsiella virus KpV475, Klebsiella virus SU503, Klebsiella virus SU552A, Pantoea virus Limelight, Pantoea virus Limezero, Pseudomonas virus LKA1, Pseudomonas virus phiKMV, Xanthomonas virus f20, Xanthomonas virus f30, Xylella virus Prado, Erwinia virus Era103, Escherichia virus K5, Escherichia virus K1-5, Escherichia virus K1E, Salmonella virus SP6, Escherichia virus T7, Kluyvera virus Kvp1, Pseudomonas virus gh1, Prochlorococcus virus PSSP7, Synechococ: virus P60, Synechococcus virus Syn5, Streptococcus virus Cp1, Streptococcus virus Cp7, Staphylococcus virus 44AHJD, Streptococcus virus Cl, Bacillus virus B103, Bacillus virus GA1, Bacillus virus phi29, Kurthia virus 6, Actinomyces virus Av1, Mycoplasma virus P1, Escherichia virus 24B, Escherichia virus 933W, Escherichia virus Min27, Escherichia virus PA28, Escherichia virus Stx2 II, Shigella virus 7502Stx, Shigella virus POCJ13, Escherichia virus 191, Escherichia virus PA2, Escherichia virus TL2011, Shigella virus VASD, Burkholderia virus Bcep22, Burkholderia virus Bcepi102, Burkholderia virus Bcepmigl, Burkholderia virus DC1, Bordetella virus BPP1, Burkholderia virus BcepC6B, Cellulophaga virus Cba41, Cellulophaga virus Cba172, Dinoroseobacter virus DFL12, Erwinia virus Ea9-2, Erwinia virus Frozen, Escherichia virus phiV10, Salmonella virus Epsilon15, Salmonella virus SPN1S, Pseudomonas virus F116, Pseudomonas virus H66, Escherichia virus APEC5, Escherichia virus APEC7, Escherichia virus Bp4, Escherichia virus EC1UPM, Escherichia virus ECBP1, Escherichia virus G7C, Escherichia virus IME11, Shigella virus Sb1, Achromobacter virus Axp3, Achromobacter virus JWAlpha, Edwardsiella virus KF1, Pseudomonas virus KPP25, Pseudomonas virus R18, Pseudomonas virus Ab09, Pseudomonas virus LIT1, Pseudomonas virus PA26, Pseudomonas virus Ab22, Pseudomonas virus CHU, Pseudomonas virus LUZ24, Pseudomonas virus PAA2, Pseudomonas virus PaP3, Pseudomonas virus PaP4, Pseudomonas virus TL, Pseudomonas virus KPP21, Pseudomonas virus LUZ7, Escherichia virus N4, Salmonella virus 9NA, Salmonella virus SP069, Salmonella virus BTP1, Salmonella virus HK620, Salmonella virus P22, Salmonella virus ST64T, Shigella virus Sf6, Bacillus virus Page, Bacillus virus Palmer, Bacillus virus Pascal, Bacillus virus Pony, Bacillus virus Pookie, Escherichia virus 172-1, Escherichia virus ECB2, Escherichia virus NJO1, Escherichia virus phiEco32, Escherichia virus Septimal1, Escherichia virus SU10, Brucella virus Pr, Brucella virus Tb, Escherichia virus Pollock, Salmonella virus FSL SP-058, Salmonella virus FSL SP-076, Helicobacter virus 1961P, Helicobacter virus KHP30, Helicobacter virus KHP40, Hamiltonella virus APSE1, Lactococcus virus KSY1, Phormidium virus WMP3, Phormidium virus WMP4, Pseudomonas virus 119X, Roseobacter virus SIO1, Vibrio virus VpV262, Vibrio virus VC8, Vibrio virus VP2, Vibrio virus VP5, Streptomyces virus Amela, Streptomyces virus phiCAM, Streptomyces virus Aaronocolus, Streptomyces virus Caliburn, Streptomyces virus Danzina, Streptomyces virus Hydra, Streptomyces virus Izzy, Streptomyces virus Lannister, Streptomyces virus Lika, Streptomyces virus Sujidade, Streptomyces virus Zemlya, Streptomyces virus ELB20, Streptomyces virus R4, Streptomyces virus phiHau3, Mycobacterium virus Acadian, Mycobacterium virus Baee, Mycobacterium virus Reprobate, Mycobacterium virus Adawi, Mycobacterium virus Banel, Mycobacterium virus BrownCNA, Mycobacterium virus Chrisnmich, Mycobacterium virus Cooper, Mycobacterium virus JAMaL, Mycobacterium virus Nigel, Mycobacterium virus Stinger, Mycobacterium virus Vincenzo, Mycobacterium virus Zemanar, Mycobacterium virus Apizium, Mycobacterium virus Manad, Mycobacterium virus Oline, Mycobacterium virus Osmaximus, Mycobacterium virus Pg1, Mycobacterium virus Soto, Mycobacterium virus Suffolk, Mycobacterium virus Athena, Mycobacterium virus Bernardo, Mycobacterium virus Gadjet, Mycobacterium virus Pipefish, Mycobacterium virus Godines, Mycobacterium virus Rosebush, Mycobacterium virus Babsiella, Mycobacterium virus Brujita, Mycobacterium virus Che9c, Mycobacterium virus Sbash, Mycobacterium virus Hawkeye, Mycobacterium virus Plot, Salmonella virus AG11, Salmonella virus Ent1, Salmonella virus f18SE, Salmonella virus Jersey, Salmonella virus L13, Salmonella virus LSPA1, Salmonella virus 5E2, Salmonella virus SETP3, Salmonella virus SETP7, Salmonella virus SETP13, Salmonella virus SP101, Salmonella virus 553e, Salmonella virus wksl3, Escherichia virus K1G, Escherichia virus K1H, Escherichia virus K1ind1, Escherichia virus K1ind2, Salmonella virus SP31, Leuconostoc virus Lmd1, Leuconostoc virus LN03, Leuconostoc virus LN04, Leuconostoc virus LN12, Leuconostoc virus LN6B, Leuconostoc virus P793, Leuconostoc virus 1A4, Leuconostoc virus Ln8, Leuconostoc virus Ln9, Leuconostoc virus LN25, Leuconostoc virus LN34, Leuconostoc virus LNTR3, Mycobacterium virus Bongo, Mycobacterium virus Rey, Mycobacterium virus Butters, Mycobacterium virus Michelle, Mycobacterium virus Charlie, Mycobacterium virus Pipsqueaks, Mycobacterium virus Xeno, Mycobacterium virus Panchino, Mycobacterium virus Phrann, Mycobacterium virus Redi, Mycobacterium virus Skinnyp, Gordonia virus BaxterFox, Gordonia virus Yeezy, Gordonia virus Kita, Gordonia virus Zirinka, Gorrdonia virus Nymphadora, Mycobacterium virus Bignuz, Mycobacterium virus Brusacoram, Mycobacterium virus Donovan, Mycobacterium virus Fishburne, Mycobacterium virus Jebeks, Mycobacterium virus Malithi, Mycobacterium virus Phayonce, Enterobacter virus F20, Klebsiella virus 1513, Klebsiella virus KLPN1, Klebsiella virus KP36, Klebsiella virus PKP126, Klebsiella virus Sushi, Escherichia virus AHP42, Escherichia virus AHS24, Escherichia virus AKS96, Escherichia virus C119, Escherichia virus E41c, Escherichia virus Eb49, Escherichia virus Jk06, Escherichia virus KP26, Escherichia virus Rogue1, Escherichia virus ACGM12, Escherichia virus Rtp, Escherichia virus ADB2, Escherichia virus JMPW1, Escherichia virus JMPW2, Escherichia virus T1, Shigella virus PSf2, Shigella virus Shfl1, Citrobacter virus Stevie, Escherichia virus TLS, Salmonella virus SP126, Cronobacter virus Esp2949-1, Pseudomonas virus Ab18, Pseudomonas virus Ab19, Pseudomonas virus PaMx11, Arthrobacter virus Amigo, Propionibacterium virus Anatole, Propionibacterium virus B3, Bacillus virus Andromeda, Bacillus virus Blastoid, Bacillus virus Curly, Bacillus virus Eoghan, Bacillus virus Finn, Bacillus virus Glittering, Bacillus virus Riggi, Bacillus virus Taylor, Gordonia virus Attis, Mycobacterium virus Barnyard, Mycobacterium virus Konstantine, Mycobacterium virus Predator, Mycobacterium virus Bernal13, Staphylococcus virus 13, Staphylococcus virus 77, Staphylococcus virus 108PVL, Mycobacterium virus Bron, Mycobacterium virus Faith1, Mycobacterium virus Joedirt, Mycobacterium virus Rumpelstiltskin, Lactococcus virus bIL67, Lactococcus virus c2, Lactobacillus virus c5, Lactobacillus virus Ld3, Lactobacillus virus Ld17, Lactobacillus virus Ld25A, Lactobacillus virus LLKu, Lactobacillus virus phiLdb, Cellulophaga virus Cba121, Cellulophaga virus Cba171, Cellulophaga virus Cba181, Cellulophaga virus ST, Bacillus virus 250, Bacillus virus IEBH, Mycobacterium virus Ardmore, Mycobacterium virus Avani, Mycobacterium virus Boomer, Mycobacterium virus Che8, Mycobacterium virus Che9d, Mycobacterium virus Deadp, Mycobacterium virus Diane, Mycobacterium virus Dorothy, Mycobacterium virus Dotproduct, Mycobacterium virus Drago, Mycobacterium virus Fruitloop, Mycobacterium virus Gumbie, Mycobacterium virus Ibhubesi, Mycobacterium virus Llij, Mycobacterium virus Mozy, Mycobacterium virus Mutaforma13, Mycobacterium virus Pacc40, Mycobacterium virus PMC, Mycobacterium virus Ramsey, Mycobacterium virus Rockyhorror, Mycobacterium virus SG4, Mycobacterium virus Shaunal, Mycobacterium virus Shilan, Mycobacterium virus Spartacus, Mycobacterium virus Taj, Mycobacterium virus Tweety, Mycobacterium virus Wee, Mycobacterium virus Yoshi, Salmonella virus Chi, Salmonella virus FSLSP030, Salmonella virus FSLSP088, Salmonella virus iEPS5, Salmonella virus SPN19, Mycobacterium virus 244, Mycobacterium virus Bask21, Mycobacterium virus CJW1, Mycobacterium virus Eureka, Mycobacterium virus Kostya, Mycobacterium virus Porky, Mycobacterium virus Pumpkin, Mycobacterium virus Sirduracell, Mycobacterium virus Toto, Mycobacterium virus Corndog, Mycobacterium virus Firecracker, Rhodobacter virus RcCronus, Pseudomonas virus D3112, Pseudomonas virus DMS3, Pseudomonas virus FHA0480, Pseudomonas virus LPB1, Pseudomonas virus MP22, Pseudomonas virus MP29, Pseudomonas virus MP38, Pseudomonas virus PA1KOR, Pseudomonas virus D3, Pseudomonas virus PMG1, Arthrobacter virus Decurro, Gordonia virus Demosthenes, Gordonia virus Katyusha, Gordonia virus Kvothe, Propionibacterium virus B22, Propionibacterium virus Doucette, Propionibacterium virus E6, Propionibacterium virus G4, Burkholderia virus phi6442, Burkholderia virus phil026b, Burkholderia virus phiE125, Edwardsiella virus eiAU, Mycobacterium virus Ff47, Mycobacterium virus Muddy, Mycobacterium virus Gaia, Mycobacterium virus Giles, Arthrobacter virus Captnmurica, Arthrobacter virus Gordon, Gordonia virus GordTnk2, Paenibacillus virus Harrison, Escherichia virus EK99P1, Escherichia virus HK578, Escherichia virus JL1, Escherichia virus SSL2009a, Escherichia virus YD2008s, Shigella virus EP23, Sodalis virus SO1, Escherichia virus HK022, Escherichia virus HK75, Escherichia virus HK97, Escherichia virus HK106, Escherichia virus HK446, Escherichia virus HK542, Escherichia virus HK544, Escherichia virus HK633, Escherichia virus mEp234, Escherichia virus mEp235, Escherichia virus mEpX1, Escherichia virus mEpX2, Escherichia virus mEp043, Escherichia virus mEp213, Escherichia virus mEp237, Escherichia virus mEp390, Escherichia virus mEp460, Escherichia virus mEp505, Escherichia virus mEp506, Brevibacillus virus Jenst, Achromobacter virus 83-24, Achromobacter virus JWX, Arthrobacter virus Kellezzio, Arthrobacter virus Kitkat, Arthrobacter virus Bennie, Arthrobacter virus DrRobert, Arthrobacter virus Glenn, Arthrobacter virus HunterDalle, Arthrobacter virus Joann, Arthrobacter virus Korra, Arthrobacter virus Preamble, Arthrobacter virus Pumancara, Arthrobacter virus Wayne, Mycobacterium virus Alma, Mycobacterium virus Arturo, Mycobacterium virus Astro, Mycobacterium virus Backyardigan, Mycobacterium virus BBPiebs31, Mycobacterium virus Benedict, Mycobacterium virus Bethlehem, Mycobacterium virus Billknuckles, Mycobacterium virus Bruns, Mycobacterium virus Bxb1, Mycobacterium virus Bxz2, Mycobacterium virus Che12, Mycobacterium virus Cuco, Mycobacterium virus D29, Mycobacterium virus Doom, Mycobacterium virus Ericb, Mycobacterium virus Euphoria, Mycobacterium virus George, Mycobacterium virus Gladiator, Mycobacterium virus Goose, Mycobacterium virus Hammer, Mycobacterium virus Heldan, Mycobacterium virus Jasper, Mycobacterium virus JC27, Mycobacterium virus Jeffabunny, Mycobacterium virus JHC117, Mycobacterium virus KBG, Mycobacterium virus Kssjeb, Mycobacterium virus Kugel, Mycobacterium virus L5, Mycobacterium virus Lesedi, Mycobacterium virus LHTSCC, Mycobacterium virus lockley, Mycobacterium virus Marcell, Mycobacterium virus Microwolf, Mycobacterium virus Mrgordo, Mycobacterium virus Museum, Mycobacterium virus Nepal, Mycobacterium virus Packman, Mycobacterium virus Peaches, Mycobacterium virus Perseus, Mycobacterium virus Pukovnik, Mycobacterium virus Rebeuca, Mycobacterium virus Redrock, Mycobacterium virus Ridgecb, Mycobacterium virus Rockstar, Mycobacterium virus Saintus, Mycobacterium virus Skipole, Mycobacterium virus Solon, Mycobacterium virus Switzer, Mycobacterium virus SWU1, Mycobacterium virus Ta17a, Mycobacterium virus Tiger, Mycobacterium virus Timshel, Mycobacterium virus Trixie, Mycobacterium virus Turbido, Mycobacterium virus Twister, Mycobacterium virus U2, Mycobacterium virus Violet, Mycobacterium virus Wonder, Escherichia virus DE3, Escherichia virus HK629, Escherichia virus HK630, Escherichia virus Lambda, Arthrobacter virus Laroye, Mycobacterium virus Halo, Mycobacterium virus Liefie, Mycobacterium virus Marvin, Mycobacterium virus Mosmoris, Arthrobacter virus Circum, Arthrobacter virus Mudcat, Escherichia virus N15, Escherichia virus 9g, Escherichia virus JenK1, Escherichia virus JenP1, Escherichia virus JenP2, Pseudomonas virus NP1, Pseudomonas virus PaMx25, Mycobacterium virus Baka, Mycobacterium virus Courthouse, Mycobacterium virus Littlee, Mycobacterium virus Omega, Mycobacterium virus Optimus, Mycobacterium virus Thibault, Polaribacter virus P12002L, Polaribacter virus P12002S, Nonlabens virus P12024L, Nonlabens virus P12024S, Thermus virus P23-45, Thermus virus P74-26, Listeria virus LP26, Listeria virus LP37, Listeria virus LP110, Listeria virus LP114, Listeria virus P70, Propionibacterium virus ATCC29399BC, Propionibacterium virus ATCC29399BT, Propionibacterium virus Attacne, Propionibacterium virus Keiki, Propionibacterium virus Kubed, Propionibacterium virus Lauchelly, Propionibacterium virus MrAK, Propionibacterium virus Ouroboros, Propionibacterium virus P91, Propionibacterium virus P105, Propionibacterium virus P144, Propionibacterium virus P1001, Propionibacterium virus P1.1, Propionibacterium virus P100A, Propionibacterium virus P100D, Propionibacterium virus P101A, Propionibacterium virus P104A, Propionibacterium virus PA6, Propionibacterium virus Pacnes201215, Propionibacterium virus PAD20, Propionibacterium virus PAS50, Propionibacterium virus PHL009M11, Propionibacterium virus PHL025M00, Propionibacterium virus PHL037M02, Propionibacterium virus PHL041M1O, Propionibacterium virus PHL060L00, Propionibacterium virus PHL067MO1, Propionibacterium virus PHL070N00, Propionibacterium virus PHL071 N05, Propionibacterium virus PHL082MO3, Propionibacterium virus PHL092M00, Propionibacterium virus PHL095N00, Propionibacterium virus PHL111MO1, Propionibacterium virus PHL112N00, Propionibacterium virus PHL113M01, Propionibacterium virus PHL114L00, Propionibacterium virus PHL116M00, Propionibacterium virus PHL117M00, Propionibacterium virus PHL117M01, Propionibacterium virus PHL132N00, Propionibacterium virus PHL141N00, Propionibacterium virus PHL151M00, Propionibacterium virus PHL151N00, Propionibacterium virus PHL152M00, Propionibacterium virus PHL163M00, Propionibacterium virus PHL171M01, Propionibacterium virus PHL179M00, Propionibacterium virus PHL194M00, Propionibacterium virus PHL199M00, Propionibacterium virus PHL301M00, Propionibacterium virus PHL308M00, Propionibacterium virus Pirate, Propionibacterium virus Procrass1, Propionibacterium virus SKKY, Propionibacterium virus Solid, Propionibacterium virus Stormborn, Propionibacterium virus Wizzo, Pseudomonas virus PaMx28, Pseudomonas virus PaMx74, Mycobacterium virus Patience, Mycobacterium virus PBI1, Rhodococcus virus Pepy6, Rhodococcus virus Poco6, Propionibacterium virus PFR1, Streptomyces virus phiBT1, Streptomyces virus phiC31, Streptomyces virus TG1, Caulobacter virus Karma, Caulobacter virus Magneto, Caulobacter virus phiCbK, Caulobacter virus Rogue, Caulobacter virus Swift, Staphylococcus virus 11, Staphylococcus virus 29, Staphylococcus virus 37, Staphylococcus virus 53, Staphylococcus virus 55, Staphylococcus virus 69, Staphylococcus virus 71, Staphylococcus virus 80, Staphylococcus virus 85, Staphylococcus virus 88, Staphylococcus virus 92, Staphylococcus virus 96, Staphylococcus virus 187, Staphylococcus virus 52a, Staphylococcus virus 80alpha, Staphylococcus virus CNPH82, Staphylococcus virus EW, Staphylococcus virus IPLA5, Staphylococcus virus IPLA7, Staphylococcus virus IPLA88, Staphylococcus virus PH15, Staphylococcus virus phiETA, Staphylococcus virus phiETA2, Staphylococcus virus phiETA3, Staphylococcus virus phiMR11, Staphylococcus virus phiMR25, Staphylococcus virus phiNM1, Staphylococcus virus phiNM2, Staphylococcus virus phiNM4, Staphylococcus virus SAP26, Staphylococcus virus X2, Enterococcus virus FL1, Enterococcus virus FL2, Enterococcus virus FL3, Lactobacillus virus ATCC8014, Lactobacillus virus phiJL1, Pediococcus virus cIP1, Aeromonas virus plS4A, Listeria virus LP302, Listeria virus PSA, Methanobacterium virus psiM1, Roseobacter virus RDJL1, Roseobacter virus RDJL2, Rhodococcus virus RER2, Enterococcus virus BC611, Enterococcus virus IMEEF1, Enterococcus virus SAP6, Enterococcus virus VD13, Streptococcus virus SPQS1, Mycobacterium virus Papyrus, Mycobacterium virus Send513, Burkholderia virus KL1, Pseudomonas virus 73, Pseudomonas virus Ab26, Pseudomonas virus Kakheti25, Escherichia virus Cajan, Escherichia virus Seurat, Staphylococcus virus SEP9, Staphylococcus virus Sextaec, Streptococcus virus 858, Streptococcus virus 2972, Streptococcus virus ALQ132, Streptococcus virus 01205, Streptococcus virus Sfi11, Streptococcus virus 7201, Streptococcus virus DT1, Streptococcus virus phiAbc2, Streptococcus virus Sfi19, Streptococcus virus Sfi21, Paenibacillus virus Diva, Paenibacillus virus Hb10c2, Paenibacillus virus Rani, Paenibacillus virus Shelly, Paenibacillus virus Sitara, Paenibacillus virus Willow, Lactococcus virus 712, Lactococcus virus ASCC191, Lactococcus virus ASCC273, Lactococcus virus ASCC281, Lactococcus virus ASCC465, Lactococcus virus ASCC532, Lactococcus virus Bibb29, Lactococcus virus bIL170, Lactococcus virus CB13, Lactococcus virus CB14, Lactococcus virus CB19, Lactococcus virus CB20, Lactococcus virus jj50, Lactococcus virus P2, Lactococcus virus P008, Lactococcus virus ski, Lactococcus virus SI4, Bacillus virus Slash, Bacillus virus Stahl, Bacillus virus Staley, Bacillus virus Stills, Gordonia virus Bachita, Gordonia virus ClubL, Gordonia virus OneUp, Gordonia virus Smoothie, Gordonia virus Soups, Bacillus virus SPbeta, Vibrio virus MAR10, Vibrio virus SSP002, Escherichia virus AKFV33, Escherichia virus BF23, Escherichia virus DT57C, Escherichia virus EPS7, Escherichia virus FFH1, Escherichia virus H8, Escherichia virus slur09, Escherichia virus T5, Salmonella virus 118970sal2, Salmonella virus Shivani, Salmonella virus SPC35, Salmonella virus Stitch, Arthrobacter virus Tank, Tsukamurella virus TIN2, Tsukamurella virus TIN3, Tsukamurella virus TIN4, Rhodobacter virus RcSpartan, Rhodobacter virus RcTitan, Mycobacterium virus Anaya, Mycobacterium virus Angelica, Mycobacterium virus Crimd, Mycobacterium virus Fionnbarth, Mycobacterium virus Jaws, Mycobacterium virus Larva, Mycobacterium virus Macncheese, Mycobacterium virus Pixie, Mycobacterium virus TM4, Bacillus virus BMBtp2, Bacillus virus TP21, Geobacillus virus Tp84, Staphylococcus virus 47, Staphylococcus virus 3a, Staphylococcus virus 42e, Staphylococcus virus IPLA35, Staphylococcus virus phi12, Staphylococcus virus phiSLT, Mycobacterium virus 32HC, Rhodococcus virus RGL3, Paenibacillus virus Vegas, Gordonia virus Vendetta, Bacillus virus Wbeta, Mycobacterium virus Wildcat, Gordonia virus Twister6, Gordonia virus Wizard, Gordonia virus Hotorobo, Gordonia virus Monty, Gordonia virus Woes, Xanthomonas virus CP1, Xanthomonas virus OP1, Xanthomonas virus phil7, Xanthomonas virus Xop411, Xanthomonas virus Xp10, Streptomyces virus TP1604, Streptomyces virus YDN12, Alphaproteobacteria virus phiJI001, Pseudomonas virus LKO4, Pseudomonas virus M6, Pseudomonas virus MP1412, Pseudomonas virus PAE1, Pseudomonas virus Yua, Pseudoalteromonas virus PM2, Pseudomonas virus phi6, Pseudomonas virus phi8, Pseudomonas virus phi12, Pseudomonas virus phi13, Pseudomonas virus phi2954, Pseudomonas virus phiNN, Pseudomonas virus phiYY, Vibrio virus fs1, Vibrio virus VGJ, Ralstonia virus RS603, Ralstonia virus RSM1, Ralstonia virus RSM3, Escherichia virus M13, Escherichia virus 122, Salmonella virus IKe, Acholeplasma virus L51, Vibrio virus fs2, Vibrio virus VFJ, Escherichia virus If1, Propionibacterium virus B5, Pseudomonas virus Pf1, Pseudomonas virus Pf3, Ralstonia virus PE226, Ralstonia virus RSS1, Spiroplasma virus SVTS2, Stenotrophomonas virus PSH1, Stenotrophomonas virus SMA6, Stenotrophomonas virus SMA7, Stenotrophomonas virus SMA9, Vibrio virus CTXphi, Vibrio virus KSF1, Vibrio virus VCY, Vibrio virus Vf33, Vibrio virus VfO3K6, Xanthomonas virus Cf1c, Spiroplasma virus 074, Spiroplasma virus R8A2B, Spiroplasma virus SkV1CR23x, Escherichia virus FI, Escherichia virus Qbeta, Escherichia virus BZ13, Escherichia virus MS2, Escherichia virus alpha3, Escherichia virus ID21, Escherichia virus ID32, Escherichia virus ID62, Escherichia virus NC28, Escherichia virus NC29, Escherichia virus NC35, Escherichia virus phiK, Escherichia virus St1, Escherichia virus WA45, Escherichia virus G4, Escherichia virus ID52, Escherichia virus Talmos, Escherichia virus phiX174, Bdellovibrio virus MAC1, Bdellovibrio virus MH2K, Chlamydia virus Chpl, Chlamydia virus Chp2, Chlamydia virus CPAR39, Chlamydia virus CPG1, Spiroplasma virus SpV4, Acholeplasma virus L2, Pseudomonas virus PR4, Pseudomonas virus PRD1, Bacillus virus AP50, Bacillus virus Bam35, Bacillus virus GIL16, Bacillus virus Wip1, Escherichia virus phi80, Escherichia virus RB42, Escherichia virus T2, Escherichia virus T3, Escherichia virus T6, Escherichia virus VT2-Sa, Escherichia virus VT1-Sakai, Escherichia virus VT2-Sakai, Escherichia virus CP-933V, Escherichia virus P27, Escherichia virus Stx2phi-I, Escherichia virus Stx1 phi, Escherichia virus Stx2phi-II, Escherichia virus CP-1639, based on the Escherichia virus BP-4795, Escherichia virus 86, Escherichia virus Min27, Escherichia virus 2851, Escherichia virus 1717, Escherichia virus YYZ-2008, Escherichia virus EC026_PO6, Escherichia virus ECO103_P15, Escherichia virus ECO103_P12, Escherichia virus ECO111_P16, Escherichia virus ECO111_P11, Escherichia virus VT2phi_272, Escherichia virus TL-2011c, Escherichia virus P13374, Escherichia virus Sp5; the first bacteriophage being different from the second bacteriophage.

In one embodiment, the first bacteriophage is selected in the group consisting of BW73, B278, D6, D108, E, EI, E24, E41, FI-2, FI-4, FI-5, H18A, Ffl8B, i, MM, Mu, 025, Phl-5, Pk, PSP3, PI, PID, P2, P4, SI, Wφ, φK13, φ1, φ2, φ7, φ92, 7 A, 8φ, 9φ, 18, 28-1, 186, 299, HH-Escherichia (2), AB48, CM, C4, C16, DD-VI, E4, E7, E28, FII, F13, H, HI, H3, H8, K3, M, N, ND-2, ND-3, ND4, ND-5, ND6, ND-7, Ox-I, Ox-2, Ox-3, Ox-4, Ox-5, Ox-6, Phl-I, RB42, RB43, RB49, RB69, S, Sal-I, Sal-2, Sal-3, Sal-4, Sal-5, Sal-6, TC23, TC45, Tull*-6, TuIP-24, Tull*46, TuIP-60, T2, T4, T6, T35, αI, 1, IA, 3, 3A, 3T+, 5φ, 9266Q, CFO103, HK620, J, K, KIF, m59, no. A, no. E, no. 3, no. 9, N4, sd, T3, T7, WPK, W31, ΔH, φC3888, φK3, φK7, φK12, φV-1, PO4-CF, φ05, φ06, φ07, φI, φI.2, φ20, φ95, φ263, φ1092, φI, φII, Ω8, 1, 3, 7, 8, 26, 27, 28-2, 29, 30, 31, 32, 38, 39, 42, 933W, NN-Escherichia (1), Esc-7-11, AC30, CVX-5, Cl, DDUP, ECI, EC2, E21, E29, F1, F26S, F27S, Hi, HK022, HK97, HK139, HK253, HK256, K7, ND-I, PA-2, q, S2, TI,), T3C, T5, UC-I, w, β4, γ2, λ, φD326, φγ, φ06, φ7, φ10, φ80, χ, 2, 4, 4A, 6, 8A, 102, 150, 168, 174, 3000, AC6, AC7, AC28, AC43, AC50, AC57, AC81, AC95, HK243, KIO, ZG/3A, 5, 5A, 21EL, H19-J and 933H.

In a particular embodiment, said first type of bacteriophage is a prophage.

In another particular embodiment, said first type of bacteriophage is a temperate bacteriophage, filamentous phage, or pseudo-lysogenic phage.

By “temperate bacteriophage” or “lysogenic bacteriophage” is meant herein a bacteriophage which infects bacteria or achaea, which can be stably maintained in the genome and/or as episomes of/in a strain, and which replicates with cells without, in their lysogenic state, producing virions. It is well-known from the skilled person which bacteriophages, in the bacteriophages listed above, are temperate phages.

By “filamentous phage” is meant herein a bacteriophage characterized by having a single-stranded DNA genome that is encased by a long protein capsid cylinder. Typically, bacteria infected by filamentous phages are not lysed during the life cycle and replication of the phage, but rather experience a reduced rate of growth. It is well-known from the skilled person which bacteriophages, in the bacteriophages listed above, are filamentous phages.

By “pseudo-lysogenic phage” is meant herein a bacteriophage being at a stage of stalled development in a host cell without either multiplication of the phage genome (as in lytic development) or its replication synchronized with the cell cycle and stable maintenance in the cell line (as in lysogenization), which proceeds with no viral genome degradation, thus allowing the subsequent restart of virus development.

In a preferred embodiment, said first type of bacteriophage is a K. pneumoniae prophage. In another preferred embodiment, said first type of bacteriophage is a C. acnes bacteriophage.

In a preferred embodiment, said second type of bacteriophage is lambda bacteriophage. In another preferred embodiment, said second type of bacteriophage is a P. freudenreichii bacteriophage.

In a preferred embodiment, said first type of bacteriophage is a K. pneumoniae prophage and said second type of bacteriophage is lambda bacteriophage. In another preferred embodiment, said first type of bacteriophage is a C. acnes bacteriophage and said second type of bacteriophage is a P. freudenreichii bacteriophage.

### Additional Bacterial Gene

As well-known from the skilled person, some phages use products produced by their bacterial host for folding and/or assembly of their structural elements, and/or for proper packaging of their DNA.

Therefore, in a particular embodiment, said production bacterial cell further comprises at least one bacterial gene, derived from a bacterial species or strain from which the first type of bacteriophage comes, involved in folding and/or assembly of phage structural elements and/or involved in DNA packaging.

As will be understood by the skilled person, bacterial genes involved in folding and/or assembly of phage structural elements depend on the particular bacteriophage from which said phage structural elements are obtained. They typically include bacterial genes encoding chaperones.

Similarly, bacterial genes involved in phage DNA packaging depend on the particular bacteriophage from which the phage DNA packaging genes are obtained. Examples of such bacterial genes include genes encoding IHF proteins.

### Payload

In a particular embodiment, said production bacterial cell further comprises a payload to be packaged into said phage particles or phage-derived delivery vehicles.

As used herein, the term “payload” refers to any nucleic acid sequence (DNA and/or RNA) or amino acid sequence, or a combination of both (such as, without limitation, peptide nucleic acid or peptide-oligonucleotide conjugate) transferred into a bacterium with a delivery vehicle. In a particular embodiment, the payload is a nucleic acid payload, more particularly a DNA and/or RNA payload, still particularly a DNA payload.

The term “payload” may also refer to a plasmid, a vector or a cargo.

The payload can be a phagemid or phasmid obtained from a natural, evolved or engineered bacteriophage genome. The payload can also be composed only in part of a phagemid or phasmid obtained from a natural, evolved or engineered bacteriophage genome.

As used herein, the term “phagemid” or “phasmid” are equivalent and refer to a recombinant DNA vector comprising at least one sequence of a bacteriophage genome and is able to permit packaging in a capsid, and which is preferably not able of producing progeny, more particularly a vector that derives from both a plasmid and a bacteriophage genome. A phagemid of the disclosure comprises a phage packaging site and optionally an origin of replication (ori), in particular a bacterial and/or phage origin of replication. In one embodiment, the phagemid does not comprise an origin of replication and thus cannot replicate by itself once injected into a bacterium. Alternatively, the phagemid comprises a plasmid origin of replication, in particular a bacterial and/or phage origin of replication.

In a particular embodiment, said payload is to be packaged in the form of a packaged phagemid.

As used herein, the term “packaged phagemid” refers to a phagemid which is encapsidated in a bacteriophage scaffold, phage-derived delivery particle or capsid. Particularly, it refers to a bacteriophage scaffold, phage delivery particle or capsid devoid of a bacteriophage genome. The packaged phagemid may be produced with a helper phage strategy, well known from the man skilled in the art. The helper phage typically comprises all the genes coding for the structural and functional proteins that are indispensable for the phagemid according to the invention to be encapsidated.

In a particular embodiment, said payload is to be delivered into targeted bacterial cells, as defined below.

In a more particular embodiment, said payload is stably maintained in said targeted bacterial cells. In an alternative embodiment, said payload does not replicate in said targeted bacterial cells.

### Sequence of Interest Under the Control of a Promoter

In a particular embodiment, the payload comprises a sequence of interest, in particular under the control of a promoter.

As known by the person skilled in the art, a promoter may be classified as strong or weak according to its affinity for RNA polymerase. The strength of a promoter may depend on whether initiation of transcription occurs at that promoter with high or low frequency. Different promoters with different strengths may be used in the present invention leading to different levels of gene/protein expression (e.g. the level of expression initiated from an mRNA originating from a weak promoter is lower than the level of expression initiated from a strong promoter).

It will be appreciated by those of ordinary skill in the art that a promoter sequence may be selected from a large number of known bacterial genes expressed by various bacterial species. Also, methods of prokaryotic promoter prediction exist, and can be based on DNA stability analysis as described in Kanhere and Bansal (BMC Bioinformatics 2005, 6:1). The choice of promoter on the payload used in the context of the present invention can thus be made based on the bacteria to target.

In some embodiments, the nucleic acid of interest may be positioned under the control of a recombinant or heterologous promoter, which refers to a promoter that is not normally associated with the nucleic acid of interest in its natural environment.

Examples of bacterial promoters for use in accordance with the present invention include, without limitation, positively regulated E. coli promoters such as positively regulated a 70 promoters (e.g., inducible pBad/araC promoter, Lux cassette right promoter, modified lambda Prm promote, plac Or2-62 (positive), pBad/AraC with extra REN sites, pBad, P(Las) TetO, P(Las) CIO, P(Rhl), Pu, FecA, pRE, cadC, hns, pLas, pLux), a “s” promoter (e.g., Pdps), a 32 promoters (e.g., heat shock) and a 54 promoters (e.g., glnAp2); negatively regulated E. coli promoters such as negatively regulated a 70 promoters (e.g., Promoter (PRM+), modified lambda Prm promoter, TetR-TetR-4C P(Las) TetO, P(Las) CIO, P(Lac) IQ, RecA_DlexO_DLac01, dapAp, FecA, Pspac-hy, pel, plux-cl, plux-lac, CinR, CinL, glucose controlled, modified Pr, modified Prm+, FecA, Pcya, rec A (SOS), Rec A (SOS), EmrR_regulated, Betl_regulated, pLac_lux, pTet_Lac, pLac/Mnt, pTet/Mnt, LsrA/cl, pLux/cl, LacI, LacIQ, pLacIQI, pLas/cl, pLas/Lux, pLux/Las, pRecA with LexA binding site, reverse BBa_R0011, pLacI/ara-1, pLacIq, rrnB PI, cadC, hns, PfhuA, pBad/araC, nhaA, OmpF, RcnR), a S promoters (e.g., Lutz-Bujard LacO with alternative sigma factor α 38), a 32 promoters (e.g., Lutz-Bujard LacO with alternative sigma factor α 32), a 54 promoters (e.g., glnAp2); negatively regulated B. subtilis promoters such as repressible B. subtilis β A promoters (e.g., Gram-positive IPTG-inducible, Xyl, hyper-spank), β promoters, and the BioFAB promoters disclosed in Mutalik V K et al (Nature Methods, 2013, 10: 354-360, see in particular the supplementary data) as well as on the BioFAB website (http://biofab.synberc.org/data). Other inducible microbial promoters and/or bacterial promoters may be used in accordance with the present invention. An inducible promoter for use in accordance with the present disclosure may be induced by (or repressed by) one or more physiological condition(s), such as changes in pH, temperature, radiation, osmotic pressure, saline gradients, cell surface binding, and the concentration of one or more extrinsic or intrinsic inducing agent(s). The extrinsic inducer or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Particularly preferred bacterial promoters for use in accordance with the present invention may be selected from constitutive promoters regulated by a 70 such as the promoters of the Anderson collection (http://parts.igem.org/Promoters/Catalog/Anderson): BBa_J23100, BBa_J23101, BBa_J23102, BBa_J23103, BBa_J23104, BBa_J23105, BBa_J23106, BBa_J23107, BBa_J23108, BBa_J23109, BBa_J23110, BBa_J23111, BBa_J23112, BBa_J23113, BBa_J23114, BBa_J23115, BBa_J23116, BBa_J23117, BBa_J23118, and BBa_J23119.

Other preferred bacterial promoters are the promoters disclosed in Stanton et al. (2014) Nat. Chem. Biol. 10:99-105, incorporated herein by reference, including in particular TetR, IcaR(A), AmtR, BetI, SrpR, Orf2, BM3R1, ButR, PhIF, PsrA, HlyIIR, AmeR, LmrA, QacR, ScbR, McbR, LitR, HapR, SmcR, TarA and variants thereof. In a particular embodiment, said promoter is SrpR and/or PhIF, or a variant thereof.

In some embodiments of the present invention, a promoter may or may not be used in conjunction with an “enhancer,” which refers to a cis-acting regulatory sequence involved in the transcriptional activation of a nucleic acid sequence downstream of the promoter. The enhancer may be located at any functional location before or after the promoter.

In some embodiments, the payload may comprise a terminator sequence, or terminator. A “terminator,” as used herein, is a nucleic acid sequence that causes transcription to stop. A terminator may be unidirectional or bidirectional. It consists of a DNA sequence involved in specific termination of an RNA transcript by an RNA polymerase. A terminator sequence prevents transcriptional activation of downstream nucleic acid sequences by upstream promoters. Thus, in certain embodiments, a terminator that ends the production of an RNA transcript is contemplated. A terminator may be necessary in vivo to achieve desirable gene/protein expression levels.

The most commonly used type of terminator is a forward terminator. When placed downstream of a nucleic acid of interest that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only. In prokaryotic systems, terminators usually fall into two categories (1) rho-independent terminators and (2) rho-dependent terminators. Rho-independent terminators are generally composed of a palindromic sequence that forms a stem loop rich in G-C base pairs followed by a string of uracil bases.

Terminators for use in accordance with the present invention include any terminator of transcription described herein or known to one of ordinary skill in the art. Examples of terminators include, without limitation, the termination sequences of genes such as, for example, the bovine growth hormone terminator, and viral termination sequences such as, for example, the TO terminator, the TE terminator, lambda TI and the T1T2 terminator found in bacterial systems. In some embodiments, the termination signal may be a sequence that cannot be transcribed or translated, such as those resulting from a sequence truncation.

Terminators for use in accordance with the present invention also include terminators disclosed in Chen Y J et al (2013, Nature Methods, 10: 659-664), and the BioFAB terminators disclosed in Cambray G et al (Nucl Acids Res, 2013, 41(9): 5139-5148).

In one embodiment, the sequence of interest is a programmable nuclease circuit to be delivered to the targeted bacteria. This programmable nuclease circuit may be able to mediate in vivo sequence-specific elimination of bacteria that contain a target gene of interest (e.g. a gene that is harmful to humans). Some embodiments of the present disclosure relate to engineered variants of the Type II CRISPR-Cas (Clustered Regularly Interspaced Short Palindromic Repeats-CRISPR-associated) system of Streptococcus pyogenes. Other programmable nucleases that can be used include other CRISPR-Cas systems, engineered TALEN (Transcription Activator-Like Effector Nuclease) variants, engineered zinc finger nuclease (ZFN) variants, natural, evolved or engineered meganuclease or recombinase variants, and any combination or hybrids of programmable nucleases. Thus, the engineered autonomously distributed circuits provided herein may be used to selectively cleave DNA encoding a gene of interest such as, for example, a toxin gene, a virulence factor gene, an antibiotic resistance gene, a remodeling gene or a modulatory gene (cf. WO2014124226 and US2015/0064138).

Other sequences of interest, preferably programmable, can be added to the payload so as to be delivered to targeted bacteria. Preferably, the sequence of interest added to the payload leads to cell death of the targeted bacteria. For example, the nucleic acid sequence of interest added to the payload may encode holins, endolysins, restriction enzymes or toxins affecting the targeted bacteria.

Alternatively, the sequence of interest added to the payload does not lead to death of targeted bacteria. For example, the sequence of interest may encode reporter genes leading to a luminescence or fluorescence signal. Alternatively, the sequence of interest may comprise proteins and enzymes achieving a useful function such as modifying the metabolism of the targeted bacteria, the composition of its environment or affecting the host subject. More specifically the sequence of interest can be an antigen triggering a host subject's immune response. The specific antigen can be released in the environment after induction of the lysis of the target cell or can be secreted by the target cell. (Costa et al. Nat Rev Microbiol. 2015 June; 13(6):343-59; Anne et al. Curr Top Microbiol Immunol. 2017; 404:267-308)

In a particular embodiment, the nucleic acid sequence of interest is selected from the group consisting of a Cas nuclease, a Cas9 nuclease, a guide RNA, a single guide RNA (sgRNA), a CRISPR locus, a gene expressing an enzyme such as a nuclease or a kinase, a TALEN, a ZFN, a meganuclease, a recombinase, a transposase, a bacterial receptor, a membrane protein, a structural protein, a secreted protein, a gene expressing resistance to an antibiotic or to a drug in general, a gene expressing a toxic protein or a toxic factor and a gene expressing a virulence protein or a virulence factor, a bacterial secretory protein or transporter, a bacterial pore or any of their combination. These proteins can also be modified or engineered to include extra features, like the addition or removal of a function (e.g. dCas9), the addition of a secretion signal to a protein not normally secreted, the addition of an exogenous peptide in a loop as non-limiting examples.

In a particular embodiment, the nucleic acid sequence of interest encodes a guide RNA-assisted targeting (INTEGRATE) system, typically as disclosed in Vo et al. Nat Biotechnol. 2021 April; 39(4):480-489, said INTEGRATE system including for example a Type I-F V. cholerae CRISPR-transposon or a Type V-K S. hofmanii CRISPR-transposon. In a particular embodiment, said nucleic acid sequence of interest includes a nucleic acid encoding a crRNA, a nucleic acid encoding TniQ cascade, cas8, cas7 and cas6 proteins, a nucleic acid encoding tnsA, tnsB and tnsC proteins, and further including a donor DNA, said donor DNA encoding a protein of interest to be added into the targeted bacteria genome. In a particular embodiment, said nucleic acids encoding TniQ cascade, cas8, cas7 and cas6 proteins, and encoding tnsA, tnsB and tnsC proteins, are in the form of a single polycistronic nucleic acid. In another particular embodiment, said nucleic acid sequence of interest includes a nucleic acid encoding a guide RNA, a nucleic acid encoding cas12k protein, tnsB and tnsC proteins and TniQ cascade, and further including a donor DNA, said donor DNA encoding a protein of interest to be added into the targeted bacteria genome.

In a particular embodiment, the payload used in the context of the invention comprises a sequence of interest that encodes a bacteriocin, which can be a proteinaceous toxin produced by bacteria to kill or inhibit growth of other bacteria. Bacteriocins are categorized in several ways, including producing strain, common resistance mechanisms, and mechanism of killing. Such bacteriocins have been described from gram negative bacteria (e.g. microcins, colicin-like bacteriocins and tailocins) and from gram positive bacteria (e.g. Class I, Class II, Class III or Class IV bacteriocins).

In one embodiment, the payload used in the context of the invention further comprises a sequence of interest encoding a toxin selected in the group consisting of microcins, colicin-like bacteriocins, tailocins, Class I, Class II, Class III and Class IV bacteriocins. The circuit may also encode the transporter needed to secrete the toxin to the extracellular space.

In a particular embodiment, the corresponding immunity polypeptide (i.e. anti-toxin) may be used to protect bacterial cells (see review by Cotter et al., Nature Reviews Microbiology 11: 95, 2013) for payload production and encapsidation purpose but is absent in the pharmaceutical composition and in the targeted bacteria in which the payload used in the context of the invention is delivered.

In a particular embodiment, the payload used in the context of the invention comprises a sequence of interest that encodes a CRISPR-Cas system.

The CRISPR system contains two distinct elements, i.e. i) an endonuclease, in this case the CRISPR associated nuclease (Cas or “CRISPR associated protein”) and ii) a guide RNA. Depending on the type of CRISPR system, the guide RNA may be in the form of a chimeric RNA which consists of the combination of a CRISPR (crRNA) bacterial RNA and a tracrRNA (trans-activating RNA CRISPR) (Jinek et al. Science. 2012 Aug. 17; 337(6096):816-21). The guide RNA combines the targeting specificity of the crRNA corresponding to the “spacing sequences” that serve as guides to the Cas proteins, and the conformational properties of the tracrRNA in a single transcript. When the guide RNA and the Cas protein are expressed simultaneously in the cell, the target genomic sequence can be permanently interrupted (and causing disappearance of the targeted and surrounding sequences and/or cell death, depending on the location) or modified. The modification may be guided by a repair matrix.

The CRISPR system includes two main classes depending on the nuclease mechanism of action:
Class 1 is made of multi-subunit effector complexes and includes type I, III and IV;
Class 2 is made of single-unit effector modules, like Cas9 nuclease, and includes type II (II-A,II-B,II-C,II-C variant), V (V-A,V-B,V-C,V-D,V-E,V-UI,V-U2,V-U3,V-U4,V-U5) and VI (VI-A,VI-B1,VI-B2,VI-C,VI-D).

The sequence of interest according to the present invention may comprise a nucleic acid sequence encoding Cas protein. A variety of CRISPR enzymes are available for use as a sequence of interest on the payload used in the context of the present invention. In some embodiments, the CRISPR enzyme is a Type II CRISPR enzyme, a Type II-A or Type II-B CRISPR enzyme. In another embodiment, the CRISPR enzyme is a Type I CRISPR enzyme or a Type III CRISPR enzyme. In some embodiments, the CRISPR enzyme catalyzes DNA cleavage. In some other embodiments, the CRISPR enzyme catalyzes RNA cleavage. In one embodiment, the CRISPR enzymes may be coupled to a guide RNA or single guide RNA (sgRNA). In certain embodiments, the guide RNA or sgRNA targets a gene selected from the group consisting of an antibiotic resistance gene, virulence protein or factor gene, toxin protein or factor gene, a bacterial receptor gene, a membrane protein gene, a structural protein gene, a secreted protein gene, a gene expressing resistance to a drug in general and a gene causing a deleterious effect to the host subject.

The sequence of interest may comprise a nucleic acid sequence encoding a guide RNA or sgRNA to guide the Cas protein endogenous to the targeted bacteria, alone or in combination with a Cas protein and/or a guide RNA encoded by the payload.

Non-limiting examples of Cas proteins as part of a multi-subunit effector or as a single-unit effector include Cast, Cas1B, Cas2, Cas3, Cas4, Cas5, Cas6, Cas7, Cas8, Cas9 (also known as Csn1 and Csx12), Cas10, Cash 1 (SS), Cas12a (Cpf1), Cas12b (C2c1), Cas12c (C2c3), Cas12d (CasY), Cas12e (CasX), C2c4, C2c8, C2c5, C2c1O, C2c9, Cas13a (C2c2), Cas13b (C2c6), Cas13c (C2c7), Cas13d, Csa5, Csc1, Csc2, Cse1, Cse2, Csy1, Csy2, Csy3, Csf1, Csf2, Csf3, Csf4, Csm1, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, SdCpf1, CmtCpf1, TsCpf1, CmaCpf1, PcCpf1, ErCpf1, FbCpf1, UbcCpf1, AsCpf1, LbCpf1, Mad4, Mad7, Cms1, homologues thereof, orthologues thereof, variants thereof, or modified versions thereof. In some embodiments, the CRISPR enzyme cleaves both strands of the target nucleic acid at the Protospacer Adjacent Motif (PAM) site.

In a particular embodiment, the CRISPR enzyme is any Cas9 protein, for instance any naturally-occurring bacterial Cas9 as well as any variants, homologs or orthologs thereof.

By “Cas9” is meant a protein Cas9 (also called Csn1 or Csx12) or a functional protein, peptide or polypeptide fragment thereof, i.e. capable of interacting with the guide RNA(s) and of exerting the enzymatic activity (nuclease) which allows it to perform the double-strand cleavage of the DNA of the target genome. “Cas9” can thus denote a modified protein, for example truncated to remove domains of the protein that are not essential for the predefined functions of the protein, in particular the domains that are not necessary for interaction with the gRNA(s).

The sequence encoding Cas9 (the entire protein or a fragment thereof) as used in the context of the invention can be obtained from any known Cas9 protein (Fonfara et al. Nucleic Acids Res. 2014 February; 42(4):2577-90; Koonin et al. Curr Opin Microbiol. 2017 June; 37:67-78). Examples of Cas9 proteins useful in the present invention include, but are not limited to, Cas9 proteins of Streptococcus pyogenes (SpCas9), Streptococcus thermophiles (St1 Cas9, St3Cas9), Streptococcus mutans, Staphylococcus aureus (SaCas9), Campylobacter jejuni (CjCas9), Francisella novicida (FnCas9) and Neisseria meningitides (NmCas9).

The sequence encoding Cpf1 (Cas12a) (the entire protein or a fragment thereof) as used in the context of the invention can be obtained from any known Cpf1 (Cas12a) protein (Koonin et al. Curr Opin Microbiol. 2017 June; 37:67-78). Examples of Cpf1(Cas12a) proteins useful in the present invention include, but are not limited to, Cpf1 (Cas12a) proteins of Acidaminococcus sp, Lachnospiraceae bacterium and Francisella novicida.

The sequence encoding Cas13a (the entire protein or a fragment thereof) as used in the context of the invention can be obtained from any known Cas13a (C2c2) protein (Abudayyeh et al. Nature. 2017 Oct. 12; 550(7675):280-284). Examples of Cas13a (C2c2) proteins useful in the present invention include, but are not limited to, Cas13a (C2c2) proteins of Leptotrichia wadei (LwaCas13a).

The sequence encoding Cas13d (the entire protein or a fragment thereof) as used in the context of the invention can be obtained from any known Cas13d protein (Yan et al. Mol Cell. 2018 Apr. 19; 70(2):327-339.e5.). Examples of Cas13d proteins useful in the present invention include, but are not limited to, Cas13d proteins of Eubacterium siraeum and Ruminococcus sp.

The sequence encoding Mad4 (the entire protein or a fragment thereof) as used in the context of the invention is disclosed in international application WO2018/236548.

The sequence encoding Mad7 (the entire protein or a fragment thereof) as used in the context of the invention is disclosed in international application WO2018/236548.

The sequence encoding Cms1 (the entire protein or a fragment thereof) as used in the context of the invention is disclosed in international patent application WO2017/141173.

In a particular embodiment, the nucleic sequence of interest is a CRISPR/Cas9 system for the reduction of gene expression or inactivation of a gene selected from the group consisting of an antibiotic resistance gene, virulence factor or protein gene, toxin factor or protein gene, a gene expressing a bacterial receptor, a membrane protein, a structural protein, a secreted protein, a gene expressing resistance to a drug in general and a gene causing a deleterious effect to the host subject.

In one embodiment, the CRISPR system is used to target and inactivate a virulence factor. A virulence factor can be any substance produced by a pathogen that alter host subject-pathogen interaction by increasing the degree of damage done to the host subject. Virulence factors are used by pathogens in many ways, including, for example, in cell adhesion or colonization of a niche in the host subject, to evade the host subject's immune response, to facilitate entry to and egress from host subject's cells, to obtain nutrition from the host subject, or to inhibit other physiological processes in the host subject. Virulence factors can include enzymes, endotoxins, adhesion factors, motility factors, factors involved in complement evasion, scavenging factors and factors that promote biofilm formation. For example, such targeted virulence factor gene can be E. coli virulence factor gene such as, without limitation, EHEC-HlyA, Stx1 (VT1), Stx2 (VT2), Stx2a (VT2a), Stx2b (VT2b), Stx2c (VT2c), Stx2d (VT2d), Stx2e (VT2e) and Stx2f (VT2f), Stx2h (VT2h), stx2k, fimA, fimF, fimH, neuC, kpsE, sfa, foc, iroN, aer, iha, papC, papGI, papGII, papGIII, hlyC, cnf1, hra, sat, ireA, usp ompT, ibeA, maIX, fyuA, irp2, traT, afaD, ipaH, eltB, estA, bfpA, eaeA, espA, aaiC, aatA, TEM, CTX, SHV, csgA, csgB, csgC, csgD, csgE, csgF, csgG, csgH, T1SS, T2SS, T3SS, T4SS, T5SS, T6SS (secretion systems). For example, such targeted virulence factor gene can be Shigella dysenteriae virulence factor gene such as, without limitation, stx1 and stx2. For example, such targeted virulence factor gene can be Yersinia pestis virulence factor gene such as, without limitation, yscF (plasmid-borne (pCDI) T3SS external needle subunit). For example, such targeted virulence factor gene can be Francisella tularensis virulence factor gene such as, without limitation, fslA. For example, such targeted virulence factor gene can be Bacillus anthracis virulence factor gene such as, without limitation, pag (Anthrax toxin, cell-binding protective antigen). For example, such targeted virulence factor gene can be Vibrio cholera virulence factor gene such as, without limitation, ctxA and ctxB (cholera toxin), tcpA (toxin co-regulated pilus), and toxT (master virulence regulator). For example, such targeted virulence factor gene can be Pseudomonas aeruginosa virulence factor genes such as, without limitation, pyoverdine (e.g., sigma factor pvdS, biosynthetic genes pvdL, pvdI, pvdJ, pvdH, pvdA, pvdF, pvdQ, pvdN, pvdM, pvdO, pvdP, transporter genes pvdE, pvdR, pvdT, opmQ), siderophore pyochelin (e.g., pchD, pchC, pchB, pchA, pchE, pchF and pchG, and toxins (e.g., exoU, exoS and exoT). For example, such targeted virulence factor gene can be Klebsiella pneumoniae virulence factor genes such as, without limitation, fimA (adherence, type I fimbriae major subunit), and cps (capsular polysaccharide). For example, such targeted virulence factor gene can be Acinetobacter baumannii virulence factor genes such as, without limitation, ptk (capsule polymerization) and epsA (assembly). For example, such targeted virulence factor gene can be Salmonella enterica Typhi virulence factor genes such as, without limitation, MIA (invasion, SPI-1 regulator), ssrB (SPI-2 regulator), and those associated with bile tolerance, including efflux pump genes acrA, acrB and toIC. For example, such targeted virulence factor gene can be Fusobacterium nucleatum virulence factor genes such as, without limitation, FadA and TIGIT. For example, such targeted virulence factor gene can be Bacteroides fragilis virulence factor genes such as, without limitation, bft. For example, such targeted virulence factor gene can be Cutibacterium acnes porphyrins genes, CAMP-factors (CAMP1, CAMP2, CAMP3, CAMP4), Hyaluronate lyase (HYL-IB/II, HYL-IA), Lipases (GehA, GehB), Haemolysins, Sialidases, Endoglycoceramidases, Endo-β-N-acetylglucosaminidase, Dermatan sulfate adhesin (DsA1, DsA2), Proline-Threonine Repeats (PTRs) or any virulence factors included on the acne associated genomic loci 1, 2, 3(plasmid), 4 such as a tight adhesion locus (tad), Streptolysin S-associated genes (sag), nonribosomal peptide synthetases (NRPS) as described in Tomida et al. mBio. 2013 Apr. 30; 4(3):e00003-13.

In another embodiment, the CRISPR/Cas system is used to target and inactivate an antibiotic resistance gene such as, without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA β-lactamase, mecA, Omp36, OmpF, PIB, bla (blal, blaR1) and mec (mecl, mecR1) operons, Chloramphenicol acetyltransferase (CAT), Chloramphenicol phosphotransferase, Ethambutol-resistant arabinosyltransferase (EmbB), MupA, MupB, Integral membrane protein MprF, Cfr 23S rRNA methyltransferase, Rifampin ADP-ribosyltransferase (Arr), Rifampin glycosyltransferase, Rifampin monooxygenase, Rifampin phosphotransferase, DnaA, RbpA, Rifampin-resistant beta-subunit of RNA polymerase (RpoB), Erm 23S rRNA methyltransferases, Lsa, MsrA, Vga, VgaB, Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, Lincosamide nucleotidyltransferase (Lin), EreA, EreB, GimA, Mgt, Ole, Macrolide phosphotransferases (MPH), MefA, MefE, Mel, Streptothricin acetyltransferase (sat), Sul1, Sul2, Sul3, sulfonamide-resistant FoIP, Tetracycline inactivation enzyme TetX, TetA, TetB, TetC, Tet30, Tet31, TetM, TetO, TetQ, Tet32, Tet36, MacAB-ToIC, MsbA, MsrA,VgaB, EmrD, EmrAB-ToIC, NorB, GepA, MepA, AdeABC, AcrD, MexAB-OprM, mtrCDE, EmrE, adeR, acrR, baeSR, mexR, phoPQ, mtrR, or any antibiotic resistance gene described in the Comprehensive Antibiotic Resistance Database (CARD https://card.mcmaster.ca/).

In another embodiment, the CRISPR/Cas system is used to target and inactivate a bacterial toxin gene. Bacterial toxins can be classified as either exotoxins or endotoxins. Exotoxins are generated and actively secreted; endotoxins remain part of the bacteria. The response to a bacterial toxin can involve severe inflammation and can lead to sepsis. Such toxin can be for example Botulinum neurotoxin, Tetanus toxin, Staphylococus toxins, Diphteria toxin, Anthrax toxin, Alpha toxin, Pertussis toxin, Shiga toxin, Heat-stable enterotoxin (E. coli ST), colibactin, BFT (B. fragilis toxin) or any toxin described in Henkel et al., (Toxins from Bacteria in EXS. 2010; 100: 1-29).

In a particular embodiment, the payload used in the context of the invention comprises a sequence of interest that encodes a base editing system.

Base editing (BE) refers to the ability to substitute a specific nucleotide base pair on a DNA or RNA molecule by another. Until recently, the only way to perform a specific substitution on DNA in vivo was using recombination of a template DNA, carrying the specific base pair change, with the locus of interest. Base editing technology relies on completely different strategies. There is no exchange of DNA, instead an enzymatic reaction converts a nucleotide to another one leading to a mismatch at the level of dsDNA that is then corrected by the cell machinery.

In some embodiments, the base editing system comprises one or more of the following enzymes and systems:

A) Cytosine base editors (CBE) and Adenosine base editors (ABE), as described in Rees, H. A. & Liu, D. R. Nat Rev Genet 19, 770-788 (2018).

So far there are seven types of DNA base editors described:
Cytosine Base Editor (CBE) that convert C:G into T:A (Komor, A et al. Nature 533:420-4. (2016))
Adenine Base Editor (ABE) that convert A:T into G:C (Gaudelli, N. M. et al. Nature 551(7681) 464-471 (2017))
Cytosine Guanine Base Editor (CGBE) that convert C:G into G:C (Chen, L et al. Precise and programmable C:G to G:C base editing in genomic DNA. Biorxiv (2020).; Kurt, I et al. CRISPR C-to-G base editors for inducing targeted DNA transversions in human cells. Nature Biotechnology (2020))
Cytosine Adenine Base Editor (CABE) that convert C:G into A:T (Zhao, D et al. New base editors change C to A in bacteria and C to G in mammalian cells. Nature Biotechnology (2020))
Adenine Cytosine Base Editor (ACBE) that convert A:T into C:G (WO2020181180)
Adenine Thymine Base Editor (ATBE) that convert A:T into T:A (WO2020181202)
Thymine Adenine Base Editor (TABE) that convert T:A into A:T (WO2020181193; WO2020181178; WO2020181195)

Base editors differ in the base modification enzymes. CBE rely on ssDNA cytidine deaminase among which: APOBEC1, rAPOBEC1, APOBEC1 mutant or evolved version (evoAPOBEC1), and APOBEC homologs (APOBEC3A (eA3A), Anc689), Cytidine deaminase 1 (CDA1), evoCDA1, FERNY, evoFERNY.

ABE rely on deoxyadenosine deaminase activity of a tandem fusion TadA-TadA* where TadA* is an evolved version of TadA, an E. coli tRNA adenosine deaminase enzyme, able to convert adenosine into Inosine on ssDNA. TadA* include TadA-8a-e and TadA-7.10.

Except from base modification enzyme there has been also modifications implemented to base editor to increase editing efficacy, precision and modularity:
the addition of one or two uracil DNA glycosylase inhibitor domain (UGI) to prevent base excision repair mechanism to revert base edition
the addition of Mu-GAM that decrease insertion-deletion rate by inhibiting Non-homologous end joining mechanism in the cell (NHEJ)
the use of nickase active Cas9 (nCas9 D10A) that, by creating nicks on the non-edited strand favors its repair and consequently the fixation of the edited base.
the use of diverse Cas proteins from for example different organisms, mutants with different PAM motifs or different fidelity or different family (e.g. Cas12a).

Non-limiting examples of DNA-based editor proteins include BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Cas12a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRY-CBE, SpG-CBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3, AncBE4.

Cytosine Guanine Base Editors (CGBE) consist of a nickase CRISPR fused to:
A cytosine deaminase (rAPOBEC) and base excision repair proteins (e.g. rXRCC1) (Chen, L et al. Precise and programmable C:G to G:C base editing in genomic DNA. Biorxiv (2020).; Chen et al. Nature Communications 12:1384 (2021))
A rat APOBEC1 variant (R33A) protein and an E. coli-derived uracil DNA N-glycosylase (eUNG) (Kurt, I et al. CRISPR C-to-G base editors for inducing targeted DNA transversions in human cells. Nature Biotechnology (2020))

Cytosine Adenine Base Editors (CABE) consist of a Cas9 nickase, a cytidine deaminase (e.g. AID), and a uracil-DNA glycosylase (Ung) (Zhao, D et al. New base editors change C to A in bacteria and C to G in mammalian cells. Nature Biotechnology (2020)).

ACBE include a nucleic acid programmable DNA-binding protein and an adenine oxidase (WO2020181180).

ATBE consist of a Cas9 nickase and one or more adenosine deaminase or an oxidase domain (WO2020181202).

TABE consist of a Cas9 nickase and an adenosine methyltransferase, a thymine alkyltransferase, or an adenosine deaminase domain (WO2020181193; WO2020181178; WO2020181195).

Base editor molecules can also consist of two or more of the above listed editor enzymes fused to a Cas protein (e.g. combination of an ABE and CBE). These biomolecules are named dual base editors and enable the editing of two different bases (Grunewald, J et al. A dual-deaminase CRISPR base editor enables concurrent adenine and cytosine editing, Nature Biotechnology (2020); Li, C et al. Targeted, random mutagenesis of plant genes with dual cytosine and adenine base editors, Nature Biotechnology (2020)).

In a particular embodiment, the base editing system comprises a Cytosine base editor (CBE) and/or an Adenosine base editor (ABE) as defined above.

B) Prime editors (PE), as described in Anzalone, A. V. et al. Nature 576, 149-157 (2019), consist of a nCas9 fused to a reverse transcriptase used in combination with a prime editing RNA (pegRNA; a guide RNA that includes a template region for reverse transcription).

Prime Editing allows introduction of insertions, deletions (indels), and 12 base-to-base conversions. Prime editing relies on the ability of a reverse transcriptase (RT), fused to a Cas nickase variant, to convert RNA sequence brought by a prime editing guide RNA (pegRNA) into DNA at the nick site generated by the Cas protein. The DNA flap generated from this process is then included or not in the targeted DNA sequence.

Prime editing systems include:
a Cas nickase variant such as Cas9-H840A fused to a reverse transcriptase domain such as M-MLV RT or its mutant version (M-MLV RT(D200N), M-MLV RT(D200N/L603W), M-MLV RT(D200N/L603W/T330P/T306K/W313F)
a prime editing guide RNA (pegRNA)

To favor editing, the prime editing system can include the expression of an additional sgRNA targeting the Cas nickase activity towards the non-edited DNA strand ideally only after the resolution of the edited strand flap by designing the sgRNA to anneal with the edited strand but not with the original strand.

Non-limiting examples of prime editing systems include PE1, PE1-M1, PE1-M2, PE1-M3, PE1-M6, PE1-M15, PE1-M3inv, PE2, PE3, PE3b.

Cas9 Retron preclSe Parallel Editing via homologY (‘CRISPEY’), a retron RNA fused to the sgRNA and expressed together with Cas9 and the retron proteins including at least the reverse transcriptase (Sharon, E. et al. Cell 175, 544-557.e16 (2018)).

The SCRIBE strategy: a retron system expressed in combination with a recombinase promoting the recombination of single stranded DNA, also known as single stranded annealing proteins (SSAPs) (Farzadfard, F. & Lu, T. K. Science 346, 1256272 (2014)). Such recombinases include but are not limited to phage recombinases such as lambda red, recET, Sak, Sak4, and newly described SSAPs described in Wannier, T. M. et al. Improved bacterial recombineering by parallelized protein discovery. Biorxiv 2020.01.14.906594 (2020) doi:10.1101/2020.01.14.906594.

The targetron system based on group II introns described in Karberg, M. et al. Nat Biotechnol 19, 1162-7 (2001) which has been adapted to many bacterial species.

Other retron based gene targeting approaches are described in Simon, A. J., Ellington, A. D. & Finkelstein, I. J. Nucleic Acids Res 47, 11007-11019 (2019).

C) CRISPR/Cas. In various embodiments, the sequence of interest encodes fusion proteins comprising a Cas9 (e.g., a Cas9 nickase) domain and a deaminase domain. In some embodiments, the fusion protein comprises Cas9 and a cytosine deaminase enzyme, such as APOBEC enzymes, or adenosine deaminase enzymes, such as ADAT enzymes, for example as disclosed in U.S. Patent Publ. 2015/0166980. In one embodiment, the deaminase is an ACF1/ASE deaminase.

In various embodiments, the APOBEC deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In various embodiments, the fusion protein comprises a Cas9 domain, a cytosine deaminase domain, and a uracil glycosylase inhibitor (UGI) domain.

In one embodiment, the deaminase is an adenosine deaminase that deaminate adenosine in DNA, for example as disclosed in U.S. Pat. No. 10,113,163. In some embodiments, the fusion proteins further comprise an inhibitor of base repair, such as, a nuclease dead inosine specific nuclease (dISN), for example as disclosed in U.S. Pat. No. 10,113,163. In various embodiments, the nucleic acid of interest encodes fusion proteins comprising a catalytically impaired Cas9 endonuclease fused to an engineered reverse transcriptase, programmed with a prime editing guide RNA (pegRNA) that both specifies the target site and encodes the desired edit, for example as described in Anzalone et al.

In some embodiments, other programmable nucleases can be used. These include an engineered TALEN (Transcription Activator-Like Effector Nuclease) and variants, engineered zinc finger nuclease (ZFN) variants, natural, evolved or engineered meganuclease or recombinase variants, and any combination or hybrids of programmable nucleases. Thus, the programmable nucleases provided herein may be used to selectively modify DNA encoding a DNA sequence or gene of interest such as, for example, a toxin gene, a virulence factor gene, an antibiotic resistance gene, a remodeling gene or a modulatory gene (cf. WO2014124226 and US2015/0064138).

In one embodiment, the base editing system or base editor is used to inactivate the expression of a gene by editing one or several nucleotides involved in transcription or translation. More specifically the base editing system or base editor is targeting one or several nucleotides of a promoter, a RBS, a start codon.

In one embodiment, the base editing system or base editor is used to introduce a premature stop codon.

In one embodiment, the base editing system or base editor is used to introduce one or several rare codons.

In another embodiment, the base editing system or base editor is used to modulate the expression of genes by editing one or several nucleotides involved in transcription or translation. More specifically the base editing system or base editor is targeting one or several nucleotides of a promoter, a RBS, a start codon. leading to an increase or decrease of gene expression.

In another embodiment, the base editing system or base editor is used to revert a mutation that leads to the inactivation, decrease or increase in activity of a gene or pathway.

In another embodiment, the base editing system or base editor is used to revert a mutation that leads to an increase of pathogenicity.

In one embodiment, the base editing system or base editor is used to modify the regulation of a gene by editing one or several nucleotides involved in its regulation such as nucleotides of operator sequence, transcription factor binding site, riboswitch, RNAse recognition site, protease cleavage site, methylation site, post translational modification site (phosphorylation, glycosylation, acetylation, pupylation . . . ).

In some embodiments, the sequence of interest encodes a RNA base editing system. RNA base editing is based on the same principle as DNA base editing: an enzyme catalyzing the conversion of a RNA base into another must be brought close to the target base to perform its conversion locally. In one embodiment, the enzyme used for RNA editing is an adenosine deaminase from ADAR family that converts Adenosine into Inosine in dsRNA structure. Several seminal studies used this specificity for dsRNA and fused the ADAR deaminase domain (ADAR_{DD}) to an antisense oligo in order to program local RNA base editing. More recently the ability of some CRISPR-Cas systems to bind RNA molecules was repurposed into RNA editing. Using catalytically dead Cas13b enzyme (dPspCas13b) fused to a hyperactive mutant of ADAR2 deaminase domain (ADAR2_{DD}-E488Q for REPAIRv1 and ADAR2_{DD}-E488Q-T375G for REPAIRv2), Cox et al improved specificity and efficiency compare to previous RNA editing strategies (Cox, D. B. T. et al. Science 358, 1019-1027 (2017)).

Non-limiting examples of RNA based editor proteins include REPAIRv1 and REPAIRv2.

In one embodiment, the RNA base editor is used to inactivate the expression of a gene by editing one or several nucleotides involved in translation. More specifically the base editor is targeting one or several nucleotides of a 5′UTR, a RBS, a start codon.

In one embodiment, the RNA base editor is used to introduce a premature stop codon.

In one embodiment, the RNA base editor is used to introduce one or several rare codons.

In another embodiment, the RNA base editor is used to modulate the expression of genes by editing one or several nucleotides involved in translation. More specifically the base editor is targeting one or several nucleotides of a 5′UTR, a RBS, a start codon leading to an increase or decrease of gene expression.

In another embodiment, the RNA base editor is used to revert a mutation that leads to the inactivation or a decrease in activity of a gene or pathway.

In another embodiment, the base editor is used to revert a mutation that leads to an increase of pathogenicity.

In a preferred embodiment, said sequence of interest only generates an effect in said targeted bacterial cells. More preferably, said sequence of interest is only expressed in said targeted bacterial cells.

### Origins of Replication

In a particular embodiment, the copy number of said payload is controlled, in said production bacterial cell, by said at least one induction mechanism defined above. In an alternative embodiment, another induction mechanism controls the copy number of said payload in said production bacterial cell.

Origins of replication known in the art have been identified from species-specific plasmid DNAs (e.g. ColE1, RI, pT181, pSC101, pMB1, R6K, RK2, p15a and the like), from bacterial virus (e.g. φX174, M13, F1 and P4) and from bacterial chromosomal origins of replication (e.g. oriC).

In one embodiment, the payload used in the context of the invention comprises a bacterial origin of replication that is functional in the targeted bacteria.

Alternatively, the payload used in the context of the invention does not comprise any functional bacterial origin of replication or contains an origin of replication that is inactive in the targeted bacteria. In such embodiment, the payload used in the context of the invention cannot replicate by itself once it has been introduced into a bacterium by the phage particle or phage-derived delivery particle.

In one embodiment, the origin of replication on the payload to be packaged is inactive in the targeted bacteria, meaning that this origin of replication is not functional in the bacteria targeted by the phage particle or phage-derived delivery vehicle, thus preventing unwanted plasmid replication.

In one embodiment, the payload comprises a bacterial origin of replication that is functional in the production bacterial cell of the invention.

### Bacteria-Specific Origins of Replication

Plasmid replication depends on host bacteria enzymes and on plasmid-controlled cis and trans determinants. For example, some plasmids have determinants that are recognized in almost all gram-negative bacteria and act correctly in each host bacteria during replication initiation and regulation. Other plasmids possess this ability only in some bacteria (Kues, U and Stahl, U 1989 Microbiol Rev 53:491-516).

Plasmids are replicated by three general mechanisms, namely theta type, strand displacement, and rolling circle (reviewed by Del Solar et al. 1998 Microbio and Molec Biol. Rev 62:434-464) that start at the origin of replication. These replication origins contain sites that are required for interactions of plasmid and/or host encoded proteins.

Origins of replication used on the payload used in the context of the invention may be moderate copy number, such as ColE1 on from pBR322 (15-20 copies per cell) or the R6K plasmid (15-20 copies per cell) or may be high copy number, e.g. pUC oris (500-700 copies per cell), pGEM oris (300-400 copies per cell), pTZ oris (>1000 copies per cell) or pBluescript oris (300-500 copies per cell).

In one embodiment, the bacterial origin of replication is selected in the group consisting of ColE1, pMB1 and variants (pBR322, pET, pUC, etc), p15a, CoIA, ColE2, pOSAK, pSC101, R6K, IncW (pSa etc), IncFII, pT181, P1, F IncP, IncC, IncJ, IncN, IncP1, IncP4, IncQ, IncH11, RSF1010, CloDF13, NTP16, R1, f5, pPS10, pC194, pE194, BBR1, pBC1, pEP2, pWVO1, pLF1311, pAP1, pWKS1, pLS1, pLS11, pUB6060, pJD4, pIJ101, pSN22, pAMbeta1, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

More preferably, the bacterial origin of replication is a E. coli origin of replication selected in the group consisting of ColE1, pMB1 and variants (pBR322, pET, pUC, etc), p15a, CoIA, ColE2, pOSAK, pSC101, R6K, IncW(pSa etc), IncFII, pT181, P1, F IncP, IncC, IncJ, IncN, IncP1, IncP4, IncQ, IncH11, RSF1010, CloDF13, NTP16, R1, f5, pPS10.

More preferably, the bacterial origin of replication is selected in the group consisting of pC194, pE194, BBR1, pBC1, pEP2, pWVO1, pLF1311, pAP1, pWKS1, pLS1, pLS11, pUB6060, pJD4, pIJ101, pSN22, pAMbeta1, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

Even more preferably, the bacterial origin of replication are ColE1 and p15a.

In one embodiment, the bacterial origin of replication is functional in Propionibacterium and Cutibacterium, more specifically in Propionibacterium freudenreichii and Cutibacterium acnes and is selected from the group consisting of pLME108, pLME106, p545, pRGO1, pZGX01, pPGO1, pYS1, FRJS12-3, FRJS25-1, pIMPLE-HL096PA1,A_15_1_R1. In a particular embodiment, the bacterial origin of replication is selected from the bacterial origins of replication disclosed in US applications US2022/135986 and US2022/135987.

### Phage Origin of Replication

The payload used in the context of the invention may comprise a phage origin of replication which can initiate, with complementation of a complete phage genome, the replication of the payload for later encapsulation into the different capsids.

A phage origin of replication can also be engineered to act as a bacterial origin of replication without the need to package any phage particles.

A phage origin of replication comprised in the payload used in the context of the invention can be any origin of replication found in a phage.

Preferably, the phage origin of replication can be the wild-type or non-wild type sequence of the M13, f1, φX174, P4, Lambda, P2, 186, Lambda-like, HK022, mEP237, HK97, HK629, HK630, mEP043, mEP213, mEP234, mEP390, mEP460, mEPx1, mEPx2, phi80, mEP234, T2, T4, T5, T7, RB49, phiX174, R17, PRD1 PI-like, P2-like, P22, P22-like, N15 and N15-like bacteriophages.

More preferably, the phage origin of replication is selected in the group consisting of phage origins of replication of M13, f1, φX174, P4, and Lambda.

In a particular embodiment, the phage origin of replication is the P4 origin of replication.

In a particular embodiment, the phage origin of replication is from Propionibacterium phages: BW-like phages such as Doucette, B22, E6, G4; BV-like phages such as Anatole, El, B3; BX-like phages such as PFR1 and PFR2; filamentous B5 phage; BU-like phages (Cutibacterium acnes phages). In a particular embodiment, the phage origin of replication is selected from the phage origins of replication disclosed in US applications US2022/135986 and US2022/135987.

### Conditional Origin of Replication

In a particular embodiment, the payload comprises a conditional origin of replication which is inactive in the targeted bacteria but is active in the production bacterial cell.

In the context of the invention, a “conditional origin of replication” refers to an origin of replication whose functionality may be controlled by the presence of a specific molecule.

In a particular embodiment, the conditional origin of replication is an origin of replication, the replication of which depends upon the presence of one or more given protein, peptid, RNA, nucleic acid, molecule or any combination thereof.

In a particular embodiment, the replication involving said origin of replication may further depend on a process, such as transcription, to activate said replication.

In the context of the invention, said conditional origin of replication is inactive in the targeted bacteria because of the absence of said given protein, peptid, RNA, nucleic acid, molecule or any combination thereof in said targeted bacteria.

In a particular embodiment, said conditional origin of replication is active in said production bacterial cell because said production bacterial cell expresses said given protein, peptid, RNA, nucleic acid, molecule or any combination thereof. In a particular embodiment, said protein, peptid, RNA nucleic acid, molecule or any combination thereof is expressed in trans in said production bacterial cell.

By “in trans” is meant herein that said protein, peptid, RNA, nucleic acid, molecule or any combination thereof is not encoded on the same nucleic acid molecule as the one comprising the origin of replication. In a particular embodiment, said protein, peptid, RNA, nucleic acid, molecule or any combination thereof is encoded on a chromosome or on a vector, in particular a plasmid. In a particular embodiment, said vector comprises an antibiotic resistance marker. In an alternative embodiment, said vector is devoid of antibiotic resistance marker.

Since said conditional origin of replication is inactive in the targeted bacteria because of the absence of said given protein, peptid, RNA, nucleic acid, molecule or any combination thereof in said targeted bacteria, said conditional origin of replication may be selected depending on the specific bacteria to be targeted.

The conditional origin of replication disclosed herein may originate from plasmids, bacteriophages or PICIs which preferably share the following characteristics: they contain in their origin of replication repeat sequences, or iterons, and they code for at least one protein interacting with said origin of replication (i.e. Rep, protein O, protein P, pri) which is specific to them.

By way of example, mention may be made of the conditional replication systems of the following plasmids and bacteriophages: RK2, R1, pSC101, F, Rts1, RSF1010, P1, P4, lambda, phi82, phi80.

In a particular embodiment, said conditional origin of replication is selected from the group consisting of the R6KA DNA replication origin and derivatives thereof, the IncPα oriV origin of replication and derivatives thereof, ColE1 origins of replication modified to be under an inducible promoter, and origins of replication from phage-inducible chromosomal islands (PICIs) and derivatives thereof.

In a particular embodiment, said conditional origin of replication is an origin of replication present in less than 50%, or less than 40%, less than 30%, less than 20%, less than 10% or less than 5% of the bacteria of the host subject's microbiome.

In another particular embodiment, said conditional origin of replication comprises or consists of a sequence less than 80% identical, in particular less than 70%, less than 60%, less than 50%, less than 40%, less than 30%, less than 20%, less than 10%, less than 5% or less than 1% identical to the sequences of the origins of replication of the bacteria of the host subject's microbiome, in particular of the bacteria representing more than 50%, more particularly more than 60%, more than 70%, more than 80%, more than 90% or more than 95% of the host subject's microbiome.

As used herein, the term “phage-inducible chromosomal islands” or “PICIs” refers to mobile genetic elements having a conserved gene organization, and encode a pair of divergent regulatory genes, including a PICT master repressor. Typically, in Gram-positive bacteria, left of rpr, and transcribed in the same direction, PICIs encode a small set of genes including an integrase (int) gene; right of rpr, and transcribed in the opposite direction, the PICIs encode an excision function (xis), and a replication module consisting of a primase homolog (pri) and optionally a replication initiator (rep), which are sometimes fused, followed by a replication origin (ori), next to these genes, and also transcribed in the same direction, PICIs encode genes involved in phage interference, and optionally, a terminase small subunit homolog (terS).

In a particular embodiment, said conditional origin of replication is an origin of replication derived from phage-inducible chromosomal islands (PICIs).

A particular conditional origin of replication has indeed been derived from PICIs.

It was shown that it is possible to derive novel conditionally replicative vectors, in particular based on the primase-helicase and origin of replication from PICIs. These origins may be relatively rare in target strains, and more advantageously the primase-ori pair may be unique for each PICT, significantly reducing the possibility of undesired recombination or payload spread events. They can further be modified to further limit recombination chances and remove restriction sites to bypass target bacteria defense systems.

In a particular embodiment, said conditional origin of replication is derived from the origin of replication from the PICT of the Escherichia coli strain CFT073, disclosed in Fillol-Salom et al. (2018) The ISME Journal 12:2114-2128.

In a particular embodiment, said conditional origin of replication is the primase on from the PICT of the Escherichia coli strain CFT073, typically of sequence SEQ ID NO: 1.

In another particular embodiment, said conditional origin of replication is the primase on from the PICT of the Escherichia coli strain CFT073, devoid of at least 1, at least 2, at least 3, at least 4, at least 5, at least 6, at least 7, at least 8, at least 9, at least 10, at least 11, at least 12, at least 13, at least 14, at least 15 or at least 16 restriction site(s) selected from the group consisting of GAAABCC, GCCGGC, RCCGGY, GCNGC, TWCANNNNNNTGG (SEQ ID NO: 2), TGGCCA, ACCYAC, YGGCCR, AGACC, GCWGC, GGGANGC, GKAGATD, GCCGGYYD, GGCYAC, RGCCGGYYD, and VGCCGGYBD.

In a particular embodiment, said conditional origin of replication is the primase on from the PICT of the Escherichia coli strain CFT073, devoid of the restriction site GAAABCC. Preferably, said conditional origin of replication is of sequence SEQ ID NO: 3.

In another particular embodiment, said conditional origin of replication is the primase on from the PICT of the Escherichia coli strain CFT073 devoid of the restriction sites GAAABCC, GCCGGC, RCCGGY, GCNGC, TWCANNNNNNTGG (SEQ ID NO: 2), TGGCCA, ACCYAC, YGGCCR, AGACC, GCWGC, GGGANGC, GKAGATD, GCCGGYYD, GGCYAC, RGCCGGYYD, and VGCCGGYBD. Preferably, said conditional origin of replication is of sequence SEQ ID NO: 4.

In a particular embodiment, wherein said origin of replication is derived from phage-inducible chromosomal islands (PICIs), said conditional origin of replication is active in said production bacterial cell because said production bacterial cell expresses a rep protein, in particular a primase-helicase, in particular a primase-helicase of sequence SEQ ID NO: 5, typically encoded by a nucleic acid comprising or consisting of the sequence SEQ ID NO: 6.

It was demonstrated that these specific conditional origins of replication were particularly compatible with lambda-based packaging, leading to sufficiently high titers (>10¹⁰/mL) required for microbiota-related applications.

Preferably, said production bacterial cell stably comprises said payload and is able to replicate said payload.

In a particular embodiment, when the conditional origin of replication of said payload is an origin of replication, the replication of which depends upon the presence of a given protein, peptid, nucleic acid, RNA, molecule or any combination thereof, said donor bacterial cell expresses said protein, peptid, nucleic acid, RNA, molecule or any combination thereof. Preferably, said protein, peptid, nucleic acid, RNA, molecule or any combination thereof is expressed in trans, as defined above.

In a particular embodiment, said production bacterial cell stably comprises a nucleic acid encoding said protein, peptid, nucleic acid, RNA, molecule or any combination thereof.

In a particular embodiment, when said origin of replication is derived from phage-inducible chromosomal islands (PICIs), said conditional origin of replication is active in said donor bacterial cell because said donor bacterial cell expresses a rep protein, in particular a primase-helicase, in particular a primase-helicase of sequence SEQ ID NO: 5.

In a particular embodiment, said production bacterial cell stably comprises a nucleic acid encoding said rep protein, in particular said primase-helicase, said nucleic acid typically comprising or consisting of the sequence SEQ ID NO: 6.

### Packaging Site

In a particular embodiment, said payload is a nucleic acid payload comprising a packaging site derived from said first type of bacteriophage.

By “packaging site” is meant herein the DNA sequence on the phage genome that is required for genome packaging into the virion. Host-specific bacteriophages (and their packaging sites) include but are not limited to SPP1 (SPP1 pac site), P1 (P1 pac site), Ti (Ti pac site), T7 (T7 concatamer junction), lambda (cos site), mu (mu pac site), P22 (P22 pac site), φ8 (φ8 pac site), Sf6 (Sf6 pac site), 149 (149 pac site), and A1122 (A1122-concatamer junction). For most bacteriophages, the packaging site is termed the pac site. In some cases, the packaging site is referred to as a concatamer junction (e.g. T7 concatamer junction). In every case, the packaging site is substantially in isolation from sequences naturally occurring adjacent thereto in the bacteriophage genome.

For some bacteriophages, the packaging site may be unknown. In these cases, pac sites can be determined by taking advantage of the property that plasmids containing a functional bacteriophage pac site are packaged. For example, the DNA sequences necessary for packaging of bacteriophage A were determined by incorporating small restriction fragments of the A phage genomic DNA into a plasmid (Hohn 1983 PNAS USA 80:7456-7460). Following introduction into an in vivo packaging strain, the efficiency of packaging/transduction was quantitatively assessed. Using a similar strategy, the pac sites for a number of bacteriophages have been determined: (Miwa 1982 Gene 20:267-279); Mu (Croenen et al. 1985 Virology 144:520-522); filamentous bacteriophages including f1, fd, M13, and Ike (Russel et al. 1989 J Virol 1989 63:3284-3295); P22 (Petri et al. 1990 Gene 88:47-55; Wu et al. 2002 Molec Microbiol 45:1631-1646); T7 (Chung et al. 1990 J Mol Biol 216:927-938), and T3 (Hashimoto et al. 1992 Virology 187:788-795).

In a particular embodiment, said packaging site is as disclosed in US applications US2022/135986 and US2022/135987, incorporated herein by reference.

### Other Components of the Payload

The payload used in the context of the invention is preferably devoid of antibiotic resistance marker.

Antibiotic resistance genes are well known in the art and include but are not limited to ampicillin resistance (Amp), chloramphenicol resistance (Cm), tetracycline resistance (Tet), kanamycin resistance (Kan), hygromycin resistance (Qiyg or hph genes), and zeomycin resistance (Zeo).

In a particular embodiment, the payload used in the context of the invention comprises an auxotrophic marker. Auxotrophic markers in bacteria have previously been described, for example, in U.S. Pat. Nos. 4,920,048, 5,691,185, 6,291,245, 6,413,768, and 6,752,994; U.S. Patent Publication No. 20050186666; Struhl et al. (1976) PNAS USA 73; 1471-1475; MacCormick et al., (1995) FEMS Microbiol. Lett. 127:105-109; Dickely et al. (1995) Mol. Microbiol. 15:839-847; Sorensen et al. (2000) Appl. Environ. Microbiol 66:1253-1258; and Fiedler & Skerra (2001) Gene 274: 111 118, and typically include DapA and ThyA. In a particular embodiment, said auxotrophic marker is ThyA.

In a particular embodiment, said payload does not comprise any restriction site recognized by restriction enzymes which are frequently encoded by said targeted bacterial cell. In another particular embodiment, said payload comprises no more than 40, 30, 20, 10, 9, 8, 7, 6, 5, 4, 3, 2 or 1 restriction site(s) recognized by restriction enzymes which are frequently encoded by said targeted bacterial cell or a population or a group of targeted bacterial cell(s).

As used herein, the terms “restriction site” and “restriction enzyme site” are equivalent and refer to locations on a nucleic acid containing specific sequences of nucleotides, which are recognized by restriction enzymes. In particular, the nucleic acid comprises specific sequences which are bound and cleaved by restriction enzymes. Restriction sites are generally palindromic sequences of 4-8 base pairs in length. More precisely, the restriction site refers to a particular sequence and a modification state, so as to be bound and cleaved by restriction enzymes. In particular, it refers to a particular unmodified sequence, so as to be bound and cleaved by restriction enzymes. Especially the sequence is not methylated, hydroxymethylated and glucosyl-hydroxymethylated. In this context, the restriction enzyme is of type I, II or III. Alternatively, it may refer to a particular modified sequence, so as to be bound and cleaved by restriction enzymes, for instance a methylated, hydroxymethylated and glucosyl-hydroxymethylated DNA. In this context, the restriction enzyme is of type IV.

As used herein, “recognized by” with respect to a restriction site and a restriction enzyme means that the restriction site is cleaved by the restriction enzyme.

In a restriction site sequence N means that the nucleotide can be A, C, G or T; B means that the nucleotide can be C, G or T; Y means that the nucleotide can be C or T; W means that the nucleotide can be A or T; R means that the nucleotide can be A or G; and D means A, G or T.

As used herein, the terms “restriction enzyme” and “restriction endonuclease” are equivalent and refer to an enzyme that cuts nucleic acids at or near restriction sites. Restriction enzymes are commonly classified into four types (types I to type IV). The REBASE database allow to list the restriction sites that a given bacterium can recognize according to the restriction enzymes that it expresses.

By “frequent” or “frequently” in a group of bacteria of interest is meant that at least 10, 20, 30, 40, 50, 60, 70, 75, 80, 85, 90, 95 or 99% of the bacteria of the group encode the restriction enzyme.

The payload according to the invention preferably comprises no more than 100 restriction sites. In a preferred embodiment, the payload according to the invention comprises no more than 10 restriction sites. In a most preferred embodiment, the payload according to the invention does not comprise any restriction site.

### Targeted Bacteria

The bacteria targeted by the phage particles or phage-derived delivery particles of the invention can be any bacteria present in a mammal organism, a plant or in the environment. It can be any commensal, symbiotic or pathogenic bacteria of the microbiota or microbiome.

A microbiome may comprise a variety of endogenous bacterial species, any of which may be targeted in accordance with the present disclosure. In some embodiments, the genus and/or species of targeted endogenous bacterial cells may depend on the first type of bacteriophage as defined in the section “Bacteriophage and gene derived from a bacteriophage” above. For example, some bacteriophages exhibit tropism for, or preferentially target, specific host species of bacteria. Other bacteriophages do not exhibit such tropism and may be used to target a number of different genus and/or species of endogenous bacterial cells.

Examples of bacterial cells include, without limitation, cells from bacteria of the genus Yersinia spp., Escherichia spp., Klebsiella spp., Acinetobacter spp., Bordetella spp., Neisseria spp., Aeromonas spp., Francisella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., Erysipelothrix spp., Salmonella spp., Streptomyces spp., Streptococcus spp., Staphylococcus spp., Bacteroides spp., Prevotella spp., Clostridium spp., Bifidobacterium spp., Clostridium spp., Brevibacterium spp., Lactococcus spp., Leuconostoc spp., Actinobacillus spp., Selnomonas spp., Shigella spp., Zymonas spp., Mycoplasma spp., Treponema spp., Leuconostoc spp., Corynebacterium spp., Enterococcus spp., Enterobacter spp., Pyrococcus spp., Serratia spp., Morganella spp., Parvimonas spp., Fusobacterium spp., Actinomyces spp., Porphyromonas spp., Micrococcus spp., Bartonella spp., Borrelia spp., Brucelia spp., Campylobacter spp., Chlamydophilia spp., Cutibacterium spp., Propionibacterium spp., Gardnerella spp., Ehrlichia spp., Haemophilus spp., Leptospira spp., Listeria spp., Mycoplasma spp., Nocardia spp., Rickettsia spp., Ureaplasma spp., Lactobacillus spp., Faecalibacterium spp., Ruminococcus spp. and a mixture thereof.

Thus, phage particles, phage delivery particles and/or phages may target (e.g., specifically target) a bacterial cell from any one or more of the foregoing genus of bacteria in particular to specifically deliver the payload according to the invention.

Preferably, the targeted bacteria can be selected from the group consisting of Yersinia spp., Escherichia spp., Klebsiella spp., Acinetobacter spp., Pseudomonas spp., Helicobacter spp., Vibrio spp, Salmonella spp., Streptococcus spp., Staphylococcus spp., Bacteroides spp., Clostridium spp., Shigella spp., Enterococcus spp., Enterobacter spp., Listeria spp., Cutibacterium spp., Propionibacterium spp., Fusobacterium spp., Porphyromonas spp. and Gardnerella spp.

In some embodiments, the targeted bacteria are anaerobic bacterial cells (e.g., cells that do not require oxygen for growth). Anaerobic bacterial cells include facultative anaerobic cells such as but not limited to Escherichia coli, Shewanella oneidensi, Gardnerella vaginalis and Listeria. Anaerobic bacterial cells also include obligate anaerobic cells such as, for example, Bacteroides, Clostridium, Cutibacterium, Propionibacterium, Fusobacterium and Porphyromonas species. In humans, anaerobic bacteria are most commonly found in the gastrointestinal tract. In some particular embodiments, the targeted bacteria are thus bacteria most commonly found in the gastrointestinal tract. Bacteriophages used for preparing the hybrid helper phage, and then the phage particles, phage delivery vehicles and/or phages, may target (e.g., to specifically target) anaerobic bacterial cells according to their specific spectra known by the person skilled in the art to specifically deliver the plasmid.

In some embodiments, the targeted bacterial cells are, without limitation, Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Enterococcus faecalis, Bacillus coagulans, Bacillus cereus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Streptomyces phaechromogenes, Streptomyces ghanaenis, Klebsiella pneumoniae, Enterobacter cloacae, Enterobacter aerogenes, Serratia marcescens, Morganella morganii, Citrobacter freundii, Propionibacterium freudenreichii, Pseudomonas aeruginosa, Parvimonas micra, Prevotella intermedia, Fusobacterium nucleatum, Prevotella nigrescens, Actinomyces israelii, Porphyromonas endodontalis, Porphyromonas gingivalis Micrococcus luteus, Bacillus megaterium, Aeromonas hydrophila, Aeromonas caviae, Bacillus anthracis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Campylobacter coli, Campylobacter fetus, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Cutibacterium acnes (formerly Propionibacterium acnes), Ehrlichia canis, Ehrlichia chaffeensis, Enterococcus faecium, Francisella tularensis, Haemophilus influenza, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Nocardia asteroids, Rickettsia rickettsia, Salmonella enteritidis, Salmonella typhi, Salmonella paratyphi, Salmonella typhimurium, Shigella flexnerii, Shigella dysenteriae, Staphylococcus saprophyticus, Streptococcus pneumoniae, Streptococcus pyogenes, Gardnerella vaginalis, Streptococcus viridans, Treponema pallidum, Urea plasma urealyticum, Vibrio cholera, Vibrio parahaemolyticus, Yersinia pestis, Yersinia enterocolitica, Yersinia pseudotuberculosis, Actinobacter baumanii, Pseudomonas aeruginosa, and a mixture thereof, preferably the bacteria of interest are selected from the group consisting of Escherichia coli, Enterococcus faecium, Staphylococcus aureus, Klebsiella pneumoniae, Acinetobacter baumanii, Pseudomonas aeruginosa, Enterobacter cloacae, and Enterobacter aerogenes, and a mixture thereof.

In some embodiments, the targeted bacterial cells are, without limitation, Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter, Weisella, Clostridium, Bacteroides, Ruminococcus, Faecalibacterium, Treponema, Phascolarctobacterium, Megasphaera, Faecalibacterium, Bifidobacterium, Lactobacillus, Sutterella, and/or Prevotella.

In other embodiments, the targeted bacteria cells are, without limitation, Achromobacter xylosoxidans, Acidaminococcus fermentans, Acidaminococcus intestini, Acidaminococcus sp., Acinetobacter baumannii, Acinetobacter junii, Acinetobacter lwoffii, Actinobacillus capsulatus, Actinomyces naeslundii, Actinomyces neuii, Actinomyces odontolyticus, Actinomyces radingae, Adlercreutzia equolifaciens, Aeromicrobium massiliense, Aggregatibacter actinomycetemcomitans, Akkermansia muciniphila, Aliagarivorans marinus, Alistipes finegoldii, Alistipes indistinctus, Alistipes inops, Alistipes onderdonkii, Alistipes putredinis, Alistipes senegalensis, Alistipes shahii, Alistipes timonensis, Alloscardovia omnicolens, Anaerobacter polyendosporus, Anaerobaculum hydrogeniformans, Anaerococcus hydrogenalis, Anaerococcus prevotii, Anaerococcus senegalensis, Anaerofustis stercorihominis, Anaerostipes caccae, Anaerostipes hadrus, Anaerotruncus colihominis, Aneurinibacillus aneurinilyticus, Bacillus licheniformis, Bacillus massilioanorexius, Bacillus massiliosenegalensis, Bacillus simplex, Bacillus smithii, Bacillus subtilis, Bacillus thuringiensis, Bacillus timonensis, Bacteroides xylanisolvens, Bacteroides acidifaciens, Bacteroides caccae, Bacteroides capillosus, Bacteroides cellulosilyticus, Bacteroides clarus, Bacteroides coprocola, Bacteroides coprophilus, Bacteroides dorei, Bacteroides eggerthii, Bacteroides faecis, Bacteroides finegoldii, Bacteroides fluxus, Bacteroides fragilis, Bacteroides gallinarum, Bacteroides intestinalis, Bacteroides nordii, Bacteroides oleiciplenus, Bacteroides ovatus, Bacteroides pectinophilus, Bacteroides plebeius, Bacteroides salanitronis, Bacteroides salyersiae, Bacteroides sp., Bacteroides stercoris, Bacteroides thetaiota omicron, Bacteroides uniformis, Bacteroides vulgatus, Bacteroides xylanisolvens, Bacteroides pectinophilus ATCC, Barnesiella intestinihominis, Bavariicoccus seileri, Bifidobacterium adolescentis, Bifidobacterium angulatum, Bifidobacterium animalis, Bifidobacterium bifidum, Bifidobacterium breve, Bifidobacterium catenulatum, Bifidobacterium dentium, Bifidobacterium gallicum, Bifidobacterium Ion gum, Bifidobacterium pseudocatenulatum, Bifidobacterium stercoris, Bilophila wadsworthia, Blautia faecis, Blautia hansenii, Blautia hydrogenotrophica, Blautia luti, Blautia obeum, Blautia producta, Blautia wexlerae, Brachymonas chironomi, Brevibacterium senegalense, Bryantella formatexigens, butyrate-producing bacterium, Butyricicoccus pullicaecorum, Butyricimonas virosa, Butyrivibrio crossotus, Butyrivibrio fibrisolvens, Caldicoprobacter faecalis, Campylobacter concisus, Campylobacter jejuni, Campylobacter upsaliensis, Catenibacterium mitsuokai, Cedecea davisae, Cellulomonas massiliensis, Cetobacterium somerae, Citrobacter braakii, Citrobacter freundii, Citrobacter pasteurii, Citrobacter sp., Citrobacter youngae, Cloacibacillus evryensis, Clostridiales bacterium, Clostridioides difficile, Clostridium asparagiforme, Clostridium bartlettii, Clostridium boliviensis, Clostridium bolteae, Clostridium hathewayi, Clostridium hiranonis, Clostridium hylemonae, Clostridium leptum, Clostridium methylpentosum, Clostridium nexile, Clostridium orbiscindens, Clostridium ramosum, Clostridium scindens, Clostridium sp, Clostridium sp., Clostridium spiroforme, Clostridium sporogenes, Clostridium symbiosum, Collinsella aerofaciens, Collinsella intestinalis, Collinsella stercoris, Collinsella tanakaei, Coprobacillus cateniformis, Coprobacter fastidiosus, Coprococcus catus, Coprococcus comes, Coprococcus eutactus, Corynebacterium ammonia genes, Corynebacterium amycolatum, Corynebacterium pseudodiphtheriticum, Cutibacterium acnes, Dermabacter hominis, Desulfitobacterium hafniense, Desulfovibrio fairfieldensis, Desulfovibrio piger, Dialister succinatiphilus, Dielma fastidiosa, Dorea formicigenerans, Dorea longicatena, Dysgonomonas capnocytophagoides, Dysgonomonas gadei, Dysgonomonas mossii, Edwardsiella tarda, Eggerthella lenta, Eisenbergiella tayi, Enorma massiliensis, Enterobacter aerogenes, Enterobacter asburiae, Enterobacter cancerogenus, Enterobacter cloacae, Enterobacter massiliensis, Enterococcus casseliflavus, Enterococcus durans, Enterococcus faecalis, Enterococcus faecium, Enterococcus flavescens, Enterococcus gallinarum, Enterococcus sp., Enterovibrio nigricans, Erysipelatoclostridium ramosum, Escherichia coli, Escherichia sp., Eubacterium biforme, Eubacterium dolichum, Eubacterium hallii, Eubacterium limosum, Eubacterium ramulus, Eubacterium rectale, Eubacterium siraeum, Eubacterium ventriosum, Exiguobacterium marinum, Exiguobacterium undae, Faecalibacterium cf, Faecalibacterium prausnitzii, Faecalitalea cylindroides, Ferrimonas balearica, Finegoldia magna, Flavobacterium daejeonense, Flavonifractor plautii, Fusicatenibacter saccharivorans, Fusobacterium gonidiaformans, Fusobacterium mortiferum, Fusobacterium necrophorum, Fusobacterium nucleatum, Fusobacterium periodonticum, Fusobacterium sp., Fusobacterium ulcerans, Fusobacterium varium, Gallibacterium anatis, Gemmiger formicilis, Gordonibacter pamelaeae, Hafnia alvei, Helicobacter bilis, Helicobacter bills, Helicobacter canadensis, Helicobacter canis, Helicobacter cinaedi, Helicobacter macacae, Helicobacter pametensis, Helicobacter pullorum, Helicobacter pylori, Helicobacter rodentium, Helicobacter win ghamensis, Herbaspirillum massiliense, Holdemanella biformis, Holdemania fdiformis, Holdemania filiformis, Holdemania massiliensis, Holdemania filiformis, Hungatella hathewayi, Intestinibacter bartlettii, Intestinimonas butyriciproducens, Klebsiella oxytoca, Klebsiella pneumoniae, Kurthia massiliensis, Lachnospira pectinoschiza, Lactobacillus acidophilus, Lactobacillus amylolyticus, Lactobacillus animalis, Lactobacillus antri, Lactobacillus brevis, Lactobacillus buchneri, Lactobacillus casei, Lactobacillus curvatus, Lactobacillus delbrueckii, Lactobacillus fermentum, Lactobacillus gasseri, Lactobacillus helveticus, Lactobacillus hilgardii, Lactobacillus iners, Lactobacillus intestinalis, Lactobacillus johnsonii, Lactobacillus murinus, Lactobacillus paracasei, Lactobacillus plantarum, Lactobacillus reuteri, Lactobacillus rhamnosus, Lactobacillus ruminis, Lactobacillus sakei, Lactobacillus salivarius, Lactobacillus ultunensis, Lactobacillus vaginalis, Lactobacillus plantarum subsp., Leuconostoc mesenteroides, Leuconostoc pseudomesenteroides, Listeria grayi, Listeria innocua, Mannheimia granulomatis, Marvinbryantia formatexigens, Megamonas funiformis, Megamonas hypermegale, Methanobrevibacter smithii, Methanobrevibacter smithiiFl, Micrococcus luteus, Microvirgula aerodenitrificans, Mitsuokella jalaludinii, Mitsuokella multacida, Mollicutes bacterium, Murimonas intestini, Neisseria macacae, Nitriliruptor alkaliphilus, Oceanobacillus massiliensis, Odoribacter laneus, Odoribacter splanchnicus, Ornithobacterium rhinotracheale, Oxalobacter formigenes, Paenibacillus barengoltzii, Paenibacillus chitinolyticus, Paenibacillus lautus, Paenibacillus motobuensis, Paenibacillus senegalensis, Paenisporosarcina quisquiliarum, Parabacteroides distasonis, Parabacteroides goldsteinii, Parabacteroides gordonii, Parabacteroides johnsonii, Parabacteroides merdae, Paraprevotella xylaniphila, Parasutterella excrementihominis, Parvimonas micra, Pediococcus acidilactici, Peptoclostridium difficile, Peptoniphilus harei, Peptoniphilus obesi, Peptoniphilus senegalensis, Peptoniphilus timonensis, Phascolarctobacterium succinatutens, Porphyromonas asaccharolytica, Porphyromonas uenonis, Prevotella baroniae, Prevotella bivia, Prevotella copri, Prevotella dentalis, Prevotella micans, Prevotella multisaccharivorax, Prevotella oralis, Prevotella salivae, Prevotella stercorea, Prevotella veroralis, Propionibacterium acnes, Propionibacterium avidum, Propionibacterium freudenreichii, Propionimicrobium lymphophilum, Proteus mirabilis, Proteus penneri A TCC, Providencia alcalifaciens, Providencia rettgeri, Providencia rustigianii, Providencia stuartii, Pseudoflavonifractor capillosus, Pseudomonas aeruginosa, Pseudomonas luteola, Ralstonia pickettii, Rheinheimera perlucida, Rheinheimera texasensis, Riemerella columbina, Romboutsia lituseburensis, Roseburia faecis, Roseburia intestinalis, Roseburia inulinivorans, Ruminococcus bicirculans, Ruminococcus bromii, Ruminococcus callidus, Ruminococcus champanellensis, Ruminococcus faecis, Ruminococcus gnavus, Ruminococcus lactaris, Ruminococcus obeum, Ruminococcus sp, Ruminococcus sp., Ruminococcus torques, Sarcina ventriculi, Sellimonas intestinalis, Senegalimassilia anaerobia, Shigella sonnei, Slackia piriformis, Staphylococcus epidermidis, Staphylococcus lentus, Staphylococcus nepalensis, Staphylococcus pseudintermedius, Staphylococcus xylosus, Stenotrophomonas maltophilia, Streptococcus agalactiae, Streptococcus anginosus, Streptococcus australis, Streptococcus caballi, Streptococcus castoreus, Streptococcus didelphis, Streptococcus equinus, Streptococcus gordonii, Streptococcus henryi, Streptococcus hyovaginalis, Streptococcus infantarius, Streptococcus infantis, Streptococcus lutetiensis, Streptococcus merionis, Streptococcus mitis, Streptococcus mutans, Streptococcus oralis, Streptococcus ovis, Streptococcus parasanguinis, Streptococcus plurextorum, Streptococcus porci, Streptococcus pyogenes, Streptococcus salivarius, Streptococcus sobrinus, Streptococcus thermophilus, Streptococcus thoraltensis, Streptomyces albus, Subdoligranulum variabile, Succinatimonas hippei, Sutterella parvirubra, Sutterella wadsworthensis, Terrisporobacter glycolicus, Terrisporobacter mayombei, Thalassobacillus devorans, Timonella senegalensis, Turicibacter sanguinis, unknown sp, unknown sp., Varibaculum cambriense, Veillonella atypica, Veillonela dispar, Veillonella parvula, Vibrio cincinnatiensis, Virgibacillus salexigens and/or Weissella paramesenteroides.

In other embodiments, the targeted bacteria cells are those commonly found on the skin microbiota and are without limitation Acetobacter farinalis, Acetobacter malorum, Acetobacter orleanensis, Acetobacter sicerae, Achromobacter anxifer, Achromobacter denitrificans, Achromobacter marplatensis, Achromobacter spanius, Achromobacter xylosoxidans subsp. xylosoxidans, Acidovorax konjaci, Acidovorax radicis, Acinetobacter johnsonii, Actinomadura citrea, Actinomadura coerulea, Actinomadura fibrosa, Actinomadura fulvescens, Actinomadura jiaoheensis, Actinomadura luteofluorescens, Actinomadura mexicana, Actinomadura nitritigenes, Actinomadura verrucosospora, Actinomadura yumaensis, Actinomyces odontolyticus, Actinomycetospora atypica, Actinomycetospora corticicola, Actinomycetospora rhizophila, Actinomycetospora rishiriensis, Aeromonas australiensis, Aeromonas bestiarum, Aeromonas bivalvium, Aeromonas encheleia, Aeromonas eucrenophila, Aeromonas hydrophila subsp. hydrophila, Aeromonas piscicola, Aeromonas popoffii, Aeromonas rivuli, Aeromonas salmonicida subsp. pectinolytica, Aeromonas salmonicida subsp. smithia, Amaricoccus kaplicensis, Amaricoccus veronensis, Aminobacter aganoensis, Aminobacter ciceronei, Aminobacter lissarensis, Aminobacter niigataensis, Ancylobacter polymorphus, Anoxybacillus flavithermus subsp. yunnanensis, Aquamicrobium aerolatum, Archangium gephyra, Archangium gephyra, Archangium minus, Archangium violaceum, Arthrobacter viscosus, Bacillus anthracis, Bacillus australimaris, Bacillus drentensis, Bacillus mycoides, Bacillus pseudomycoides, Bacillus pumilus, Bacillus safensis, Bacillus vallismortis, Bosea thiooxidans, Bradyrhizobium huanghuaihaiense, Bradyrhizobium japonicum, Brevundimonas aurantiaca, Brevundimonas intermedia, Burkholderia aspalathi, Burkholderia choica, Burkholderia cordobensis, Burkholderia diffusa, Burkholderia insulsa, Burkholderia rhynchosiae, Burkholderia terrestris, Burkholderia udeis, Buttiauxella gaviniae, Caenimonas terrae, Capnocytophaga gingivalis, Chitinophaga dinghuensis, Chryseobacterium gleum, Chryseobacterium greenlandense, Chryseobacterium jejuense, Chryseobacterium piscium, Chryseobacterium sediminis, Chryseobacterium tructae, Chryseobacterium ureilyticum, Chryseobacterium vietnamense, Corynebacterium accolens, Corynebacterium afermentans subsp. lipophilum, Corynebacterium minutissimum, Corynebacterium sundsvallense, Cupriavidus metallidurans, Cupriavidus nantongensis, Cupriavidus necator, Cupriavidus pampae, Cupriavidus yeoncheonensis, Curtobacterium flaccumfaciens, Devosia epidermidihirudinis, Devosia riboflavina, Devosia riboflavina, Diaphorobacter oryzae, Dietzia psychralcaliphila, Ensifer adhaerens, Ensifer americanus, Enterococcus malodoratus, Enterococcus pseudoavium, Enterococcus viikkiensis, Enterococcus xiangfangensis, Erwinia rhapontici, Falsirhodobacter halotolerans, Flavobacterium araucananum, Flavobacterium frigidimaris, Gluconobacter frateurii, Gluconobacter thailandicus, Gordonia alkanivorans, Halomonas aquamarina, Halomonas axialensis, Halomonas meridiana, Halomonas olivaria, Halomonas songnenensis, Halomonas variabilis, Herbaspirillum chlorophenolicum, Herbaspirillum frisingense, Herbaspirillum hiltneri, Herbaspirillum huttiense subsp. putei, Herbaspirillum lusitanum, Herminiimonas fonticola, Hydrogenophaga intermedia, Hydrogenophaga pseudoflava, Klebsiella oxytoca, Kosakonia sacchari, Lactobacillus delbrueckii subsp. bulgaricus, Lactobacillus modestisalitolerans, Lactobacillus plantarum subsp. argentoratensis, Lactobacillus xiangfangensis, Lechevalieria roselyniae, Lentzea albida, Lentzea californiensis, Leuconostoc carnosum, Leuconostoc citreum, Leuconostoc gelidum subsp. gasicomitatum, Leuconostoc mesenteroides subsp. suionicum, Luteimonas aestuarii, Lysobacter antibioticus, Lysobacter koreensis, Lysobacter oryzae, Magnetospirillum moscoviense, Marinomonas alcarazii, Marinomonas primoryensis, Massilia aurea, Massilia jejuensis, Massilia kyonggiensis, Massilia timonae, Mesorhizobium acaciae, Mesorhizobium gingshengii, Mesorhizobium shonense, Methylobacterium haplocladii, Methylobacterium platani, Methylobacterium pseudosasicola, Methylobacterium zatmanii, Microbacterium oxydan, Micromonospora chaiyaphumensis, Micromonospora chalcea, Micromonospora citrea, Micromonospora coxensis, Micromonospora echinofusca, Micromonospora halophytica, Micromonospora kangleipakensis, Micromonospora maritima, Micromonospora nigra, Micromonospora purpureochromogene, Micromonospora rhizosphaerae, Micromonospora saelicesensis, Microvirga subterranea, Microvirga zambiensis, Mycobacterium alvei, Mycobacterium avium subsp. silvaticum, Mycobacterium colombiense, Mycobacterium conceptionense, Mycobacterium conceptionense, Mycobacterium farcinogenes, Mycobacterium fortuitum subsp. fortuitum, Mycobacterium goodii, Mycobacterium insubricum, Mycobacterium Ilatzerense, Mycobacterium neoaurum, Mycobacterium neworleansense, Mycobacterium obuense, Mycobacterium peregrinum, Mycobacterium saopaulense, Mycobacterium septicum, Mycobacterium setense, Mycobacterium smegmatis, Neisseria subflava, Nocardia lijiangensis, Nocardia thailandica, Novosphingobium barchaimii, Novosphingobium lindaniclasticum, Novosphingobium lindaniclasticum, Novosphingobium mathurense, Ochrobactrum pseudogrignonense, Oxalicibacterium solurbis, Paraburkholderia glathei, Paraburkholderia humi, Paraburkholderia phenazinium, Paraburkholderia phytofirmans, Paraburkholderia sordidicola, Paraburkholderia terricola, Paraburkholderia xenovorans, Paracoccus laeviglucosivorans, Patulibacter ginsengiterrae, Polymorphospora rubra, Porphyrobacter colymbi, Prevotella jejuni, Prevotella melaninogenica, Propionibacterium acnes subsp. elongatum, Proteus vulgaris, Providencia rustigianii, Pseudoalteromonas agarivorans, Pseudoalteromonas atlantica, Pseudoalteromonas paragorgicola, Pseudomonas asplenii, Pseudomonas asuensis, Pseudomonas benzenivorans, Pseudomonas cannabina, Pseudomonas cissicola, Pseudomonas congelans, Pseudomonas costantinii, Pseudomonas ficuserectae, Pseudomonas frederiksbergensis, Pseudomonas graminis, Pseudomonas jessenii, Pseudomonas koreensis, Pseudomonas koreensis, Pseudomonas kunmingensis, Pseudomonas marginalis, Pseudomonas mucidolens, Pseudomonas panacis, Pseudomonas plecoglossicida, Pseudomonas poae, Pseudomonas pseudoalcaligenes, Pseudomonas putida, Pseudomonas reinekei, Pseudomonas rhizosphaerae, Pseudomonas seleniipraecipitans, Pseudomonas umsongensis, Pseudomonas zhaodongensis, Pseudonocardia alaniniphila, Pseudonocardia ammonioxydans, Pseudonocardia autotrophica, Pseudonocardia kongjuensis, Pseudonocardia yunnanensis, Pseudorhodoferax soli, Pseudoxanthomonas daejeonensis, Pseudoxanthomonas indica, Pseudoxanthomonas kaohsiungensis, Psychrobacter aquaticus, Psychrobacter arcticus, Psychrobacter celer, Psychrobacter marincola, Psychrobacter nivimaris, Psychrobacter okhotskensis, Psychrobacter okhotskensis, Psychrobacter piscatorii, Psychrobacterpulmonis, Ramlibacterginsenosidimutans, Rheinheimera japonica, Rheinheimera muenzenbergensis, Rheinheimera soli, Rheinheimera tangshanensis, Rheinheimera texasensis, Rheinheimera tilapiae, Rhizobium alamii, Rhizobium azibense, Rhizobium binae, Rhizobium daejeonense, Rhizobium endophyticum, Rhizobium etli, Rhizobium fabae, Rhizobium freirei, Rhizobium gallicum, Rhizobium loessense, Rhizobium sophoriradicis, Rhizobium taibaishanense, Rhizobium vallis, Rhizobium vignae, Rhizobium vignae, Rhizobium yanglingense, Rhodococcus baikonurensis, Rhodococcus enclensis, Rhodoferax saidenbachensis, Rickettsia canadensis, Rickettsia heilongjiangensis, Rickettsia honei, Rickettsia raoultii, Roseateles aquatilis, Roseateles aquatilis, Salmonella enterica subsp. salamae, Serratia ficaria, Serratia myotis, Serratia vespertilionis, Shewanella aestuarii, Shewanella decolorationis, Sphingobium amiense, Sphingobium baderi, Sphingobium barthaii, Sphingobium chlorophenolicum, Sphingobium cupriresistens, Sphingobium czechense, Sphingobium fuliginis, Sphingobium indicum, Sphingobium indicum, Sphingobium japonicum, Sphingobium lactosutens, Sphingomonas dokdonensis, Sphingomonas pseudosanguinis, Sphingopyxis chilensis, Sphingopyxis fribergensis, Sphingopyxis granuli, Sphingopyxis indica, Sphingopyxis witflariensis, Staphylococcus agnetis, Staphylococcus aureus subsp. aureus, Staphylococcus epidermidis, Staphylococcus hominis subsp. novobiosepticus, Staphylococcus nepalensis, Staphylococcus saprophyticus subsp. bovis, Staphylococcus sciuri subsp. carnaticus, Streptomyces caeruleatus, Streptomyces canarius, Streptomyces capoamus, Streptomyces ciscaucasicus, Streptomyces griseorubiginosus, Streptomyces olivaceoviridis, Streptomyces panaciradicis, Streptomyces phaeopurpureus, Streptomyces pseudo venezuelae, Streptomyces resistomycificus, Tianweitania sediminis, Tsukamurella paurometabola, Variovorax guangxiensis, Vogesella alkaliphila, Xanthomonas arboricola, Xanthomonas axonopodis, Xanthomonas cassavae, Xanthomonas cucurbitae, Xanthomonas cynarae, Xanthomonas euvesicatoria, Xanthomonas fragariae, Xanthomonas gardneri, Xanthomonas perforans, Xanthomonas pisi, Xanthomonas populi, Xanthomonas vasicola, Xenophilus aerolatus, Yersinia nurmii, Abiotrophia defectiva, Acidocella aminolytica, Acinetobacter guangdongensis, Acinetobacter parvus, Acinetobacter radioresistens, Acinetobacter soli, Acinetobacter variabilis, Actinomyces cardiffensis, Actinomyces dentalis, Actinomyces europaeus, Actinomyces gerencseriae, Actinomyces graevenitzii, Actinomyces haliotis, Actinomyces johnsonii, Actinomyces massiliensis, Actinomyces meyeri, Actinomyces meyeri, Actinomyces naeslundii, Actinomyces neuii subsp. anitratus, Actinomyces odontolyticus, Actinomyces oris, Actinomyces turicensis, Actinomycetospora corticicola, Actinotignum schaalii, Aerococcus christensenii, Aerococcus urinae, Aeromicrobium flavum, Aeromicrobium massiliense, Aeromicrobium tamlense, Aeromonas sharmana, Aggregatibacter aphrophilus, Aggregatibacter segnis, Agrococcus baldri, Albibacter methylovorans, Alcaligenes faecalis subsp. faecalis, Algoriphagus ratkowskyi, Alkalibacterium olivapovliticus, Alkalibacterium pelagium, Alkalibacterium pelagium, Alloprevotella rava, Alsobacter metallidurans, Amaricoccus kaplicensis, Amaricoccus veronensis, Anaerococcus hydrogenalis, Anaerococcus lactolyticus, Anaerococcus murdochii, Anaerococcus octavius, Anaerococcus prevotii, Anaerococcus vaginalis, Aquabacterium citratiphilum, Aquabacterium olei, Aquabacterium olei, Aquabacterium parvum, Aquincola tertiaricarbonis, Arcobacter venerupis, Arsenicicoccus bolidensis, Arthrobacter russicus, Asticcacaulis excentricus, Atopobium deltae, Atopobium parvulum, Atopobium rimae, Atopobium vaginae, Aureimonas altamirensis, Aureimonas rubiginis, Azospira oryzae, Azospirillum oryzae, Bacillus circulans, Bacillus drentensis, Bacillus fastidiosus, Bacillus lehensis, Bacillus oceanisediminis, Bacillus rhizosphaerae, Bacteriovorax stolpii, Bacteroides coagulans, Bacteroides dorei, Bacteroides fragilis, Bacteroides ovatus, Bacteroides stercoris, Bacteroides uniformis, Bacteroides vulgatus, Bdellovibrio bacteriovorus, Bdellovibrio exovorus, Belnapia moabensis, Belnapia soli, Blautia hansenii, Blautia obeum, Blautia wexlerae, Bosea lathyri, Brachybacterium fresconis, Brachybacterium muris, Brevibacterium ammoniilyticum, Brevibacterium casei, Brevibacterium epidermidis, Brevibacterium iodinum, Brevibacterium luteolum, Brevibacterium paucivorans, Brevibacterium pityocampae, Brevibacterium sanguinis, Brevundimonas albigilva, Brevundimonas diminuta, Brevundimonas vancanneytii, Caenimonas terrae, Calidifontibacter indicus, Campylobacter concisus, Campylobacter gracilis, Campylobacter hominis, Campylobacter rectus, Campylobacter showae, Campylobacter ureolyticus, Capnocytophaga gingivalis, Capnocytophaga leadbetteri, Capnocytophaga ochracea, Capnocytophaga sputigena, Cardiobacterium hominis, Cardiobacterium valvarum, Carnobacterium divergens, Catonella morbi, Caulobacter henricii, Cavicella subterranea, Cellulomonas xylanilytica, Cellvibrio vulgaris, Chitinimonas taiwanensis, Chryseobacterium arachidis, Chryseobacterium daecheongense, Chryseobacterium formosense, Chryseobacterium formosense, Chryseobacterium greenlandense, Chryseobacterium indologenes, Chryseobacterium piscium, Chryseobacterium rigui, Chryseobacterium solani, Chryseobacterium taklimakanense, Chryseobacterium ureilyticum, Chryseobacterium ureilyticum, Chryseobacterium zeae, Chryseomicrobium aureum, Cloacibacterium haliotis, Cloacibacterium normanense, Cloacibacterium normanense, Collinsella aerofaciens, Comamonas denitrificans, Comamonas terrigena, Corynebacterium accolens, Corynebacterium afermentans subsp. lipophilum, Corynebacterium ammonia genes, Corynebacterium amycolatum, Corynebacterium aurimucosum, Corynebacterium aurimucosum, Corynebacterium coyleae, Corynebacterium durum, Corynebacterium freiburgense, Corynebacterium glaucum, Corynebacterium glyciniphilum, Corynebacterium imitans, Corynebacterium jeikeium, Corynebacterium jeikeium, Corynebacterium kroppenstedtii, Corynebacterium lipophiloflavum, Corynebacterium massiliense, Corynebacterium mastitidis, Corynebacterium matruchotii, Corynebacterium minutissimum, Corynebacterium mucifaciens, Corynebacterium mustelae, Corynebacterium mycetoides, Corynebacterium pyruviciproducens, Corynebacterium simulans, Corynebacterium singulare, Corynebacterium sputi, Corynebacterium suicordis, Corynebacterium tuberculostearicum, Corynebacterium tuberculostearicum, Corynebacterium ureicelerivorans, Corynebacterium variabile, Couchioplanes caeruleus subsp. caeruleus, Cupriavidus metallidurans, Curtobacterium herbarum, Dechloromonas agitata, Deinococcus actinosclerus, Deinococcus antarcticus, Deinococcus caeni, Deinococcus ficus, Deinococcus geothermalis, Deinococcus radiodurans, Deinococcus wulumuqiensis, Deinococcus xinjiangensis, Dermabacter hominis, Dermabacter vaginalis, Dermacoccus nishinomiyaensis, Desemzia incerta, Desertibacter roseus, Dialister invisus, Dialister micraerophilus, Dialister propionicifaciens, Dietzia aurantiaca, Dietzia cercidiphylli, Dietzia timorensis, Dietzia timorensis, Dokdonella koreensis, Dokdonella koreensis, Dolosigranulum pigrum, Eikenella corrodens, Elizabethkingia miricola, Elstera litoralis, Empedobacter brevis, Enhydrobacter aerosaccus, Enterobacter xiangfangensis, Enterococcus aquimarinus, Enterococcus faecalis, Enterococcus olivae, Erwinia rhapontici, Eubacterium eligens, Eubacterium infirmum, Eubacterium rectale, Eubacterium saphenum, Eubacterium sulci, Exiguobacterium mexicanum, Facklamia tabacinasalis, Falsirhodobacter halotolerans, Finegoldia magna, Flavobacterium cutihirudinis, Flavobacterium lindanitolerans, Flavobacterium resistens, Friedmanniella capsulata, Fusobacterium nucleatum subsp. polymorphum, Gemella haemolysans, Gemella morbillorum, Gemella palaticanis, Gemella sanguinis, Gemmobacter aquaticus, Gemmobacter caeni, Gordonia jinhuaensis, Gordonia kroppenstedtii, Gordonia polyisoprenivorans, Gordonia polyisoprenivorans, Granulicatella adiacens, Granulicatella elegans, Haemophilus parainfluenzae, Haemophilus sputorum, Halomonas sulfidaeris, Herpetosiphon aurantiacus, Hydrocarboniphaga effusa, Idiomarina marls, Janibacter anophelis, Janibacter hoylei, Janibacter indicus, Janibacter limosus, Janibacter melonis, Jeotgalicoccus halophilus, Jonquetella anthropi, Kaistia geumhonensis, Kingella denitrificans, Kingella oralis, Klebsiella oxytoca, Knoellia aerolata, Knoellia locipacati, Kocuria atrinae, Kocuria carniphila, Kocuria kristinae, Kocuria palustris, Kocuria turfanensis, Lachnoanaerobaculum saburreum, Lachnoanaerobaculum saburreum, Lactobacillus crispatus, Lactobacillus iners, Lactococcus lactis subsp. lactis, Lactococcus lactis subsp. lactis, Lactococcus piscium, Lapillicoccus jejuensis, Lautropia mirabilis, Legionella beliardensis, Leptotrichia buccalis, Leptotrichia goodfellowii, Leptotrichia hofstadii, Leptotrichia hongkongensis, Leptotrichia shahii, Leptotrichia trevisanii, Leptotrichia wadei, Luteimonas terricola, Lysinibacillus fusiformis, Lysobacter spongiicola, Lysobacter xinjiangensis, Macrococcus caseolyticus, Marmoricola pocheonensis, Marmoricola scoriae, Massilia alkalitolerans, Massilia alkalitolerans, Massilia aurea, Massilia plicata, Massilia timonae, Megamonas rupellensis, Meiothermus silvanus, Methylobacterium dankookense, Methylobacterium goesingense, Methylobacterium goesingense, Methylobacterium isbiliense, Methylobacterium jeotgali, Methylobacterium oxalidis, Methylobacterium platani, Methylobacterium pseudosasicola, Methyloversatilis universalis, Microbacterium foliorum, Microbacterium hydrothermale, Microbacterium hydrothermale, Microbacterium lacticum, Microbacterium lacticum, Microbacterium laevaniformans, Microbacterium paludicola, Microbacterium petrolearium, Microbacterium phyllosphaerae, Microbacterium resistens, Micrococcus antarcticus, Micrococcus cohnii, Micrococcus flavus, Micrococcus lylae, Micrococcus terreus, Microlunatus aurantiacus, Micropruina glycogenica, Microvirga aerilata, Microvirga aerilata, Microvirga subterranea, Microvirga vignae, Microvirga zambiensis, Microvirgula aerodenitrificans, Mogibacterium timidum, Moraxella atlantae, Moraxella catarrhalis, Morganella morganii subsp. morganii, Morganella psychrotolerans, Murdochiella asaccharolytica, Mycobacterium asiaticum, Mycobacterium chubuense, Mycobacterium crocinum, Mycobacterium gadium, Mycobacterium holsaticum, Mycobacterium iranicum, Mycobacterium longobardum, Mycobacterium neoaurum, Mycobacterium neoaurum, Mycobacterium obuense, Negativicoccus succinicivorans, Neisseria bacilliformis, Neisseria oralis, Neisseria sicca, Neisseria subflava, Nesterenkonia lacusekhoensis, Nesterenkonia rhizosphaerae, Nevskia persephonica, Nevskia ramosa, Niabella yanshanensis, Niveibacterium umoris, Nocardia niwae, Nocardia thailandica, Nocardioides agariphilus, Nocardioides dilutus, Nocardioides ganghwensis, Nocardioides hwasunensis, Nocardioides nanhaiensis, Nocardioides sediminis, Nosocomiicoccus ampullae, Noviherbaspirillum malthae, Novosphingobium lindaniclasticum, Novosphingobium rosa, Ochrobactrum rhizosphaerae, Olsenella uli, Ornithinimicrobium murale, Ornithinimicrobium tianjinense, Oryzobacter terrae, Ottowia beijingensis, Paenalcaligenes suwonensis, Paenibacillus agaridevorans, Paenibacillus phoenicis, Paenibacillus xylanexedens, Paludibacterium yongneupense, Pantoea cypripedii, Parabacteroides distasonis, Paraburkholderia andropogonis, Paracoccus alcaliphilus, Paracoccus angustae, Paracoccus kocurii, Paracoccus laeviglucosivorans, Paracoccus sediminis, Paracoccus sphaerophysae, Paracoccus yeei, Parvimonas micra, Parviterribacter multiflagellatus, Patulibacter ginsengiterrae, Pedobacter aquatilis, Pedobacter ginsengisoli, Pedobacter xixiisoli, Peptococcus niger, Peptoniphilus coxii, Peptoniphilus gorbachii, Peptoniphilus harei, Peptoniphilus koenoeneniae, Peptoniphilus lacrimalis, Peptostreptococcus anaerobius, Peptostreptococcus stomatis, Phascolarctobacterium faecium, Phenylobacterium haematophilum, Phenylobacterium kunshanense, Pluralibacter gergoviae, Polymorphobacter multimanifer, Porphyromonas bennonis, Porphyromonas endodontalis, Porphyromonas gingivalis, Porphyromonas gingivicanis, Porphyromonas pasteri, Porphyromonas pogonae, Porphyromonas somerae, Povalibacter uvarum, Prevotella aurantiaca, Prevotella baroniae, Prevotella bivia, Prevotella buccae, Prevotella buccalis, Prevotella copri, Prevotella corporis, Prevotella denticola, Prevotella enoeca, Prevotella histicola, Prevotella intermedia, Prevotella jejuni, Prevotella jejuni, Prevotella maculosa, Prevotella melaninogenica, Prevotella melaninogenica, Prevotella micans, Prevotella multiformis, Prevotella nanceiensis, Prevotella nigrescens, Prevotella oris, Prevotella oulorum, Prevotella pallens, Prevotella pleuritidis, Prevotella saccharolytica, Prevotella salivae, Prevotella shahii, Prevotella timonensis, Prevotella veroralis, Propionibacterium acidifaciens, Propionibacterium acnes subsp. acnes, Propionibacterium acnes subsp. acnes, Propionibacterium acnes subsp. elongatum, Propionibacterium granulosum, Propionimicrobium lymphophilum, Propionispira arcuata, Pseudokineococcus lusitanus, Pseudomonas aeruginosa, Pseudomonas chengduensis, Pseudonocardia benzenivorans, Pseudorhodoplanes sinuspersici, Psychrobacter sanguinis, Ramlibacter ginsenosidimutans, Rheinheimera aquimaris, Rhizobium alvei, Rhizobium daejeonense, Rhizobium larrymoorei, Rhizobium rhizoryzae, Rhizobium soli, Rhizobium taibaishanense, Rhizobium vignae, Rhodanobacter glycinis, Rhodobacter veldkampii, Rhodococcus enclensis, Rhodococcus fascians, Rhodococcus fascians, Rhodovarius lipocyclicus, Rivicola pingtungensis, Roseburia inulinivorans, Rosenbergiella nectarea, Roseomonas aerilata, Roseomonas aquatica, Roseomonas mucosa, Roseomonas rosea, Roseomonas vinacea, Rothia aeria, Rothia amarae, Rothia dentocariosa, Rothia endophytica, Rothia mucilaginosa, Rothia nasimurium, Rubellimicrobium mesophilum, Rubellimicrobium roseum, Rubrobacter bracarensis, Rudaea cellulosilytica, Ruminococcus gnavus, Runella zeae, Saccharopolyspora rectivirgula, Salinicoccus qingdaonensis, Scardovia wiggsiae, Sediminibacterium ginsengisoli, Selenomonas artemidis, Selenomonas infelix, Selenomonas noxia, Selenomonas sputigena, Shewanella aestuarii, Shuttleworthia satelles, Simonsiella muelleri, Skermanella aerolata, Skermanella stibiiresistens, Slackia exigua, Smaragdicoccus niigatensis, Sneathia sanguinegens, Solirubrobacter soli, Sphingobacterium caeni, Sphingobacterium daejeonense, Sphingobacterium hotanense, Sphingobacterium kyonggiense, Sphingobacterium multivorum, Sphingobacterium nematocida, Sphingobacterium spiritivorum, Sphingobium amiense, Sphingobium indicum, Sphingobium lactosutens, Sphingobium subterraneum, Sphingomonas abaci, Sphingomonas aestuarii, Sphingomonas canadensis, Sphingomonas daechungensis, Sphingomonas dokdonensis, Sphingomonas echinoides, Sphingomonas fonticola, Sphingomonas fonticola, Sphingomonas formosensis, Sphingomonas gei, Sphingomonas hankookensis, Sphingomonas hankookensis, Sphingomonas koreensis, Sphingomonas kyeonggiensis, Sphingomonas laterariae, Sphingomonas mucosissima, Sphingomonas oligophenolica, Sphingomonas pseudosanguinis, Sphingomonas sediminicola, Sphingomonas yantingensis, Sphingomonas yunnanensis, Sphingopyxis indica, Spirosoma rigui, Sporacetigenium mesophilum, Sporocytophaga myxococcoides, Staphylococcus auricularis, Staphylococcus epidermidis, Staphylococcus epidermidis, Staphylococcus hominis subsp. novobiosepticus, Staphylococcus lugdunensis, Staphylococcus pettenkoferi, Stenotrophomonas koreensis, Stenotrophomonas rhizophila, Stenotrophomonas rhizophila, Streptococcus agalactiae, Streptococcus canis, Streptococcus cristatus, Streptococcus gordonii, Streptococcus infantis, Streptococcus intermedius, Streptococcus mutans, Streptococcus oligofermentans, Streptococcus oralis, Streptococcus sanguinis, Streptomyces iconiensis, Streptomyces yanglinensis, Tabrizicola aquatica, Tahibacter caeni, Tannerella forsythia, Tepidicella xavieri, Tepidimonas fonticaldi, Terracoccus luteus, Tessaracoccus flavescens, Thermus thermophilus, Tianweitania sediminis, Tianweitania sediminis, Treponema amylovorum, Treponema denticola, Treponema lecithinolyticum, Treponema medium, Turicella otitidis, Turicibacter sanguinis, Undibacterium oligocarboniphilum, Undibacterium squillarum, Vagococcus salmoninarum, Varibaculum cambriense, Vibrio metschnikovii, Xanthobacter tagetidis, Xenophilus aerolatus, Xenophilus arseniciresistens, Yimella lutea, Zimmermannella alba, Zimmermannella bifida and/or Zoogloea caeni.

In other embodiments, the targeted bacteria cells are those commonly found in the vaginal microbiota and are, without limitation, Acinetobacter antiviralis, Acinetobacter baumannii, Acinetobacter calcoaceticus, Acinetobacter johnsonii, Actinobaculum massiliense, Actinobaculum schaalii, Actinomyces europaeus, Actinomyces graevenitzii, Actinomyces israelii, Actinomyces meyeri, Actinomyces naeslundii, Actinomyces neuii, Actinomyces odontolyticus, Actinomyces turicensis, Actinomyces urogenitalis, Actinomyces viscosus, Aerococcus christensenii, Aerococcus urinae, Aerococcus viridans, Aeromonas encheleia, Aeromonas salmonicida, Afipia massiliensis, Agrobacterium tumefaciens, Algoriphagus aquatilis, Aliivibrio wodanis, Alistipes finegoldii, Alloiococcus otitis, Alloprevotella tannerae, Alloscardovia omnicolens, Altererythrobacter epoxidivorans, Ammoniphilus oxalaticus, Amnibacterium kyonggiense, Anaerococcus hydrogenalis, Anaerococcus lactolyticus, Anaerococcus murdochii, Anaerococcus obesiensis, Anaerococcus prevotii, Anaerococcus tetradius, Anaerococcus vaginalis, Anaeroglobus geminatus, Anoxybacillus pushchinoensis, Aquabacterium parvum, Arcanobacterium phocae, Arthrobacter aurescens, Asticcacaulis excentricus, Atopobium minutum, Atopobium parvulum, Atopobium rimae, Atopobium vaginae, Avibacterium gallinarum, Bacillus acidicola, Bacillus atrophaeus, Bacillus cereus, Bacillus cibi, Bacillus coahuilensis, Bacillus gaemokensis, Bacillus methanolicus, Bacillus oleronius, Bacillus pumilus, Bacillus shackletonii, Bacillus sporothermodurans, Bacillus subtilis, Bacillus wakoensis, Bacillus weihenstephanensis, Bacteroides barnesiae, Bacteroides coagulans, Bacteroides dorei, Bacteroides faecis, Bacteroides forsythus, Bacteroides fragilis, Bacteroides nordii, Bacteroides ovatus, Bacteroides salyersiae, Bacteroides stercoris, Bacteroides uniformis, Bacteroides vulgatus, Bacteroides xylanisolvens, Bacteroides zoogleoformans, Barnesiella viscericola, Bhargavaea cecembensis, Bifidobacterium adolescentis, Bifidobacterium bifidum, Bifidobacterium breve, Bifidobacterium dentium, Bifidobacterium logum subsp. infantis, Bifidobacterium longum, Bifidobacterium pseudocatenulatum, Bifidobacterium scardovii, Bilophila wadsworthia, Blautia hydrogenotrophica, Blautia obeum, Blautia producta, Brachybacterium faecium, Bradyrhizobium japonicum, Brevibacterium mcbrellneri, Brevibacterium otitidis, Brevibacterium paucivorans, Bulleidia extructa, Burkholderia fungorum, Burkholderia phenoliruptix, Caldicellulosiruptor saccharolyticus, Caldimonas taiwanensis, Campylobacter gradlis, Campylobacter hominis, Campylobacter sputorum, Campylobacter ureolyticus, Capnocytophaga ochracea, Cardiobacterium hominis, Catonella morbi, Chlamydia trachomatis, Chlamydophila abortus, Chondromyces robustus, Chryseobacterium aquaticum, Citrobacter youngae, Cloacibacterium normanense, Clostridium cavendishii, Clostridium colicanis, Clostridium jejuense, Clostridium perfringens, Clostridium ramosum, Clostridium sordellii, Clostridium viride, Comamonas terrigena, Corynebacterium accolens, Corynebacterium appendicis, Corynebacterium coyleae, Corynebacterium glucuronolyticum, Corynebacterium glutamicum, Corynebacterium jeikeium, Corynebacterium kroppenstedtii, Corynebacterium lipophiloflavum, Corynebacterium minutissimum, Corynebacterium mucifaciens, Corynebacterium nuruki, Corynebacterium pseudogenitalium, Corynebacterium pyruviciproducens, Corynebacterium singulare, Corynebacterium striatum, Corynebacterium tube rculostearicum, Corynebacterium xerosis, Cryobacterium psychrophilum, Curtobacterium flaccumfaciens, Cutibacterium acnes, Cutibacterium avidum, Cytophaga xylanolytica, Deinococcus radiophilus, Delftia tsuruhatensis, Desulfovibrio desulfuricans, Dialister invisus, Dialister micraerophilus, Dialister pneumosintes, Dialister propionicifaciens, Dickeya chrysanthemi, Dorea longicatena, Eggerthella lenta, Eggerthia catenaformis, Eikenella corrodens, Enhydrobacter aerosaccus, Enterobacter asburiae, Enterobacter cloacae, Enterococcus avium, Enterococcus durans, Enterococcus faecalis, Enterococcus faecium, Enterococcus hirae, Erwinia persicina, Erwinia rhapontici, Erwinia toletana, Escherichia coli, Escherichia fergusonii, Eubacterium brachy, Eubacterium eligens, Eubacterium nodatum, Eubacterium recta/e, Eubacterium saphenum, Eubacterium siraeum, Eubacterium sulci, Eubacterium yurii, Exiguobacterium acetylicum, Facklamia ignava, Faecalibacterium prausnitzii, Filifactor alocis, Finegoldia magna, Fusobacterium gonidiaformans, Fusobacterium nucleatum, Fusobacterium periodonticum, Gardnerella vaginalis, Gemella asaccharolytica, Gemella bergeri, Gemella haemolysans, Gemella sanguinis, Geobacillus stearothermophilus, Geobacillus the rmocatenulatus, Geobacillus thermoglucosidasius, Geobacter grbiciae, Granulicatella elegans, Haemophilus ducreyi, Haemophilus haemolyticus, Haemophilus parahaemolyticus, Haemophilus parainfluenzae, Hafnia alvei, Halomonas meridiana, Halomonas phoceae, Halomonas venusta, Herbaspirillum seropedicae, Janthinobacterium lividum, Jonquetella anthropi, Klebsiella granulomatis, Klebsiella oxytoca, Klebsiella pneumoniae, Lactobacillus acidophilus, Lactobacillus amylovorus, Lactobacillus brevis, Lactobacillus coleohominis, Lactobacillus crispatus, Lactobacillus curvatus, Lactobacillus delbrueckii, Lactobacillus fermentum, Lactobacillus gasseri, Lactobacillus helveticus, Lactobacillus iners, Lactobacillus jensenii, Lactobacillus johnsonii, Lactobacillus kalixensis, Lactobacillus kefiranofaciens, Lactobacillus kimchicus, Lactobacillus kitasatonis, Lactobacillus mucosae, Lactobacillus panis, Lactobacillus paracasei, Lactobacillus plantarum, Lactobacillus pontis, Lactobacillus reuteri, Lactobacillus rhamnosus, Lactobacillus salivarius, Lactobacillus ultunensis, Lactobacillus vaginalis, Lactococcus lactis, Leptotrichia buccalis, Leuconostoc carnosum, Leuconostoc citreum, Leuconostoc garlicum, Leuconostoc lactis, Leuconostoc mesenteroides, Lysinimonas kribbensis, Mageeibacillus indolicus, Maribacter orientalis, Marinomonas protea, Marinospirillum insulare, Massilia timonae, Megasphaera elsdenii, Megasphaera micronuciformis, Mesorhizobium amorphae, Methylobacterium radiotolerans, Methylotenera versatilis, Microbacterium halophilum, Micrococcus luteus, Microterricola viridarii, Mobiluncus curtisii, Mobiluncus mullens, Mogibacterium timidum, Moorella glycerin, Moraxella osloensis, Morganella morganii, Moryella indoligenes, Murdochiella asaccharolytica, Mycoplasma alvi, Mycoplasma genitalium, Mycoplasma hominis, Mycoplasma muris, Mycoplasma salivarium, Negativicoccus succinicivorans, Neisseria flava, Neisseria gonorrhoeae, Neisseria mucosa, Neisseria subflava, Nevskia ramosa, Nevskia soli, Nitriliruptor alkaliphilus, Odoribacter splanchnicus, Oligella urethralis, Olsenella uli, Paenibacillus amylolyticus, Paenibacillus humicus, Paenibacillus pabuli, Paenibacillus pasadenensis, Paenibacillus pini, Paenibacillus validus, Pantoea agglomerans, Parabacteroides merdae, Paraburkholderia caryophylli, Paracoccus yeei, Parastreptomyces abscessus, Parvimonas micra, Pectobacterium betavasculorum, Pectobacterium carotovorum, Pediococcus acidilactici, Pediococcus ethanolidurans, Pedobacter alluvionis, Pedobacter wanjuense, Pelomonas aquatica, Peptococcus niger, Peptoniphilus asaccharolyticus, Peptoniphilus gorbachii, Peptoniphilus harei, Peptoniphilus indolicus, Peptoniphilus lacrimalis, Peptoniphilus massiliensis, Peptostreptococcus anaerobius, Peptostreptococcus massiliae, Peptostreptococcus stomatis, Photobacterium angustum, Photobacterium frigidiphilum, Photobacterium phosphoreum, Porphyromonas asaccharolytica, Porphyromonas bennonis, Porphyromonas catoniae, Porphyromonas endodontalis, Porphyromonas gingivalis, Porphyromonas somerae, Porphyromonas uenonis, Prevotella amnii, Prevotella baroniae, Prevotella bergensis, Prevotella bivia, Prevotella buccae, Prevotella buccalis, Prevotella colorans, Prevotella copri, Prevotella corporis, Prevotella dentalis, Prevotella denticola, Prevotella disiens, Prevotella intermedia, Prevotella loescheii, Prevotella marshii, Prevotella melaninogenica, Prevotella micans, Prevotella nigrescens, Prevotella oris, Prevotella pleuritidis, Prevotella ruminicola, Prevotella shahii, Prevotella stercorea, Prevotella timonensis, Prevotella veroralis, Propionimicrobium lymphophilum, Proteus mirabilis, Pseudomonas abietaniphila, Pseudomonas aeruginosa, Pseudomonas amygdali, Pseudomonas azotoformans, Pseudomonas chlororaphis, Pseudomonas cuatrocienegasensis, Pseudomonas fluorescens, Pseudomonas fulva, Pseudomonas lutea, Pseudomonas mucidolens, Pseudomonas oleovorans, Pseudomonas orientalis, Pseudomonas pseudoalcaligenes, Pseudomonas psychrophila, Pseudomonas putida, Pseudomonas synxantha, Pseudomonas syringae, Pseudomonas tolaasii, Pseudo propionibacterium propionicum, Rahnella aquatilis, Ralstonia pickettii, Ralstonia solanacearum, Raoultella planticola, Rhizobacter dauci, Rhizobium etli, Rhodococcus fascians, Rhodopseudomonas palustris, Roseburia intestinalis, Roseburia inulinivorans, Rothia mucilaginosa, Ruminococcus bromii, Ruminococcus gnavus, Ruminococcus torques, Sanguibacter keddieii, Sediminibacterium salmoneum, Selenomonas bovis, Serratia fonticola, Serratia liquefaciens, Serratia marcescens, Shewanella algae, Shewanella amazonensis, Shigella boydii, Shigella sonnei, Slackia exigua, Sneathia amnii, Sneathia sanguinegens, Solobacterium moorei, Sorangium cellulosum, Sphingobium amiense, Sphingobium japonicum, Sphingobium yanoikuyae, Sphingomonas wittichii, Sporosarcina aquimarina, Staphylococcus aureus, Staphylococcus auricularis, Staphylococcus capitis, Staphylococcus epidermidis, Staphylococcus haemolyticus, Staphylococcus hominis, Staphylococcus lugdunensis, Staphylococcus saprophyticus, Staphylococcus schleiferi, Staphylococcus simiae, Staphylococcus simulans, Staphylococcus warneri, Stenotrophomonas maltophilia, Stenoxybacter acetivorans, Streptococcus agalactiae, Streptococcus anginosus, Streptococcus australis, Streptococcus equinus, Streptococcus gallolyticus, Streptococcus infantis, Streptococcus intermedius, Streptococcus lutetiensis, Streptococcus marimammalium, Streptococcus mitis, Streptococcus mutans, Streptococcus oralis, Streptococcus parasanguinis, Streptococcus phocae, Streptococcus pseudopneumoniae, Streptococcus salivarius, Streptococcus sanguinis, Streptococcus thermophilus, Sutterella wadsworthensis, Tannerella forsythia, Terrahaemophilus aromaticivorans, Treponema denticola, Treponema maltophilum, Treponema parvum, Treponema vincentii, Trueperella bernardiae, Turicella otitidis, Urea plasma parvum, Urea plasma urealyticum, Varibaculum cambriense, Variovorax paradoxus, Veillonella atypica, Veillonella dispar, Veillonella montpellierensis, Veillonella parvula, Virgibacillus proomii, Viridibacillus arenosi, Viridibacillus arvi, Weissella cibaria, Weissella soli, Xanthomonas campestris, Xanthomonas vesicatoria, Zobellia laminariae and/or Zoogloea ramigera.

In one embodiment, the targeted bacteria are Escherichia coli.

In one embodiment, the targeted bacteria are Cutibacterium acnes more specifically the acne related Cutibacterium acnes from the phylogroup IA1 or RT4, RT5, RT8, RT9, RT10 or Clonal Complex(CC) CC1, CC3, CC4, more specifically the ST1, ST3, ST4.

Thus, the first type of bacteriophage disclosed herein, and therefore the phage particles or phage-derived delivery particles of the invention, may target (e.g., specifically target) a bacterial cell from any one or more of the foregoing genus and/or species of bacteria in particular to specifically deliver the payload.

In one embodiment, the targeted bacteria are pathogenic bacteria. The targeted bacteria can be virulent bacteria.

The targeted bacteria can be antibacterial resistance bacteria, preferably selected from the group consisting of extended-spectrum beta-lactamase-producing (ESBL) Escherichia coli, ESBL Klebsiella pneumoniae, vancomycin-resistant Enterococcus (VRE), methicillin-resistant Staphylococcus aureus (MRSA), multidrug-resistant (MDR) Acinetobacter baumannii, MDR Enterobacter spp., and a combination thereof. Preferably, the targeted bacteria can be selected from the group consisting of extended-spectrum beta-lactamase-producing (ESBL) Escherichia coli strains.

Alternatively, the targeted bacterium can be a bacterium of the microbiome of a given species, preferably a bacterium of the human microbiota.

In a particular embodiment, said targeted bacterial cells are from a species or strain different from the production bacterial cell.

### Hybrid Helper Phage System and Hybrid Helper Phage

The present invention also concerns a hybrid helper phage system comprising:
(i) at least one phage DNA packaging gene(s), as defined in the section “Production bacterial cell” above, derived from a first type of bacteriophage, as defined in the section “Bacteriophage and gene derived from a bacteriophage” above,
(i′) at least one phage structural gene(s), as defined in the section “Production bacterial cell” above, derived from said first type of bacteriophage, and
(ii) at least one gene, derived from a second type of bacteriophage, as defined in the section “Bacteriophage and gene derived from a bacteriophage” above, involved in phage excision/insertion, phage DNA replication, and/or phage regulation, as defined in the section “Production bacterial cell” above,
   wherein said genes (i), (i′) and (ii) are comprised in a unique nucleic acid molecule or in separate nucleic acid molecules,
   wherein said first type of bacteriophage comes from and/or target bacterial species or strain different from the bacterial species or strain from which said second type of bacteriophage comes and/or that said second type of bacteriophage targets, and
   wherein said hybrid helper phage system does not comprise any expressed phage structural gene, as defined in the section “Production bacterial cell” above, derived from said second type of bacteriophage.

In the context of the invention, the term “hybrid helper phage system” is meant a group of at least one nucleic acid molecule, preferably of at least two separate nucleic acid molecules, comprising the genes (i), (i′) and (ii) defined above, which enables the production of phage particles and/or phage-derived delivery vehicles by the production bacterial cell comprising said system, wherein when the system comprises at least two separate nucleic acid molecules, said genes (i), (i′) and (ii) are distributed on said at least two separate nucleic acid molecules.

As used herein, the term “nucleic acid” refers to a sequence of at least two nucleotides covalently linked together which can be single-stranded or double-stranded or contains portion of both single-stranded and double-stranded sequence. Nucleic acids of the present invention can be naturally occurring, recombinant or synthetic. The nucleic acid can be in the form of a circular sequence or a linear sequence or a combination of both forms. The nucleic acid can be DNA, both genomic or cDNA, or RNA or a combination of both. The nucleic acid may contain any combination of deoxyribonucleotides and ribonucleotides, and any combination of bases, including uracil, adenine, thymine, cytosine, guanine, inosine, xanthine, hypoxanthine, isocytosine, 5-hydroxymethylcytosine and isoguanine. Other examples of modified bases that can be used in the present invention are detailed in Weigele et al. Chem Rev. 2016 Oct. 26; 116(20):12655-12687. The term “nucleic acid” also encompasses any nucleic acid analogs which may contain other backbones comprising, without limitation, phosphoramide, phosphorothioate, phosphorodithioate, O-methylphophoroamidite linkage and/or deoxyribonucleotides and ribonucleotides nucleic acids. Any combination of the above features of a nucleic acid is also encompassed by the present invention.

In a particular embodiment, said genes (i), (i′) and (ii) are comprised in a bacterial chromosome, in particular in a production bacterial cell chromosome. In a more particular embodiment, said genes (i), (i′) and (ii) are comprised in a bacterial chromosome in a same region. In an alternative embodiment, said genes (i), (i′) and (ii) are comprised in a bacterial chromosome in distinct regions.

In an alternative embodiment, said genes (i), (i′) and (ii) are comprised in separate plasmids. In another particular embodiment, said genes (i), (i′) and (ii) are all comprised in a same plasmid.

In another particular embodiment, said genes (i), (i′) and (ii) are each independently comprised in a bacterial chromosome or in a plasmid.

In a more particular embodiment, said genes (i), (i′) and (ii) are comprised in a hybrid helper phage.

Therefore, in a particular embodiment, said hybrid helper phage system consists of a hybrid helper phage comprising:
(i) at least one phage DNA packaging gene(s) and at least one phage structural gene(s), as defined in the section “Production bacterial cell” above, derived from a first type of bacteriophage, as defined in the section “Bacteriophage and gene derived from a bacteriophage” above, and
(ii) at least one gene, derived from a second type of bacteriophage, as defined in the section “Bacteriophage and gene derived from a bacteriophage” above, involved in phage excision/insertion, phage DNA replication, and/or phage regulation, as defined in the section “Production bacterial cell” above,
   wherein said first type of bacteriophage comes from and/or target bacterial species or strain different from the bacterial species or strain from which said second type of bacteriophage comes and/or that said second type of bacteriophage targets, and
   wherein said hybrid helper phage does not comprise any phage structural gene, as defined in the section “Production bacterial cell” above, derived from said second type of bacteriophage.

By “helper phage” is meant herein an engineered phage providing all the necessary gene products for particle formation when using phagemid vectors. Helper phages typically have a defective origin of replication or packaging signal, and hence, are inefficient in self-packaging.

By “hybrid helper phage” is meant herein an engineered helper phage which is constituted of elements derived from at least two different types of bacteriophage.

In a particular embodiment, the hybrid helper phage of the invention is integrated in the genome of the production bacterial cell as a prophage.

### Production Method

The present invention further concerns a method for producing phage particles or phage-derived delivery vehicles, comprising:
(a) providing the production bacterial cell of the invention, and
(b) inducing, in said production bacterial cell, expression of said at least one of said phage structural gene(s) and at least one of said phage DNA packaging gene(s), and assembly of the products expressed by said at least one phage structural gene(s) and said at least one phage DNA packaging gene(s), thereby producing phage particles or phage-derived delivery vehicles.

The inducing step (b) can be carried out by any technique well-known from the skilled person. In particular, as will be understood by the skilled person, said inducing step will depend on the particular induction mechanism controlling the expression of said at least one of said phage structural genes and phage DNA packaging genes, in said production bacterial cell.

More particularly, it will be understood by the skilled person that, when said induction mechanism comprises at least one gene, derived from a second type of bacteriophage, involved in phage excision/insertion, phage DNA replication, and/or phage regulation, said inducing step will depend on the bacteriophage from which said sequences are derived. Typically, said inducing step can be a thermal induction (for phages that are naturally triggered by this signal or engineered repressors such as lambda cl), small molecule inducers (depending on the phage), any signal triggering SOS response (for instance addition of mitomycin), etc.

### Production of Tailocin and/or Pyocin

The approach disclosed above can also be applied to the production of tailocins and/or pyocins, enabling the safe and efficient production of such bacteriocins in manipulable production cells.

The present invention thus also concerns a production bacterial cell for producing tailocin and/or pyocin, said production bacterial cell stably comprising at least one tailocin and/or pyocin structural gene(s) derived from a bacterial species or strain containing tailocin and/or pyocin genes,
wherein the expression of at least one of said tailocin and/or pyocin structural gene(s) in said production bacterial cell is controlled by at least one induction mechanism, and
wherein said production bacterial cell is from a bacterial species or strain different from the bacterial species or strain containing tailocin and/or pyocin genes from which said tailocin and/or pyocin structural gene(s) is derived.

By “tailocin” is meant herein a multisubunit bacteriocin that resembles bacteriophage tails. There are two classes of tailocin particles, the flexible noncontractile F-tailocins and the rigid contractile R-tailocins, which resemble and are evolutionarily related to Siphoviridae and Myoviridae phage tails, respectively. Examples of tailocins typically include F-type and R-type pyocins, carotovoricin, xenorhabdicin, and maltocin.

By “pyocin” is meant herein a bacteriocin formed by Pseudomonas aeruginosa. They can be produced spontaneously or induced by certain chemicals, such as mitomycin C. Three different types of pyocins have been identified: R-type, S-type, and F-type (Nakayama et al., (2000) Mol. Microbiol. 38:213-231). They differ by their morphology and mode of killing. Their bactericidal activities are strain specific. R-type pyocins resemble inflexible and contractile tails of bacteriophages, belong to the tailocins disclosed above, and are further classified into five groups: R1, R2, R3, R4, and R5. F-type pyocins also resemble phage tails, flexible but noncontractile rod-like structure, with distal filaments, and also belong to the tailocins disclosed above. They are similar in structure and serological properties, but they are different in receptor specificities. Three subtypes of F-type pyocins were reported: F1, F2, and F3. In a particular embodiment, said pyocin is a R-type or F-type pyocin.

By “tailocin and/or pyocin structural gene” is meant herein genes from a tailocin and/or pyocin producing bacteria which are involved in the building of the tailocin and/or pyocin. Tailocin and/or pyocin structural genes include genes encoding the subunits and/or components of said tailocin and/or pyocin, as disclosed above, and genes encoding bacterial proteins involved in the assembly of the tailocin and/or pyocin subunits and/or components.

In a particular embodiment, said tailocin and/or pyocin structural genes are pyocin structural genes as defined above. In that embodiment, said bacterial species or strain containing tailocin and/or pyocin genes from which said pyocin structural genes are derived, is preferably a Pseudomonas aeruginosa bacteria.

In a particular embodiment, said tailocin and/or pyocin structural genes are tailocin structural genes as defined above. In that embodiment, said bacterial species or strain containing tailocin and/or pyocin genes from which said tailocin structural genes are derived, is preferably selected from the bacteria, defined in the section “Targeted bacteria” above, which naturally contain said tailocin structural genes, and preferably produce tailocins.

In a particular embodiment, said tailocin and/or pyocin structural gene(s) are comprised in at least one plasmid, chromosome, and/or helper phage.

In the context of the invention, said induction mechanism is as defined in the section “Production bacterial cell” above.

More particularly, in an embodiment, the at least one induction mechanism controls the expression of all said tailocin and/or pyocin structural gene(s).

In a particular embodiment, said at least one induction mechanism further controls the copy number of said at least one of said tailocin and/or pyocin structural gene(s).

In a particular embodiment, said at least one induction mechanism comprises at least one gene involved in tailocin and/or pyocin regulation, said gene involved in tailocin and/or pyocin regulation being derived from a bacterial species or strain containing tailocin and/or pyocin genes which are different from those from which said tailocin and/or pyocin structural genes are derived.

Therefore in a particular embodiment, said production bacterial cell further comprises at least one gene involved in tailocin and/or pyocin regulation, said gene involved in tailocin and/or pyocin regulation being derived from a bacterial species or strain containing tailocin and/or pyocin genes which are different from those from which said tailocin and/or pyocin structural genes are derived.

By “gene involved in tailocin and/or pyocin regulation” is meant herein genes encoding regulatory elements controlling induction and/or expression of tailocin and/or pyocin in a natural tailocin and/or producing bacterial cell.

Alternatively, said at least one induction mechanism comprises at least one gene, derived from a bacteriophage, as defined in the section “Bacteriophage and gene derived from a bacteriophage” above, involved in phage excision/insertion, phage DNA replication, and/or phage regulation, as defined in the section “Production bacterial cell” above, said bacteriophage coming from and/or targeting bacterial species or strain different from the bacterial species or strain from which said tailocin and/or pyocin structural gene(s) is derived.

Therefore, in a particular embodiment, said production bacterial cell further comprises at least one gene, derived from a bacteriophage, as defined in the section “Bacteriophage and gene derived from a bacteriophage” above, involved in phage excision/insertion, phage DNA replication, and/or phage regulation, as defined in the section “Production bacterial cell” above, said bacteriophage coming from and/or targeting bacterial species or strain different from the bacterial species or strain from which said tailocin and/or pyocin structural gene(s) is derived.

In a particular embodiment, said tailocin and/or pyocin is intended to lyse targeted bacterial cells, as defined in the section “Targeted bacterial cells” above.

In a particular embodiment, said targeted bacterial cells are from a species or strain different from the production bacterial cell.

In a particular embodiment, said production bacterial cell is from the same bacterial species or strain as the bacterial species or strain from which said bacteriophage comes and/or that said bacteriophage targets.

In a particular embodiment, said production bacterial cell is an E. coli bacterial cell.

The present invention further concerns a method for producing tailocin and/or pyocin, comprising:
(a) providing the tailocin and/or pyocin production bacterial cell defined above, and
(b) inducing, as defined in the section “Producing method” above, in said production bacterial cell, expression of said at least one of said tailocin and/or pyocin structural gene(s), as defined above, and assembly of the products expressed by said at least one tailocin and/or pyocin structural gene(s), thereby producing tailocin and/or pyocin.

The present invention also concerns a hybrid production system comprising:
(i′) at least one tailocin and/or pyocin structural gene(s), as defined above, derived from a bacterial species or strain containing tailocin and/or pyocin genes, as defined above, and
(ii) at least one gene, derived from a bacteriophage, as defined in the section “Bacteriophage and gene derived from a bacteriophage” above, involved in phage excision/insertion, phage DNA replication, and/or phage regulation, as defined in the section “Production bacterial cell” above; or at least one gene involved in tailocin and/or
pyocin regulation, said gene involved in tailocin and/or pyocin regulation being derived from a bacterial species or strain containing tailocin and/or pyocin genes which are different from those from which said tailocin and/or pyocin structural genes are derived,
wherein said genes (i′) and (ii) are comprised in a unique nucleic acid molecule or in separate nucleic acid molecules, as defined in the section “Hybrid helper phage system and hybrid helper phage” above,
wherein said bacteriophage comes from and/or targets bacterial species or strain different from the bacterial species or strain from which said tailocin and/or pyocin structural gene(s) is derived, and
wherein said hybrid production system does not comprise any expressed phage structural gene, as defined in the section “Production bacterial cell” above, derived from said bacteriophage.

By “hybrid production system” is meant herein a group of at least one nucleic acid molecule, preferably of at least two separate nucleic acid molecules, comprising the genes (i′) and (ii) defined above, which enables the production of tailocins and/or pyocins by the production bacterial cell comprising said system, wherein when the system comprises at least two separate nucleic acid molecules, said genes (i′) and (ii) are distributed on said at least two separate nucleic acid molecules.

In a particular embodiment, wherein said genes (i′) and (ii) are comprised in a bacterial chromosome.

In an alternative embodiment, said genes (i′) and (ii) are comprised in separate plasm ids.

In still an alternative embodiment, said hybrid production system consists of a hybrid helper phage comprising:
(i′) at least one tailocin and/or pyocin structural gene(s), as defined above, derived from a bacterial species or strain containing tailocin and/or pyocin genes, and
(ii) at least one gene, derived from a bacteriophage, as defined in the section “Bacteriophage and gene derived from a bacteriophage” above, involved in phage excision/insertion, phage DNA replication, and/or phage regulation, as defined in the section “Production bacterial cell” above,
wherein said bacteriophage comes from and/or targets bacterial species or strain different from the bacterial species or strain from which said tailocin and/or pyocin structural gene(s) is derived, and
wherein said hybrid helper phage does not comprise any expressed phage structural gene, as defined in the section “Production bacterial cell” above, derived from said bacteriophage.

Unless defined otherwise, all technical and scientific terms used herein have the same meaning as commonly understood by one of ordinary skills in the art to which this invention belongs.

It must be noted that as used herein, the singular forms “a”, “an” and “the” include plural referents unless the context clearly dictates otherwise. Thus, for example, reference to “a cell” includes a plurality of such cells (e.g., a population of such cells). Similarly, reference to “a nucleic acid” includes one or more of such nucleic acids.

Although the invention has been described in conjunction with specific embodiments thereof, many alternatives, modifications and variations will be apparent to those skilled in the art. Accordingly, it is intended to embrace all such alternatives, modifications and variations to fall within the spirit and broad scope of the appended claims.

All publications, patents and patent applications mentioned in this specification are herein incorporated in their entirety by reference into the specification, to the same extent as if such individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

The following examples are given for the purpose of illustrating various embodiments of the invention and are not meant to limit the present invention in any fashion.

Sequences

| SEQ ID | | |
|---|---|---|
| NO: | Description | Type |
| 1 | primase ori from the PICI of the Escherichia coli | DNA |
| | strain CFT073 | |
| 2 | Restriction site | DNA |
| 3 | Primase ori deltaGAAABCC | DNA |
| 4 | Primase ori devoid of restriction sites | DNA |
| 5 | PICI primase-helicase | Protein |
| 6 | PICI primase-helicase | DNA |
| 7 | Kappa structural operon region | DNA |
| 8 | Sequence upstream of small terminase gene of | DNA |
| | Kappa prophage | |
| 9 | p1866 payload | DNA |
| 10 | Candidate HNH protein ORF | DNA |
| 11 | Candidate HNH protein | Protein |
| 12 | p1869 plasmid | DNA |
| 13 | Larger region upstream of Kappa prophage terminase | DNA |
| 14 | p1867 plasmid | DNA |
| 15 | Predicted ORF | DNA |
| 16 | Predicted protein with 2 Zn fingers | Protein |
| 17 | Short cos site | DNA |
| 18 | p1868 payload | DNA |
| 19 | p1872 plasmid | DNA |
| 20 | AD1334 primer | DNA |
| 21 | AD1335 primer | DNA |
| 22 | AD1336 primer | DNA |
| 23 | AD1337 primer | DNA |
| 24 | AD1322 primer | DNA |
| 25 | AD1323 primer | DNA |
| 26 | BW4 genome | DNA |
| 27 | PAC7 genome | DNA |
| 28 | pANS514 plasmid | DNA |
| 29 | PAC7 cos of pAN594 | DNA |
| 30 | operon of gp15-gp19 + gp45 | DNA |
| 31 | pAN241 vector | DNA |

### BRIEF DESCRIPTION OF THE FIGURES

FIG. 1: Lambda genome organization (Lambda packaged phagemid variant). The structural operon is marked with a red line as well as the antitermination protein Q that allows transcription of the late structural operon. Figure adapted from Rajagopala et al. BMC Microbiol 11, 213 (2011).

FIG. 2: Klebsiella pneumoniae prophage genome organization (prophage variant). The structural and regulatory operons are marked with a red line and some structural elements labeled.

FIG. 3: Lambda-K. pneumoniae prophage hybrid. The complete structural operon from the K. pneumoniae prophage (marked with a red line) now occupies the place of the lambda late structural operon. The rest of the Lambda prophage regulating all other functions is intact.

FIG. 4: Titrations of Kappa packaged phagemids with payload pTEST. From top to bottom and left to right, MG1656-OmpCO157, MG1655, MG-Kpne OmpC G1, Kpne F3, MG-Kpne OmpC 7, MG-Kpne OmpC G2, MG-Kpne OmpC G16, MG-Kpne OmpC G15, MG-Kpne OmpC G18. No hits were observed

FIG. 5: Titrations of Kappa packaged phagemids on Klebsiella pneumoniae collections with payload pTEST. Top panels: Kpne collections treated with PBS only and plated on chloramphenicol to see background Cm resistance (left Kpne plate 1; right, Kpne plate 2). Bottom panels: Kpne collections treated with a lysate from Kappa+pTEST. No hits were observed.

FIG. 6: Titrations of Kappa packaged phagemids with payloads pTEST or pTEST-2 in the presence of the HNH protein. Top panel, pTEST+HNH in trans. Bottom panel, pTEST-2+HNH in trans. From top to bottom and left to right, Kpne F3, MG1656-OmpCO157, MG1655, MG-Kpne OmpC G7, MG-Kpne OmpC G2, MG-Kpne OmpC G1, MG-Kpne OmpC G18, MG-Kpne OmpC G16, MG-Kpne OmpC G15.

FIG. 7: Titrations of Kappa packaged phagemids with payloads pTEST or pTEST-2 in the presence of the HNH protein on Klebsiella pneumoniae collections. Top panels: Kpne collection 1 treated with: left) PBS; middle) pTEST+HNH in trans; right) pTEST-2+HNH in trans. Bottom panels, Kpne collection 2 treated with: left) PBS; middle) pTEST+HNH in trans; right) pTEST-2+HNH in trans.

FIG. 8: Titrations of packaged phagemids on Kpne collections. Top panels: Klebsiella pneumoniae collection 1 treated with left, PBS; right, pTEST-3+HNH-Operon in trans. Bottom panels, Klebsiella pneumoniae collection 2 treated with: left, PBS; right, pTEST-3+HNH-Operon in trans.

FIG. 9: Identification of P. freudenreichii phages with PCR. PCR on ORF3 and ORF5 was performed on all phage suspensions. BW4 from plaques 1-3 give a band at the expected size for both orf3 and orf5. Ladder is GeneRuler 1 kb plus.

FIG. 10: Immunity to superinfection of lysogen Pf0s14253. Left panel: Top agar of Pf0s2841 with spots of 4 different BW-like phage suspensions. Right panel: Top agar of Pf0s14253 with spots of 4 different BW-like phage suspensions.

FIG. 11: High induction of BW4 phage after mitomycin C treatment. Left panel: Top agar of Pf0s2841 with spots of culture supernatant from Pf0s14253 without mitomycin C (MMC) induction (ND: non diluted to dilution 10⁻³). Right panel: Top agar of Pf0s2841 with spots of culture supernatant from Pf0s14253 with 0.5 μg/ml of mitomycin C induction (ND: non diluted to dilution 10⁻⁷).

FIG. 12: Genome organization of BW4 and PAC7 bacteriophages. BW4 and PAC7 genome organization is similar with both putative structural operons (represented by the arrows) containing the packaging, head, tail and lysis modules.

FIG. 13: Construction of chimeric BW4-PAC7 prophage. Transformation of the pAN514 suicide plasmid into strain Pf1 s22499 containing the BW4 prophage. Selection on chloramphenicol was used to select for double crossover at the Left Homology Arm (LHA) and Right Homology Arm (RHA). The prophage obtained is a chimer containing a structural operon with first BW4 gp1 followed by gp1-gp14 of PAC7 and after the chloramphenicol selection cassette (CmR) the leftover of BW4 structural genes (gp15-gp25).

FIG. 14: Plasmid map of cosmid pAN594.

FIG. 15: Titration of PAC7 phage-derived particles. Left Panel: Titration from Pf1s22904 plated on erythromycin. Right Panel: Titration from control suspension of strain Pf1s22903 that does not carry any cosmid plated on erythromycin.

FIG. 16: Confirmation for 8 colonies streaked from phage-derived particles titration of Pf1s22904 production by PCR. Top Panel: SLTS PCR (Scholz 2014) on 8 colonies streaked from the phage derived titration assay. Expected size is 612 bp. Bottom Panel: pAN594 specific PCR on 8 colonies. Expected size is 769 bp. Ladder is generuler 1 kb plus.

### EXAMPLES

### Example 1: Exchange of the Structural Operon of Lambda with that of a Phage from a Different Species

The inventors considered that phages can be viewed as more or less large genetic circuits whose final output is the generation of more phage particles. To do this, no matter if the phage is lytic, temperate or chronic (for instance filamentous phages such as M13), the information encoded in their genomes can be roughly categorized depending on the function it performs:
Genes devoted to insertion/excision (for temperate phages).
Genes devoted to DNA replication, RNA transcription, etc. . . . . Some lytic phages encode their own RNA or DNA polymerases, for instance. Some genes modify the host's RNA polymerases to be able to work past terminators, and some other genes are involved in the segregation of the prophage sequence if it exists in a plasmid or linear plasmid form.
Genes related to defense from host's anti-phage mechanisms, degradation/modification of host's elements to complete the lytic cycle, super-exclusion mechanisms or genes that are advantageous for the host.
Genes devoted to DNA packaging: terminases and accessory proteins, ligases, etc.
Structural genes devoted to building a protein capsid for the DNA: apart from strictly structural genes, such as capsid genes, tape measure, fibers, baseplate etc, many other genes are needed to assemble the components (chaperones, proteases) as well as proteins that can be packaged inside the capsid, be it as scaffold or as pilot proteins injected into the cell (for instance, the RNA polymerase of phage N4 or some minor pilot proteins in other phages).

The last two categories (DNA packaging and structural genes) are deeply connected, since the packaging machinery recognizes the pre-assembled heads and the DNA to be packaged, initiates and terminates DNA packaging.

The inventors hypothesized that by abstracting and differentiating all the modules defined above, in principle a system could be built that contains all excision/insertion, replication and regulation elements from one phage and encodes the packaging/structural elements from another one, since, in principle, they could be viewed as independent genetic modules.

In the present example, it is referred to “structural elements” for proteins needed for DNA packaging and structural proteins needed to assemble a mature virion.

Such a system could be very advantageous for different approaches, because:
the structural module from a phage that is not easily amplified or induced could be transferred to another one (i.e. prophages with unknown inducers; prophages found in strains with PICI/SaPI systems; phages for which the host is not known, etc.);
a species which is more amenable for laboratory work/large scale production/safer could be used to produce such particles where the structural genes come from another species;
pure phagemid producing strains could be constructed using the regulatory elements of a well-characterized phage (for instance, Lambda) driving the production of capsids of a different phage, etc.

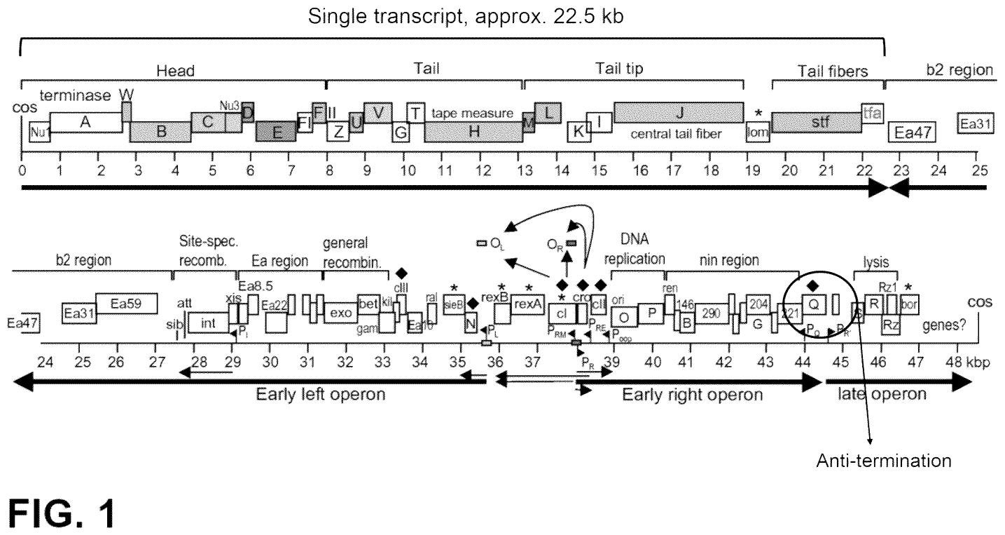

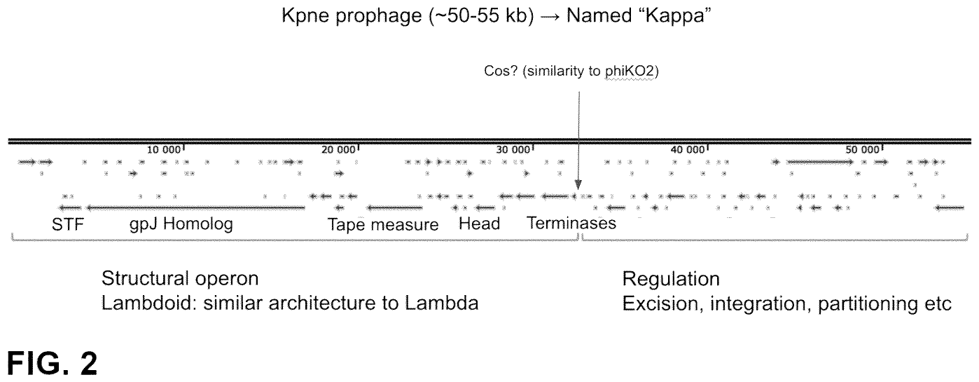

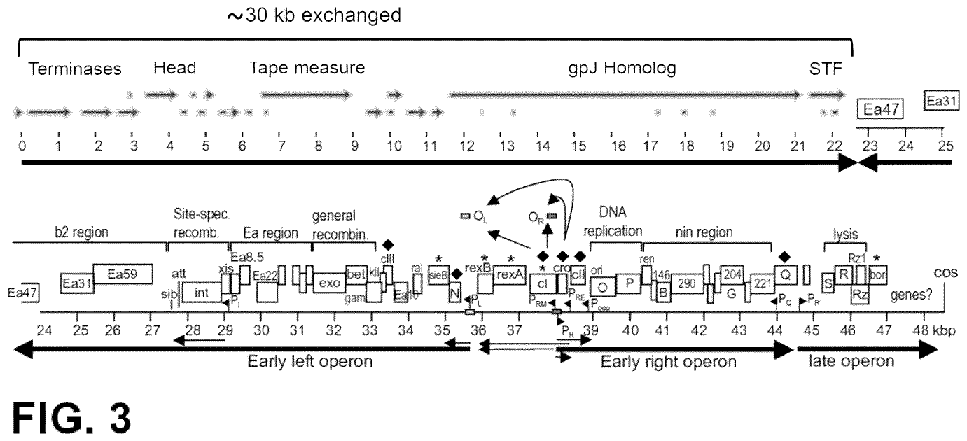

This is the approach that was developed herein. Using a production strain encoding a system to generate pure Lambda phagemids, its structural operon has been exchanged (from the small terminase to the STF gene, about 23 kb) with the structural operon of a prophage coming from a different species (Klebsiella pneumoniae). A schematic diagram shows the changes made for the Klebsiella pneumoniae hybrid variant (FIGS. 1-3).

In this system, the thermolabile version of the prophage Lambda contains all regulatory elements needed to excise the prophage, replicate the circularized excised genome and drive the expression of the long, late operon, including the presence of the antitermination protein Q. This should drive the assembly and packaging of pure phagemid particles completely based on other phages when supplemented with a plasmid containing the correct packaging signals (cos site for the Klebsiella pneumoniae phage).

Analysis of the Klebsiella pneumoniae Prophage

The Kpne strain LMR_3612 (s17699) was analyzed with PHAST to extract prophage regions. One of them contains a predicted lambdoid prophage with some similarities to HK97. This prophage was called Kappa. Of note, a similar Klebsiella oxytoca prophage has been described in the literature and named phiKO2 (Casjens et al. J Bacteriol. 2004 March; 186(6):1818-32). The structural operon was found to span a continuous region encoding terminases, structural genes (capsid, tail, fibers, etc), assembly proteases and chaperones. This region is about 30 kb in length (SEQ ID NO: 7).

### Construction of the Hybrid

The Lambda prophage structural operon was exchanged with the Kappa one using the lambda red recombineering system, starting from a production strain containing a Lambda prophage without the cos site (s1965). Of note, some codons were recoded to be efficiently recognized by E. coli.

### Production and Titrations

The produced strain containing a completely exchanged structural operon was labeled Kappa. Next, a payload suitable to be packaged by this phagemid was constructed. To do this, a 350-bp long sequence (SEQ ID NO: 8) right upstream of the small terminase gene of the Kappa prophage, a candidate to encode the cos signal recognized by the Kappa terminases, was inserted in a payload containing a chloramphenicol marker and a sfGFP gene. This payload was labeled pTEST (p1866, SEQ ID NO: 9). Of note, several restriction sites found in the Klebsiella pneumoniae REBASE database were removed, where possible. The strain Kappa harboring the p1866 plasmid was grown overnight in LB+chloramphenicol and the next day a production following the protocol below was performed.

Overnight cultures were diluted 1:6 in a final volume of LB+5 mM CaCl₂) supplemented with chloramphenicol and grown for 30 min at 30° C. with shaking. After that, a 45-minute-long heat shock at 42° C. was performed. Finally the cultures were grown at 37° C. for 3 hours with shaking. After this period, cells were recovered by centrifugation and lysed using 3 mL of B-PER protein extraction reagent, 600 mg of detergent removal bio-beads were added and an incubation at room temperature with mild shaking performed for 1 hour. After that, the lysates were centrifuged for 10 min at 10,000 g and the supernatants filtered through a 0.2 micron pore-size membrane.

Two collections of Klebsiella pneumoniae strains belonging to different ST types (192 strains in total) were used to verify if phagemid particles were produced. Overnight cultures of Klebsiella pneumoniae strains were diluted 1:100 in LB+CaCl₂), grown for 2 hours at 37° C. and diluted 1:20 before the transductions. 10 μL of phagemid lysate was added to 90 μL of each of the Klebsiella pneumoniae dilutions and incubated for 30 min at 37° C. Finally, 10 μL of each transduction were plated on LB agar supplemented with chloramphenicol and incubated overnight at 37° C. Additionally, Klebsiella pneumoniae strain F3 (s19091), MG1655 (s003), MG1656-OmpCO157 (s14269) and MG1656-dOmpC-dLamB harboring 6 different Klebsiella pneumoniae OmpC variants in trans were used to verify the titers of the productions.

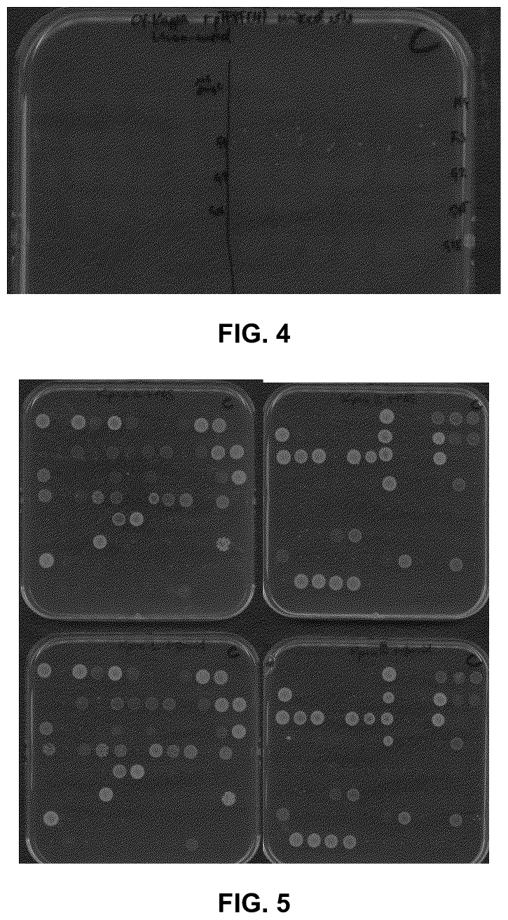

In this first assay, absolutely no hits were observed, either in the 192 Klebsiella pneumoniae strains or in any of the other 9 strains used for titrations (FIGS. 4-5).

After these results, a more detailed analysis of the Kappa prophage was performed to see if some structural or packaging element had been missed. Since this prophage seems to belong to the same family as HK97, a literature search was conducted in order to find any missing elements, and it was identified that HK97 (and in general, many other phages but not Lambda) need a small protein containing an HNH nuclease domain that assists in the processivity of the cos site cutting by the terminase complex (Moodley et al. Protein Sci. (2012) 21(6):809-818 and Kala et al. Proceedings of the National Academy of Sciences April 2014, 111 (16) 6022-6027). An analysis of the Kappa prophage region right upstream of the terminase genes (and the putative cos site) revealed the presence of an operon containing an HNH protein, some other small ORFs with unknown functions and a Zn-finger domain-containing protein right upstream of the putative cos site.

To test the hypothesis that the HNH protein was the element missing in the production strain, this ORF (SEQ ID NO: 10 and SEQ ID NO: 11) was cloned in a plasmid under the control of the inducible repressor PhIF (p1869, SEQ ID NO: 12) and used to complement the productions. Additionally, a second payload was built that contained a larger region upstream of the terminases (SEQ ID NO: 13), in case the cos site present in pTEST was not complete. This second plasmid was labeled pTEST-2 (p1867, SEQ ID NO: 14).

Phagemids were produced as for the initial experiment, but DAPG was added to the productions when the cultures were shifted to 42° C. in order to induce expression of the HNH protein. Screening of the collections was done as described above.

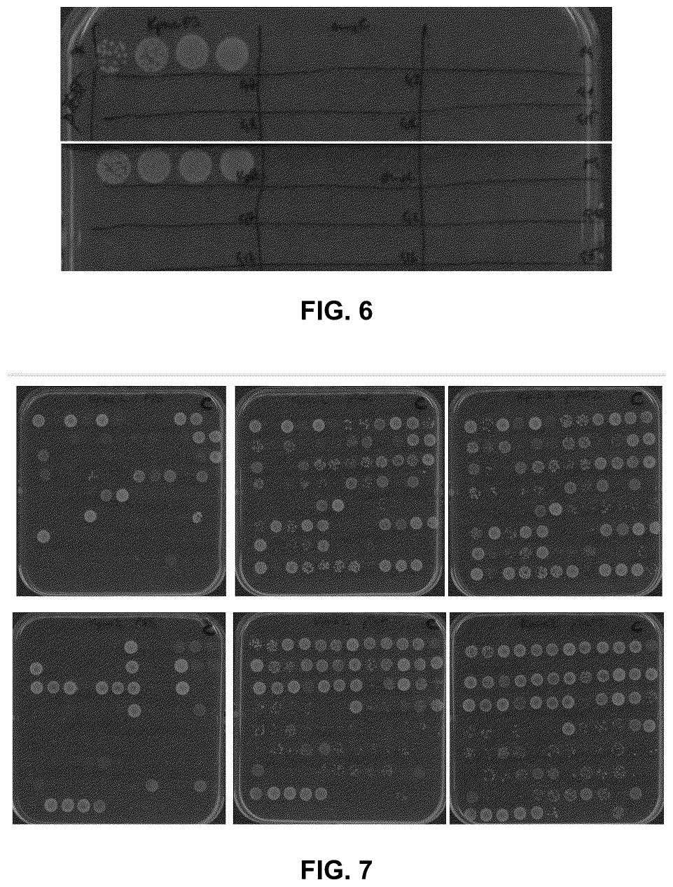

The results showed that in the presence of the HNH protein, phagemids are readily produced (FIG. 6). In this case, the titration in single MG1655 or Klebsiella pneumoniae F3 strains revealed that the phagemid is specific to Klebsiella and that it does not recognize E. coli at all. This is proof that the phagemids being produced are structurally based on Kappa, but regulated and maintained in the lysogenic state by the Lambda prophage.

Similarly, when titrated on both Kpne collections, this time many hits were observed (FIG. 7).

Two observations can be made from this experiment:
Titers are different when produced with pTEST or pTEST-2 payloads, both carrying the HNH protein in trans (estimated 5×10⁵ TU/mL for pTEST and 5×10⁶ TU/mL for pTEST-2).
Even at low titers, spots in the Klebsiella pneumoniae collection are dense, indicating that the delivery efficiency must be high.

Concerning the difference in titers for the payloads tested, the only change between both productions is that the putative cos site present in pTEST-2 is longer than pTEST; but also, pTEST-2 encodes a small ORF (which is part of the longer cos region) with two Zn fingers predicted (SEQ ID NO: 15 and SEQ ID NO: 16). If this protein is involved in packaging, it may be the reason why titers are higher in lysates containing pTEST-2 than in those containing pTEST, and not because of the length per se. For this reason, a third system was built in which a payload encoding a short cos site (to avoid encoding any proteins in that region) (SEQ ID NO: 17) was used, labeled pTEST-3 (p1868, SEQ ID NO: 18). To complement the putative packaging proteins in trans, the so-called “HNH-Operon” plasmid was built (p1872, SEQ ID NO: 19), encoding the HNH protein (SEQ ID NO: 10 and SEQ ID NO: 11) and the putative Zn-finger containing protein (SEQ ID NO: 15 and SEQ ID NO: 16). Productions were made the same way as described above.

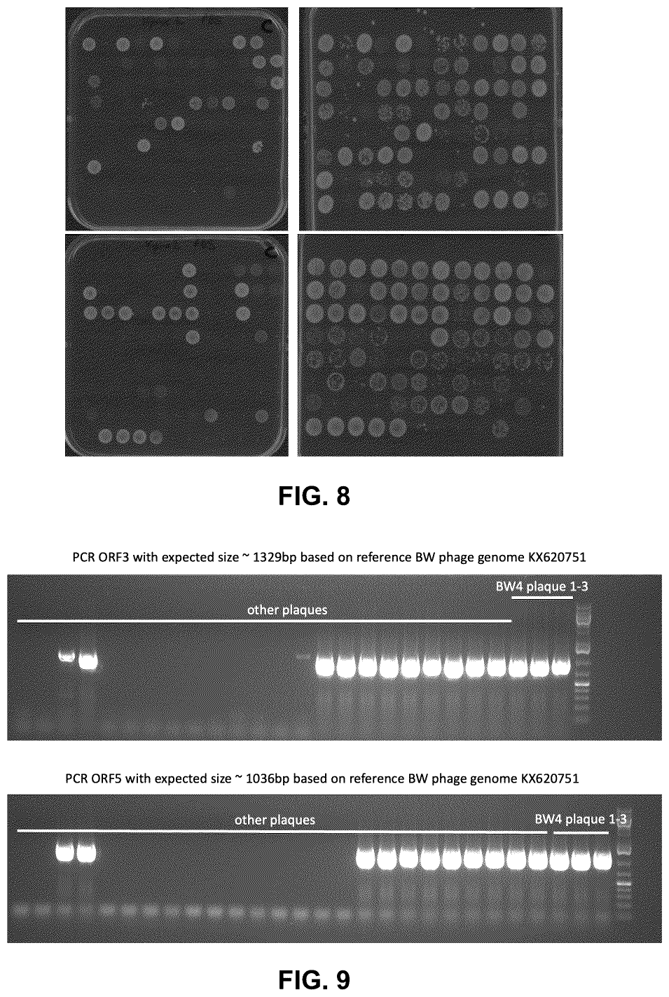

In this third case, a similar behavior as for pTEST-2 was observed: many hits in the Kpne collections, confirming successful productions of phagemids and titers of about 5×10⁶ TU/mL (FIG. 8). The titers were obtained by analyzing dense spots on the Klebsiella pneumoniae plates and using those strains to titrate the lysates. These results show that the minimal cos site is encoded in a shorter region than pTEST and pTEST-2 and that the HNH-Operon machinery is necessary and improves the packaging reaction.

These results show that an E. coli production strain is able to produce Klebsiella-specific pure phagemid particles. The specificity does not come solely from the addition of a single structural element, such as a tail fiber. The complete protein composition of the phagemids produced is from K. pneumoniae origin, proving that phagemid particles for other species can be assembled using E. coli as a production strain.

### Example 2: Production of Cutibacterium acnes Phage-Derived Particles

Cutibacterium acnes is one of the most prevalent and abundant species of the skin (Kashaf et al. Nat Microbiol 7, 169-179 (2022)) where it colonizes the pilosebaceous unit (PSU). Unlike on the stratum corneum, bacteria present in the PSU are surrounded by living cells notably keratinocytes, sebocytes and different immune cells (Kabashima et al. Nat Rev Immunol 19, 19 30 (2019)). Close contact between C. acnes and these cells might lead to either beneficial or detrimental interactions. (Bruggemann et al. Front Microbiol 12, 673845 (2021)). Being able to genetically modify C. acnes was notoriously challenging before the applicant's new tools disclosed in US applications US2022/135986 and US2022/135987. In these patent applications, the inventors described, for the first time, the production of C. acnes phage-derived particles using C. acnes as a production strain.

In the present example, the inventors used P. freudenreichii strain to produce C. acnes phage-derived particles by swapping the structural genes from a P. freudenreichii prophage for the structural genes of a C. acnes phage.

### Results

### Isolation of BW4 Phage

P. freudenreichii and associated bacteriophages are known to be present in some dairy products (Gautier et al. (1995) Lait 75:427-434; Gautier et al. (1995) App. Environ. Microbiol. 61:2572-2576; Cheng et al. (2018) BMC Microbiology 18:19). The inventors therefore screened for the presence of both Propionibacterium phages or P. freudenreichii lysogens in cheese samples.

Different types of cheese samples were grinded, resuspended in Reinforced Clostridial Medium (RCM) and incubated at 30° C. in anaerobic conditions for 2 days. After incubation, a dilution of the culture was performed in lithium glycerol broth, a media selective for Propionibacteria (WO1994017201), and incubated for 6 days at 30° C. A final dilution in RCM+mitomycin C was incubated for 1 day at 30° C. in order to induce potential prophages. The induced cultures were filtered (0.2 μm) and spotted on different indicator strains. One of the samples led to turbid plaque formation on top agar of the P. freudenreichii strain Pf0s2841. Three individual plaques were isolated by two successive picking and streaking on Pf0s2841 and amplification was performed on top agar of Pf0s2841. For the three different plaques, amplification led to phage suspension ˜10¹⁰ PFU/mL.

Two clusters of temperate dsDNA P. freudenreichii phages (BW and BV) have been previously identified (Cheng et al. (2018) BMC Microbiology 18:19). Using PCRs designed on BW genome from Doucette phage (KX620751) two different fragments were extracted:
ORF3 with AD1334 (SEQ ID NO: 20)/AD1335 (SEQ ID NO: 21)
ORF5 with AD1336 (SEQ ID NO: 22)/AD1337 (SEQ ID NO: 23).

The inventors could classify the isolated phages as BW-like (FIG. 9). Sequencing of ORF5 revealed that all phages were most probably identical and therefore were coming from the same BW-like phage that was named BW4.

### Isolation of Pf0s2841 Lysogen Carrying the BW4 Phage

The inventors then isolated P. freudenreichii lysogen carrying the BW4 phage as a prophage. For that, BW4 phage suspension was spotted on strain Pf0s2841 and incubated for 3 days. Turbid plaques were picked, resuspended and streaked. After 5 days, single colonies were obtained, several colonies were streaked and incubated a second and third time and presence of the phage genes was checked, at each streaking, by PCR, after DNAse treatment, across the cohesive ends (AD1322 (SEQ ID NO: 24)/AD1323 (SEQ ID NO: 25)) to ensure presence of the phage but absence of phage particles.

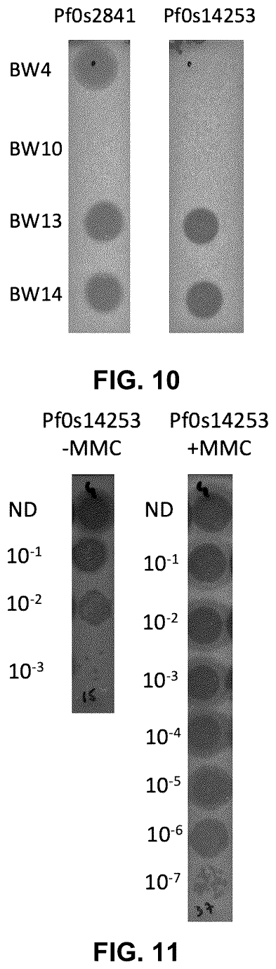

After the third streak, colonies were grown as a top agar and a spot of non diluted BW-like phages suspensions were spotted on the putative lysogene strain (Pf0s14253) and on the ancestor strain (Pf0s2841). After incubation, clearance was observed for both strains for BW13 and BW14 spots whereas clearance was only observed for Pf0s2841 in the case of BW4 spot (FIG. 10). This indicates that the strain Pf0s14253 is immune to BW4 phage superinfection and carries the BW4 prophage. The absence of immunity for BW14 and BW13 indicates that these phages have likely a different immunity repressor.

BW4 prophage induction

In order to use the BW4 lysogen strain as a production strain for phage-derived particles the inventors first had to test the ability to produce high concentration of the BW4 phage upon induction of the lytic cycle. To do so, Pf0s14253 was grown in absence or presence of mitomycin C (MMC), an antibiotic known to induce prophages, and the culture supernatant was titered for the presence of BW4 phage particles on the indicator strain Pf0s2841. A high amount of BW4 phage particles was observed in the condition supplemented with mitomycin C (FIG. 11) with 7.4×10⁷ PFU/μL against 3.0×10³ PFU/μL for the condition without mitomycin C. This indicates a high dynamic range between lytic and lysogenic cycle for BW4 prophage under such conditions and confirmed the potential of BW4 for the production of phage-derived particles.

### Sequencing and Annotation of BW4 Phage

To engineer the BW4 prophage towards production of C. acnes phage-derived particles, the BW4 phage was sequenced. DNA isolation (Promega Wizard DNA Clean-Up System) followed by Illumina sequencing was performed on BW4 phage suspension. Raw reads were assembled into a single contig using Spades and termini were corrected by sanger sequencing (SEQ ID NO: 26). Annotation was performed using Phaster and manually curated based on homologies with other BW-like phages (Cheng et al. (2018) BMC Microbiology 18:19).

As described in Cheng et al. (2018) BMC Microbiology 18:19, BW-like phages have typical genomic architecture of other temperate phages with a large putative structural operon (also called lytic operon) organized in different functional modules with, in order of transcription: packaging, head, tail, and lysis module. Surprisingly, the first gene of the putative operon (gp1) appears to be related to DNA replication based on HHpred as it contains a domain similar to bifunctional primase and polymerase proteins. Other parts of the BW4 phage genome contain the genes necessary for prophage integration/excision, DNA replication, DNA recombination, regulation of the lytic/lysogenic cycle and other accessory proteins. This modular architecture confirms the possibility to swap the genes necessary for the production of BW4 phage capsid and the packaging of the phage genome by their equivalent from a C. acnes phage genome.

Isolation of C. acnes PAC7 Phage

C. acnes phages were isolated from skin of healthy volunteers. Briefly a patch (Biore) was applied to the nose allowing to extract comedones that were resuspended in RCM, plated on MRS and incubated at 37° C. in anaerobic conditions. For some of the plates, plaques could be observed in the dense lawn of C. acnes. DPBS (Dulbecco's Phosphate Buffered Saline) was poured on the plate to resuspend potential phages and filtered to remove bacteria. This phage suspension was streaked on plate and a top agar of strain Ca0s2345 was added. Plates were incubated for 2 days and plaques were reisolated by three successive picking, streaking and top agar plating. Finally a plaque was amplified on top agar with Ca0s2345 strain and the resulting phage suspension was PEG precipitated. High titer (>10⁶ PFU/μL) phage suspension was obtained when titered on Ca0s2345.

### Sequencing and Annotation of PAC7 Phage

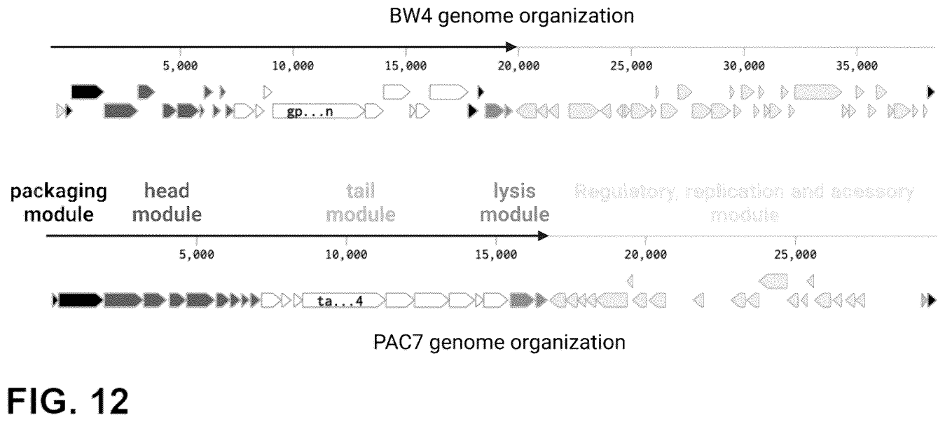

DNA isolation (Promega Wizard DNA Clean-Up System) followed by Illumina sequencing was performed on PAC7 phage suspension. Raw reads were assembled into a single contig using Spades and termini were corrected by sanger sequencing (SEQ ID NO: 27). Annotation was performed using Phaster and manually curated based on homologies with other C. acnes phages (Marinelli et al. (2012) mBio 3:e00279-12). Similar to the P. freudenreichii BW4 phage, a structural operon comprising modules for packaging, head and tail assembly and cell lysis was identified (FIG. 12). An HNH endonuclease was identified as the last gene of the phage (gp45). Such endonuclease has already been shown to be essential for efficient packaging (Quiles-Puchalt et al. (2014) Proc Nat. Acad. Sci. 111:6016-6021).

Construction of Lysogen Strain with a Chimeric BW4-PAC7 Prophage

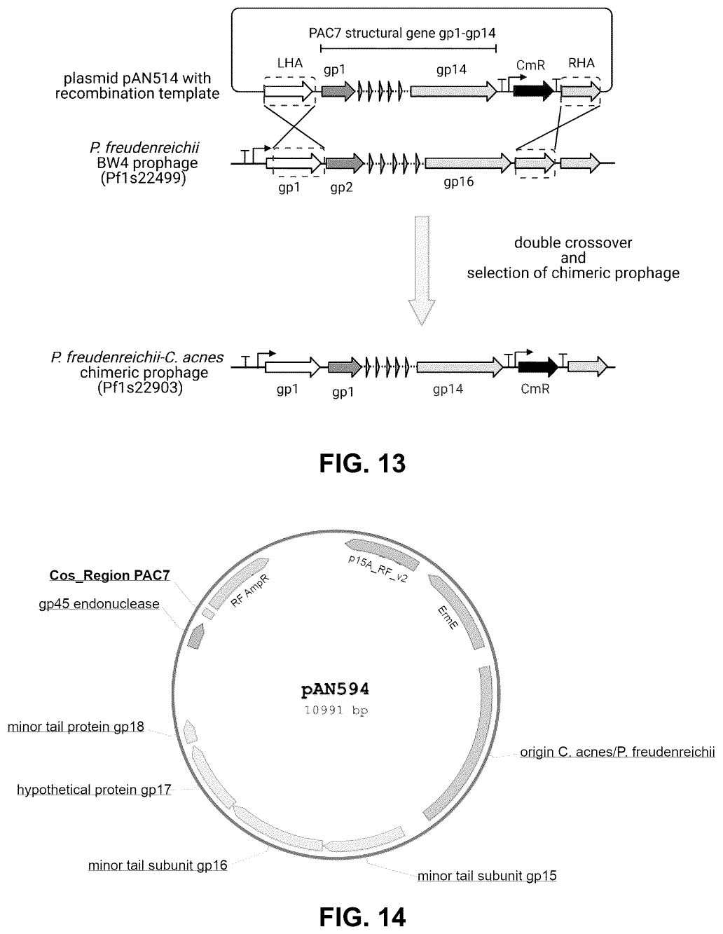

The genes in the structural operon of BW4 prophage, from the small terminase gp2 to the tape-measure protein gp16 included, were replaced by the structural PAC7 genes from gp1 to gp14 (FIG. 13). This was performed by homologous recombination using plasmid pAN514 (SEQ ID NO: 28), a P. freudenreichii suicide vector that was cloned in E. coli DH10B. After transformation of the vector, a double crossing over event was selected in P. freudenreichii (Pf1s22499) by selection on chloramphenicol. The chimeric BW4-PAC7 structural operon integrity was globally confirmed by PCR and sanger sequencing of the entire chimeric structural operon.

Production and Titration of PAC7 Derived Particles from a Lysogen Strain Carrying a Chimeric BW4-PAC7 Prophage

In order to produce C. acnes phage-derived particles from a P. freudenreichii BW4-PAC7 chimeric lysogen, the pAN594 cosmid (FIG. 14) containing the packaging signal of the PAC7 phage (SEQ ID NO: 29), an operon expressing five genes of the PAC7 tail module (gp15-gp19) and the gp45 endonuclease (SEQ ID NO: 30) and an origin of replication functional in P. freudenreichii and C. acnes (as disclosed in US2022/135986 and US2022/135987) were transformed into Pf1 s22903. Transformants were streaked and grown in presence of both chloramphenicol (1 μg/mL) to select for the presence of the prophage and erythromycin (2.5 μg/mL) to select for the presence of pAN594. At OD_{600 nm}˜0.4, culture was supplemented with 0.5 μg/ml of mitomycin C and grown overnight at 30° C. in anaerobic conditions. After incubation, cells were collected by centrifugation, lysed by bead beating (2×20 min at 30 Hz with 0.1 mm glass beads), supernatant was filtered and the presence of phage derived particles was titered on C. acnes Ca0s2258.

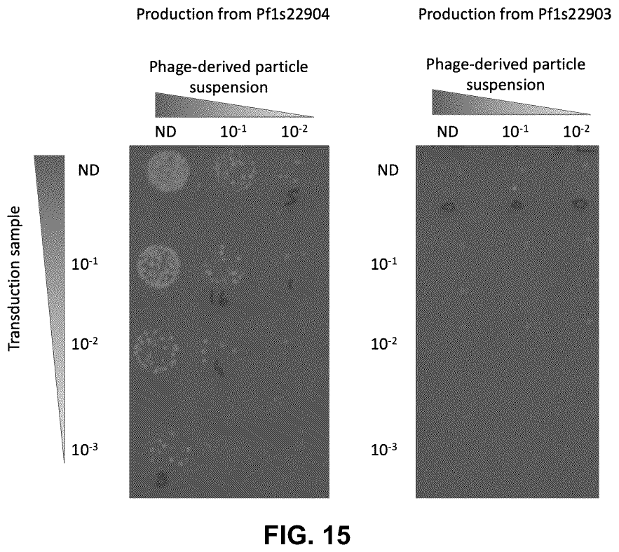

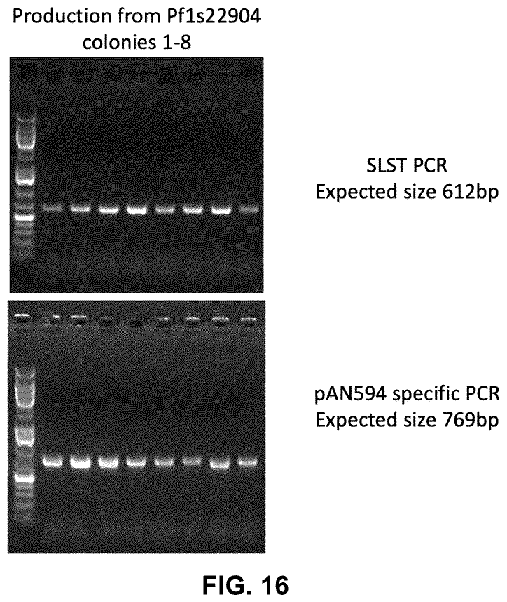

Up to ˜10² potential transductants per μL were obtained (FIG. 15). 8 colonies were streaked on Brain Heart Infusion (BHI) erythromycin (5 μg/mL) and confirmed to be C. acnes and transductants carrying pAN594 using PCR (FIG. 16).

The inventors thus demonstrated for the first time that C. acnes phage-derived particles able to deliver DNA into Cutibacterium acnes can be produced by swapping structural genes of a P. freudenreichii prophage for the structural genes of a Cutibacterium acnes phage.

### Material and Methods:

### Strain Used and Generated

TABLE 1
Strains used and generated

| Eligo ID | Description |
|---|---|
| Pf0s2841 | Indicator strain for P. freudenreichii BW4 phage |
| | (CIRM-BIA 509, TL110 belonging to INRAE) |
| Pf0s14253 | Strain Pf0s2841 with a BW4 prophage |
| Pf1s22499 | Strain Pf0s14253 with the packaging signal of BW4 deleted |
| Pf1s22903 | Strain Pf1s22499 with the BW4 genes gp2-gp16 replaced by |
| | PAC7 gp1-gp14 |
| Pf1s22904 | Strain Pf1s22903 with pAN594 |
| Ca0s2345 | Indicator strain for C. acnes PAC7 phage |
| Ca0s2258 | Cutibacterium acnes ATCC 11828 |

### Culture Conditions

All incubations of P. freudenreichii strains were performed at 30° C. in anaerobic conditions (Thermo Scientific™ Sachet Oxoid™ AnaeroGen).

All incubations of C. acnes strains were performed at 37° C. in anaerobic chamber.

### Construction of Strain Pf1s22499

Deletion of the packaging signal from BW4 prophage was performed by homologous recombination and CRISPR-Cas selection of the recombinant using the pAN241 P. freudenreichii vector that was cloned in E. coli and then transformed into Pf0s14253 strain. The pAN241 vector contains a template for homologous recombination (SEQ ID NO: 31) and a FnCpf1 transcriptional cassette with a crRNA targeting the cos of the BW4 prophage.

Transformation Protocol for P. freudenreichii

Transformation of P. freudenreichii was adapted from Brede, D. A. et al. App Environ Microb 71, 8077-8084 (2005), replacing SLB (sodium lactate broth) media for BHI.

### Phage-Derived Particles Titration

Strain Ca0s2258 was streaked on BHI agar plate. Once dense growth on plate was obtained, a liquid culture was set up in BHI. After overnight incubation, the turbid culture was concentrated 10× in BHI. 90 μl of cells were mixed with pure, diluted 1/10 and diluted 1/100 solutions of 10 μL of phage-derived particles produced from either Pf1s22904 or Pf1s22903 as negative control. Samples were incubated 2 hours at room temperature and then 1/10 serial dilutions were performed in BHI, samples were incubated 2 h at 37° C. in anaerobic conditions before spotting 4 μL on BHI+5 μg/mL erythromycin. Plates were incubated for 7 days at 37° C. in anaerobic conditions.

1. Production bacterial cell for producing phage particles or phage-derived delivery vehicles, said production bacterial cell stably comprising at least one phage structural gene and at least one phage DNA packaging gene, said phage structural gene(s) and phage DNA packaging gene(s) being derived from a first type of bacteriophage,
wherein the expression of at least one of said phage structural gene(s) and/or at least one of said phage DNA packaging gene(s) in said production bacterial cell is controlled by at least one induction mechanism, and
wherein said production bacterial cell is from a bacterial species or strain different from the bacterial species or strain from which said first type of bacteriophage comes from and/or that said first type of bacteriophage targets.

2. The production bacterial cell according to claim 1, wherein said bacterial cell further comprises a payload to be packaged into said phage particles or phage-derived delivery vehicles.

3. The production bacterial cell according to claim 2, wherein said payload is a nucleic acid payload comprising a packaging site derived from said first type of bacteriophage.

4. The production bacterial cell according to claim 2, wherein said payload is to be delivered into targeted bacterial cells.

5. The production bacterial cell according to claim 4, wherein said payload comprises a sequence of interest.

6. The production bacterial cell according to claim 5, wherein said sequence of interest only generates an effect in said targeted bacterial cells.

7. The production bacterial cell according to claim 6, wherein said targeted bacterial cells are from a species or strain different from the production bacterial cell.

8. The production bacterial cell according to claim 1, wherein the same induction mechanism controls the expression of the at least one of said phage structural gene(s) and the at least one of said phage DNA packaging gene(s).

9. (canceled)

10. The production bacterial cell according to claim 1, wherein the at least one induction mechanism controls the expression of all said phage structural gene(s).

11. The production bacterial cell according to claim 1, wherein the at least one induction mechanism controls the expression of all said phage DNA packaging gene(s).

12. The production bacterial cell according to claim 1, wherein said phage structural gene(s) and phage DNA packaging gene(s) derived from said first type of bacteriophage are comprised in at least one plasmid, chromosome, and/or helper phage.

13. The production bacterial cell according to claim 1, wherein said production bacterial cell further comprises at least one gene, derived from a second type of bacteriophage, involved in phage excision/insertion, phage DNA replication, and/or phage regulation,
said second type of bacteriophage coming from and/or targeting bacterial species or strain different from the bacterial species or strain from which said first type of bacteriophage comes from and/or that said first type of bacteriophage targets.

14. The production bacterial cell according to claim 13, wherein said production bacterial cell is from the same bacterial species or strain as the bacterial species or strain from which said second type of bacteriophage comes from and/or that said second type of bacteriophage targets.

15. The production bacterial cell according to claim 1, wherein said production bacterial cell is an E. coli bacterial cell.

16. The production bacterial cell according to claim 1, wherein said production bacterial cell is a P. freudenreichii bacterial cell.

17. The production bacterial cell according to claim 16, wherein the bacterial species or strain from which said first type of bacteriophage comes from and/or that said first type of bacteriophage targets is C. acnes.

18. A method for producing phage particles or phage-derived delivery vehicles, comprising:
(a) providing a production bacterial cell for producing phage particles or phage-derived delivery vehicles, said production bacterial cell stably comprising at least one phage structural gene and at least one phage DNA packaging gene, said phage structural gene(s) and phage DNA packaging gene(s) being derived from a first type of bacteriophage,
wherein the expression of at least one of said phage structural gene(s) and/or at least one of said phage DNA packaging gene(s) in said production bacterial cell is controlled by at least one induction mechanism, and
wherein said production bacterial cell is from a bacterial species or strain different from the bacterial species or strain from which said first type of bacteriophage comes from and/or that said first type of bacteriophage targets, and
(b) inducing, in said production bacterial cell, expression of said at least one of said phage structural gene(s) and said at least one of said phage DNA packaging gene(s), and assembly of the products expressed by said at least one phage structural gene(s) and said at least one phage DNA packaging gene(s), thereby producing phage particles or phage-derived delivery vehicles.

19. Hybrid helper phage system comprising:
(i) at least one phage DNA packaging gene(s) derived from a first type of bacteriophage,
(i′) at least one phage structural gene(s) derived from said first type of bacteriophage, and
(ii) at least one gene, derived from a second type of bacteriophage, involved in phage excision/insertion, phage DNA replication, and/or phage regulation,
wherein said genes (i), (i′) and (ii) are comprised in a unique nucleic acid molecule or in separate nucleic acid molecules,
wherein said first type of bacteriophage comes from and/or target bacterial species or strain different from the bacterial species or strain from which said second type of bacteriophage comes from and/or that said second type of bacteriophage targets, and wherein said hybrid helper phage system does not comprise any expressed phage structural gene derived from said second type of bacteriophage.

20. The hybrid helper phage system according to claim 19, wherein said genes (i), (i′) and (ii) are comprised in a bacterial chromosome or wherein said genes (i), (i′) and (ii) are comprised in separate plasm ids.

21. (canceled)

22. The hybrid helper phage system according to claim 19, wherein said hybrid helper phage system consists of a hybrid helper phage comprising:
(i) at least one phage DNA packaging gene(s) and at least one phage structural gene(s) derived from a first type of bacteriophage, and
(ii) at least one gene, derived from a second type of bacteriophage, involved in phage excision/insertion, phage DNA replication, and/or phage regulation,
wherein said first type of bacteriophage comes from and/or target bacterial species or strain different from the bacterial species or strain from which said second type of bacteriophage comes from and/or that said second type of bacteriophage targets, and wherein said hybrid helper phage does not comprise any expressed phage structural gene derived from said second type of bacteriophage.

23-30. (canceled)


SEQUENCE LISTING (31 sequences)

SEQ ID NO: 1 - DNA, 282 bp, synthetic construct
  source 1..282: mol_type = other DNA; note = primase ori from the PICI of the Escherichia coli strain CFT073
tttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagccgcgccattactggcatggtggccagtaaggtagataaggtagacaaggggaggcacaactcaaaactttttaaacgagggggtaaaa

SEQ ID NO: 2 - skipped

SEQ ID NO: 3 - DNA, 282 bp, synthetic construct
  source 1..282: mol_type = other DNA; note = Primase ori deltaGAAABCC
tttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagcagcgccattactggcatggtggccagtaaggtagataaggtagacaaggggaggcacaactcaaaactttttaaacgagggggtaaaa

SEQ ID NO: 4 - DNA, 282 bp, synthetic construct
  source 1..282: mol_type = other DNA; note = Primase ori devoid of restriction sites
tttgttgcaatggctgtctaccctgtctacctgagtaaagaaaaatacatttaattcagtatattaacttgggtagacagcctttttttactgtctaccttctgtctaccctctctacctgattttacctgaatcagacagggaggtagacacggggtagacagtggataaaagcactctaccccactgaaagcagtgccattactggcatggttgccagtaaggttgataaggtagacaaggggagggacaactcaaaactttttaaacgagggggtaaaa

SEQ ID NO: 5 - protein, 584 aa, synthetic construct
  source 1..584: note = PICI primase-helicase
MKLAPNVKQQSRGIKHKETEVIIFAGSDAWSHAKQWQEHDARMAGDNEPPVWLGEQQLSELDKLQIVPEGRKSVRIFRAGYLAPVMIKAIGQKLAAAGVQDANFYPDGMHGQKVENWREYLARERQNLSDGLVIELPVKQKAQLSQMADSERAQLLADRFDGVCVHPESEIVHVWCGGVWCPVSTMELSREMVAIYSEHRATFSKRVINNAVEALKVIAEPMGEPSGDLLPFANGALDLKTGEFSPHTPENWITTHNGIEYTPPAPGENIRDNAPNFHKWLEHAAGKDPRKMMRICAALYMIMANRYDWQMFIEATGDGGSGKSTFTHIASLLAGKQNTVSAEMTSLDDAGGRAQVVGSRLIVLADQPKYTGEGTGIKKITGGDPVEINPKYEKRFTAVIRAVVLATNNNPMIFTERAGGVARRRVIFRFDNIVSEAEKDRELPEKIAAEIPVIIRRLLANFADPEKARALLIEQRDGDEALAIKQQTDPVIEFCQFLNFLEEARGLMMGGGGDSVKYTTRNSLYRVYLAFMAYAGRSKPLNVNDFGKAMKPAAKVYGHEYITRKVKGVTQTNAITTDDCDAFL

SEQ ID NO: 6 - DNA, 1752 bp, synthetic construct
  source 1..1752: mol_type = other DNA; note = PICI primase-helicase
atgaaactggcaccgaacgtaaaacagcagtcacgcggcataaaacacaaagaaacagaagtcattatttttgcgggtagtgatgcctggtcacacgcaaaacaatggcaggaacatgacgcgcgtatggccggagataatgagcctcctgtgtggcttggggagcagcagttatccgaactggataagctgcaaattgtgccggaaggcagaaaatccgtgcgcatattcagggccggatatcttgcgccagtaatgataaaggcgattggtcagaagctggcggcggcaggcgtacaggatgcaaatttttaccctgatggtatgcacggtcagaaggtggagaactggcgcgaatatctggcccgtgagcgccagaatctttctgatggtctggtcattgagcttccggtaaagcaaaaggcgcaactttcgcagatggcggacagtgagcgcgcgcagctgcttgccgatcgctttgatggcgtttgcgtacatcctgaaagtgaaatcgttcacgtatggtgcggcggggtatggtgtccggtcagcacaatggagctgagccgcgaaatggtggcgatctattcagagcacagggccactttcagcaagcgcgtaatcaataacgccgtggaagcgttaaaagttattgccgaaccaatgggcgagccgtccggcgatttgctgccgttcgccaatggtgcgcttgacctgaaaacgggggaattttccccgcacacgccggagaactggatcaccacgcacaacggcattgagtacacgccaccagcacccggggagaacatccgcgataacgcgccaaactttcataaatggcttgagcacgcagccggaaaagacccgcgcaagatgatgcgtatatgtgccgcgctgtacatgattatggcgaaccggtacgactggcagatgtttattgaggccaccggagacggcgggagcggtaaaagtacattcacacacatagccagccttctggcagggaaacaaaacacggtaagcgctgaaatgacatcgcttgatgatgctggtgggcgtgcgcaggttgtcgggagtcgtcttatcgtcctggcagaccagccgaaatatacaggcgaaggaacgggcatcaagaaaatcacgggcggcgaccccgtggaaattaacccgaaatatgaaaagcgttttacggcggtaatcagggcggtggtgctggcaaccaataacaatccgatgatattcaccgaacgggccggaggtgtggcacgtcgtcgggtgatattccggttcgataacatcgtaagcgaggcagaaaaagacagggagctaccggaaaagatcgcggctgaaatccctgtcattatccgccgcttgctggcgaactttgccgaccctgaaaaggcacgggctttactcattgaacagcgtgacggtgatgaagcactggcaataaagcaacagacggatccggttattgagttttgccagttcctgaattttctggaggaagcacgcggcctgatgatgggcggcggtggcgattcagtgaagtacacgaccagaaacagcctttaccgcgtctatctggcgtttatggcgtacgcaggcaggagcaaaccgctaaacgtaaatgactttggcaaggctatgaagccagccgcgaaagtttacggacatgaatatattacgcggaaagttaaaggagtaacgcagactaacgcaataacaacagacgattgcgacgcgttttta

SEQ ID NO: 7 - DNA, 29808 bp, Kappa phage
  source 1..29808: mol_type = genomic DNA
gctgttttggggtgcttttgaatgagtacagggatgcgatcacctggtggcggacgcaaatcgaataacactggaaatcaggttagttctttaaccagagcggtttctccgccggatgaattactgggcgatatggctatcgatgcctggaaacggacgtgcaaaattcttattaaccgtggcacgttcgaaatggaagattgttatttgctgatggaatactgcaacaccgtgcagctgctgtacgacgccaaccaggaaattaaaagcgatggccttggtgatgataccgctgccggcggtcagaaacttggtgcggcagtgaaggcgcgtagccgttatatcagcgaattaattcgactctccgttgtgttaaagctggaccccaatagccgcatcctgaagaaacagcccggagataatgacaaatccagcggtgagttcgacgagttttaattttggtgcggccctaatgacttaaggatggagcatggccgcatatccaaacgtcaatgtggcgaacaaatatgcgcgggatatcatagacgggaaaatagtcgcctgcagagctattcggctggcatgtcagcgccattttgacgatttaaaaaaatcactcgataacaattacccttaccggttcgacagagatttagctgagcgggcctgccggtttgttcagaaattaccgcactccagtggcgatttggcggggcagaaattaaaactggaaccttggcaaagttttattttttgttcgatttttggctgggtcacgaaaaaggataaaaaacgccgatttcgcgaagcgtatatccgggtagccaggaaaaacgggaaatcgttttttgctgccgggattggcacctacatgttttgcgctgatggcgaaaacagcgcagaagtgtattgcggtgcgacaactatggcgcaggcgaaaaaggtcttcaccccagccaggcagatggccagccgcctgccggcacttcgctccagatttgatatttcggtatggaccgacagcctgacacgcccggatggttccgttttcgcacctatggcggggaaacccggcgatggtgacagcccacattgcgcgatcattgacgagtatcacgaacacgatacggatcatatgtacgaggccatgacaatggggatgggcgcccgttcgcagccgttaacgctcattatcacgacagccggctcgtcactggagtccccttgctatgacaaggacaaggaagtcaaagaggttatcgaaggcataacccgtaatgatcgcctgtttggcatgatttacgaactggatgctggcgatgactggaccgacccgaaaaacttaatcaaagctaacccaaatctggacgtttcggttaagtacagcgacctggttgagcttctggaagtagcgaaacaggttcctcgcaaggttaacgccttcaaaaccaaacgcctcaatatttgggtatccggtaaatccgcgttctacaacatggagcagtggaaggctgctgaagaccccaaccttgagctggctgattttgcgaatgacagctgcaatatcggtctcgatctcgccaaaaagctggatatgaacgccgggatacggctatttacgcgggaaattgaaggtaaacggcattattactgcatcaaacctaaattttgggtcccggaagacacgatccatacaaccgatccaaaactgctgaaaactgctgacaggtatcagaagttttatgaaatgggcgtgctggaagcgacggatggagcagaggcagactatcgcgagattctggccagtattatcgatatgcaggacgaaaaccgcattgacgagattgatatcgaccctgccggcgcaacagcacttcgccaccagttggaggacaacggatttaccgtagtcgatatccggcaggattacaccaatatgtcaccggcgatgaaagagcttgaagcggctctggccggtggtcgattccaccatgatggcaatcccattctgacctggtgtatcagcaatgttatcgggaaatttatacccggtagcgatgatctcgttcgcccgacaaagggagacaatcaaagcaaaatcgatggagctacagcgttatttaacgccatgactcgcgcaatgctgcacgaaagcagcggcggcacatcggtatatgatgaggaagacatagcgtgttaatcacaattctgagtttcattattggcctggccggggctgtactcatatccgccggagcctggttgattttgcctgctgccggtcttattacgggtgggtcaatatgtcttatctggtcatatctaactgcgcgggcggtttcagccggtgccaaatttaacgggggtgaataatgtttatcccccaaatgttcagagggcgccagcagtcgagggatggcctctgggaagccatgctgggcggggttcgttcaagccagagcaaaactggcatcataatcacgccggaaaccgctctgggactttcagcggtccgggcctgtgtcactctcctggctgagtccgtcgcgcagctgccgtgcgaactttaccggcgggataaaaatggcgggcgccagcgtgcgacggaccacccggtttatgacctgattcactcccagcccaacaggaaagacacctcattcgagtatttcgagcagcagcaggggttgctggggcttgagggaaattgctactcgatcatcgaacgggacggaaaaggctacccgaaagagctgatccccattaacccgaaaaaggtcattgtgctgaaagggccggacggtatgccgtattaccaactcccggaagtcggcgaaattctgccgatgcgcatgatgcaccatgtgaaggtcttttctctggatggctatatcggcagctcccccattcagacgaacgccgatgttcttggactgaatttggccgttgaggagcatgcggcagcgacattccggcgcgggacaacgatgagcggagtgatagagcgtccgagagaagccgcgaccattaaaagccaggatgctattgatcgcctgctggcgaaatggaccgagcgccattccggtattcacaatatgttctctgtggcactgctgcaggagggtatgagctacaaacaactgtcgcaggataacgaaaaggcgcagctgctacagtcgcggcagtggggcgtggaagaggtctgccggctctataaaatcccgccacatatggtgcagatgctggcgaaagcgaccaacaacaacattgagcaccagggcctgcagttcgtgatgtatacgctgctggcatggctgaaacgccatgagggtgcgctgcagcgcgatctgcttctgcccagcgaacgccgcgatttgtacatcgagttcaacgtttccgggctgctgcgaggcgatcagaaatcacgctatgaatcttatgcgctgggccgccagtggggatggctatccactaacgatatccggcgtatggagaatctgccgcccattgccggcggggataaatacctgacgccgctcaatatggtcgacagtgcgaagatccttcctggcgataaatcgccgacagcaaaacagctggccgaaatcgaaacccttctggccagagcctgattatttcccgccgcgcgggatgacctggaagacaacatgacaacgaaattaattaacctgccgcacctggcagatatggtctttggtgtgccgcattacgtgacgcggcaaacaatggactccgtgaaagcggtgctcatccctcgtattcaggggatcaccgaagataccgtcattcagatggcgctaaatccggataaatcacctgctgctgagcaggtccagcccaccggcggggtggcggtgatccccgttcacggcatactcgttccacgccgggggcagattacagcgatgtgctccgagctgaccagctacgagcggatacgcgggcagttgcaggcggcgttaaacgacccctcaatcagcgaaatcgttctggatattaactccggcggcggcgcagcggtggggtgcaaggagctggccgattacatttatcagtctcgcgacacgaagcccatcacggcgattgtgaactacagcgcgtactccgccgcgtatttcatcgcatcggcctgcagcaaaatcatcgtcagccagaccagtggcgtggggtcgattggtgtgatcatggagcacctcgatacgtcgaagatggaagaaaaaatggggctgacgttcaccaccatttaccggggagataacaaaaataacggtacccaacatgaaccactgagtgaagagtcgcggggtatgttccagggcatgatcgacgaaatgtacgagacgtttacggggtcggtggccgaatatcgcggcctgaagcagcaggccgtcattgatacgcaggcggggctgtattttggccctggcgctgtgtctgccggcctggcggatgaagtctctgacccccaggcggcgctcaatgctatcgcggcaaagtatcagcaaccccgtcaaaaaacctccattcagatgcaggcagccgcgatggacctgcaaaccaaaatgtaacccggcgcaaacacaaaccgcgtcaccttaagcagccagcaggctgctttttttatgtctaaaaagagagaaataaaatgccacatattgaagaattgcgtcgtcagcgtgcgggtatcaacgaacagattcaggccctggcaaccattgacgccagcggcgtcacgctgactgcggagcagatgacggagttttcgaacctgcagcagcagttcactgatatcagcgccaaaattgaacgcctggaagccgccgaacgtgctgcggcgctggtcgcaaaacccgtgaaagcgactcagcaggcccccggcattattgttaagcaggagccgaaacagtacaccggtgctggcatgacccgactggttatgtctgtcgccgcaggcgcagggaatctgcaggacgcggcaaaattcgcttcagaagagctgaatgaccagtccgtatcgatggccatttccaccgcagcggcgtccggtggtgtgcttattccgcagaacctccacagtgaggtgatcgagctactgagcgaccgaaccatcgtccgcaagctgggtgcccgtcccgttccgctgcctaacggtaatatgacgctaccacgcgtggccggtggagcaacggcaagctacacaggagaaaacaaagacgccaagacatcagaaacacgctttgatgatgtaaaactgacggcgaaaactctgattgcgatggtgcctatttccaatgcactgattggccgcgccggattcaacgtcgagcagctggtcctgcaggatattctgaccgccatctcagtgcgtgaggataaagcctttatgcgcgatgacggtaccggcgatacaccgattggtatgaaggcgcgcgcgacgcagtggaaccgcctgctgccgtgggaagctgatgcagcgatcaacctgaacacggttgacgagtacctggacaagatcattttgatggcgatggacggcaacagcaatatgatcagcagcggctggggcatgtcgaaccgtacctatatgaagttgtttgggctgcgtgacggcaacggcaacaaagtctatccggaaatggctcagggattacttaaaggatatccggttcagcgtaccagcgcgatccctgcgaatctggggaccgggggtaaggagactgagatttactttgctgacttcaatgatgtggttatcgctgaagacggcaatatgaaagtcgacttctcgaaggaagcctcttacatcgatgccgatggcaccctggtatctgcgttttcccgtaaccagtcgctaatccgcgttgttactgagcatgatattggcttccgtcatccggaaggcctggtgctgggtaccggcgtcctgttctaacccatccctcagtaaatacggcccgcatatgcgggcttttccttttcaggagaatgttatggctgtgaaaaataaagcagtggagccggaagaaacaggcacacaggacaaccatgcgaccgtggtcgcacaggcagagcgtaaatccgttgtgttccttgggccgcaccaccgttattcccgtggagatatcgcgtgctttgaaggatcgcgcgccgaagaactggttaagcggcgtatcgcggtatggccggaggatgccgaacgtgcgctgaaaccgaagccgggagacagcgattttgatactgaaattggatgatgtgaaaacccagctacgcctggaactggatttcacggagcatgacgccatgctcacgcaaatggtgaacgccgcgcagcggagcatcgagcgtgattattactgcaagctggtcaccagtgatgaagagctgcaggcactcccggagaccgtccgcggatttatcgcggatgaagatatccggctggccattcagtttctggtcagcgatgcgtatctgaatggccataccggacagtggctggaaaccgctgcggtgaggcatcttcttttccccctgcaggagcatacgctatgagcctgaaaccgggtgatatgaactgtcgcattgcgattagctacgttcagtccggtcgggggccgctgggcgaaccgctaccggaaaagcaggttgaatcgggaaaagcgtgggcaaaacgggagctggtatcggggcggaaagtccgcacgctggatcagcagcaggtggtggaaacctgcctgtttacggtctatccgggtgtgctggttgatattgactggaaaatcacgacgaaaaatctggtttataccgtccggaatatcgaccgcaaaacagaccggatcattatcacgggggaggctgacgggcggcatgatagagctggcgattaagggtgcgctggagcgcatcaccggcatgaatgcgtatccgcttttactgccggacacggtccaggaaggtgcgacctttcagcgtatctctgacccggaaatggtctcgggaatgttgcgaacggggatcgtctctgcccgtatccaggtgaatctgtaccgtctcgatgattacacctcactgctgcagctggataaaaaaatctggacggaactgaagtccgtcgttcatggccagctggagggtatcccggttcagtatgtggagcgaggcggtatccatcaggataaaaaccagctgacgaatcgtcgcattcagtatcgcctgacccgcgatttcatcattcactacgtggaggactcctcgtgatccgaatggaaattaaagggctggatgagctggagcggcagttaacggccctgggcgaaaaagtggcgacaaaggtattgcgggatgccgggcgcgaagcgctaaaggtcgtcgaggaagatatgaagcagcatgccggctttgacgaaacgtctgccggaccgcacatgcgggactcaatcaaaatccgctcttccactcgcaagggtaaagggaacgcggttgtaacgctccgtgttggccccagcaagcagcaccatatgaaggcgctggcgcaggagtttggcacggttaaacaggttgcagacccctttatccgacccgccctggattacaacctccagaccgttttgcgcgtgttaaccgtggaaatccgaaacggcattgaaaacaggtagcatccgctgccgtataaaaagagagagaaacatggctgataaaacttcacctgaatatgcgatgttgccagcgggcaccattgtgaaatacggggagcctggcgctgccacgtcagcgctgaaaccgctgattaactgtaaagcgctgggtgcaatggggcagacggggggctttgtcgactgcaccacgttactggataagcagaaacagtccatcagcgatctgcctgacgggcctgaaaagtcgctgggctttattgatgatccaggcaataccgattttgccgcgctgctgaacgcagcagaggcccgcaagaccatccagttatacgtcgaattacccaacaagcgaacagcgacgatgctccttgcgctgtccgggtggcagatgaatgaaatcgccgctccggcgaatgaggtcatccagatcactgttcagggtaagcagaacaagatcacctggggaaccgtcgctgataccagcggcgcctgattaacttaacttttaaaccgccaccttcgggtggctttttatttttaaggactacctgtgaaagacaaagattacctgtccacgctgaaatccgcgttgcttaaatcggagccaaccgtcattaaaaccgagttgtttggcgccaccgtattcatccgccgcctgaccggggattacctcatcagctacgaagagaaaatggctgaaaccgcaaaagctggcgcagcgcgcgaggcatcggagcaagtcatccagatcgtcatcgatgcactggttcagccggatggaacggccattccggatgagtttaaacccacggcagccgagctgctgaaggcccatgaaaaccccgaactgctggctgcagtggaaaaagtgaagcaacacgcaatcggcaagctggaggaagcggaaaaaaactgagtgactcgccctggctggagctgattttctggctggccgaccgctggggcgagcctgacccatccaaaattgccgcattgccggcaaacactctgtaccactggcgagcctacttcctgaaacagggcactttccgccgtcctggcgatgaaaacacgccacctaccgaaaccacacctgcgccatcccgggtcgatgatgaatgcgcggcagtcatgagggcattaatgtaatggcagacgtcgcatctttagcggtcgggctgcacctgaacgcagccagttttaaatcccagctgctgggagcgtatggcgatgcggagaaccagtcacgacggtttaaccgtaatgcccaggcggacgcgaaaaagacggaggacgcctataagaaggtcggtctgtcgatatccgggatggccagccggctggcggggctggcaggagccggtctttccatcggtacgatcgtcaccacgtccagacaatatggacaggcattatcagacctgcaggccatcaccggtgcgactgctgctgaaatgaaagcgctggatctggctgcgcaggaaatgggacgcacgacagagtacagcgccagccaggccgccgaggcgctaaagctgatggcgtcggctaaaccggagcttttaaaaacgtccgatggactgcagaaggctacgaacagcgcgcttatcctggcgcaggccgccggcacaacgctgcccgatgcgaccagaacgctggcgctctccttaaaccagtacggggcgagcgcgcaggaggcggatcgttatatcaacgtgctggccgccggcgcgaagtacgggtcgtcggagattgtgaatacagcggccgccattaaaaatggtggcgtcgcagccgcacaggccggcgttggttttgagcagctgaatgccgcgattcaggtgctggcagagcgtgaaattaaaggcggtgaagccggcacggcgctgcgtaacgtcatcctgaatctggaaaagggcacggacaagagtctcaagccgtccgtggttggtctcagccaggcgctgaccaatctatccgggaaaaatctctccacggcccaggccgtaaaactgtttggcgtggagaacctgaatgcggcgtctatcctggtccagaaccgttcaaggcttgatgagctgaccgcttccctgaccggtaccaaaacggcgcatgagcaggcatccatcagggttaacaacctgaacggcgatttgctgggtctgagcagtgcgtttgaagggatggtcattaagatcggccagagcagtaacgggccactccgcagcgggattcaggttgccacggaggcactgaacagcctggcagacaatttcaacaccgtctccagcgtggcgctttacagtctgatccccgtgctatccacgaaactgaccgcagggctgcgggagaatatcgcggcctggcgggaaagccaggcggcggtaaaagcgcgggcgcaggctgatgcggatattgcccgcaaaacgctggattcgacagctgccatcctgaaacagaacgacgctgagtttggccactaccggcagatggagcggacggctaaacagtacgggatgaatatcagttaccaagatgagtttgcccgcctcatccgacaggaaactgaacaaaccaatctggccagccaggcgaaactgaaactggcggcggcaaaccggcaattgtcgatatcagcccgcgcggcctctgttgcggtgggcctggcaagaggcgccctggcttttgttggtggtccggttggcgcggcgacgctggctggatctgcattactgtatttccatcaacaggcaaaagaagcccggcaatcggccattgatttaaaagatgccgtagtggaaaccagtgaagcgctgatgcgcctctcgcttaaccagttaaatgtgaagcagttcgacctggaggataagtacgaaaaccaggtcgtgcagcgtaaccagctgatgaaagagattcaggatgccgacagccgtatcgacagcctgaaagggtttgaccccttcggccagctggaaggggtgacaaaaggccaggcgcgtgcacgggcggatctcgaaagcgttaacgagggactccgcaaaactgaggaaaacattaagcgtgtcagtgatgcaaaaacactggctcagctgggtttatcgggaaaaataacctcccttacggacgatctgaaaggagcgttaagcacgccccccaaagagaccggagagggaaatccctggggcggcgatggcggtaccggcacggggaaaggcagtaagtcccaggtcgaccagttcaaaacgctgcggcagcaaattgaagaagcccatgcatccagcctggccagaattaacctgcaggaaaaggacagcaacagggagctgcaggaagcggcgaagaaaaatggcgccagtgatgctgacctgcagcgcgcgctgttaatgaatgcagagaactaccagaaacagcgactggatctggccgcgcagtattcccccgcccgggaaactctgcgaaaagagcaggaagccagccgggacctggctgagcttttcaaagcccgccttcttgatgaaaaagagtaccaggccgcacgaataacgctggccagagataccgcgaaagagctgctgcaggcaaaggctgacgaaatcgcggcgcctaaactggatatcgccggtgaggttgatccactggtagcactccgcaatcagttaacgcagcggcaggctttgctgctgtcgtactatcagagcagcgcgatcagcaaagaacagtacgaaatgctgatgaagaaggcgacgaaggattctgcagattcgcaatatcagacgtcgctggagttatatcgatcacagggcgaattccagagcctggccgtcgggttatttgaaacggcccatgagcgctctagcaacttcctgacgagcatgttgacgcggacgagaagctttaaggagaacatggctgacctgttttcctcgctcacgcagtcggtcataaaaaacctcgttgatatggccgctcaggctcttgttaccagcactatcatgcaaaccatcatgggtgtggtgggagctggagtgagtattgcaggtggtgtttctggagcggctgatgtcggcgcaggaactgcgattcagaatgcgggtaataactttaactttcaaataccgggttatgccaaaggcggtgtcttcgattctccttcattaagtgcctacagcaaccaggtctacgactctccgcagttcttcgctttcgcaaaaggggccggcgtatttggcgaggccgggccggaggccatcatgccgctgacgcgtgccggtgatggttcgctgggtgtacgcgcggtgggtggtggtcagaacgccggcgcgtcggaagggccaaaagtctatatcacgattgaaggcggaaacacctcaacgcaggcaccgtctggttttgagcagtttggccagcagatcggctcgtttgtggagaaaaaatacagggagctgatggcgcaggatatgcgccctggcgggatggtctggaatgcagttaaagggcaacgttgatggctattgagatattcacctggagtccgcgggttaatccccagcagaccgttaactttcgtgtccggaaggcgcagttcggtgacgggtatgcgcaggtatccggcgatggtattaacacccgatcacaggactgggagctgagttttgtcggtacggaggactatatccgtccgattaagcagttcttcgaccgtcatgccggcacccgcgcgtttcagtggaccccgcctctggaagaggtggggctttaccgctgcgaacaatataaaccggtgccgctgggcggcggaaattactcactttcagccacttttattcaggcatttaaaccatgagccttaacgcgaattatcagaagttagagccaggcgatgaggttcgtctcctggagatcgatggccaggcgtttggcctggacgaggttttgtatttccacggctataacgtcccccatactgcagccgaaatcctcgccgctgacggcgacctggataagctgcctgcgaaaagcatctggtggcaggggcgggagtataaagcctggccatgtgtaatcgaagggatcgagtcatccaccactggcagcgacgcgcagccaacgctgcgggtagggaacatcgacgggaaaatatccgcgctctgtcttcattacgacgatctggctctggcgcgggttgtcatccacgacacgcaaaaacagtatctcgatgcgaagaactttccggacgggaatgcctcagctgatccgacgcaggagaaacggcgccttttcttcattgacgtaaagcattatgaagacgatgagaaggtggaatttactctctccagcccgtttgccctgcaggggatgatgatccccactcgccagctgcatgcgatttgcacctggtgtatccgcaatcaataccgcagcggtaacgggtgcgactatgccggcacccggtattttgacaggaacaatcagccagttgatgacccgtcgcaggatgtctgcaacggaacgctcacggcctgcaaattacgtcatggtgagaatagtgaactgccgtttggcgggttccccggcacctcattaatcaggagctgatatgcgtcagaaaacgattaaggccatccaggaacatgcggcggcagaatatccgcgcgaggcctgcggcctcgtcgcccagaggggccgagcggagcgttatttcccctgccggaacctggccacagagtcgaaagataattttgtgctggcgccggaggattatgcggaggttgaggaatggggaacgatcaccggtattgttcacagccatcctgatgccaccacccagccgagcgaactggataaagcgcaatgcgacgcgacccttcttccctggcatattatcagctggccagaaggcgatctccgtaccatccacccgcgtggtgagttgccgctcctcgagcgaccattcgtgctgggccactacgattgctggggcctggtgatgagctattttcggcaaacccacggcatcgagctgcacgattaccgcgtcgattatccgtggtgggaaaaggagtatccggacaatttttatcaggactgctggtatgaatgcgggttccgtgagtttgatggtccaccacaaccgggtgatatggtgatcatgcaggtgcaggcggataagtggaaccacgccgggattctgctggaagaaaatatgctcctgcatcatctgtatggccatctcagcaatcgcgtgccgtatggtggatactggatggaaaggacgatgaaaatcgtgcgacatcattcactattttaaggtgcctgtatattatgctcctaattcaatttacaggagcatgcgaatgaatgagccaatcactgagcagttatacaataaagttgtaaatttcgttaataaaatggatgggagtgtgcgctttgattatatttctaagtccttgcgcattcctcctgacactcttgatgaaattgtggacaggatgattgctgatggagtcgttgttaatacaggaacggtaggtgaatatttaccagtaaaacatgcatcttccaaaaacactgataaagataatcgatgctgtacatctcaaaaaaacaatgatagtagtggactaaaatttcttgttgggagagtagcattagttatagccataatattttttattataacctgcttttatgcttacagagcaccgatatcgtttgtttttctaatcccagccataatggctgcatttgtttttacaaaccaaaatcatggtggagaaggtttcctagggttgagttcaatctctgcatgtttaatctttttgctccttactaatgcgcagacaccaatatttggagaagcatatgaattaagaaagcagagggatgaaattaaaaatgaagttactaggaaatcaaaggaagaagatcagcaacagctaaatgatatccttaacgccagagagcatataaaagggatgctaaaagacccctcctcagctaaattttttggtgaattcattggaaaaaatggtgctatctgtggccatgtcaacgctaaaaatagttttggtggttacactggggagtctcgttacatattttctgtcaatttttctgcaatagatgaaggcaccacctcctttaataaggagtgggagagacagtgttatcttaattgaatggcaaggataaagcatgcaagaaaccatgacgagaatagaactgtctggagtcctgggtaaaacctttgggaaggttcattatcgtttaataaagaacatcaatgaagccggagaggcattatctgcgacgatccctggatttgaaaggttcatgatatccagtgaggagcgtggattgacctatgcagtatttaaagggaataagaatatcgggcatgatgatttaggattccctgtaagtggcgaaattatccgcatagtccctgttatcattggcagtaagaaggcaggaattctccaaacaatccttggtgcagttattgttgcggcaagtgttgcctatggttttttcacagaggattgggctaatgccgcgtatggtattcaagctggcggcgccatgatgctcggcggcgtcgttcagatgctctccccacagccagctggcctggcacgaaaagaatccgctgacaataaagcgtcctacgcctttgggggcgtgacgaacactgcctctcagggatacccggtccctttgctttatggcaaacggcgaattggcggcgccattatatctgccggtatttacgtagaagaccagcaataagttttattcagtaaaccatccaattcaggccaccttgcggtggctttttttatgggcgtaatatggcaaataacataattaaagggcgcaagggtggcggctcaaagcagcgtacaccgacggaacagccggatgatttacagtccgttgcaaaagccaaaattctgctcgcattaggtgagggtgaatttgcaggtggtttaaccgggaaagatatttatcttgatggcaccccgcttgaaaatgctgatggttcgcaaaacttcagtggcgtgtcctgggaatttcgccccggcacgcaggctcagacttatattcagggtattcccggtactgaaaatgaaatcagtgtaggaacggaagtttccagcaagacagcctggacccatacctttactaatacccagctttctgccgttcgtgtccgcctgaaatggccgtccctgatgaaacaggaagatgacggcgacgtggtgggcaataccgtcaagtatgcgattgacctgcagaccgacggcggcgcctggcagacggtgctggaaaccgctgtcacgggtaaaaccacctccggttatgagcggagccatcgtattgatctgccccaggccggcagtacctggacgctacgcctgcgtaaaatctctccggatgcaaacagtgtcaaagttggcgacgtgatgacgctgcagagctataccgaagtgattgacgcgaagctgcgttatcccaacaccgcgctgctttatatcgagttcgactccagccagtttaatggctccattccgcaaatttcctgtgagccgcgtgggcgcgtgattcgtgtgccggataactacaatccggaaacccgcgaatataccggcgtctggaccggcgggtttaaatgggcctggacggataacccggcctggatctattacgacattgttacagctgaccgttttggtctcggtaatcgtctgagcagcgccaatatttcgaaatggacgttgtaccagattgcacagtactgcgatcagctggttcctgacgggcgcggtggtgacggcatggagccgcgctatacctgtaacgtctacgtccaggaacgcaacgacgcttacaccgtgctgcgagactttgccgccattttccggggcatgacctgctggaacggtgagcagattgttgtgcaggctgatatgccgcgtgatgtcgattttacctatacgcgcgccaatattgtcggcaaaccccgttattcgagcagcagcagccaggttcggtacaccaacgccctggtttcctggtctgatccggataatgcttatgctgatgcgatggagccggcgtttatcccggaactggtttcccgctacagttttaaccagctcgaactgaccgcgattggctgtacgcgccagagcgaagcccaccgtaaggggttgtggggcatactgaccaacaacaaagaccgggtcgttgagtttgatgtggggctggacggtcgcattcctcaacccggttatatcattgccctggcggatgagttgctggccggacgggtcaacggcgggcgaatcagcgcggtgaatggccgggtgattactctggatcgtgatgtggatgccaaacctggcgaccgtctccagctaaacctgccatccggtatctcacagagccggaccattcaggctgttaacggacgccggcagattacggtcacaacggcgtacagtgagacaccagaacgggaatgcgtctgggccgttgaatccgatgacctcttcctgcagcagtaccgggttacaggggtaaaagagaacagcgatgcaaccctcacgatcaccggcgtggcacatgacccggataaattcgcccgcatcgataccggcgctattatcgaccagcgcccggttagcgtattgccggcgggcaaccagtcacctcctgacgatattgtcatcacatcccgctcggtcgtgaatcaggggatcagcgtcgaaacgatgcaggttaactggtcagcggtcagcggcgctattgcctacgaggcacagtggcgccgtaacgacgggaactggattaatgtgccgcgcagctcgaccacctcgtttgaggtcagcggcatttatgccggtcgttacctggttcgcgtccgtgcgatcaatgcggcggagatctcgagcggctgggcgtattccgaagagaaaaccctgaccggcaaggtcggcgagccgctggcaccgctggcgctggcaacccgttcgctggttcatggggtccaggttagctgggagttcccgaccggctccggggatacgctgcgcacggaactgcagtacagcaaaaaccaggacggcagtgcgccaatgccgttatcagacgtggcctatccggggaaaagctatcagcagatgggcctcagtatgggcgcagaattctggtaccgggcgcgccttgtggatcgtcttggcaatgaaagcccgtggaccggctgggtccaggggatggccagcgataactttgatgactactacgaaaacctgaccgacgcgatcaaggatacggctgcctgggaggaaacgcagcgcaccattagcgaaacgcaggaaggtatccgcaatacgcagcaggaactggagcagaccgctgaagctctgcgtaaggaagccgaagaccaggcgaagcaggtcagccaggatattgatgcatcggcgaaaagcatcactgctgatgttgacgggaagatctccgccgtgaataaaaccatcacggatgagatcacctcggtcaatgaggctctcgattctggtctggctcaggcaaacaaaggcgttcaggaggcaaaatccgccgtcgcagatgcgaacaagcagatcgcaactgtgaacaagtcgttgaccgacagcatcacccaggtaagacagtcagtcaccgatacggctgcggaaatcaacgccaccatcgacctggagattgccagggtcagcaaaacgctggccgacggcgatgccgcattgaatgcgcagataaagactgccgaaaatggcctgaagcagtcgctgtctcaggttaacaccacgctgaccaatgcagtgaagcaggagaccgcggatcgtatcgccgatgttaacgcgaaggcggcacaggccgctgatgaactgctggcggcaacgcaggggattgaggcgagtatcgagagcctgtctgaggccgtgacctctggtgacgaaaatctggcacgccagatctcacagattgccgctggcacaggggagcagtttgactctctggaaatctggtatttcgacaaagatgccgaaggctggacggaagacgacaacggctacacgccaatgagcgtcaccagcgatggctggcttaaagccaacaattcgacttcaacctgtcgatcccctaacggcctgacgatcgatgcccatgcttatcgtttcattaagatgcgcattaaaaaggttggtagcccaacctggaatgccaaaatgttctggatcggcgctgatgaaaccggctggaatgctggtcgctccgtggttatcagtgagccggaatacgatgacaagggtattgcgattctgaccctgcacgacattgagtggcgggattcgacaacgattcgtcgtttccgcttcgatttcacaacgggccaggatgcggacaactatctgttatttgactggatcgccgttggccggccagcacccggcgccagtacggcggctctgcaggatgtgcgcagtacgctgagcaacgcgctgactgccgaagcgcaggcacgcagcacgctggcggcgcagatgcgtggctcctatgagggcagcgatctggataaagtcacctccgggctgctgtaccaggaaaaaaccgcgcgcgttaccgccatctcggcggaagttaaggccagagagtccctgcagacgcagtttaacgacaacaaagctgctgtttctggtgaactgagttctctgacgacagagcagagcgcgcaggcgagccgtatcggtggcctggaaaccagcctcgggaaaaaagccgatgcggccgcgctgacgtccctgacgcagaaagttgagcaacagggcgccacgctgacatcgcagggcgccgcgttaacatcgctcactaaccgggttggccagacggaaacgggcctggctggtacgaatgaggcgctgagcgggctgcagtctgttgttacccagcagggcgacaggataaccagccagggtcagtccatcacgaaactgacgagcgatttgggcacgacaaatgccgcgctggcgaagaaagccgaagcggctgcggtcactgccttaacgcagcaggtagagcaaaacgggcgggatattcgcagcaatactgacagcatcaccagcctgtcgaatcaactggtcaatggccagccgaatcgctggtcccgtcgactctatccggtgcagctggctaacgccgggacagtcccgtcattcagcgatgttcgcgccgtggcgccaacggtcgtggatgaggtggccgacgcggccaaactggactttacgtccgccggcagctatctgatcgcgctgtattcctgccaggtgaaagtggtcgcagataccaccatcacactggcgcccggcgccagggtttttgatgataccggcgccatatttgtgaatggggtacaggtcgcctggggtaacgccagctggaataccgtcagttttgaactgaaagccggctggaacaccgttgagtttctggtgaatcagtggacgggccaggcgtatatcaacctgggtctgaagctgtcagacaaggttgctgagatgtactccggtctcggggtttccgcgctggcaaacgcagccggcgtgctcagctcgaatgtcagccagattggcaacgatgtggtcagcaattcgcagagtatcacccagctccggaatgcgctgacgcagacagacgcgaacgtggccagcaaagcggatcagacggcgatgaactcgctaaccggacgagtggagaagacggaatccgggctgacggctgctaacgccaacattacctcgctgaaatccgctgtacgggccggaaacgcatcaggcggggatttaattcccaacccgacgtttgacccggcgtatgaccagatggggttcagcgtggtagccacgacggctgaggaggtcccgccgggctgcccgtatggttatgcggcccgaattgccagccgggatcaccatcctaactttgccgcgttcccggccacgcttaacgatgtgattgagatcagcgcactggttgcctgcggcgccggcacggcgaattttaatctgtatgttggcaccgccgttcggccagatacgagcaccggtgcgccactcatggcggggggcgggaaatcaccctccgcgacctggcagagaaccacctggcgcttcaaggtcacgcaggcgatggtggacaggggttatatccgcccgttcctgcagatctcgcagaacagcccgtatggcaccgtatggttcgttacggactggcatatgcgaaatgtgacagcggcgcaaaaggttcaggatactgcggatgccacggcggcggcggttgactctctgaccaccaccgtgacgcaacagggtaatctgctgacctcgaccggcaaccggacaacccagctggaaaacgggctggcaaccatcaatgccgcagtggccaaaaaggctgatgcgacagcagtgcaggatttgaccaataccgtcacacaactgggcaacgatctgactgctgcgaacagcgccatcacgaaactgaccggaaatctggcgaataccgataaagcgctggcgcagaaagccgatgcgactgcgctggccacgctcgacacgaaagtgacgcagcagggtaaaacgctggagagccagagcaattcgctgacgaacctgtcgaacagtctctcgcaggttgcggcagatatcgatgccagcggtcagataccgggtaacctggtcgtgaatccctcgtttgaacgtgggctggatggctacaccgggcggtcaaccgcgaccagtgtggtggaggtttccgctcctcacagcgggacgcgggcgctgaaggttgatccggggagcgtgtctccggggcaatacatcccgtttgttcaggggcgaacctatgaaatcggggtgtgggtcaaggaacccggagcgacgacggataatggcgcggggaacaacaagttgcggatcggtaactctgccggccagccggtctttgagcgtccgtacaacagcggcacggtggggacaaactggaccctggtttccggtcgctggaaagcgacggagacagccagcctgccggtgacgctgagcaactatctgattaatggcagccgctacttcgatgatttttacgtcactgacgttaccgaccgggtggacatcgatgccaccgccggcgccgttaccggactgacgagccgggtcagcacagcggaaggggctatcacctcgcaaagccagcagctgacgaacctgcagaacagcctgaacacgaccaacagcaatgtgtcgaagaaggccgatgcaacggcactgacttcggtcgataaccgggtgacagaggcggaagggaaactgaccacacagagccagcagctgacaaatctggcgaatgtgctgacggccacccgcaacgctggcgacaacctgatcccgaactttgattttctacagggcagcactgcctgggatattcagtatccagccggtgtgacttttggcgatttcggggacgggaaagcgggggtccggctgaaccggacgactaacaccagtccggggatcttctccaacaacaacaagccggtgccgctgaatggccagcgcaagtaccgcgtggtggtgaaggccaaaggtgtttccggcgcgatgagtctgctgatccgtcgccagaacaaaatcggccagacggacagtacgtatgaggataaaacggtcacgctgaccactgactggcaaaccatcacctgggaaaccggattgacggctgccggcgcggacgggcagaacttcaaactttattctcatccgacaaacggtgaaatctggctcgattccgtccgggtttttgatatcaccgatgaaaccaacatcaaggcgaccagcgatgctgtttcgtctctgaccgggacggtgacgaaccaggggaacaccctgacatcacaggggcaatccatcacggcgctgaataacgcgctggaaggggtcaaaggcgatgtggcgaagaaggctgatgcgtcggcggtcagttcactgaccaaccgggttacccagactgaaaaggatatccgtagccaggccgacagcctgaccaggctgaatacatcgctgaagcaacaggcaacacggggagccaatgtactgccggacggcagttttgaatcctatgccgtcggcgatgttctcagtaatgcccgcgctgttatcaccagtgaagctgcgcacagcgggaccaaaagcctgcgtgttacgcgcagtacggagtacaacccgaacgcgacggataataacgatacccatatcttttctggtatgcaggttcgcgataatgcggtctattacgtggaggcgtgggttaagttgccggctggctcgaccgccgatccgaccgtttatatggtgctaggattttccttccaggattctgccaatggctggtcgtggcctggcctgaacgtgaaagtctccgagttgtcggtggacaactggacaaaggtcagtggctatctgaccaacaaccgaaccgcactgaaacaggcaatggtgaggatctccatcccgaatacaccaaaagttcgcctgggtgacgccttcctgattgatgatctgatcatcactgacgtgaccgatgcgaaagcggcgctcgatgccgccgatgcgaatgcgcaggcgctttccagtctgtccgcgtcagtcacgcagaacgggaagaatattacgtctcagggcagcgcgatcacgaagctgcagtcggatgtgacgcaacttggtaaggatatcagcggcaaggccgatgccagcgcgctgacgaatctgacgacccgcgtgacggctaccgaaggcagcctgaaatcgcagggagacagcctgaccaacctgcagaacagcctgaacacgactaacagcaatgtggcgaagaaggctgatgcaacggcgctgcagagcctgcagaacaccgttgaacagcatggcagggatctgaccacgcaaagcagcgcgctgacgaacctggaaaacaacttttcctccctggccgttggcgggaccaaccttatccgcaatgcggacacactggagggatggagcagccgccacgccacagagacgtatctgggcgaccgcgtggcctacacccggctggcgaaaggtgcatccggttatatccagctggatgaacagacgctggatgttaccgggcgtactgaatttgtattcagcttctatgcgaaaggtgcctataacggacaggaaatggcgagttatttttataacccgtcgaacactaccaccacggaaaccagccagggggttaaaggcggggccggtgacggcaaggcggtcacgaaactgaccaccgcatgggcgcgttactgggtgaaatgggttattcctgccaccagtggcaccaaacggctgattgccgcgcgtctggaaagcgcgacgtctgccgacaaagaagtctggctctgtcgccctcagctggaaaccgggaccgtgatgactgactggtcaccgagtccggatgatgcggccagcggtattaccgcgaacacatcggccattaacagcctcaccagtcgggtgacgaatgccgaggggcaactgaccgcgcagtctcagagcatcacgaatctgcagaacagcctgaacaccaccaacaacaacgtggcacaaaaggccagcgcgcagtcggtgagtgatctcaccagccgggtcaccagtgcggaaggcaaaatcacctcccaggggcaggctatcacgaagctgcagggcgatttgagcagcaccaccgataaggtcaacaccaaagcggatcagacggcgcttaacgcgctgactggccgggtggagaaaaccgaggcaggcctcacggcagccaacagcaacatcgtcagcctgacggcggcggtgaacgccgggaatgctgccggggatgattacatcccaaacccgtcatttgatccggcgtatgaccgcatgggttatgacgtggtggagaccactgctgcaggtgtgccggctgactgcccgttcaggtatgccgtccggctggccgggcgagaccatgtgccaaaaatcaacaacatcgctgtgacgccgggcgacgtttacgaaatgtctgctctggtagcgtgtggtaccggcagcgctgactttaatttctacatcggtcgggccaccactgctactggtggtattggggcgagagcgtccgggggaaacaccaagaccaccaccgcgtggaaacgagccacctggcgctttactgtgccggcagacacgaacttcctgcgaccgttcctgcaggttaatcagagcagcccgttcggcactgtctggtacgctgccgactggcatatgcgtaacgtgacggcggcgaacagtgcgcagaaaaccgcagatgcgaccgcaaaagcggtggattcactgaccaccacggttagccagcagggcgatacgctcagcagcatcggcacgcggaccacctcgctggagaacagcctccggtcgacaaacgatacggtgagtaaaaaggctgacacgacagcggtgacgcagctgcagggcacggtgacgcagcaggggaatgacatcgcggcagccaacagcgcgctgacaaaactcagcagcgatctggccacgacgaatgcgaatgtgaacaaaaaagcggacgcaagcgcgatgaacaccctgcagaaccaggtcactgagcagggcaaaacactcagtgcgcaaggggattctctaacgcaacttagtaacagcctgagccagacggcagcggatattgacgccagcgggaaaatgccgggcaacctcattgtcaacggcagttttgagcgcggcgcggcgggctttaccggctggagcagtaccgcgacggtggccgatttacaggttccgcattcgggtaacaaggcgctgaaaatgtccgccggccagtcgaacctggtcgggcaggaaatcagtatcacgcagggtcgtacctaccgcatgggggtatgggcgaagcaggacccgggaaccactattaaagatgcgggtaacacgaagtttcgtgtggccgacagcactggcctgctggtcggctcaaactacggaccgtttagttctggctggcaactggtaacgtttgactggaaagccacgaagaccacgacggccagtttccagctgacgaccttcctcagcgcgggggcaatgtatttcgatgatttccatgtcctcgatgttacggatgaaaaggatatcgcagctaatgccggggccatttctcagatgaatacccgcgtcaccgctgctgaaggggctatcaccacccaggcgcagcagctgacgaaactcagcggcgatctggccgtcacgaatgcggcggtcagtaagaaggccgagcaaagcgctgtcaccgggttgaccacccggatgacgtctgccgagggtaaactggattcgcagtcgcagcagctcaccagtctgcagaacagcctgaccacgatgaatactgagctgggtaaaaaggctgacacgtccgcggtgagttcactgaccggtcgcgtaagccaggtggaaaacaccatcaccagccagtcgcagagcatcacgtcgctgaccagcaccatcaataccatccgcactcagggagctaatccgtgggttgacggtacgtttgaaagctacagcgatggccaggtgctgggcgggaacggcacagccgttgtggtggcgtctcagaaattcaccggcggtaagagcctgaagttgagacgggatgagaacaacagcggcaacagtgataaacagcttggcacctggcagtcagtccgtgaggacgcgaagttccggtttgagttctgggccatgatgccggcggatcaggcgccctcctccgggtggacaacgctggtcggtatccagtcgcagaatgctgccgggcaaaatgcgtggcaggcggcggtcactgtcagcgaagcctctctgggcgcgcgcgataagtgggtgaaattcacgggtatcgccagtaacaacggggcaggcagaacacgcgcggtggtctggatctccactcgtggcgccaccggcaacggtacccctggctattcactgtatatcgacgatctggtcatcacggatgttaccgatgcgaaagcggcacaggatgcctctgacgcgacggcgagcgccgtgagcggcctgacggcgcgcgtaacggatgccgaagggaaaatcactgcccaggcgcagcagcagacagcactggcctcgaaagtggataacgccaactcccgcgtcgataacatggcgaagacgctgagcgacagccagagcacacaggccagcctgaatacctcgcttcagtcgcagattgacgcgcaggcggccgccaacatcaaaaaccagacgacgctggacaacacgattaaatcggtggccagtatcaccagtacccagcagacgcatgcaacggcactggaggcgctggcaacgcagcagacgaccctgacatccagtgtcggggatctcagcgcttccgttcagaacaccgccaaaaccgtggcggatgtgaatggtacggtgagttcgctgtggtcgatgaaggttgagacggttaacgggaagaatgttggcgcggggattacgctgggcagcaatggtgaaacaagcgatatgatcctctacgctgaccgcttctcgctgtttaaccgtaataatgcgacggctgttccggtgatggttgccgaaggcaatgagctgtatatcgatacggcacgtattaaaaacagttccctgacctcaaccaaaatcgcggacggttccatcacgaacgcgaagatcggcaacgagatccgctcgaatgactttgttgacgggtcacgcggctggcgtatcgccaaggatggctcttcgcagttcaacaacgtgatcgttcgcggtgcggtttatgcgactgacggctggttccagggtacggtatatgcgaaccacatcgagggcgacatcgggtcatttgcgatcaacatcgctcagcaccgcacgcgcaaggtgccgaaggctacatggcagtggtttgagctggcccggttccggcggcagaatttcgaccaggtgatcaatattcgcggtggactcctccagacggatagcatcactatcgacggcggcgcgaaactcagagcggggatgtcctacgcgccaggggctgacggcggactgaatcctggctatctgtcgtatgcaatgcttcttcgtggcacaggcgctacgtctggtggcggcagtatggagctaggcattgagcttatgtatgaaacaggtggagcaacacgcctgttaacggcgcaagagtcaatgaacgtagacaacatgtcatttgtcgtccctgccggtactggcgacgctgttctgcgatatggctgttacctggaccgtaacggacagatggtattaaccatcctctcaagattcgacgccttcgccgcgcgcaataacaacgtaattcgcggttcatcaacctgataacaatatatggccccgcaaggggccttttctttttccagggaaaaccatccaggaggaactttattatggcgatgtatgaagtcggtaccgtcacgggtgccgcgtcgcaggcacgggtgacaggtgcgacaacaaaatggtcacaggaggcgctggggatactgcccgggtcgattctagtggtctaccgcagcggtagtgctgacctgtatgcgatcaaatccgtggacagcgacacgcaactgacgctgacccggaatatcaccaccgcattttccggtgccagttacggcattattaccgctgaaaccgccagcacctcgtcgtttgctaaccagctggccagcgcgtttgcattctggcgtagtgtagtggagggctggtcgatggccctgaccggcagcggcaatatcaccctgacagacccgatcacaggaaagcaggtgatcgtgccggcgatagccgggatggcgaaggcatcggatcttaacgcactggcaaaactcacaggaggaaacaaactcgacggctcgcaggttataaccagcgataatgccggttttattctcggtaagaactcagatctggctctgctcaaaaaacaggggcaaggcgggacaattgccgttggctcgggaacaccgttcagggttcagcgttcaagagcgaccactgtatcaccggcagacacctttgatgacatcctcgttattgatgccaacaaccaaacgacactgcctggcgcgctgtctgccggcggcaatatcgataacacgtcgaagggtaaagtgttgacgcaggcgattgagctctcatttagcacgccatacatcgactttcattttaactacagcaccgacgacttcaccgggcggattatggccactgccgccgatcaaattagtgtgcaaggtagtcattggcgagttgacagggatcttcgtgttggtggcatggcagatattggaggctggacgcaatgccgcgacgacctttcggccaacaaaacagactttggatcccctgctattggttcgttggtttcaggcggaaggattcgatctcgaatgctcgggcgcggcggtaacgttgacacctccggggcgtggggcggtttctatcttgaagagtacgtgggaaccgaacacaggattgtcatgtatatggacggcttcgggagaacggacgcatggtcattccgcgcaggggggacaatctccacacctaaaggcgacgtcctgaccactggttccgacgtgcgcctgaaaacagacttcacacaagcgtctgaaaacgcctcagagcgcattgagcgcttaggggtgtgtgagtaccggatgaagggggaaacgcgccggaggcgtggttttatcgctcagcaggctgaaaaagctgatgatctgtatactttcctcggcatcgagcaggagattgatggcgaaaaatttaaggtgatgaatgtggattacacggcaatcattgctgacctggttacggtggcgcagggtttactggttaaaaatcaggaactggaaaggcgtatatctgtactggaggggatc

SEQ ID NO: 8 - DNA, 350 bp, Kappa phage
  source 1..350: mol_type = genomic DNA
cgggctaactgcccgaaaacatggggcggttcatcgcgtcgcgccaatctgcgctgcagctattgtggccagtccgggcataactcgaatgcctgcccacataatgcgagcagcggtcgacggcgcagcctgaatgacgactttaccctcgactgaaccgatagcacgatggcggcaggcggaattgatttcaatgtgaaattattcaatgtcaagtcattgcattgcgcgatgatgataatagatatcatttgagggggtaggggggatcaaatccctaacccctttcgcgcttcgggactgccgcttcaggtagatttttgcgcgtgagaaataaaaacttttttttg

SEQ ID NO: 9 - DNA, 4469 bp, synthetic construct
  source 1..4469: mol_type = other DNA; note = p1866 payload
tgattataagaaggcgccccaagcacccccattttagctataaaaaaacccgccgaagcgggttttttcgaaaattgtaaggtcacattacgccccgccttgccactcatcgcaatattgttgaagctcattaagcatacggcctacatggaagccatcacacacggcatggtgtacctggatcgccagaggcattaacaccttgtcgccttgcgtataatatttacccatagtgaaaacaggggcgaagaagttgtccatatttgctacgtttaaatcaaaactggtgaaactcacccagggattagcactgacgaaaaacatattttcgataaaccctttagggaaatatgctaaattttcaccgtaacacgccacatcttgtgaataaatgtgcagaaactgacggaaatcatcatggtattctgaccataacgaactaaacgtttcagtctgttcatggaaaacggtgtaacaagggtggacactatcccaaatcactaattcaccgtctttcattgccatacgaaactccgggtgtgcattcatcaggcgggcaagaatgtgaataaaggccggataaaacttgtgcttatttttctttacggtttttaaaaaggccgtaatatccagttgtacggtttggttataggtgcactgcgcaactgactggaatgcctcaaaatgttctttacgatgccattgactaatatcaactgtagtatatccggtaattttcttctccattttagattccttaggttgcgaaatctcgataactcaaaaaatagtagtgatcttatttcattatggtgaaagttgtcttacgtgcaacattttcgcaacaagttggtttcccgaggcctaacttttgttgcaatggctgtctaccctgttttatactagctcagcccttggtacaatgctagcgttttcattaaagaggagaaaggaagccatgagtaaaggtgaggaattatttactggtgttgttccgatcttagttgagctggacggcgatgttaacggtcataaattcagtgttcgtggtgaaggtgaaggcgatgcaaccaacggtaagctgaccctgaaattcatctgcactactggaaaattaccagtaccctggcctactctggtgactaccctgacctatggtgttcagtgtttttctcgttaccctgaccacatgaagcaacatgacttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccataaactggaatataacttcaactcccataacgtttacatcaccgcagacaaacaaaagaacggtatcaaagctaactttaaaattcgacataacgttgaagatggtagcgtccagcttgcggatcattaccaacaaaacactccgattggagacgctcctgttttactgccggataaccactacctgtccacccagtctaaactgtctaaggatccgaacgaaaagcgcgaccacatggtgttattagagttcgttaccgctagtggtattacgcacggtatggatgaactctacaaataataatcagacagtttcacctgttttacgttaaaacccgcttcggcgggtttttacttttgggtttagccgaacgcccgggctaactgcccgaaaacatggggcggttcatcgcgtcgcgccaatctgcgctgcagctattgtggccagtccgggcataactcgaatgcctgcccacataatgcgagcagcggtcgacggcgcagcctgaatgacgactttaccctcgactgaaccgatagcacgatggcggcaggcggaattgatttcaatgtgaaattattcaatgtcaagtcattgcattgcgcgatgatgataatagatatcatttgagggggtaggggggatcaaatccctaacccctttcgcgcttcgggactgccgcttcaggtagatttttgcgcgtgagaaataaaaacttttttttggcgttcggctaaacgtgctacatttgaagagataaattgcactgaaatctagaaatattttatctgattaataagattatcttcttgagatcgttttcgtctgcgcgtaatctcttgctctgaaaacgaaaaaaccgccttgcagggcggtttttcgaaggtcctctgagctaccaactctttggaccgaggtaactggcttggaggagcgcagtcgccaaaacttgtcctttcagtttagccttatccggcgcatgacttcaagactaactcctctaaatcaattaccagtggctgctgccagtggtgcttttgcatgtctttccgggttggactcaagacgatagttaccggataaggcgcagcggtcggactgaacggggggtttgtgcatacagtccagcttggagcgaactgcctacccggaactgagtgtcaggcgtggaatgagacaaactcggcagtaacagaggaatgacaccggcaaaccgaaaggcaggaacaggagagcgcacgagggagccgccagggggaaacgcctggtatctttatagtcctgtcaggtttcgccaccactgatttgagcgtcagatttcgtgatgcttgtcaggggggcggagcctatggaaaaacggctttgccgcgaccctctcacttccctgttaagtatcttcctggcatcttccaggaaatctcagccccgttcgtaagccatttccgctcgccacagtcgaacgaccgagcgtagcgagtcagtgagcgaggaagcggaatatatcctgtatcacatattctgctgacgcaccgatgcagccttttttctcctgccacatgaagcacttcacttacaccctcatcagtgccaacatagtaagccagtatacactccgctagcgcagatgtccggcggtgcttttgccgttacgcactactttagtcagttccgcagtaccgtcaggcgctgacatagctatttactttgtattgcctgcaatcgaatttctgaactatcatatagtggggataacgggaaagttactatatttgcgaactaacttaggcgtccacctcgaagctacctaatcacacccaacccgcgcggggtaaataaggcactaatccgagcttaaagcttgcgtagcacttagacacaagttaattaccaattgtctggtagtttggcggtattagcgagatcccagacgcaaggcagagttaattttaacctaaagccacaaataagacaggttgcacaagcccgccggaaattaaatcttgctcacttcggtaacggagtttccctcccgcgtacttaattcccaataagaaacgcgcccaagtcctatcaggcaaaattcagccccttcacgtcttagaacgagggtaaaaatacaagccgattgaacaagggttgggggcttcaaatcgtcgtttaccccactttacaacggagggtaactagttcaccctatagtacgaagcagaactatttcgaggggcgtgcaataatcgaatcttctgcggttgacttaacacgctagggacgtgccctcgattcagtcgcaggtactccgactcacactgcctcacacccagctagtcactgaccgataaaattgacccgccctctaaggtagcgagtacgtcctaaaaggcttcggacagggctatataggagagtttgatctcgccccgacaactgcaaccctcaactcccttagataatattgttagccgaagttgcacgacccgccgtccacggactgctcttagggtgtggcttcttaatctgacaacgtgcaacccctatcgagggcgattgtttctgcgaaaggggttgccctaatagtcgcgacaattggcccttgtaggggtgaaaccacttagtttcgcgccgtagtcctaaaggcccacctattgactttgtttcgggtagcactaggaatcttaacaatttgaatttggacttgttttaggggcgttattcgagggcaatcggagctaacttcaagactacttctttgttgaatactaaatagtgcaaaggtcgtgtttcctcaaggatactccgctaacaatataggattccaatcagattcagcactggctgtacgggtgttacggtgaggttttcgggtttacggctggaagctagcacggtaggaagcctttcaatcacaaagcaaaagggccgtcgaaggcccacaagatacgaaagctctcgaagccttatccttgaacgatccacctatttaggcagttacgcacaaaagctacccaataatccgtgacaggcacaatatcacggaacaaaggcgaaaactctcgtacacggttaggttttcgctaggaagaataaacctctatct

SEQ ID NO: 10 - DNA, 363 bp, synthetic construct
  source 1..363: mol_type = other DNA; note = Candidate HNH protein ORF
atgccagcccggtctaaacgtccatgccgccaccgggggtgtccggcgataaccaacgaccccagcggttactgcgatgctcaccggcagcaacatgctggcgacggctggcgcaactaccagggcgggaaaagccggcatgaaaggggctacggtcgcccctgggaaatccgccgcgccagaatcctccagcgcgataaatatctatgccaaaactgccggcgtcatggcatcgccaccaaagcgaccagcgtcgaccacatcatacccaaagcgcgtggcggtacagacgacgattccaatctggagtcgttgtgctggccctgccatagagcgaaaacagcaacagagagaacccgatga

SEQ ID NO: 11 - protein, 120 aa, synthetic construct
  source 1..120: note = Candidate HNH protein
MPARSKRPCRHRGCPAITNDPSGYCDAHRQQHAGDGWRNYQGGKSRHERGYGRPWEIRRARILQRDKYLCQNCRRHGIATKATSVDHIIPKARGGTDDDSNLESLCWPCHRAKTATERTR

SEQ ID NO: 12 - DNA, 4269 bp, synthetic construct
  source 1..4269: mol_type = other DNA; note = p1869 plasmid
tcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttacgtttccacaaccaattaaccaattctgatttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccctgcaggatttcggaggcctgcgttatcccctgattctgtggataaccgtattaccgcctttgagtgagctgataccgctcgccgcagccgaacgccgactagtggattttacggctagctcagtcctaggtacaatgctagcgaattcattaaagaggagaaaggtacccatggcacgtaccccgagccgtagcagcattggtagcctgcgtagtccgcatacccataaagcaattctgaccagcaccattgaaatcctgaaagaatgtggttatagcggtctgagcattgaaagcgttgcacgtcgtgccggtgcaagcaaaccgaccatttatcgttggtggaccaataaagcagcactgattgccgaagtgtatgaaaatgaaagcgaacaggtgcgtaaatttccggatctgggtagctttaaagccgatctggattttctgctgcgtaatctgtggaaagtttggcgtgaaaccatttgtggtgaagcatttcgttgtgttattgcagaagcacagctggaccctgcaaccctgacccagctgaaagatcagtttatggaacgtcgtcgtgagatgccgaaaaaactggttgaaaatgccattagcaatggtgaactgccgaaagataccaatcgtgaactgctgctggatatgatttttggtttttgttggtatcgcctgctgaccgaacagctgaccgttgaacaggatattgaagaatttaccttcctgctaattaatggtgtttgtccgggtacacagcgttaactagggcccatacccccaattattgaaggccgctaacgcggcctttttttgtttctggtctgcccgacgtacggtgaatctgattcgttaccaattgacatgatacgaaacgtaccgtatcgttaaggttcagaacgtatcactggtgacgtacatgccagcccggtctaaacgtccatgccgccaccgggggtgtccggcgataaccaacgaccccagcggttactgcgatgctcaccggcagcaacatgctggcgacggctggcgcaactaccagggcgggaaaagccggcatgaaaggggctacggtcgcccctgggaaatccgccgcgccagaatcctccagcgcgataaatatctatgccaaaactgccggcgtcatggcatcgccaccaaagcgaccagcgtcgaccacatcatacccaaagcgcgtggcggtacagacgacgattccaatctggagtcgttgtgctggccctgccatagagcgaaaacagcaacagagagaacccgatgatgacgcatcctcacgataatatccgggtaggacgaacaataaggccgcaaatcgcggccttttttattgataacaaaaggacagttttccctttgatatgtaacggtgaacagttgttctacttttgtttgttagtcttgatgcttcactgatagatacaagagccataagaacc

SEQ ID NO: 13 - DNA, 1189 bp, Kappa phage
  source 1..1189: mol_type = genomic DNA
ataatttcaaaattgaatacgttgatggcgctttgaccgttctggagacggatggccagtcacggatgaatgaagccgtacatggcatccattttgagcatgtccagggcggacgccccctgctgaaactgacgattgcgcatgatattgccccggcctcgaccccggccccggctgctgcgtcggctcaggaacctttagagggtgagctggtacaggagcaacaatccccgctgcctggcggtcgccgttcccgccatcgccgtggaggtaagcaatgatgtatcaacgcacggatctgacgctctccatgttctatgcatccagcgctgatgcagacgggaacaaagtggctacgttgacgatgcaggtaattgcggcagaggttggtgccgtccagaccagtcaactgctatgcatcaccgatagcgcgaagaaaaaaacgtataccgtgggcgagcaatctatcagtaatggttccgatccgttgctggtcgcgattgagaattactggcgccagagtacggatgtcgtggttaaaggactgatcgccgaggtgaccgatttcatcgcagggaatatcaactcagtgagcacctggatcggccagtttgggatgaaggtatttgagaatcagccattagctgagcggctgccagaaagcgtgctacaggctgatggtagctccgctacagcgacagggtcctgacagcaggcattacaacaggcgctcacagagcgcctgtgataatggctgaatgcttcaccagcgcggcgttttatgggagatcatgatgagttacaccagctgtacttattgcggttcacgtctccatacgcgggctaactgcccgaaaacatggggcggttcatcgcgtcgcgccaatctgcgctgcagctattgtggccagtccgggcataactcgaatgcctgcccacataatgcgagcagcggtcgacggcgcagcctgaatgacgactttaccctcgactgaaccgatagcacgatggcggcaggcggaattgatttcaatgtgaaattattcaatgtcaagtcattgcattgcgcgatgatgataatagatatcatttgagggggtaggggggatcaaatccctaacccctttcgcgcttcgggactgccgcttcaggtagatttttgcgcgtgagaaataaaaacttttttttg

SEQ ID NO: 14 - DNA, 5308 bp, synthetic construct
  source 1..5308: mol_type = other DNA; note = p1867 plasmid
tgattataagaaggcgccccaagcacccccattttagctataaaaaaacccgccgaagcgggttttttcgaaaattgtaaggtcacattacgccccgccttgccactcatcgcaatattgttgaagctcattaagcatacggcctacatggaagccatcacacacggcatggtgtacctggatcgccagaggcattaacaccttgtcgccttgcgtataatatttacccatagtgaaaacaggggcgaagaagttgtccatatttgctacgtttaaatcaaaactggtgaaactcacccagggattagcactgacgaaaaacatattttcgataaaccctttagggaaatatgctaaattttcaccgtaacacgccacatcttgtgaataaatgtgcagaaactgacggaaatcatcatggtattctgaccataacgaactaaacgtttcagtctgttcatggaaaacggtgtaacaagggtggacactatcccaaatcactaattcaccgtctttcattgccatacgaaactccgggtgtgcattcatcaggcgggcaagaatgtgaataaaggccggataaaacttgtgcttatttttctttacggtttttaaaaaggccgtaatatccagttgtacggtttggttataggtgcactgcgcaactgactggaatgcctcaaaatgttctttacgatgccattgactaatatcaactgtagtatatccggtaattttcttctccattttagattccttaggttgcgaaatctcgataactcaaaaaatagtagtgatcttatttcattatggtgaaagttgtcttacgtgcaacattttcgcaacaagttggtttcccgaggcctaacttttgttgcaatggctgtctaccctgttttatactagctcagcccttggtacaatgctagcgttttcattaaagaggagaaaggaagccatgagtaaaggtgaggaattatttactggtgttgttccgatcttagttgagctggacggcgatgttaacggtcataaattcagtgttcgtggtgaaggtgaaggcgatgcaaccaacggtaagctgaccctgaaattcatctgcactactggaaaattaccagtaccctggcctactctggtgactaccctgacctatggtgttcagtgtttttctcgttaccctgaccacatgaagcaacatgacttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccataaactggaatataacttcaactcccataacgtttacatcaccgcagacaaacaaaagaacggtatcaaagctaactttaaaattcgacataacgttgaagatggtagcgtccagcttgcggatcattaccaacaaaacactccgattggagacgctcctgttttactgccggataaccactacctgtccacccagtctaaactgtctaaggatccgaacgaaaagcgcgaccacatggtgttattagagttcgttaccgctagtggtattacgcacggtatggatgaactctacaaataataatcagacagtttcacctgttttacgttaaaacccgcttcggcgggtttttacttttgggtttagccgaacgccataatttcaaaattgaatacgttgatggcgctttgaccgttctggagacggatggccagtcacggatgaatgaagccgtacatggcatccattttgagcatgtccagggcggacgccccctgctgaaactgacgattgcgcatgatattgccccggcctcgaccccggccccggctgctgcgtcggctcaggaacctttagagggtgagctggtacaggagcaacaatccccgctgcctggcggtcgccgttcccgccatcgccgtggaggtaagcaatgatgtatcaacgcacggatctgacgctctccatgttctatgcatccagcgctgatgcagacgggaacaaagtggctacgttgacgatgcaggtaattgcggcagaggttggtgccgtccagaccagtcaactgctatgcatcaccgatagcgcgaagaaaaaaacgtataccgtgggcgagcaatctatcagtaatggttccgatccgttgctggtcgcgattgagaattactggcgccagagtacggatgtcgtggttaaaggactgatcgccgaggtgaccgatttcatcgcagggaatatcaactcagtgagcacctggatcggccagtttgggatgaaggtatttgagaatcagccattagctgagcggctgccagaaagcgtgctacaggctgatggtagctccgctacagcgacagggtcctgacagcaggcattacaacaggcgctcacagagcgcctgtgataatggctgaatgcttcaccagcgcggcgttttatgggagatcatgatgagttacaccagctgtacttattgcggttcacgtctccatacgcgggctaactgcccgaaaacatggggcggttcatcgcgtcgcgccaatctgcgctgcagctattgtggccagtccgggcataactcgaatgcctgcccacataatgcgagcagcggtcgacggcgcagcctgaatgacgactttaccctcgactgaaccgatagcacgatggcggcaggcggaattgatttcaatgtgaaattattcaatgtcaagtcattgcattgcgcgatgatgataatagatatcatttgagggggtaggggggatcaaatccctaacccctttcgcgcttcgggactgccgcttcaggtagatttttgcgcgtgagaaataaaaacttttttttggcgttcggctaaacgtgctacatttgaagagataaattgcactgaaatctagaaatattttatctgattaataagattatcttcttgagatcgttttcgtctgcgcgtaatctcttgctctgaaaacgaaaaaaccgccttgcagggcggtttttcgaaggtcctctgagctaccaactctttggaccgaggtaactggcttggaggagcgcagtcgccaaaacttgtcctttcagtttagccttatccggcgcatgacttcaagactaactcctctaaatcaattaccagtggctgctgccagtggtgcttttgcatgtctttccgggttggactcaagacgatagttaccggataaggcgcagcggtcggactgaacggggggtttgtgcatacagtccagcttggagcgaactgcctacccggaactgagtgtcaggcgtggaatgagacaaactcggcagtaacagaggaatgacaccggcaaaccgaaaggcaggaacaggagagcgcacgagggagccgccagggggaaacgcctggtatctttatagtcctgtcaggtttcgccaccactgatttgagcgtcagatttcgtgatgcttgtcaggggggcggagcctatggaaaaacggctttgccgcgaccctctcacttccctgttaagtatcttcctggcatcttccaggaaatctcagccccgttcgtaagccatttccgctcgccacagtcgaacgaccgagcgtagcgagtcagtgagcgaggaagcggaatatatcctgtatcacatattctgctgacgcaccgatgcagccttttttctcctgccacatgaagcacttcacttacaccctcatcagtgccaacatagtaagccagtatacactccgctagcgcagatgtccggcggtgcttttgccgttacgcactactttagtcagttccgcagtaccgtcaggcgctgacatagctatttactttgtattgcctgcaatcgaatttctgaactatcatatagtggggataacgggaaagttactatatttgcgaactaacttaggcgtccacctcgaagctacctaatcacacccaacccgcgcggggtaaataaggcactaatccgagcttaaagcttgcgtagcacttagacacaagttaattaccaattgtctggtagtttggcggtattagcgagatcccagacgcaaggcagagttaattttaacctaaagccacaaataagacaggttgcacaagcccgccggaaattaaatcttgctcacttcggtaacggagtttccctcccgcgtacttaattcccaataagaaacgcgcccaagtcctatcaggcaaaattcagccccttcacgtcttagaacgagggtaaaaatacaagccgattgaacaagggttgggggcttcaaatcgtcgtttaccccactttacaacggagggtaactagttcaccctatagtacgaagcagaactatttcgaggggcgtgcaataatcgaatcttctgcggttgacttaacacgctagggacgtgccctcgattcagtcgcaggtactccgactcacactgcctcacacccagctagtcactgaccgataaaattgacccgccctctaaggtagcgagtacgtcctaaaaggcttcggacagggctatataggagagtttgatctcgccccgacaactgcaaccctcaactcccttagataatattgttagccgaagttgcacgacccgccgtccacggactgctcttagggtgtggcttcttaatctgacaacgtgcaacccctatcgagggcgattgtttctgcgaaaggggttgccctaatagtcgcgacaattggcccttgtaggggtgaaaccacttagtttcgcgccgtagtcctaaaggcccacctattgactttgtttcgggtagcactaggaatcttaacaatttgaatttggacttgttttaggggcgttattcgagggcaatcggagctaacttcaagactacttctttgttgaatactaaatagtgcaaaggtcgtgtttcctcaaggatactccgctaacaatataggattccaatcagattcagcactggctgtacgggtgttacggtgaggttttcgggtttacggctggaagctagcacggtaggaagcctttcaatcacaaagcaaaagggccgtcgaaggcccacaagatacgaaagctctcgaagccttatccttgaacgatccacctatttaggcagttacgcacaaaagctacccaataatccgtgacaggcacaatatcacggaacaaaggcgaaaactctcgtacacggttaggttttcgctaggaagaataaacctctatct

SEQ ID NO: 15 - DNA, 234 bp, synthetic construct
  source 1..234: mol_type = other DNA; note = predicted ORF
atgcttcaccagcgcggcgttttatgggagatcatgatgagttacaccagctgtacttattgcggttcacgtctccatacgcgggctaactgcccgaaaacatggggcggttcatcgcgtcgcgccaatctgcgctgcagctattgtggccagtccgggcataactcgaatgcctgcccacataatgcgagcagcggtcgacggcgcagcctgaatgacgactttaccctcgac

SEQ ID NO: 16 - protein, 78 aa, synthetic construct
  source 1..78: note = Predicted protein with 2 Zn fingers
MLHQRGVLWEIMMSYTSCTYCGSRLHTRANCPKTWGGSSRRANLRCSYCGQSGHNSNACPHNASSGRRRSLNDDFTLD

SEQ ID NO: 17 - DNA, 209 bp, synthetic construct
  source 1..209: mol_type = other DNA; note = Short cos site
tttaccctcgactgaaccgatagcacgatggcggcaggcggaattgatttcaatgtgaaattattcaatgtcaagtcattgcattgcgcgatgatgataatagatatcatttgagggggtaggggggatcaaatccctaacccctttcgcgcttcgggactgccgcttcaggtagatttttgcgcgtgagaaataaaaacttttttttg

SEQ ID NO: 18 - DNA, 4330 bp, synthetic construct
  source 1..4330: mol_type = other DNA; note = p1868 payload
tgattataagaaggcgccccaagcacccccattttagctataaaaaaacccgccgaagcgggttttttcgaaaattgtaaggtcacattacgccccgccttgccactcatcgcaatattgttgaagctcattaagcatacggcctacatggaagccatcacacacggcatggtgtacctggatcgccagaggcattaacaccttgtcgccttgcgtataatatttacccatagtgaaaacaggggcgaagaagttgtccatatttgctacgtttaaatcaaaactggtgaaactcacccagggattagcactgacgaaaaacatattttcgataaaccctttagggaaatatgctaaattttcaccgtaacacgccacatcttgtgaataaatgtgcagaaactgacggaaatcatcatggtattctgaccataacgaactaaacgtttcagtctgttcatggaaaacggtgtaacaagggtggacactatcccaaatcactaattcaccgtctttcattgccatacgaaactccgggtgtgcattcatcaggcgggcaagaatgtgaataaaggccggataaaacttgtgcttatttttctttacggtttttaaaaaggccgtaatatccagttgtacggtttggttataggtgcactgcgcaactgactggaatgcctcaaaatgttctttacgatgccattgactaatatcaactgtagtatatccggtaattttcttctccattttagattccttaggttgcgaaatctcgataactcaaaaaatagtagtgatcttatttcattatggtgaaagttgtcttacgtgcaacattttcgcaacaagttggtttcccgaggcctaacttttgttgcaatggctgtctaccctgttttatactagctcagcccttggtacaatgctagcgttttcattaaagaggagaaaggaagccatgagtaaaggtgaggaattatttactggtgttgttccgatcttagttgagctggacggcgatgttaacggtcataaattcagtgttcgtggtgaaggtgaaggcgatgcaaccaacggtaagctgaccctgaaattcatctgcactactggaaaattaccagtaccctggcctactctggtgactaccctgacctatggtgttcagtgtttttctcgttaccctgaccacatgaagcaacatgacttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccataaactggaatataacttcaactcccataacgtttacatcaccgcagacaaacaaaagaacggtatcaaagctaactttaaaattcgacataacgttgaagatggtagcgtccagcttgcggatcattaccaacaaaacactccgattggagacgctcctgttttactgccggataaccactacctgtccacccagtctaaactgtctaaggatccgaacgaaaagcgcgaccacatggtgttattagagttcgttaccgctagtggtattacgcacggtatggatgaactctacaaataataatcagacagtttcacctgttttacgttaaaacccgcttcggcgggtttttacttttgggtttagccgaacgcccgtttaccctcgactgaaccgatagcacgatggcggcaggcggaattgatttcaatgtgaaattattcaatgtcaagtcattgcattgcgcgatgatgataatagatatcatttgagggggtaggggggatcaaatccctaacccctttcgcgcttcgggactgccgcttcaggtagatttttgcgcgtgagaaataaaaacttttttttggcgttcggctaaacgtgctacatttgaagagataaattgcactgaaatctagaaatattttatctgattaataagattatcttcttgagatcgttttcgtctgcgcgtaatctcttgctctgaaaacgaaaaaaccgccttgcagggcggtttttcgaaggtcctctgagctaccaactctttggaccgaggtaactggcttggaggagcgcagtcgccaaaacttgtcctttcagtttagccttatccggcgcatgacttcaagactaactcctctaaatcaattaccagtggctgctgccagtggtgcttttgcatgtctttccgggttggactcaagacgatagttaccggataaggcgcagcggtcggactgaacggggggtttgtgcatacagtccagcttggagcgaactgcctacccggaactgagtgtcaggcgtggaatgagacaaactcggcagtaacagaggaatgacaccggcaaaccgaaaggcaggaacaggagagcgcacgagggagccgccagggggaaacgcctggtatctttatagtcctgtcaggtttcgccaccactgatttgagcgtcagatttcgtgatgcttgtcaggggggcggagcctatggaaaaacggctttgccgcgaccctctcacttccctgttaagtatcttcctggcatcttccaggaaatctcagccccgttcgtaagccatttccgctcgccacagtcgaacgaccgagcgtagcgagtcagtgagcgaggaagcggaatatatcctgtatcacatattctgctgacgcaccgatgcagccttttttctcctgccacatgaagcacttcacttacaccctcatcagtgccaacatagtaagccagtatacactccgctagcgcagatgtccggcggtgcttttgccgttacgcactactttagtcagttccgcagtaccgtcaggcgctgacatagctatttactttgtattgcctgcaatcgaatttctgaactatcatatagtggggataacgggaaagttactatatttgcgaactaacttaggcgtccacctcgaagctacctaatcacacccaacccgcgcggggtaaataaggcactaatccgagcttaaagcttgcgtagcacttagacacaagttaattaccaattgtctggtagtttggcggtattagcgagatcccagacgcaaggcagagttaattttaacctaaagccacaaataagacaggttgcacaagcccgccggaaattaaatcttgctcacttcggtaacggagtttccctcccgcgtacttaattcccaataagaaacgcgcccaagtcctatcaggcaaaattcagccccttcacgtcttagaacgagggtaaaaatacaagccgattgaacaagggttgggggcttcaaatcgtcgtttaccccactttacaacggagggtaactagttcaccctatagtacgaagcagaactatttcgaggggcgtgcaataatcgaatcttctgcggttgacttaacacgctagggacgtgccctcgattcagtcgcaggtactccgactcacactgcctcacacccagctagtcactgaccgataaaattgacccgccctctaaggtagcgagtacgtcctaaaaggcttcggacagggctatataggagagtttgatctcgccccgacaactgcaaccctcaactcccttagataatattgttagccgaagttgcacgacccgccgtccacggactgctcttagggtgtggcttcttaatctgacaacgtgcaacccctatcgagggcgattgtttctgcgaaaggggttgccctaatagtcgcgacaattggcccttgtaggggtgaaaccacttagtttcgcgccgtagtcctaaaggcccacctattgactttgtttcgggtagcactaggaatcttaacaatttgaatttggacttgttttaggggcgttattcgagggcaatcggagctaacttcaagactacttctttgttgaatactaaatagtgcaaaggtcgtgtttcctcaaggatactccgctaacaatataggattccaatcagattcagcactggctgtacgggtgttacggtgaggttttcgggtttacggctggaagctagcacggtaggaagcctttcaatcacaaagcaaaagggccgtcgaaggcccacaagatacgaaagctctcgaagccttatccttgaacgatccacctatttaggcagttacgcacaaaagctacccaataatccgtgacaggcacaatatcacggaacaaaggcgaaaactctcgtacacggttaggttttcgctaggaagaataaacctctatct

SEQ ID NO: 19 - DNA, 5264 bp, synthetic construct
  source 1..5264: mol_type = other DNA; note = p1872 plasmid
tcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttacgtttccacaaccaattaaccaattctgatttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccctgcaggatttcggaggcctgcgttatcccctgattctgtggataaccgtattaccgcctttgagtgagctgataccgctcgccgcagccgaacgccgactagtggattttacggctagctcagtcctaggtacaatgctagcgaattcattaaagaggagaaaggtacccatggcacgtaccccgagccgtagcagcattggtagcctgcgtagtccgcatacccataaagcaattctgaccagcaccattgaaatcctgaaagaatgtggttatagcggtctgagcattgaaagcgttgcacgtcgtgccggtgcaagcaaaccgaccatttatcgttggtggaccaataaagcagcactgattgccgaagtgtatgaaaatgaaagcgaacaggtgcgtaaatttccggatctgggtagctttaaagccgatctggattttctgctgcgtaatctgtggaaagtttggcgtgaaaccatttgtggtgaagcatttcgttgtgttattgcagaagcacagctggaccctgcaaccctgacccagctgaaagatcagtttatggaacgtcgtcgtgagatgccgaaaaaactggttgaaaatgccattagcaatggtgaactgccgaaagataccaatcgtgaactgctgctggatatgatttttggtttttgttggtatcgcctgctgaccgaacagctgaccgttgaacaggatattgaagaatttaccttcctgctaattaatggtgtttgtccgggtacacagcgttaactagggcccatacccccaattattgaaggccgctaacgcggcctttttttgtttctggtctgcccgacgtacggtgaatctgattcgttaccaattgacatgatacgaaacgtaccgtatcgttaaggttcagaacgtatcactggtgacgtacatgccagcccggtctaaacgtccatgccgccaccgggggtgtccggcgataaccaacgaccccagcggttactgcgatgctcaccggcagcaacatgctggcgacggctggcgcaactaccagggcgggaaaagccggcatgaaaggggctacggtcgcccctgggaaatccgccgcgccagaatcctccagcgcgataaatatctatgccaaaactgccggcgtcatggcatcgccaccaaagcgaccagcgtcgaccacatcatacccaaagcgcgtggcggtacagacgacgattccaatctggagtcgttgtgctggccctgccatagagcgaaaacagcaacagagagaacccgatgaataatttcaaaattgaatacgttgatggcgctttgaccgttctggagacggatggccagtcacggatgaatgaagccgtacatggcatccattttgagcatgtccagggcggacgccccctgctgaaactgacgattgcgcatgatattgccccggcctcgaccccggccccggctgctgcgtcggctcaggaacctttagagggtgagctggtacaggagcaacaatccccgctgcctggcggtcgccgttcccgccatcgccgtggaggtaagcaatgatgtatcaacgcacggatctgacgctctccatgttctatgcatccagcgctgatgcagacgggaacaaagtggctacgttgacgatgcaggtaattgcggcagaggttggtgccgtccagaccagtcaactgctatgcatcaccgatagcgcgaagaaaaaaacgtataccgtgggcgagcaatctatcagtaatggttccgatccgttgctggtcgcgattgagaattactggcgccagagtacggatgtcgtggttaaaggactgatcgccgaggtgaccgatttcatcgcagggaatatcaactcagtgagcacctggatcggccagtttgggatgaaggtatttgagaatcagccattagctgagcggctgccagaaagcgtgctacaggctgatggtagctccgctacagcgacagggtcctgacagcaggcattacaacaggcgctcacagagcgcctgtgataatggctgaatgcttcaccagcgcggcgttttatgggagatcatgatgagttacaccagctgtacttattgcggttcacgtctccatacgcgggctaactgcccgaaaacatggggcggttcatcgcgtcgcgccaatctgcgctgcagctattgtggccagtccgggcataactcgaatgcctgcccacataatgcgagcagcggtcgacggcgcagcctgaatgacgactttaccctcgactgatgacgcatcctcacgataatatccgggtaggacgaacaataaggccgcaaatcgcggccttttttattgataacaaaaggacagttttccctttgatatgtaacggtgaacagttgttctacttttgtttgttagtcttgatgcttcactgatagatacaagagccataagaacc

SEQ ID NO: 20 - DNA, 20 bp, synthetic construct
  source 1..20: mol_type = other DNA; note = AD1334 primer
ggacctcccaccattccaag

SEQ ID NO: 21 - DNA, 20 bp, synthetic construct
  source 1..20: mol_type = other DNA; note = p1335 primer
acggcgatgttcaggttctt

SEQ ID NO: 22 - DNA, 20 bp, synthetic construct
  source 1..20: mol_type = other DNA; note = AD1336 primer
ggcgaaagaagacctggtca

SEQ ID NO: 23 - DNA, 20 bp, synthetic construct
  source 1..20: mol_type = other DNA; note = AD1337 primer
tagccggcgaaatggatgtt

SEQ ID NO: 24 - DNA, 20 bp, synthetic construct
  source 1..20: mol_type = other DNA; note = AD1322 primer
catcagaccgcattcgcttg

SEQ ID NO: 25 - DNA, 20 bp, synthetic construct
  source 1..20: mol_type = other DNA; note = AD1323 primer
ggacgaagatgtggaagcca

SEQ ID NO: 26 - DNA, 32767 bp, BW4 phage
  source 1..32767: mol_type = genomic DNA
agcgatatctccccgggtttttccacagggtgtccgcccagggcgtcgctgtcgagctcacagcgcacgctgaacgcccgccagcactcgaggcgatccgaacagcctcgctccgagtgcgtcgggcctgtgtggatcgctcatgagtttcgtaacaagcccctagccacagcccgattcagatagaataggagcatggaagggcagtgcggatggtgcggtcgggcattcgatcgtgcccggacgggtcgcccgcgacgcttctgctcggcccgctgtcgggtcgccgcgtcccggtgtgcgatcccgctggccatgaggtcccgcactgcgtgggtccgctgcgacggcaagcgccccatcaccctggctggcgctccggcctcatccacggacccgggcacatggtctggctggtcgcaggtgcgacgcgccacggccggcgatggcttcgggaccatgctcggtgacgggctggggtgctgggatctcgaccacttcgacgatcagggcgcccgggccttcatcgaccggatcgataagccgatcatcttcgccgagcggtcggtgtcggggcatggcttccacatcttcgtccggactgacgaggcccccggacgccgcaccggaaacatcgagttctactcacgccatcggttcatcagggtcacaggagaccagttcgtctgaagaagggggtgcgccatggctgcacaggtcagggccgtggaccccgatgagcgcccacccgcccgcaagcgggccaagaccatcacccaggccgcgaagtccggcactgaggttgaactgttggaggcactgcaggctcgcgtggcccgcgccgtgcaggaccgtgacactccgccgcgcgatctggcagcgctgacgaagcggctgatggacatcacccgggagctcgaggcggcccgggtcaaggatcaggaggcgggatctgatggtgccgtcaccgcagacgaaacatggcgaccgcaagctctctgaggtcgccaagcacctgatccttcctgaagggatcgtctcgacgggctggccggccgtgcgtgaccggtgtggcgagtggggtgtggtcttcgaccgttggcaggacggcatgggccgggtgatcctgtcgaagcgcggcagcggcctgttcgccgctggtgtgggcggggtcggcatgtcgatcccgcgccagaccggcaagaccttcaccgtcggcatgatcatcctcgggctgtgctcgctgagcgaggagctcacggtgctgtggacctcccaccattccaagacgaccaccaagactttcgagtcgctgcggggcatggcccagcgtaagaaggtcgccccgttgatccgtcaggtccgaacaggaaacggtgaccagcagatcattttcagcaacggttcgaggatctacttcggtgcccgggaacagggcttcgggcgtggcttcgacgacgtggacatcgagatctttgacgaggcgcagatcctgtccgagcaggccctctccgacatggttcccgcggcgaatgtgagcaccaatccgctgatcatcttcatgggcaccccgccgcgtccctcggacccgtcggaggcgttcgcgaaccgccgcgccgaagctctggcgggcgacgccccggacgccgcctggatcgaattcggagcggacgagcacgccgacccgaccagccgcgcccaatggcgtaaggcaaacccatcctttcctcaccgcacgtcggagacctccattctgcggatgaagaagatgctcgggcccgagtccttcaaacgcgagggcttgggcatctgggatgagacggcatcggtccgcgcgatcccagccgaagggtggcgcgtcctgaccgtcaaggaaccacccgccgacgcgatccagtccttcggcatcaagttcgccatcgacgggagtgcggtcgccctggcagccgccctgaaacccaaggacgggccgatctatgtcgaaggaatcgagcagcgctcggcatccgacggcatcgaatggctcgccgactacctgacgcccctgtggcgcaacacggcccagatcgtcatcgatggcaagtccggcgccggtgccctggttgatgcgctgcgccgtggtggcgtggctgcgaaggtgatcctcaccccgagcgtcgccgacgtgatcaccgcccacagcctgactctggaggccatcaagaccggtggactgtcgcacctggctgacccggagctggatcggcaggtccgcatcgccacgaagcgaaagatcggggccgccgggggcttcggctggcaggcccccgaaggcgacaccgtcgccctcctcgacgccatcacgcttgcccactgggcggccctcaccacgaagcgacatcccggcaggaaggcggtggcactggcatgagcctcctcgtcaacccctatgcgtcgccgtccttcttctcgtccccgtccgtggtcggactcggagcagacgagcaggagctcctggacgagctggtggccctgtgggcacgcaagaagccccgcaacgtgctgcgcggcctgtaccttgacggcaagcagcagatcaagaacctgaacatcgccgtgcccgacgagatcgccgacagtctccagatcgtggtcggctggcccgagaaggccgtcttcgggctatcgaacctgtgcatgtgggatggcgtcgtcactcccacaggcgacgagaatcccttcgggcttgacgatctcctgtcggccaaccgcttcgacgtcgagatcaatgaaacgatcacctcggccatggcgaactccgtggccttcctgaccgtatcggcgggcaacgtgtccataggtgagccgccggtggtgatcatgccgttctccgccgaatgggcctcagccctgtgggaccggcgcacccgctcaatcaaggcgggactgaccatcggcgacatcgactacctgggccgccccaccagcctctcgctcttcacccgcaccgccaccatcacctgcgtggggtcccggctgggatggatgatcgaagatcgcgccgagcacgggctgaaccgcgtcccgatggagccggtcccgttccgcccaacccttgaccgccccttcgggcgctcgcggatctcgcgccaggtgatgaccatcgtggaccgcgccatgcgcgcggccctgcgcatggacatctcctcagagctgttcaccgcacccggcctgctcctcaacggaatcaccccggagcagtgggcagagatccagaagtggacatggaagctcggcacggtgcgcggcctgactcgcgacgaggatggcgagaccgcatcggtcgagacgatcccccagcagtcgatggaaccgttcatcgcgcagctgcgcgagctggccgaggaattcgcctcagccacatccatgccgctgtctgcattgggggtcgtccaagacaacccctcctcggctgacgccatctacgcggcgaaagaagacctggtcatcgaggccaccaacgccaaccggatcaccggctacgcgctatcccgggtcttccaagacgcggtgatgatgcgcgacggcctgaccgagatgcccgacgagctcggcggggtcgccgccaagtggcgcaacccggcgatgccgtcgatcgtgtcccagtccgacgcgatggtcaagcagatttcggcgatccccgggctggccgctaccgacgtcgccttcgaacagctcggctattcggcggctgacatcgtgcggattcgtacccagatgcgccgagcccaggctgcggacggcctgacttcgttgctggccaaaccagccacgtcgtcaacgcctggcgcggagccctctcagtccgcaagtccgacggagccagctgcaagcactccgctgccggacctcgaaggggcccctggtgaccgatcgtgatgacctgaaccatttccacgaggccaatgacgcgatccagcggcgcgcaatcaacgacctgaacaagttttgggcgcggcttgccaagtcagacccgaaagccgttcgcgcagccatggacttattcgtcccccagctcatcgcctcctacggagagttggccgccgaagccgctgcccgttggtatgaggaactacggcccgccgacaagaagaacttccaggccgaactcgcggaccctgtgtccgacgacatcatcgaggcagatgtggctgaggccctggggaccagcggcgcctgggacaccgaggcggtgcgagggagcctggccgatgcgatcaggcgtcagatcttctacatggcgcgggcgactgtcgcacgcaacatcgctcacgacccgaagcgtccaaggtttgcacgagttcctcggggcgcggtcacgtgcgcgttctgcaccatgctcgcctccagggggtgggtgtactacaccgcgaagactgccgggatcacacgaccctggcatcgcaagtgcgactgccagatcgtgcctgagtggaaacgcggcaacatccatttcgccggctacgaccctgacaagatgttcgagcagtatgccgaatcggtcgatgcggtggggtcgagcttcgacacgaaggcaatcctcgccgacatgcgccgacgccatcccgaagcgctgaccgacggggtcgtcaacatgagtgaaggacagggtccggtgaccagtgattagacagtcggtgaacggatgactacgccgtcggcgctgcgccgccgcctggaatggctactggagaaccgtgaacggcttctcaggagccatggcgagtcggactttgccgagatgctggatggcgcccgtcacgagcttgatgaggcccgcgagcaggcaggcctggccgcgcagtcaaacccaatctgtagcaagccccgttccaccttcgggtgggcggggctttgtcatgcccgcatccgggcatccaattccgtcccaccgcgagggtggggcgtcgacctggtggcgcgatgccgccgaactaatccctggaaggggaaactgctatgcacaagaagctcatgccgtgggtccgtctcatcgaggcggtcgagactcctgctggagccgcccccacgcccgcgatcgatccgaaggatccggcagccaatcccaccactgagccgaagccggccgacgcgacgtcggagaagcctctcggcgaggcgggcaaggttgcgttggatcgcgagcgcgaggctcgccgcagcgccgacaagcgcgccagtgagttggaggcccgtgtgcaccagctcgaggacgcgggcaagaccgaggcccagaagcaggccgacgaactcaagcgcacccagtccgagctggagacgctgaggggcgagaaggcacggctggaggtggcgtccgcgacgggcgtcccggtcgatctgctcgctggccccggcgacgatctggatgcctacgcgcaggccctgaacgcctggcgcgacaagcagtccgaaaagccagccgcccctgcggtggacaccccttccccttcgccgtccggggtgaccggacagcccgtgcagccgaaccggacggtcgatgaactcatcgcggccgccgagaagaacggcgatctggcaaccgcgaagcaactcaaattgatgaagctcgacgcactgcgtcggacgtcctgatcagaaaggcaccactatgccgggcattaccggacagggcaccacctacaaccttccgaactatgtgggggagctttttgcggcatctcccgaagacaccccgctgctgtcggcgatcgggggactgaccggcggcgagtcggtcggcgcccgccagttcgaatggcagggctacgacctgcgcgacgccgacggttcgcgccagcgcctcgagggagccaacgcccccgacggtgaggagcgcacccgctactccgcctccaatgtggtcgagatccaccaggagtcggtggaggtgtcctacaccaagcaggccgcgaaccgtgagcgggctaccaacggtgccgccacggtccagctggcgggctccgtgctgccggccgatgagctcacctggcagatcgaccagcagctcaagcaggtcgcccgcgatgtcgagaagtccttcatcgcgggcacctaccagctgcccaccgacaacgccaagccgcgccgcacgcgtggcctgctggaggcgaccaccacgaacgtggccgcctcgacccacaccgcaaaggaactcaccgtggaggagatcctcgacctgttccagaaggtgtgggagaacggcggcatccaggaagccgagacccgcaccgtcattgtcggtgccgccctgaagcggaccctgacgcgcctgttcatcaccgacgtcaagtaccaggaagaatcccgcaacgttggcggtgtgaacctgcagaccttcgaaaccgacttcggcaaggcgaacatcatgctcgaccgcttcatgccgagcgacaccctcgtggtcgcgtcgctggaggacctgaagccggccttcctcgacatccccggcaagggccacttcttcgccgagccgctcgccaagaccggtgcagccgacaaggtgcagatttacggcgaggtcgggctgcagtacgggaaccagcgcaagcacggaaagctcactgtcgcacccgcaacccccgccaagtaatcacggatcggtttgaggttgcctgatgaaagtcacctcgaccatcccgaacctgactgttctcgacctggacatccagttcgttgacggtcaggccgatgtggacccgcatctcgccgagaggctgcgtcgcctcgagcctctcggcgtgcgggtccccacagccagccgcaagccgcccacgcggtcgcggcgtaagcagggggtcagccatggtcgcacctgatccggaactgccgttcgccaccgtctccgatatggagagccggtggcgttctttgtctaaggacgagcacacgcgggccgaggcccttctggacgatgcgagcgggttgatcgttgatacctgcccgcgctgggaacaggcctcaccggccaccctgcggcgtgtgacgtgctctgtcgtgcgccgggcgatggccgcagacgatgaggacatcggcgcaacctcgctcatggacacgacgggccccttcaccactcagcgcgcctactcatcaccggccggggatctcttcttgaccaaggccgagaaggccgcgctcggcggggtcaccggcgcattcgagacgagccttctggggctgacatgaagcgctcatggccgacacccgtggaacgtctccgcgagggtccgcccgagattgaccgtgacggtgatccgattgccggctccggagtgatcaccaaggatcctctccctgatgccctgttcgcgccgggcggctcgcagatcctcgtcgcccccggcgtggcggcagtcgtggacgaacccaccctctactggcgcggatcagaagtgatcgatgtggtggccaccgacaaggtccggatagccggccgagtctggacccctgaaggaaatcctgcgcgatggccgaagggcgtcgtgctcaagctcaaggcccaggaggcaaagaatcgtggctaatttccgtttcgaacccaatacgaaggcgttcaccgagtgggcgcagcgcgactgcgacgcgcacctgatcgccggcatcacggcctcgatgggggccaaggcgggcgagggtttctcgacgatggtctccaacaatggcgaccgcacccgcggttatctcgcgacggcctccacgaagggccgtatgcggcaggcgcaggggcatgtcatcgagcgggtcatcggatcgagcggcgtgtgaaaccgcccgacctccacacgctcgtcgcccaccatctggctgagctcctcgacgtgccggtcgtctccacccgccccgagggagagacggcgccgtccaagttcgttcggatcatctcgaccggcggagcgggccgctatggccgggtcttccagggcatccagctgacgatcggctcctacgcgggatcggcggcgaccgcccgtgatctcgcgatgcaggtggacgaggccatgaatgggctgccggtctcgccgttgccggtctccaaggtcaccggcaacaccccgtcggacgaccccgatcccgacactcagcaggcccgccacacggccacctaccaactcaccacccttatctcttaggagtcattcatggctgtcaattccgtcaacgtgcacgtcttcgggtccgatgacgacgtgctctacctgggcccgtcaggtctgaatctgggcaacatttcgctggaaaccgcgatcccgaaggagatgatcgacaccggctggctcactgatgacggtgtgaccctcggcatgaaggactctgtcaaggccatccagggccaccagggccacgcgaatgtgcttcagttcatggactcgtcggataccaccctcgaggcgaccctcatggagtctcagctgcagaccttcctgtggaacctcgacgcggacgctgaggacatcgacggggtcaccaagatcaccgcggccagctcccgcaaggtcctcaacctgtgcgcgatctgggacaccttcgacacccagcacagcggcatccattggcgctacgtcttcccctcgctcaccctgggcgagcgcgatgacatccccttcaaggtgggcgaagccagcgcttacaagtattcgctgggtgtgctggagaagttcttcgtcttcaccaacgcggcagcgatgaaggccggtggagcatccgccaagacggtgaccggtgtgaagatcaccaccaccgacggtgcgaccgtgggcctcccgtcgtcgctgaaggtgggggagaaggtgtccctcgccgccgagatctcctacagcgacgggacgaaggcggtcaagcagaccaatgccgtgggcctcacctggacgtcctcggacaaggccaaggccaccatcgatggcggcgtggtcaccggagtctcggcaggcaaggccgacatcaccgcctcgatcgacggcaagacttccgaagcgctgtcgctgaccatcaacaccgccgcctgaccaaccctcaaaccctccgccccggtcgtcctctcgcgccggggcggagccttgccacacccgcgagaggtcaacttttctgcgagaggaaaccatcatggccgaggccaagaagatcagcgccgccgagaaggcgcgccgcgagacccagtccgcgaaggacaccggcacgatcaccgacaccaccgtgcagatcggcgatatcgagttgaccgtgcccgccgccgtcttcgaagacgactgggaattccaggaggcgatcctgatggccaacgatcccgatgccaccgacgaggatcgggccagggcaagcatgacgctgttccgtcgtctggtcggaaaccgccaccgcgaagtgcttgaccagctgcgcgacgagtcggggcgtgtgccggtgtctaaggtcaccgagaccgtcaagaaggtcatggacgcggtcaacccaaactgatgagcctcttccagctcctcgccacacattgggaggagctggagggggacttccaagaggcctaccgcgtcgacctgcgggacttgtggcgtggtcggctgagcccggcgcgctgctgggtgctgctgacacaactgccacccgggtctcggctctggcggatgctcggcggccccatggcgtggggcatggtcgagcgcgccgtccgtgaagagggctggcgactcgcctcccagaacgctggtaaggaactgcctcggccggagccgcctgcgccgggatggcgcgacaagcaggacgacctgcgacgccgcgaagagcgccgtcttgcccgcttcatgcaacgccacgcagaacgcaacaactgaacagtgcaccgtcccgggaggtttccatggctctagatctcggtaccgcctgggtgcaggtgtctccgtccttcaggggcttcgcctccacggtgaacaaagaggtcggttcggcagtgggcggggccttcaagtctgcggccaaggtcggcaccaccgcgatcgccacgatcggtgcggccgtcggtgggctggcgctcaagggcggcatcgaccgcgccctgtcgatcgagcaggcgcaggccaagctgaagggcctgggccacgacgcagggtcgatcaccgagatcatgaacgacgccctcgcctcggtgaagggcaccgccttcggtctgggcgatgccgcgacggttgccgcgtcgatgtcggctgccggcgtcaagtcgggcgagcagatgaccggtgtgctgaagacggttgccgacaccgcccagatttcggggcgctcgctcaccgatatcggtgcgatcttcgggtcggtggcggcccgcggcaagctgcagggcgacgacatgctgcagctcatgagctccggcgtgccggtgctccaattcctttccgaccagctcggcgtcaccaccgccgacgtgtcggacatggtgtccaaggggcagatcgacttcgccactttctccgccgccatgcagaagggtcttggtggtgcggcactggctggcggcgaaaccttcaccggtgccatggccaacgtccgcgccgccctgtcccggctgggtgaggctgccgccaagcctgccctggacgggctgcgcaatgtcttcaacgcgctgatcccggcgattgatgccgccacaaatgcgctcaagcccatcgccagcgccctggcgaaccgaatttcgcaagcagcagaggcggcttccgcctcgatcgggcgcctcaccggctccctcacgagcatcacgaatctcaatacagggatgctcggcgcggccttctcatcgatgctgccgatcatcggagcactgtcggggcagcttggctccttgcttggcgggatcccggtcgtcgggcaggccttcgcagggatcactgggccggtgggattggctgccggcgtgctggtcgagatcgtggcggcttcatcgtcgctgcgtcaggccctgggcacgctggtcggggtcgtcgggtctcagttgtccggtgtgatgacgggcatcgtcgcggtgtttgccggcttcaggtccgtgcttggtgccgtcggtgacgttctggccccgttcgtggaccgtgcggcggacgccgccaatgtggtcctgcccttgctggggggtgcgctgtcggctgccggtggcatcctgcagtcttttgcgggtttcatcgagcgcaaccatgtggcgctctccattcttgcgggtgcggtggttgcggccgcgacgagttggaagatctataccggcgcgcaagatcttgcgcggctggcaacgacgaagctcgggctcgcgacaacggtcctgaagggcaagctgtcatcgatgggggcggcgttcaagacgaatccgttcggtgtcatcctcatggcgatctcggcgctggtgggggcgttctcgattgcctaccagtcctctgagacgttccgcaacggtgtgcaggggattctcggctcgctggcgccggtgttttcctccctgatggggacgctgtcggggctattccagcaggtcgcgggcgctgtcgggccggtgctgtcgtcgatcgtctcgacgctggcgtcggtgttctcggcgatcggtcccgtcctgtcgcagctggccggcaccatcggatctgtcttctcggcgatcggtcccgtcctggcgtcggtcttcgggtcgatcgggtcggttctggcgagtgtcttctccggggtgatgagtgtcgtggcgccgatgctcaccgcgttgcagccgctgttcacgcagctgtcggcttcggcggggcagatcggtgcggcgttcggtcctgttggtcaggcgctgtcgtcgtccttccagcaggtcggtgccgcgctggcgccgctgctgccgatgcttggtcagcagttcggggcgatcctgtctcagctggctgcggccctggctccggtcatgggtcagttgctggctgcggctgctcaggtgttgccgacgttggcgcaggccttcgggcaggtcgccggggtgctgatcgggtcgctgggtcaggctctgacccagatcgctccgctgataggccagctggtgggggtgctgatcgggtcgctgggtcaggctctgacgcagattgccccgctggtgggcaccctggtcggggtggtcgcgcagctgttcgcccagctggcccctttggtgggtcagctgctggtgcagcttgttccggttgtcgcgggaatccttgtggcgatcgtgccgatcgtcgggatgctgattagtcagctcgttccggtgatcgtcacgctgctccaggtgatcaccccgattatcaccatgctaatcagcgcgctggtgccggtgatccaggtcgtgacccagctggtgctggcgatcatccaggcggtgatcccgttgatctcggcgatcctgccggcgatctcggcactcatctcggcgctgctgccggtgatcgtcatgatcatccaggtggtggcgcaggtgctgcagtggctggcgccgctgatctccaccctgatcacggcactgattccggtgatcaccacgatcatccaggtggtcatcacggtcgtgtcgacaatttggtcggtggtcggggcggtcattggctggttccagtccacggttgtgcccatcatcggcaccgttgttggtgcgatcgcgaacgctttcggttgggtgcgcgaccgtatttccgatgcctggaactggattaaggaccgcattgtcgccccggttgtcgagtggttccagtccacggtggtgccgaagttcgaggcggtgcgcgactccgtggtgcgggccttcgagacgctgaaggatggcgttggtcgcgcctgggatgcgttgaaggatctcgcaaagaagccggtcgaattcgtcgtgaacacggtggctgccgggttggtgcgggcctacaactgggtggcgacgaagttcggtgccgacgaggtcaaggagcctcatgtcgagttcgccaacggcgggttcgcgggacgtgaggccggcttcgcgtcgtcgccgatcctgtgggccgaggccggcccggaagcctatatcccgttggatccggccaagcggacacgctcgctggggatctgggccaagaccgggcagatgctcggcgctctacccatggctgacggcgggatcatcgggaacatcattggcgggatcggcaacgccgctgcggcgatcggcaatttcatcaagtcaccgatcgagtggctcatgggccgggtccgggacctgatcgatgatgtgggcagctcaccgttcgcccagatcgccgcgaagatccccggcaagatcgccgacgatatcggcgcctgggtcaaggaacacatggcctccatattcggcggcggcggttccggatcggaagcgttcgacggctggtggaacgcggctgtcgccatcaatcctgatatggcccccttcaagcagatcgccgccacggtcgcccagaacgaatccggattcaacccgaacgtcatgaacaactgggattcgaacgctgcggcgggcacgccgtcgggtgggctgatgcagttcatccagcccaccttcgaggcctacaagtggcccggattcgacaattggatgggtgcggtcgatcagatcctcgcctggtggaagtacgtgaatgcccgctatggcgggccgttcaatattcccggaattgcctcgctggcgggtggcggcggatatgtcggctacgccggaggcaccctgaacgcggctgccggcacggcatgggtgggggagaacggccccgagctggtcgatttcggtggcggcgagtcggtctacaaccgctcccagattgacggtctggaggatcggatcgctgaccggacgatttcccggctgcagcagctgagggtggcgctgatcgtggacggacatcagatgggtcaggtcatcgacggccgcatctccatggctggcgctgctgcacacggatcgaggtggtgacatggcgatcattgcgacgcgccgcgactggcctgaggctccgcaacgcttccagtccgccgatgggcggctggtggcggagctggaccctgaccggtgcggagtgcgactgcgcggcaccgacctggaggcgtggagcgtcaccctcacccgtgatggcgaggtgatccacaccggcgaccccatggtcacaccgggaggaacaggaatcgcctacgacctgtctgcaccgttggatgctgatgtcgtctacgaggcgcacgcgggtggggcggtgctcacgcaggtggccgtccacaccggcggcttgcctttcgagtgggggatggtgaccccgctggccgaccccgacaagggcctgatgctacggaccgtcgccgacacccccacgctgggcaggtcggcacgccagaagctgtctgcggtgccctcatcgaggctgcaggcaggtggctgggacgtccccaccgacgcggcacagggatggacgtggctcgcgggattccccgacgcctccaaagcgctcgccgagcgcgacgcgatcatggaggccctatcgctggggccggtctacttccggcccgaaacctcgatcggcttcccgcccatgtgggcactgcccggcgacgtgtcagcgaccaagcagggcgacgcctggacggtgtcgtgcacgctgacgccgatcaccgctcccgcgaccgccgacctgcccgcctgggcgcccggcaacagctatgcgcgtgtggcggccacccgggggagcctcgccgagctcgcccgcacatccaagacattcctcgagctagtggggttctgatgattgaagtatccaagcgatgggcctcctcagtaggggccggtgcacgctggtcggtgatggtctcctggtcctccgacggaggccagacctggcatgacgtggtgcccaccgcctgctcggtggacgagtctaccggccagcaggtgcggtggaagctgtcctgcaccctgcgcaaggccgacgccgagggcctgaccgtcttcggttgcagggcgcgcgtcttcgtgtcgatgcatcacaccgacagctgggaggagacgatccagctcggcgaattccgcattgacaccacctctgacaccaccctcgccgggccgtccggtgcgcaggtcgcggcagttcaggtgagcggttcgagctgggagcagcagctgatggactcgcggctggttgagccgcgtgaggtgtcgggtgccgcgatcgatgtgctcggcggcctgatccgggaggtgctccctgacgcagagatcgtcttcgacggcgggatcgatccgggccgcaacattccggcgacggtggtggagcgtgaccggtgggccttcattgacggctcgaattcgtcggagacgtcggtggcgcggatgctcggcgcccaggtctcgaccgacgcacggggcgtgtggcatgtggccccgcctccggtgctggacgggacggcggcgtggacgatcgaggccggcaagggcggtgcgctcctgtcggcggtggccagcgaggaccgctccacgatccgtaacgccgtcatcgcgcgcggcgagtcaaccgataagagcgtgccggtgttgggtccggtgaccgtggctgatcacaatgcgtggtcaccaaccaacgtggacactccggtctccaggggcggcttcggcacagtcccgatcttctacacttcgagccttttcaccgacacgacgcaggtggaggcggcagcgaaggcgatgctgcagccgcgcctgggcgtcaaacgcaccctggacctgacaacgctcttcgaccctgccaaacgcgccggggatgtgggtgtggtgcagaccactgatggtccggtcaccgtcgtgctcgaatcagtgtcgtgcgacctggtggcggcgtcgatgacctgccagacgcgcggcacgaccggcaccgagctgatcacgaccgaaaccacgacaaccactggggagaagatctcatgagtgcaccagacattgccctgcaaggactgatcggggaagacaccgagcaggtggcgcttgcccaggtgctcggcgtgggcgtcgacgggcggtcggtgcgtgtccagcgcggcactctcacccacgaggtccgccggctcgatagctacaagccttcagcgggagaccgggcgctgctgttacggctatctggcggcgaatgggtgctgatcggcgccctcgcctgaccttgacgacctaacctctgacaacctgaaaaggagccctccatggcaaccgtctatggccctgacaaattcaccgtcccgactggtccggacgcaccggacgtgccggcgacgatcatcacgctgctggactcgatgcgtccctcgctgatcgggcatgcgtcttcgatcgctgaccgcaccgcgaaatatgggcgggcatccgcgtcgagcattcaggcgccgaagggcacagtggtggtgtctgccgagctgaacgcaatttgggtgaaaacatcggacacgctggatgagtgggcgacgatcattcagcactcggatgaggtggcgaccgtgtcggtggtgtccacccagtccgaccaggtgaccacggtccagaagttcacgattcccgagtcgggcatctatgcgctgtatgcatcgatgaatgaccagaacggcttggatgtcgatgggtcgatccgtgagatacatgttctggtgaacgggacctggaagttcggtgggatcttcccggcgagcaagttctggctctggtcgggttcgcggacgacctttctcaataagggcgacacctatcagatcgactttatgcaacgctcaggcggggagaggtccctgaaggtaacgctgtcttatcaaaggattttgtaatggcgacgtgggattacgggtatgcgccggctgatgtggtgaccgatgcggccggggatgtgctggccggcatcgaactgcgggtgtgggacgccgaggtggcagggaaagccgtcgccgtccagcaggaccgtggcgacggatggaaacccgcgtcaagagtcctcaccgacgacgtgggccgctaccgatttcgtgccgaagcgggccccacggtgtgggtggaggacgtgtcagggcggcgctggcggatggatgcctggcagacgctcggcacgatgatcgactccgcacagagcgccaccgccgcggccgagtcggccaactcaatcgcccacgaagccatgtcagtcgcccaacaagcccagacgtcggcgaaggccgccgccgactccgccgccgccgtgcagggggttgccccgtccgacgcgaatgtgtcgccgatcatcaccggcggggcgaagactgctgaggcggtgcggaaggcggcgctggctgctttcccgacgaccgggccgacgatcttcacgcacttcttgacgcgcgacgaggccctgcatgtggcgatctccaccgacggtgtgacggtggaggacaccggcctgcggtggaagccgaagaacgacaccaccctgggggagtgcttcgtgcgcgacccatcggtgtgtttctggaagggtgcctattgggtcgccttcacccggcccacgaagggcgggggtgacgctttcgggacgaccaagtcgttcggactgatgaagaccacggactggcggaccttccaggagctcccgccggtcgtgatgccgagtcaatttcagcagacgtgggcgccgcagtggttcatcggctccgacggggtgccgcatatctttgtggccctcggcaccaccaccacgcccaacgcgtacttcacccagtatgagctgcggccgctcgatgacgcgatgacgtcctggtcggacccggtggtcatgtctggactgccagcgaattgcatcgatgtcgcggtgatcgaggacgccggtaccttccacgcctttccgtccaaccagaagacgtcaacggtcgagcagtggacgtcaaccgggctcaccggcccctacacgaagctggcggccagcgactttcccggtgccggtgtcgaaggaccccagccagtgccgctgaagacgggcggctggcggatctacgtcgacaattacgcggagaccgactcgatctatttcgccgagagcacggacttgctgcattggtcggcgctcaggccggtcaccctgccgatgcgtcacgtcggcgcggtcgcggtggactccttcggtgcgctacgcacccgcgagctgtggcagccgaacatcccgggcatgagggggatgggggcacccttctggggcgtacccttcgccgcggggaacgtgctgaaggaattcgcgcagatcgtgtccatgcgcaccgacggcggcggcgaaatcgatctggcaaaggcggccacgctgggcttcaccggcatcgattacatctcggcgacggctgtcgcgaacgtcgagattctgcagatcgagcccgacattcgcgctgtcgacagcatgatccacggcgtcgccctgcgaggaccgagtacgccgcagatcgatacagacgtgaaggtcgcctggcgggtgctcggctggggcgatccgagcacgccatgagcagggacgctgacgtgaccaagcagggatccttgcctcggcgggtctgggacatgctggcagagccgaagtcggtgacggtcctcatgacgattgcctacgcggcgctcgtcgcgctcggcttctgggcgatcgacgacgcctccacgatgggggtccgcgacatgatgggcggcctgctcatcgctggtggcgtgtgcgggctgatcggatgcccgtggggccagtggtggatcgagcgcgccggtctggtggcgatcggtgccgctttcgcggtacacctgtctttcgtcgtggcgatctccccgcccgacggaccgtgggaagtggcctcggcgctggggctgctgcttctcgtggcgacacgctggatcaggatcaggacgctgccagccgacccgacgctgcctcggcccgggcctccagaggcgggggatgaatgaatgacttccagacctggatcacagtgctgggcggcgccggattccttggcgcgctcgtcacgctcatcaaggggctggttgggtggcgcaccggcaagtccggccgcaaaatgagggccgcccacgacgccatcgactcgctgaatctggcgggcttgtgggctgaagcctactggcacgctcgcggctattgccgcagccaccatgaatggaccagcgattacgccgacggctatccacccccacccgacgacaccaacacccctgactgagccccgccttgtgcggggctttctcattcctcaaagacttggagacattcatggactggaccaatctgaacgctgacgtgacgaagctgatgggcgtgcacttcacccccggacgtgaaggcaggacgatcgacaagatcgtgatccaccacaacggcggcaacctgagcatcgaccagatctggaatgtgtggcagacccgtgaagcctccgcgcattatcaggtggaggcgggtggccgtatcggccagctcgtcaacgatttggacaccgcgtggcatgccggcgactgggacgccaacctgacctcgatcggcatcgagcatgccgacgactcgaccgacccgtggcatgtgtctgatgctgccgtcgatgccggcgcgcacctggtggctgcactgtgtcgcggctacaaccttggccggccggagtggatgcgcaacgtcttcccgcactctcagttcacgtccacgtcgtgcccggcgtcgctggcccgggaccagctcggcgactacatggggcgcgcacaagcctacttcgatggcgcgccggtggctgcggtccatcagtcggtccctgcccccgccccagcgcccagccgtcatgtggacctgcccgcgtggaatctccccgagggcaacttctacggcctcgtcagcggcggaaacgactcccacggcggcttctatcccgccgagcgtcccgctgtgagggccatccagctgtggctcatccgtcacggctacgccggcgcggtgcctgacagttgggcggacggcatctacgagcagccgaccgccgacgccgtgaccgctttccagcacgccgagcgccccaacagcacggaccggtggggcgaggtctgggccgacgacctggccaccatggccgccaacaactgacaaggagctgatgccaagtgatctggactctcgcattctggaagggcgcaggcgagcgcgccatcaaaaccgccgcgcagaccgccgtcggcctcatgggtacctcgacgctcatcgaacaggtgccgtggactgtcgtcgcctccggcaccgccatggctgtggtgctgtcgctgatcacctcgatcggcaacgccgacttcaccgccggcgtccccactaccgccaaggggctcgaggcgacgaccgtgggcaagacggacaccacgcccgtcacgccaccggcgcgcgtcgccgaagaggtcccagccggcttcgtcccggacacggccccggatcccgtgccgaccgtctgacctgagggggtgacggcgaccctgcgccggatagccactcaagcaacctgagcgacacaagaccgcccactctgaccttcgcgggtcggagtgggcggcctttttgcgtctcaggggcgcagatgatgactcgtcgtctttaattctagcagtacgcgttcagcgtcgccagaccatgactttctcggctgcctggagcggcgcaccttcggggcctttgaggtagggggcgatgtagatgagcttgcgcagtccatgcttggggccgtgcgcctggtgggtccagtgcccgcggaccatgaaccgcacggtgagcttgtgcccggttccgtcgtcgcggtcggtgaccacggtgcgcacgggacgcagatcgaccagggtgacgtgacggtcggggcgtggcgtgcggggcctgtgctcggtgcccggggccttgccggtgcgggagtcgatcgtgcgccgctcggcgacggtgggggtgtccatgagcacgctcatcgccatcagcagcgacgcggacatgcgggcctcggggctcagcgtggcatccagatcggattcggggcggatgagaatcgacaggatctcgacaaggggaccgtccacgtcagcgaagccgggcggatagtcgcccagccgccccagcagctggatcatggtgcccccaccgggagcgggaagccaggcgatggcccacacgggaggattcccctgccaggtgcggccgccgggcaggtcgaaggtgcgcggcttgggccccggcaggggtttggcgaagcaggccagtccggtcggggtgatcagctggctcgggctccactcgggcacgtccagggcagcatccagagcgagcgccgccatctcgccactcacccagaacagcgacgcattaccgagccgctcggcactccacccgaagccggacatcggcagtgccttgtcgcccatggcttcagccaccgcatcagggtgggtggcggccagttgctccaggcgctcgtcgaggtggcgggagtcccgcacaaagcggcgacgcagcccgggcactccgcggggtgtccacgtccagctctccggggctgccatgtcaggcgacgactcgaatgcccaggctccgccacgcctcggcggtgtggtcgagccccagatcccaccactgggtcagcgcgtgttccatggcgatcgtggcgcaggccgacagctgcgaggccttgtcgcgagccgacagcaggtcggcgctgtcggtgccgtcgggcgtgaagtcctgcagggcgatagtgatgtctccgccgggaagctgggagaccgtgtcggcggtcatgcctgcctgggtggcgtcggcgatgatgcccagctcgaccagcgtccagccggtgcgggccaggtcgatgcgctgggcctcgcgccacaggtccatctcggtgcgggtgcgtgccgacaggctcgggggtgcggccacatcggctccgcgtcgggccatccatgcggccatgtcgtcggtgggccgccaggtgattgtgctggccatgaagatcctcctcggagtggaaagtggaaaggggaggggccggagcccctcccctgatggttgatgtggtcagcggatccaggtgaagggctggtcgccgatgatcgagcggaccgcgaggtcgtagggggcctcgtcctcgctggactcgacatcgaccgcgtcgagctcgacaccgtcgcgggtgatggtgatggtgtcggtcgtggtggcccgaccgtcgatcaccgtcggagtgttgatgctggtgaactctgcggtgatgccgaccagctcgtggtcggcgaccgcttcccagaaggcgtcctcgtcggcttcgatggagaagtgcacgctggaggcgatcgtcgcaccctcggaggtctcctcgctgtggagcgtgaccagctcgtcggcgatggcgtcgaggtcatactcggcgcgggcgtcggcgactgcacctccggcctcgatggcatcgatgatagaggcgatggcctcgccgcgggtggagaaggtagtgtcggtagaatcggtcatgatcctgtccctttcagggtcttggcctcatcggggtgcttcccggtggggcctcttgctttgtccttgtgacacccactgtcgtcacagtgttggaacatgtcaagccagtgggccacctttctttaaagagatttcagcgggcgaccgcgccgcctcggacacctgggcccagtagccctggagggcgcactgaatcaattggttgtcaattggttgtcaaacctgacccgtcgacggggagtgaggaggtggtaccggctgatctacgcctgaaacagatggagcgggcgacgggaatcgaacccgcgtgtctagcttgggaaacgggcatcgtgctagtctggggaccgccgaaatgacgatttcaggcgtaaaccggcctccggtgtcttaccctgatagctgggtgatagcaccgaattggttgtcagattggttgtcagatcgccccaggaggatggtcgcattgtcacgcgcaagctacggggacggcacccagccgacccggcgttccgacgggcgctgggcagcatcggcctatgacggctggcaggcgaacgggaaccgccggcgccgatgggtgtacggccgcacccaggccgaatgcaagcggaagctgcgcgacctgaagcgggagatctggtcagacacccagcagatgaatgtgaaccccagggagaccgtcaagagctggacggcatcatggctggacgactaccgatcgattgccagaccaacaaccttcgccaccgacgagtccatggtgcgcaactggatcgtcccagccatcggtgcccggcgcctgtccgaactgacagcgcgcgacgcctcgaagctgcaacgggtctgccgagacgggggactgtcggcgacaacgtctcactatgccgggctgctcctgcggcgcatcctgaaggctgcccgcgcgaacggctaccgcatccccgactccgtcatgctggcccggatcccgggcatcggcgcatccaacaggtccgccctgagcgccatccaggcggccaacctgctctcgacggcaaacgcacgcgacacctggccggagccgcccagccttcccgacctgccctacggggccatctcgaagctcgcaccagcagaagcgcagaagcgtgaacaactcaagatggagcggttggaatggactgccgcccaaaacacggacccctccaggtgggctgccgcactcatgcagggacttcggtcaggagaggctcgaggcctcacgtgggatcgtgtcgatctcgataaggggacgatcaccattgatcgtcaactccagcgcatcaagcccgacgcggcgcttccaccgggatacaaggtcacccggctggaaggcagccactgcctcgtggcaccgaaatctcgatcagggatccgccgcgtcccgatcgtcccctggatgggccaggctctcacccgctggcgcgacatacagggcgacagccccttcgggctcgtgtggccgctgcccaccggggcgccgcccacgcgggtccatgacctgcgggcatggcgtggactccagcgcgtcgccggggtccacaaggaggatggaaacctctacgtcctccacgaagcacgacactccaccgtgtcgctgctgcttgctgccggggtcccggaatcagtggtcatcgcgatcgtcgggcatgcaagcttcgcggcgaccgagcactacgcccacaccgacctcgaagcagcacgcgccgccctcatgaaggtgcaggaccgcctcgggctggagctcgagagctgagcatgcaaagagccgcccaccggaccaatcgcggtctggtgggcggctcttttgcgccttagagcacgtccgtcaccacgcctggaagttgctgacgacgggtgcctggtcggtgccggtcacgtcgcagtgaaccgtgtatttggcggagccgacgtcggcgccgatgttgacgttccacagatcgtcggtcttattgagggcggcgaccgaatcgacggttgagtggaccttgatcttaagcgatgggtattgcttgcccaaggcatccctcgcataggtgccacagccagaggttgcgccggtcatggtgagtccagtggtcgttgcctcgacgggtgtgggcgtggcggacgcagtgggcgtcttcgtggccgtcggcgtcttcgccgctttcggagtcttggaggctgatgatgacgatcctgatgtctggggatcgcacgcggtcagcgcgccggcgaggcagagtgacgcaagcagggcgatggggacgagcgccttgcggcgcatggtgtgggtcattcgggttccttggttggttggttcacatgctgtccacagcaacctagccgcggaacctgcccgcctgggggtgatgagggcaagtggcaagaattacatcgatgggattctcgccaccccctgaagagcgtgggtggcgaggtctacattcgtacgcatgtacgaaacatggaagcctctcggacacggctcgatctctggtggatcggcccgcacaatggagtgcactgaagtagcggagtgggcagagcgtcgtgcgcgtgggtggggctcagcgctggtatcgcgcctgcgcggcgtccatgaagacgccgggttcaaagtcgagcacgcgagcaagctcaaagaggagtgcgacagggagatcccgcttgccctgctcgatcctgatgatggtggattcgctgactccagcgagtcgagcggtctcgacctgggttaagcccttggcggctcgctcggctcgaagctgggcggcgatcgcggcgcgaattgcatcacgcttgctggcctggttctggtccatgctgtcagcatagccgccacattggacagttttccggtccgattgggatgctcggcacttgcatctggccatatggcatggcaagctgtccatatggccagttcagacatcaacctggaggctgcggacatgatctccgccgccatcgagcgaagcgacaccagtcgggctgaagtcgccacgctgacgggaatcccgttgaccactctgcgtcggaagctcatgggccgatcgcccgtcaacatcgaggacatcttcctgatcgccggcgcgctcgggataccgcctgtgagtatcacgcccgacgttctcacgagtgaagccgccgcctagcccccaaacagaagaagcccccgcctgctgtcacagacgggagccaaccaaaggagtttccaatgagcattctacccttcgactaccacggtcaggaagtccggttcatcaccgatgagtccggcgagcctcaggtcgtcgcgtcagatctcgcgaaggccctcaactatcggaacgcacccgacatgatgcgttccatcgacctagaggaaaggggtacgcgtccggtgcgtacccctggcggtgagcaggagatgctcacgctgaccgaggccggcatgtaccaagccatcctgcaacgccagacaggccggatggtcgacgtcgcccaacgagccgctgtgaagcgattccagcattgggttacccacgaggtgattccctcgatccgcaagcgcggcatgtatgccactccggatgcagtcgaggcgatgctggccgatccggacgttatgatccggacgctcaccgagctgaaggcccagcgggccagggtggcccagctgcagcccaaggccgactacgttgacgccttcgtggccgacgaggatctgcggctcctgcgcaatgtggccaagtcgatcggagtgcaggagggcgccattcgcgacgccttgctcgcacacgagtggatctacgcggaggagtcctcgcgctggtcgaactctcagggctgcaaggtcatcgagcaccgctattcaccgcgctctgacaaggcccgatactttcgcccggtcccgaatcaccaggcaccccgatttaagggcgaggtaatgcacaccctgaaggtcactccggcaggggctgaggcgatctccaagatggcaaagcgctggggcctcgtcgtccaggaggtggcggcatgacctcgactctcaccggcaacatcatcgccctgctgatcgtggccggcgtgatcgtcctcgcgatgggggtgcgccgtgaaggtcgatgacttcgacgatgtgcgccccctgacgcagaaggacgtcgccgagctactccacgcaagcgtcggttacgtgcgctcctgccgcctggcgacgaagccgaaaggccgggtcttcccgatgcccggctggaagaccgacggaaagcgctatctgcttcccgcttggcggttccgcgagtgggtcgaaagcttgcccgatgcctagcccgcgccgcttcctaatcctgatcgccctgggtgccgccgccgtcggtttcgcgccctcctcaattcaatttctcttcatggccgcgcttgtgctcggcctcaccatcacatgcctcaaggagtccaaccatgcctgacacacagccccgtcgtgcgcgtcgtcgcacgctgtccgagatcctcgcccccgcgccggcgccccgcagagcggaggcaacggcatgaggccaccagccgttgaaacccctgatgtgaaggcgccggccacgcctgctggttcccggctcttcaaggctgtccgtcctgacggcttcgacttccacagcgggactgtccggtggctccctgctgatggcgcaccgatcccggagggcgggtggcttgtcgagcatccgcatcctggtgaggttggcagctgggatgcagctttttatctgtcggcgtcgtcggtggagacggactgcacaggtttccagtggcctgctctcctcctgtccgtggagcccgtaggtgccatgtggacccctcgccccgacaaatttcctcgcaagcgggccgcgcacgcgtggcgcgtcatagaagagctccccgcatggcggcttttcggtccccaggggcggacggtcctggacatcatcgagcaaaccgctcatctgaccaaacgccagatcgcggccctgaacagggctctggacgccgcacgggacaccgtttgggacgttgcttggaacgccgcgtggcacgccgctcgggtcgctgctcgggtcgctgctcggggcgctgctcggggcgctgctcggtacgccgcttgggacgctgctcggggcgctgcttggtacgccacttgggtcgctgctcggggcgctgctctcggatggctcgtcaaggacctgatctccgtcgaggacttccgcaccctgacgggcccgtgggagcaggtcatgggtccgatcgaggtggcggcatgaaccgcacctatttcaaggccgttagggcggacggcactgacttctacaccggcaaggtccgctggctgcccgatgatggcgcaccgatccctgccgggggttgggtcgttgagcatccgacgagcgaacgcgtgggggacgacgcccgcacctatctctcggtttcgacggtggaaaccgactgcgccgggatgggctggccgtgccgtctcctgcgggtcgtccccgacggcagacaggtgagcatccctgaacccgtggggctgcccagcacgagggcctcgatcaggtggcgcgtcatcgaagagctccccgcatggcaggcgcttggaccccaggggcgcgagattgaggcgctgctcggacaggttgagagtctcacggaggaccagaccctcgaaatgtctgccgctcggggcttcgctcggggcttcgctcgggacgtcgcgcggttcgccgctctggtcgcctctcggggcggtgctctgaacgctgcccagggcggtgctttgggcactgctctgaacgctgttcgggacgctgttctcggatggctcgtcaaagattttatctctgatgaggaattccgcaccctcgtgggcccgtgggagcaggtcatgggtcgggtgatcgcatgatgccgatcaccaagccgtgcgcggttaaggacatgccggagggcgagtatcactcggatccctgcgtcgagccgtccctgtcgtccacgatggcgaaaaccattgtttcgggtgaggctggcccggcccgtctgcgagagatcatgtctcacgggcaggaacataaggccgtcttcgatttcggcagcgccgcgcacgagaaggtgctgggacgcggcgccggtgtcgaggtgctggatttccctgcctggaccacgaaggcttcgcgtgaggcgcgtcaggccgtgtgggatgccggcggaactcccgtgctggcgaaggattccgcccaggtggatgcgatggctgaggcgatcctgtccaatcctgtggcaggtgagctgttcacgcgcggggctggttctcctgaattgtcgatgttcaccattgacgaggagacgggacgctggcagcggggacggctcgacttcctggcggaccgcaagaccatcgtcgacttcaagacatctggacagtccgtcgagctgcccgactggatcaagcacagctggcagttcggctaccacatccaagccgccgcctatatggaccaggcgatctcgctggatctggtcgatgaggacgccatcttcctgcatgtcgtgcaggagacgaagccgcccttcttgctcgcgatctatcaggtttcagctgaccagctggccgagggcaggcgtcagatgcgtcgtgccctggacctgtgggaccgctgcctgaccctcgacgaatggcccgcgatccctgcggtgatccaactatccaagctgcccgattgggtgcacaccactgatgacgaaaaggactcctgacatgaccgaaaccacacctagcaccgacattgaaaccaccgcccccaccccgtcggggtcgatcgcggcggtcggctccgagacggcaggcctgacgcttcagcagaagctcgactatgcctctgccctggccgactccgagctcctgcccgccgcctacaagggcaagcccgcgaatgtgttggtggcgatggagtacggcggcgagctgggcatcggcacgctcgtcgcggtgaaccagatcacggtgatcaacggcggcgtctccatggaggcgaagctcatgatgacgctcgcccgccgagccgggcacatcgtgcgcctgtccggcgacgacaagcaggccacctgcatcatcatccgcgccgacgatcccgggcacgaatcggtcgtcacttgggacgaggccaaggcgaagaccgccggactgtggggcaagggccactggcagaagaacccgggcttgatgttgaagtaccgggcggcctcggagaacatccggctcacctgccctgaggtgctggcggggattgtctacacacccgaagagctcgatgagcgcaccgagcgtgcaggccggtccacgatgcgtgtccatcaggtcgtggccgagccggagaagaccgctgcctacttcatgaaggccctccacctgaacggcggccagttcaaggagtttgcccagcgcgtgctgggacatccgttgaagagctgggaatcgctggccaaggcagacaagcagcgtgtcctgggcgctctcgccagctgggagaacagcggggccgatcccaccactggcgaggtcctcgacgccgagccggtcgagggcggtgcggcatgagcaccttgcctgcggatgctgccgagaggtggcagcagtgggatggcctggcccgcacgatcctcgcccttcatctcggcctgactgatcttgagatggtcgagctggtgggcgggctcatcggtgccggctggcatcaggatgggccggtggagtcatgagctggcccgaggagcaccacgacgtgtgggcgggtgtcgaggacgccatccctgagtgggtgagcgacaaggtggcctgctcggtgcggtcggatgccgattggaatgccgacgaggacagccgcaaggccgtggcggcggtgaggatctgcgagcggtgcgccttaaccgagcagtgcctggattgggcgctggcccaccacgaggccggcatctggggtgggctcaccgcctccgaccgcgagcgcatcgagcgtggcgcgccggtgcggcgggtccgcgagattcgtcggcgtcgcacggcggttaggcaggtgcaggagtcatgagcgcaccactgaccaaggcccagaaggtcgcggcggtcgtcgagcagctgttgcgtggcggcgccgacaccagcacgctcctggaggcgacgggggccgaccggcccggacgattgcgggacacccttcgccgcgctggccgtgacgacctcgccgcccggatcatcaccaccgaccgggcagcccagcgcagacgggaagtcatcgaggcggtcgagaagctggtctgggtggacagggccgacgagatcgccgccgaactcggctacagctcgcgctacggcctgcaacagtccttgcgcggctgggggcgtcgggaccttgccgatcagatcgtgctgacccgcgagacgcaccgcgacagggtcatcgctgacgtggaatggatcgccggtacacggggccccgaggatgtcgcccgggcgaccggataccgcaacgcggcggcgctgcaggccgccctgaccgggtggggccgcaaggacctcgccgaccggatcgtcggagcatcacgcaacgacacgggccgcttccgcttcacatggagggccgcatgagcgccaaccgctcccgccgcgccacgtacaaccacacggggatcttcgtccatctgcgcgaagccgccgagccgtccgaacagccaccctccgaccagacatgcccagccctgcatgtcatcgccggactgacaccctgggccgaccaccagccccgccacgccctcggcgtcgacgggcgatgccggcactgccacaccaccatcaaaggaaacccatgatcttcaaagacaccacgatcgggccgctcgaaacacggttcacctggtcgatgaggtgcgaccgctgcgggacgccgctcgactggctcgtcgccgcttcgtgcaagaccgagcgtagtgaggtaatcgccgtcaagttcctgagggagcgtgcccgcgatggcgggggcctcagagagtggggggagctggacctttgcccttcatgcttctcggtgatggacgcatgattaccaccacacaactcggagaagcagaccggtggggccgtggcctccaagtccgctcgatcctgtgcaacggctgcggcatagctctggcgaccgacatcggccttcgtggagacgccaccgccctccaagtgcaatccgacctgcacgcccgagcacgcaccgccggctggacacaccccgcctggcgcgtcgacctctgcccgcaatgcaccaccacaaccaaaggagcatgaccatgaaggccacccagtacgccaaatcgaccgaccctgaagtcatcgccaccatcgaagagaacgagctgtcacgacgggcatggatcgacgacaccaaggcgtggttcggcaagacgatccggacaggaatcccgggcgccaaattgttcctcttttccacccggaccgctatcaggctgttggggatcgtgacgtcggacgagaagaagcctgccgggtggaagttctgctggcgttcacgctctcggttcgagccacgaaagaacaatcccttgcgcgccacatgggacgcacgccggtggcaagcagcgtcgatcccaggtctgcccgtggttctcacgtcctccgtgtcgggagagttacagagctggttgaggatgtatccctgccccttcatctctagtggtgccgcatggctggacctggagcacatgcctgaccctgacagtccgcacttcggaccgcagtggactgaagtccgtgcatcgcaggcaatggcagccaaggaagcattgaaggacgcgtcatgagcactccgggatcactgcgcgccgcgctcgaccagctggacgagatcggcatcgccgaccatgtgcagtccttggaatgggatcgggccggcgcccgcaccacagcctggctcgagacctgcggcgacttcgctgcggcctgccagtggggcgatgccgcgggcgaatgggtcacgtgggacatcaccgacgtggccgaggcggacgtcagcccccggctgcgcgtcaagcacatgcacctgcgagccaggccctgtgctgatgcgcccgcgaaggcggtggcggcatgagcaaggcccttgacccactggatcaccttc

SEQ ID NO: 27 - DNA, 29768 bp, PAC7 phage
  source 1..29768: mol_type = genomic DNA
tcgtacggcttagtgaaatacctcccttttgttgttttatcgttttgtcgactttttgtttggtggtgtgtgtggtgcagcctgagcttcctgatagtcgtgattggtgtggggagacgcgtcggtggtggtgtgtgtggggcgaggatccgcgtgccgggtttgtgtctgatgaggagtggttgtttctcatggatgctgcggtgattcatgatgtggtgtggcgtgagggtcgcgcggatttggtggcttcgttgcgtgctcatgtgaaggcttttatgggtatgttggataggtattcggttgatgtggcgtctggtggccgtggtgggggttctgcggtagcgatgattgaccggtataggaagcgtaggggggcttgagtaggtgtctggtgttgttgggtctcaggttcctcgtcaccgggtggctgtggcgtattcggtgtctgctggcggggatgctggggagcttggtagggcttatgggttgacgcctgatccgtggcagcagcaggtgttggatgattggcttgctgtgggtggtaatggcaggcttgcttcgggtgtgtgtggggtgtttgttccgcggcagaatggcaagaatgctattttggagattgtggagttgtttaaggcgactattcagggtcgccgtattttgcatacggctcacgagttgaagtcggctcgtaaggcgtttatgcggttgcggtcgttttttgagaatgagcggcagtttcctgacttgtatcgtatggtgaagtcgattcgtgcgacgaatggccaggaggctattgtgttgcatcatccggattgtgccacgtttgagaagaagtgtggttgtccgggttggggttcggttgagtttgtggctcgtagccggggttctgctcgcgggtttacggttgatgatttggtgtgtgatgaggctcaggagttgtcggatgagcagttggaggctttgcttcctaccgtgagcgctgccccgtctggtgatcctcagcagatttttttgggtacgccgccggggccgttggctgacgggtctgtggtgttgcgtcttcgcgggcaggctttgtcgggtggtaaacggtttgcgtggacggagttttcgattcctgacgagtctgatccggatgatgtgtcgcggcagtggcggaagttggcgggtgacactaatccggcgttggggcgccgcctgaatttcgggacagtctcggatgagcatgagtcgatgtctgctgccgggtttgctcgggagcggcttggctggtgggatcgtggccagtctgcttcgtctgtgattccggcggataagtgggttcagtcggctgtggttgaggcggctctggttggcgggaaggtttttggtgtctcgttttctcgctcgggggatcgtgtcgcgttggctggtgctggtaaaacggattctggtgtgcatgttgaggttattgatggcctgtctgggacgattgttgatggtgtgggccagctggctgattggttggcgttgcgttggggtgacactgaaaaggttatggttgcagggtctggtgcggtgttgttgcagaaggctttgacggatcgtggtgttccgggtcgtggcgtgattgtggctgatactggggtgtatgtggaggcgtgtcaagccttcctggagggtgtcaggtctgggagcgtgtctcatcctcgtgccgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaagcgaagcggcgtgaacggtctggtaggaagcgggtgtctgtggtatgaactcggatgagttggctctgattgagggcatgtacgatcgtattcaagggttgtcttcgtggcattgccgtattgagggctactatgagggctctaatcgggtgcgtgatttgggggttgctattccttcggagttgcagcgggtgcagacggtggtgtcatggcctgggattgcggtggatgctttggaggagcgtctggattggcttggctggactaatggtgacggctacggtttggatggtgtgtatgctgcgaatcggcttgctacggcgtcgtgtgatgttcaccttgatgcactgatttttgggttgtcgtttgtggcgatcattccccaagaggatgggtcggtgttggttcgtcctcagtcgccgaagaattgtactggccggttttctgccgatgggtcttgtttggatgctggccttgtggtgcagcagacgtgtgatcctgaggttgttgaggcggagttgttgcttcctgatgtgattgttcaggtggagcggcggggttcgcgtgagtgggttgagacgggccgtatcgagaatgtgttgggtgcggttccgttggtgcctgttgtgaatcgtcgccgtacttctaggattgatggccgttcggagattacgaggtctattagggcttacacggatgaggctgttcgcacactgttggggcagtctgtgaatcgtgatttttatgcgtatcctcagcgttgggtgactggcgtgagcgcggatgagttttcgcagccgggttgggttctgtcgatggcttctgtgtgggctgtggataaggatgatgatggtgacactccgaatgtggggtcgtttcctgtgaattctcctacaccgtattctgatcagatgcgtttgttggcgcagttgactgcgggtgaggcggctgttccggaacgctatttcgggtttatcacttctaacccgccttctggggaggctttggctgcggaggagtctcggcttgtgaagcgtgctgaacgcaggcagacgtcgtttggtcagggctggctgtcggttggtttcctggctgcccgggcgttggattcgagtgttgatgaggccgcgttttttggtgatgttggtttgcgttggcgtgatgcgtcgacgccgactcgggcggctacggctgatgctgtgacgaagcttgtgggtgctggtattttgcctgctgattctcggacggtgttggagatgttgggtttggatgatgtgcaggttgaggctgtgatgcgtcatcgtgccgagtcttcggatccgttggcggcactggctggggctatttcccgtcaaactaacgaggtttgataggcgatggcttcgggtgctgtgtcgaggcttgctgcgactgagtatcagcgtgaggctgtcaggtttgctgggaagtatgcgggctattatgccgagttgggtcgtttgtggcgtgccggcaggatgagtgacacgcagtatgtgcgtttgtgtgtggagttggagcgtgccggccatgacggttcagcagctatggcgggcaaattcgtttcagattttcgccggttgaatggtgtcgatcctggtttgatcgtgtatgacgagtttgatgctgcggcggctttggctaggtcgttttcgactatgaagattatgaatagtgacccggatagggcgaatgatacgattgatgcgatggctgcgggtgttaatcgggctgttatgaatgctggtcgtgacacggttgagtggtcggcgggtgcgcagggtaggtcgtggcgtcgggtgactgatggtgatccgtgtgctttttgtgccatgttggctacgaggtcggattatacgactaaagagcgggcgcttactactggtcatacgcggcgtcataagcgtgccggtaggcgtccgtttggttcgaagtatcatgatcattgtggttgtacggtggttgaggttgttggtccttgggaaccgaatagggctgatgccgagtatcagaggacgtatgagaaggctcgtgagtgggttgatgatcatgggttgcagcagtcgtctggcaatattttgaaggctatgcgtactgttggtggcatgagataatttgatgtggtttccggttgtgtgccgccggttatcggtgcacagggttgtctcccgcacgggggtcaacaatgttgtgttgttttccgcaaggagtgtagggttaggctatggccgatcagagtattgaggaacagaatgttgacaatgatgttgtggagtccggaaaggataacggcattgttgatacagtaaaagacgatggcgggcaggaggtagccgacaatcagttgaagaatgaaggcgagggtaaatcgccggggactgattggaaggcggaggcccgtaagtgggagtctcgtgctaaaagtaatttcgccgagttggagaagcttcgtacatcgagtgacgattctggatctactattgatgagcttcgccgcaagaatgaggaactcgaagaccggattaacgggtttgttcttgagggtgtgaagcgcgaggtggctgccgagtgtggcctgtcgggtgatgcgatcgcttttcttcacggtagcgataaggagtcgcttgccgagtctgctaaggctttgaagggtttgatcgaccatagtagtggtggtggcgcgggtgtgcgccgtcttgcggggagtgcccccgttgatgatgttaaacgacgtgagggtgtcgcgtttgtggatgctcttgtcaataattctaggagatgatttatcatggctgacgattttctttctgcagggaagcttgagcttcctggttctatgattggtgcggttcgtgaccgtgctatcgattctggtgttcttgctaaactgtcaccggagcagccgactattttcgggcctgttaagggcgccgtttttagtggtgttccgcgcgctaagattgttggcgagggcgatgttaagccttccgctagcgttgatgtttctgcgtttactgcgcagcctatcaaggttgtgactcagcagcgtgtctcggacgagtttatgtgggctgacgccgattaccgtctgggtgtgcttcaggatctgatttccccggccctgggtgcttctattggtcgcgccgttgatcttattgctttccatggtattgatcctgctacgggtaagcctgctgcggctgtcaaggtgtcgctggataagacgaataagacggttgatgccaccgattccgctacggctgatcttgttaaggctgttggtctgattgctggtgctggtttgcaggttcctaacggtgttgctttggatccggcgttctcgtttgctctgtcaactgaggtgtatccgaagggttcgccgcttgccggtcagccaatgtatcctgccgccgggttcgccggcctggataattggcgcggcctaaatgttggttcttcttcgactgtttctggtgccccggagatgtcgcctgcttctggtgttaaggctattgttggtgatttctctcgtgtccattgggggttccagcgtaacttcccgattgagctgatcgagtatggtgacccggatcagacggggcgtgacttgaagggccataatgaggttatggttcgtgccgaggctgtgctgtatgttgcgattgagtcgcttgattcgtttgctgtcgtgaaggagaaggctgccccgaagcctaatccgccggccggtaactgattcatttgttgcgataatgtttatgctgtgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatatagggatgccgctaaggctattctgcgtagggctttgttgcgctggaatgatactggcgtgtcgggtcaggtgcagtatgagtctgcgggcccgtttgctcagactacacggtcgaatactcctacgaatttgttgtggccttctgagattgccgcgttgaagaagttgtgtgagggtgatagtggggctggtaaggcgttcactattacaccgaccatgaggagtagtgtgaatcattctgaggtgtgttccacggtgtggggtgagggttgctcgtgcgggtcgaatattaacggctatgctggcccgttgtgggagatatgatatgaccggttttccttacggtgaaacggttgtgatgcttcagccgactgttcgtgtcgatgatcttggtgacaaggtggaggattggtctaagcctgtcgagactgtgtaccataacgtggccatctatgcttccgtttcgcaggaggatgaggccgcggggcgtgactcggattatgagcattggacactgctgttcaagcagcctgtcaaggctgctggttatcggtgtcgttggcgtattcggggtgttgtgtgggaggctgacgggtctcctatggtgtggcatcatccgatgtctggctgggatgctggtacgcaggttaatgtgaagcgtaagaagggctgatgggttgtggcacgtgatgttgatgtgaagctgaacttgccgggtattcgtgaggtgttgaagtcttctggggtgcagggcatgttggctgagcgtggtgagcgtgtcaagcgtgcggcctcggcgaatgtgggcggtaacgcttacgatagggcccagtatcgtgccgggttgtcgtctgaggtgcaggttcaccgtgttgaggctgtggcgcgtattggcaccacctataagggtggtaaaaggattgaggctaagcatggcacgttggcgaggtcgattggggctgcgtcgtgatcgtttacggtgatcctcgaatatgggctaaacgtgtgttggcggatgatggttggctgtctgatgtaccgtgcacgggtactgtgccggatacatttgagggtgatctgatttggttggcgttggatggtggcccggagttgcatgttcgtgagcgtgtttttttgcgtgtgaatgtgttttcggatacgccggatcgtgctatgtctttggctcgccgggttgaggctgtgctggctgatggtgtggatggtgatccggtggtgttttgcaggcgttcgactgggcctgatttgctggtggatggtgcacgttttgatgtgtattcgctttttgagctgatatgtaggcctgcggagtctgaataagcttattgtttttgttttaatgtaattgtttgatatttaatgggggttgtgatggctgctacacgtaaagcgtctaatgttcgttcagcggttactggcgacgtttatattggtgacgcgcacgcgggtgattctattaagggtgtggaggcggttccttccgggcttacagctttggggtatctgtctgatgacgggtttaagattaagcctgagcgtaaaacggatgatttgaaggcttggcagaatgcggatgttgttcgcactgtggctacggagtcgtctatcgagatttctttccagctgattgagtcgaagaaggaggttatcgaactgttttggcagtcgaaggttactgccggatctgattcgggttcgttcgatatttctcctggtgccacaacaggtgttcacgccctgttgatggatattgttgatggcgatcaggttattcgctactatttccctgaggttgagctcattgatcgtgacgagattaagggcaagaatggcgaagtgtacgggtatggtgtgacgttgaaggcgtatcctgcccagattaataagactggtaatgcggtgtcgggtcgggggtggatgacggctttaaaagctgatactcctccgactcctccgccggccccggttcctccgaagcctcagccggatccgaatccgccgtccggtaactgatacacgattttaggggattgttaatagatgagtgacactggtttcacgttgaagattggtgatcgtagctgggtgttggcggatgcggaggagacggctcaggctgttcctgcccgcgttttccgtcgtgccgccaggattgcccagtcgggggagtctgcggatttcgcccaggttgaggtgatgttttctatgttggaggctgccgccccagctgacgcggtggaggccctggaggggcttcctatggttcgtgtggcggaggttttccgtgagtggatggaatacaagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccaccttaatggttggcagcgcccgtttgagtggtgcgagtgggctgtgttggacatgttggatcattacaggtctgctaatagtgaggggcagccggagcctgtggcgaggccgactgatgagcgtcgggcaaggtttacgtctgggcaggtggacgatattttggcgcgtgttcgtgccggtggcggggtgtctcgcgagattgatattatggggtgaatagtgtatgtctggtgagattgcttccgcatatgtgtcgttgtatacgaagatgcctggccttaaaagtgatgttggtaaacagttgtcgggtgttatgcctgctgaggggcagcgttcgggtagcctgtttgctaaaggcatgaagttggcgcttggtggtgcggcgatgatgggtgccatcaatgttgctaagaagggcctcaagtctatctatgatgtgactattggtggcggtattgctcgcgctatggctattgatgaggctcaggctaaactgactggtttgggtcacacgtcttctgatacgtcttcgattatgaattcggctattgaggctgtgactggtacgtcgtatgcgttgggggatgcggcgtctacggcggcggcgttgtctgcttcgggtgtgaagtctggcggtcagatgacggatgtgttgaagactgtcgcggatgtgtcttatatttcgggtaagtcgtttcaggatacgggcgctatttttacgtctgtgatggctcgcggtaagttgcagggcgatgacatgttgcagcttacgatggctggtgttcctgtgctgtctttgcttgccaggcagacgggtaaaacctcggctgaggtttcgcagatggtgtcgaaggggcagattgattttgccacgtttgcggctgcgatgaagcttggcatgggtggtgctgcgcaggcgtctggtaagacgtttgagggcgctatgaagaatgttaagggcgctttgggctatttgggtgctacggctatggcgccgtttcttaacggcctgcggcagatttttgttgcgttgaatccggttattaagtctatcacggattctgtgaagccgatgtttgctgccgtcgatgctggtatccagcggatgatgccgtctattttggcgtggattaaccgtatgccggctatgatcacgagaatgaatgcacagatgcgcgccaaggtggagcagttgaagggcatttttgcgagaatgcatttgcctgttcctaaagtgaatttgggtgccatgtttgctggcggcaccgcagtgtttggtattgttgctgcgggtgtggggaagcttgttgcagggtttgctccgttggcggttgcgttgaagaatctgttgccgtcgtttggtgctttgaggggtgccgccggggggcttggtggcgtgtttcgcgccctgggtggccctgtcgggattgtgatcggcttgtttgcggcaatgtttgccacgaacgcccagttccgtgccgctgttatgcagctggtggctgtggttggtcaggcgttgggccagattatggcagctgtgcagccgctgtttggtttggttgctggcgtggttgccaggttggcgccggtgttcggccagattatcggtatggttgctggtttggctgcccggctggtgcctgttattggtatgcttattgcccggctggttcctgttatcacccagattattggtatggtaacccaggttgctgccatgttgttgcctatgctgatgccggttattcaggctgttgttgctgtgatacggcaggttattggtgtcattatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgctgtgctacaggtggtggcccgtattattccggttgttatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatattaaatcgtttacgtctgcgtttattaacggttttaagtcgatcatttctggcggcgtgaacgcggttgtggggttttttacgcggcttggtttgtcggttgcttcccatgtgaggtccggttttaacgctgcgaggggtgctgtttcttccgccatgaatgctattcggagtgttgtgtcttcggtggcgtctgctgttggcgggtttttcagttcgatggcgtctcgtgttcggaatggtgctgtgcgcgggtttaatggtgcccggagtgcggcttcttctgctatgcatgctatggggtccgctgtgtctagtggtgtgcatggtgtgctgggttttttccggaatttgcctgacaatattcggcgtgcgcttggtaatatggggtccctgttggtgtcggctggccgtgatgtggtgtccggtttaggtaatggtatcaagaatgctttgagtggcctgttggatacggtgcgtaatatgggttctcaggttgctaatgcggcgaagtcggtgttgggtattcattccccgtctcgggtgtttcgtgacgaggttggccggcaggttgttgccggtttggctgagggtattactggtaatgctggtttggcgttggatgcgatgtcgggtgtggctgggaggctgcctgatgcggttgatgcccggtttggtgtgcgatcgtctgtgggttcgtttaccccgtatggcaggtatcagcgcatgaatgataagagtgttgtggtgaatgtgaatgggcctacttatggggatcctgccgagtttgcgaagcggattgagcggcagcagcgtgacgctttgaacgcgttggcttacgtgtgattttgggggtgtggtgcatgtttattcctgacccgtctgatcgttctggtttgactgtgacttggtctatgttgccgttgattggtaatgatccggagcgtgtgcttcatttgacggattatacggggtcgtctccgataatgttgttgaatgattcgttgcgcggtttgggtgttcctgaggtggagcatttttctcaaactcatgttggggtgcatggctcggagtggcgcgggtttaatgtgaagcctcgcgaggtgacgctaccggtgttggtgtcgggtgttggcccggatccggtgggcggttttcgtgacggttttttgaaggcgtatgacgagttgtggtctgcttttcctcctggcgaggtgggggagttgtctgtgaagactcctgccggtcgtgagcgtgtgttgaagtgccggtttgattcggtggatgacacgtttacggtggatccggtgaacaggggttatgcgcgttatctgttgcatttgacggcttatgacccgttttggtatggggatgagcagaagtttcgtttcagtaacgctaagttgcaggattggttgggtggcggccctgtcgacggtaagggtaccgcgtttccggtggtgttgacgcctggtgttggttcgggttgggataatctgtctaataagggtgatgtgcctgcgtggcctgtgattcgtgttgaggggccgttgtcgtcgtggtctgtgcagattgatggtttgcgtgtgtcctcggattggccggtggaggagtatgattggatcactattgatacggatcctcgtaagcagtctgcgttgttggacgggtttgaggatgtgatggatcgtttgaaggagtgggagtttgcgcctatcccgcctggcggttctcggagtgtgaatattgagatggttggtttgggtgccattgttgtgtcggtgcagtacaggtttttgagggcttggtgaatagttgatggctggttttgttccgcatgtaacattgtttacaccggattatcgccgtgtggcgcctatcaatttttttgagtcgttgaagttgtcgttgaagtggaatggtttgtccactttggagttggtggtgtctggtgatcattctaggcttgacgggttgactaggccgggtgcgcggcttgtggttgattatggtggtggccagattttttctgggcctgtgcgtcgggtgcatggtgtgggtccgtggcgttcttcgcgtgtgactatcacgtgtgaggatgatattcgtctgttgtggcgtatgttgatgtggcctgtgaattatcgtcctggtatggttggtatggagtggcgtgcggatcgggattatgcccattattcgggtgcggcggagtcggtggctaagcgggtgttgggggataatgcttggcgttttccgtctggtttgtttatgaacgatgatgagagtcgtggccgctatattaaggattttcaggtgcggtttcacgtgtttgccgataagttgttgccggtgttgtcgtgggctcggatgactgtcacggtgaaccagtttgagaatgcgaagtttgatcagcgtggtttggtgtttgattgtgtgcctgctgtgacccggaaacatgtgttgactgccgagtcgggttcgattgtgtcgtgggagtatgtgcgtgacgccccgaaggcgacatctgtggtggttggtggccgtggcgagggtaaggatcggctgttttgtgaggatgttgattcggcggccgaggatgattggtttgatcgtgtcgaggtgtttaaggatgcccgtaacacggattccgagaaggtgtctctcttcgatgaggctgagcgggtgttgtccgagtcgggggctacgtcggggtttaagattgagttggctgagtcggatgtgttgcggtttggtcccggcaatctgatgcctggggatttgatctatgtggatgtgggttctgggcctattgcggagattgtgcggcagattgatgtggagtgtgtatcgcctggtgatggttggacgaaggtgactccggttgcgggggattatgaggataatccgtcggccctgttggctcgccgtgtggctggtttggctgcgggtgtgcgggatttgcaaaagttttagtaagtgattggggtttgttgtgggtattgtgtgtaaagggtttgatggtgtgttgaccgagtatgattgggctcaaatgtctggtctgatgggtaatatgccgtctgtgaaggggcctgacgattttcgtgtcggcacgacgattcagggttctacggtgttgtgtgagatcctgccggggcaggcttgggctcacggggtgatgtgcacgtcgaatagtgttgagacggtgacgggtcagcttccgggcccgggtgagactcgatacgactatgtggtgttgtctcgggattggcaggagaatacggccaagttggagattgttcccggtgggcgtgcggagcgtgccagggatgtgttgagggctgagcctggcgtgtttcatcagcagctactggcgactttggtgttgtcgtctaacgggttgcagcagcagttggataggcgtgctgtggcggctagggttgcgtttggggagtctgctgcgtgtgatcctacccctgtggagggtgaccgtgtgatggttccttcgggggctgtgtgggctaaccatgccggcgagtggatgttgttgtctcccaggattgagacgggttcgaagtcgatcatgtttggtggttctgctgtgtatgcttacacgatcccgtttgagcgccagttcagtagtccgcctgttgtggtggcgtctatggctacggcggctgggggcacggcacagattgatgtgaaagcctacaatgtgactgcccaaaattttagtttggcgtttattacgaatgatggttcgaagccgaatggtgtgcctgcggtggcgaattggattgctgtcggcgtgtgactgcacgggtgttgtggcggatggtgtgatgttggggggctgtggtgtcgtggtttactcctgcactggtggcctctatttgtaccgcgttggccacggttttgggttctgttcaggctgtcacatcccggtctaggaagcgtttacgcaggctgtcggctcaggtggatgcgatggaagagtatacgtggggtgtgcggcgcgaggtgcgaaggtttaacgccgggcttcctgatgatgtggagccgatgcatcttcctgatttgcccgagtttttgaaagatactgttgatggtggaggtgagtagggttgagggagttggaggaggagaagcggcagcgccgcaattttgagaaggcttcactggtgttgttgtttttgtcgcttgtgttgttggcggtggttgctgcgggtgctttgcgtttcggggctgtatcctctgagcgggattcggagcaggcgagggcccagtcgaatggtacggctgccaggggtttggctgcccgtgtgaagcaggcgtgtgcttcgggtggggtggagtctgtgcgtcttcaccgttctggtttgtgtgtggatgctgtgcgtgttgagcagcgtgttcagggtgtgccgggtcctgccggtgagcgcggcccgcaaggcccttcaggtcctgccggccgggatggtgttaatggttcggctgggctggttggccctgttggtccgcaaggttctccgggtttgaatggtgtgaaaggtcctgacggcttgcctggcgctaacggttcggatggccgtgatggtgttccaggtcgtgcaggtgctgacggtgtgaacggcgttgacggcgctgatggtcgggatggttctgccggtgagcgcggcccgcaaggcccttcaggtcctgccggcccgcaaggtgcacagggtgaacggggtgagcgtggtcccgccggtgcgaatggatcggatggccatgatggtaaggatgggcgctcggtggtgtctgtgtactgttccgggggccgcctggttgtgaaatatagtgacggtgtggcttccacgatatcgggttcggcggcctgccagggtgtgaaaccgtcgcctctagtgactatatcatcccacaaatagaaaggagtggctgtgatggtggtgtttggtggtggtgtgttgtgagatatattcctgcggcgcatcattctgccggctcgaatagtccggtgaatagggttgtgattcatgcgacgtgcccggatgtggggtttccgtccgcctcgcgtaaaggacgggctgtgtccacggcaaactatttcgcttccccatcgtctggtggttcggcgcattatgtgtgtgatattggggagacggtgcaatgcttgtcggagtctacgattgggtggcatgccccgccgaatccgcatagtttgggtatagagatttgcgcggatgggggttcgcacgcctcgttccgggtgccggggcatgcttacactcgtgagcagtggctggatcctcgcgtgtggcctgcggttgagcgtgccgccatcctgtgtagacgtttgtgtgacaagcatggtgttccgaaaaggaaactgtctgtggccgatttgaaggccggtaaacggggtgtttgcgggcatgtggatgttacggatgcgtggcatcagtcggatcatgacgatccggggccgtggtttccgtgggacaaatttatggctgtggttaatggccacggcggcggttcaagtagtgaggagttgagtatggctgatgtacaagcgttacataatcagattaaacagttgtcggcacaggtggcccagtcggtgaataagctgcatcacgatgttggtgtggttcaggttcagaatggtgatttgggtaaacgtgttgatgccttgtcgtgggtgaagaatcctgtgacggggaagctgtggcgcactaaggatgctttgtggagtgtctggtattacgtgttggagtgtcgtagccgtcttgacaggctcgagtctgctgtcaacgatttgaaaaagtgatggtggtttgttgtgggtaaacagttttggttaggtttgctggagcgtgccctgaaaacttttgttcaaacgtttgttgccgtgttgggggttactgcgggtgtcacctatactgcggagtcgtttcgtggtttgccgtgggaatccgcgctgatcacggcaacggttgctgctgtcctgtcggttgctacctcgtttggtagcccgtcgtttgtggccggcaagcccggcaagcagccccaggtggatgcgggtttggttccaccggatgatgggggcttggttgagccgcatatggtggatgtgtcggatcctggcatgatcgagccgacggatgatgcggatcttgccggctatgagcctcggcgtgcagccgagtcggaggttggcacggtagagtctactgttgcataattgaatatagatgtgtgccccagcggtgctgccacgattgtgtggtggcggctgctggggcactatttttgtatatgcggtgtggctatgattcgttgctgtcgatggtgtcttcgagcatctgatacaggtggaggcaggtagagatagtttcgctggcctgatcgagaacgttccggccgataacgtttttgtggttgtcgcggtggcggatgatagcccacatgatctcgtcggctgccgcctgtaatagtttggcctggtatgcgattccggcgagccagtctagtgcttcctggcttgtataggggctctggtcctcgctgttgccgcgggtgttgctgttgtttgtggggtgtcctgcactgtcgcatagccacaggatttcgctgcactcgtctagcgtgtcttggtcgatagcgagatcgtcgaggctgacattgttgacggtaaggttcacgttgtcgagggagatgggtacaccgtactggttttcgacactgtcaacaatgttttccagctgttgcatgttggtgggctgttgttggacgatacggtgtatcgctgtgttgagggtggtgtaggtgatgttgtgtgtgttgtccatggtttttatgccattccttcgttatcgtctggcatgtagtatgtgctgtttgcgtactcggttaacgtcatcagtgtttggtctgcccactgtttcacggtttgccgggtgactccgagtcgttgggcggctgtggcgtaggtttgatcatacccgtatacttcccggaatgctgccaacctagctaggtgtttcctctgtttggatggttcacaggtgagggtgtagtcgtcgatggctagctgtagatcgatcatggagacgatgttgttgccgtggtgttgtggcgcggttggtgggggtggcattcctggctccacggagggtttccaggggccgccgttccagatccattgggcagcttggatgatgtcggcggtggtgtaggttcggttcactggtcaccccctgaacaggtcgttggtgttgttggtgtcgaatcgtccgacgcagtggcagtagtcgtacatgagtttaataatgtgttggtggtctcccaaataggtgtttccgctgatgctgtatgtggctgtgccgtctttcgcgatggtgtatttggcggtgatggtttcggggttttcggtgtcggtgatgattgctgtggtggtggcgcctactgtttggagtatggtggtttgggttccgtcgtcgatggtggttttaaccatggtgtgtgttttcccttttgttagttgcttgtttggttgtcggctagatgaataatatcgggtaaaggtttcggctggtctaggtgttgtatggttttgttggctagccgtttggctaccctgtaacacattttggtgtagtgtttgttgtctaggttgtggtattgttcccgcaccgcaatatatagcagggagtcttggtacaggtcgtctgcactgattgcggggtagtgtgcggctgttttggtgcatgcccggttgagtgtgcgaagatgatggtctgtggcccacacccacgatgcggtggtggccaggtcggcttttgttggtcgtctgctcatggcactatttcatctcgctatctgatagttgtttggtgttttgttgtggatagtgtagcacactagtcctgggtggccggtggtgcctgtgcggtgacggaaccatgtggattcgccttccatggatgggcattggatgaaggtgcgttgtccttgctcggagatttctaggtggtgccggtgcccggccatgagaatattagatacggtgccgttgtggaattcttggccgcgccaccaatcatagtgtttaccggtgcgccattggtgcccgtgggcgtgcagtatccgtgtgcctgccacatcaacggtggtggtcatttcgtctcggctggggaagtggaagtgtaggttggggtattggttattgagctggtaggcttctgcgatggcccggcagcagtccacgtcgaatgagtcatcgtaggtggtgactcctttaccgaagcgcacggcttcaccatggttgccggggatggatgtgatggtcacatttttgcagtggtcgaattggtggatgagttgcatcatggccatgcgggtgagcctgatttgttcggtgaggggtgtttgtgttcgccaggcgttgttgcctccttgtgacacgtatccttcgatcatgtcgccgaggaaggcgatgtggactcgttcgggtttgcctgcttgttgccagcagtgttttgcgactatgagggagtgtaggtagttgtcggcgaagtgtgctgtttctccgccggggatgcctttgccgatttggaagtctcctgccccgatgacgaaggctgcggtgctgtagtcggtgtgggtgtcttgttcgggttttgggggtgtccattcggctagtttatcgacgagttcgtctaccgggtaggggtttgttgcgggttggtggtcgatgattttttgtatggatcggcctgtttctccgttggggagtgtccattcggagatgcgtgtgcggcgcacggtgccgttggctagattgtcgtcgatggtgtcgatggcgttgtcgtggttggctagctgtgtgagtagccggtcaatattgtctatcactgggtatcctcctcttgcggggtggtgctggcttgtttgcggcgatagtctttaataacggtggcggagatggggtatcctgcctgggtgagctgttttgctagccatgaggcggggatagacctgtcggcgagcacgtcggcggctttgttgccgtagcgttgaataagggtttcagttttggttgccatgatgtcctatcggttgtgtggtgggctgccatcctgtgcggcagtcgccgtcgtgtcctggtttgcgtgtgcaccacgatacggttccgtctgtgtggttgagtgttttaccgcacatgacgtttcggagatgctccggcagctggtcatcctggttgctggtttgtgtgtcgaagagtgttttctggttggtgaaatgttctgacacggtgccgttatgcacgggtagtatccatgttttccattgttgttgtagcctggtgttccagtggaattgtttggcggcgttttcggcctgttttaaggttttgtggtagccgactagtatgcgttgatgctgctggtctggagggtttgggcctcgccagtattgtgccgccacggcgtagcggttgctgtctgtgaaggcgtcccagcagtattcgataatgtgttgcaacatactgtctggcaggctgtcagggttgatgttgatgttttgggtgataatgtcacggatggcttgccggtttttggtggtgggtttgaacgagatgctcacgatagtaccggctggtcgtcttgcatgaactggttgaaggtgttgttcccggcgtgttgggcttgtgttatttgttggtcggtccagtctgggtgttgctgtttcagatagtgccagtggcacgcattgtaggtttcgtcttgtagccgtgtgagatggttttcggtgatgatttgtttccacatggcccatgacacgtcgagccggtcgaggatttcgagggctgggatgttgaattggttcaggaagaggatttcgtgggtgtagtagtttttctcgtaggcgtcccatccgcttcggtgcctgttgggctggtttttggggtaggcttcccggcatactttgtgtaaacgcttggccatgtcgtcgggtagtttaatgtcggggttggcgcggatcatggatcgcatcccatcataggtggtgccccaggtgtgcatgatgtaggtggggtcttctccgtcggcccatttttctgcacagatggcgaggcggatacgcctcctggcagcttggctggtgttgcgccggttggggattgggcacgtgtcgaggggatccatgatgttttagtgtacctttctggtttcgtgttgttgacaggttttactgtagcacagtgtctagtgcgtgtgtcaaccctgtttttccggcttgaaggtaggtgtctgtgacatcccctagggtgaggggcacgtgcacagcttgggggagtgccgcctggagggtttgggccatctggtcgcctgcggggtctgggtctgaccagatgtagatgtggtcgtagccttcaaaaaatttggtccaaaaaatttgccacgaggttgcgccgggtagggcgacggccgaccatccgcattgttcgaggatcatggagtcgaattcgccttcgcaaatgtgcatttcggctgccgggttggccatggcggccatgttgtagatggagcctgtgtctcctgccggggttaggtatttggggtggttgtgggttttgcagtcgtgcgggagtgagcagcggaaacgcatttttcttatttcggctgggccgccccaaacggggtacatgtatgggatggtgatgcactggttgtagttttcgtggcctgggatggggtcattgtcgatgtatccaaggtggtggtagcgggctgtttcttcgctgatgcctcttgctgagagcaggtcgagtatgttttcgaggtgggtttcgtagcgggctgaggctttctggattcggcggcgttccgcaatgttgtatgggcgtatgctgtcgtacatttgggttttcttcttctaatcgttgttgtagcttggcgagtccgcctccgacaccgcatgtgtggcagtaccagacgcccttgtcgaggttgatgctcatggagggctggtggtcgtcgtggaacgggcagagtatgtgttgctcgttcctggacggattgtaccgtatctgataatggtcgaggaggcggcaggtgtcagaggtgtgggaggagctcgttgagggttgataccacataggcttcactccatggcttgttgcgctgtttcatcactacgagtccgatggtggaattgttttgtttgtttcggtgtgtttcgtagttgcgtgcctcccggctggcttgtttcacgaattgggctaggtgtggttgcccggctttcgcctcgataatgtaggttttatggccggttgtgaggatgaggtcgccttcgtcttcgcggccgttgaggtggaggcgttcgatattgtgtccggtgtcgcgtagctggtggaggagtcttgtttcccattcggctccggcccgccggttgcgtgcctgctgtgtggccatagttttttagagtcctttgtgtgttgtggtcatgttccagggctgtttttcggcgagtggcccgaagaatgtgtattcggggtatgctctgagtcgttcgtatcgggtgccgtcggggctggatttgcctgtgcgctgtttgagtacggcgatgcgtgcctctgccggtatcgatagcccgttgccgttatcctcgccaccatacaatgagactccgaggatgagttgtggtttttcggagaggccgtttttgatttctcgccgtgctggcgggtgttcgatgtcggttccggttttgtcggttgcgtggtgtgtgacaataatggtggagccagtatccctgcccaatgctgtgatccattgcatggcttcttgctgtgcctggtagtcggattcgcagtcttgaatgtccatcaggttgtcgataacaatgagtggtgggaaggtgttccacatttccatgtaggcttgtaactccatggtgatgtctgtccatgtgatgggtgactggaatgagaatgtgatgtgttggccgtggtggatgctgtctcgatagtattctggcccgtagtcgtcgatgttttgttgtatttgttgggtggtgtgttgtgtgttgagggagatgattcgtgtggaggcctcccagggtgtcatgtcccctgatatgtagagggcgggctggttgagcatcgctgtgatgaacatggctagccctgatttttggctgccggaccgccccgcgatcatcaccaagtcgcccttatggatgtgcaaatcttggttatcatatagtggtgcgagttgtggtatgcggggtagttcggctgcggtttgggaggctctctcgaaggatcgttgtagagagagcatcgggaccttaatctatctgtctgttggttgtgtggctggtcagatggagtcgatatcgatatcagcatcagcagaggctgaagtgtcatctagctgaccattatcgcgcttgtctacgtattcggcaaccttatcgtagatggcgtcgtccaatgttttgagcacgaccgcgttgaaaccgtttttggtgcgcacggtggctagtttgaaggcctgctcctcgccaaggtatgcctctagttcgcggatcatggagtgtgggcggtcgttattgccgcgggctttctcaataatagcgttggggatggtttctggggtgccgttgttgagatcgtctagggtgtggaagatggtgacatcagcgtagatgcggtctgcgacctgtccaccgtagccttcagtgttgtgctggacgtcgtgcactttgaaggcgatggccgtggcgtcctggtttcgggaggggttgaagaaggtgctgttgctgttgttgcggtagtttgcgagtcccataactattgtttccttttactgttgtgtctgtttttgttggcttatattggtttatcgggtgaggctgtttcgcttagtgcggaaagcgtcggaaacatcactgttactggtgatgatcttcttgtactgttttagaaggtctgctagctgtgccttgcttgttgcattgttgattttgttgatgacgatggtgttttctttggatgcgattttgttgacgtagtctttggctgcctggttgtatcggtcttggaggatgattgatgcgctcgctacgagtgttgctagatcccagtctttggacacgtcatcgtttttgagtccgcctagcaggtcgatgatggcctgttttgtctgctctgctgtgtctcctcggatgaccgcccatggtgcagcatagtctccaccatatttgagtgtgatcgtgagtcgatcattgtcgatcttgtctttatctgtcatttggtgtccttttctttattgtctgtttctggtggctgtacggtggattctaccgggtatctgtacgagtttttgccgttgacggcccagcaggcgtctcgtacggggcatcctttacagagtgttgtgacgtgggggacgaagatgccttcgctgattcctttcattgcttgactgtacatggatgatacatgccggtaggtgttgttgtcaaggtcgtagagttcggtggatgtgccttgtgtcggggacttgtcgtcgttgcggctggtggctggcgtccaaaacatgcctttcgtgacatggatgtcgtgttggttgagcatgtaccggtatgtgtgcagctgcatactgtcggcgggtaggcgtccggttttgaggtcgaggatgaaggtttcgccggtgtcggtgtcggtgaaaacacggtcgatgtagccgactatttttgtgtcatcgtcgaggatggtttctaccgggtattcgatgcctggtttaccgtccaggattgcggtgatgtattctgggtggttgcgcctccatgttttccagcggtccacaaaggtggggccgtaaaccatccaccagtcgtagtctttcttgtgtggtccgcctgactcgcacatgtttttgcatattctgccggagggtttgatttctgtgccttcggattcggcgagggctacctgggtgtcgaaaatgtttttgaaggatgagagtttgtctggcagtgcagggtattcggcgggattgtacaggtgtaggtcgtattgttcggtgatgtggtgtatggcgcttccggcgatggtggcgtaccaggtgtggtgttgggcgtgatagccgtgggataggcgccatttttctccgcattcggcccactgggtgagtgaactgtaggagatgtgtcctgggtggctgatggttttcgggtattgtgctagaggcattacttgtcgcttgtgttccatgtgttgcgggtgtcttggccggcgtggtgttgctggtaggcgaggagtgcgaggcagtgccaggctgcgtgtgctagatggggtagcccggattcgtggtcgaggttgttgccttgctgccatgatagtagatgcctgtagagggcgtcgacactgtggctccacgggtatcctccggtccagttgttgtcgccatatttggtggcaccgtatccggctacttcgcctagggcgtgaagggatgctgggtcgatgagggagagcctgcagagtttcaattcttttcgggcaccgctgttggggtcggtgtacatgcgggtgggctcatccatggggtgtgtgctccttaagggtgggttactggttgttgttgtgggctagggcggcggcgagaataatgatggcgagggtttcggctatcagtatgggtgttgtgatcatttggtgtctcggggattgttggtgagtgttgaggcacccaggagggtggcgagggcgcatgcggcaataatggcgagggctgccttgtgtggggtgccggttgcgtacatccatgtgatgatggcaccttggatccaggctaggctggtgaagaaggtttcgtagctgtgcagctcaatgttgttgttgggtgtgttcatgcttgctcctgaagaatggtgttgatggttttataaatgttgtacaggtcggtttcgatagataacagttggttgatttggtggtcgagatcaatgtctgggttgagtgtgttgatgcgggaggcaatatcggtggctgtgcgtagtgtgccgccggtgtggtgaataatgtgtgccgtgtcggcgagtccggtggtgacggcgtagtgggataggagaggcatagcggggatgctccttggcgggttactgttgcgggttgatgttgaggtcggtgacgtgcggtgagttttctgttccggtgacgaggcagtggacggtgacgggtagtttggatgctcccggctggcggacggtggcgccgtagacgatgctgaatgtgtctttaccgatggttttgtggagttggaggtcgatgtcggggttgccgttccagttgacaccttgcgctgcggcctgttgttcggctttgtggttgcaggtgtgtgctgccgtgatcatggtgagtccggtggcggtttcttcaccccttgcttgggcttgcttgtgggctttggcctgctcggcttgtagggatcgggtggcggctgcctgccgtgccgctttctcggctttgcgctgttgggtagtcttgggggtccatgtggtgttggctgtggttgcctgtggggctggctgtgaggtgagtggcgggttgtcgtctggtgctggcatgaatgaggcggcggcaatgatggcggctgtgatgcctgcgatggtgtagccgtttttcttgttcatgttttgtgtcccctttccggggtgttgttcgttgctgacatggttaatactttcagcggctgggcccactgtcaaggctgcgctcagtttgtgtgagcgtttggtgtgtggctaggggttttgtcatgtaagcgtgacatgtcactaccttgcgtccagtatccatggcggttgcgagccatccctttggcgagcatctcgtccacagtgaggcacctgcggcgattggggccttccttgaccccgtgatcgcctatgcggtgcatgtccccggcataagtgccattaaatgtttcgtggcagactgtgcagtgttctggtcggtatccgatgattgtgctatcgcacttgtggcatgtccattgcatgattggtccttctttcgtgttttaagcttgtgctctgaggattagagcgactttcagcccttgggggtaggattatataggtcaggtatttctaggcgattctaggctcattgtgtgtggttggggttttatcgggcgcatagggttagcaggtggcccacattggtgcggctcacattccagtagagttgcgtggcttccttactggtgagcggcttccactcgtcatggctgaacacggtgccatcggatgcgatgaacgtgttggggcgtagcttgtgaagctcggcttccacatgctgccggtaggcttcggcgaggctctcaaaatccatgtggtcgcaggagaggttttcgaggcgtgtcaggtcgaaaggctccgggcagtcgtagctggctggagtgtagagctgggtgaagtggtcggcgatcttctgcatggcgggttcctttctggtgtgtggatggtttttatcgtgtggatgcgacaaggatggcgtctacgtcgatcatgtcgatcatgtcgttgagttcctcggcctcattctcggagaggtggcgccagtcgggtggcccgtatacggcgccgtcgagggtgacagtccacaggggccggatgagtcgtatggcttcttcgactttggcgtggtacatgcggcgcaccatatccagatcgatgtcgtctgaatggtttccggtgaggctgtggaggctgagcgggtcgatttctgtctgcctgtagaggctggtgaatgatggtgtgatgagtgtgccatccatgagtgtgctcctttctaggggttgttgtggtttctagagtgtgtgggctgtgaccccacagtcaaggctacgctcatttggattgagcgtttcatatgggtgtggcatggaatctacaccctcatactgtgtgagatgtatcacatccccctggcttggtgtgcacccctcaagactactctgccgacctggcgtggagggtgtagcccagaaatgccgtttaaagcttcaggggtacgcctaggagcgccttacagggtgggggctaggtatttatacccccagcatattctgatcgattctagacgactcccagagcccgatacacgatcaaccatctcgacatagaccatcagcccctatcctggttagctaagcctcaactatgtggacagtgtgggacactgtgggggaagaaggacacggtacaagaaagaggggggagcatcagccttaaagccttaagatcttagcgcttagcaccgatggtcttagcagttagcaccgagcccttgagggggctcggcatcagcctcatcgggctcagctcatcaggcacagccctgaaaagggtacacgccatcagggaaggcttgagagtacgaggagccctagcgacgagtactcgaaagcctgagggaacaccctcagtactgatgagcctagcgtattcggaaaggacgcaagagtaaagtgtgacagctatccgggagtgaaacccgttccgactaggggtttcagccttaaccaccctcaaaggttacaagactctaagaaaatttaagaaacttcttaggaagaaagttgtgttcatatccccctaaaaacacccaaaatagtcctcaaacccgcctatagagccaaacagtcaagtttgactcgtctagacggcgtatgataggctggacaggtagccagctggacgcaaggccagaaagtgctgacgcacttcccgacctcgcttaccatcagtctaccaaacactttaaagcttcaaggcttagcgctaagcccttaagatcttaacgcttagcaccgagcccccctcaagggctcgacatcagtcttaaagtcttaaacactttaagtaactttaaagcttcaaggcttagcccttaaggatctaagttactataaaagctttaaacacttaaagtaactataaagctttaagagcttaacatttaaggatataaataaacattaaagctttaaagtcttaaagtaaatatataaccttaacacttaagttaagtataaaaccttaaaggcttagcacttaaggatataaacttaacatcagtgtttaagacttaaagagttaaagtaactattaagacttaaaggcttataagctttaatactttaagtagctataagactttaaaaacctgaagtacttaaagttaaccatcagtcttaaactttaatattataagtattaaagcttataagttataaaagtttttagaagagttaaagggttaacttctttacttctcttctctctttggttctttctctcttctcttcttttcttcatcaggggagaagaggaacctttaaccgtcaacgctgatggacttttcaccgtgtgactcgtgtgcttctggtcgcaagctcccatcgcacactccccacactctttcacccgtgcccctttacggcttagcgtgttcgtcggaaggcgtacggcgtgtcacgcttaaacccttaacaccaggtaagacttaaagtgcatattataagtagaagactttaaaacctataaggtgttcccgcttagcccgtgttcctttaacgctaggcgctcagcgctaagatgtgaaacgtgaacacccatccacccccatttttcttccgtgtccttctccttttgacaccgctggggggcgatgtgatatttctcacatgccagggggtagtggagaaaacaaccaccccggaacgtttaagacaccccctcaaacgaacaaaacagggcctagaatcgatcagcagggcaccggtagggtattcctacccccagacgattcaaggccattacaggagcaatgagaggctcacaggggccatgggagattggggggcgtgatggcacacaccaaccgcacagccagccaagcccaccggcgctggcgggcaaggctcatcacccaagcccgacaacaaggccaaaccgaatgcccactctgcggagtcaccatcacctggaacacccacgacctgccaaccagccccgaagccgaccacatcacacccgtcagccggggaggactcaacaccctcgacaacgggcaaatcatctgcagaacatgcaacagaagcaaaggcaacagaacacaaccaaacatcaaattccaacaacaaaccacaaaaacattgattccatggtgaaaaacccgccaacccccaccgggcacaccccctgcacacccgtgcaagacc

SEQ ID NO: 28 - DNA, 11979 bp, synthetic construct
  source 1..11979: mol_type = other DNA; note = pANS514 plasmid
catggttgcgccatgcagcacaggccaagcgtgaggcccaagcacgaggactcgcccgctgcccactgtgcggcgtctggatggactacgaggtcggcaagcgacccaactcggccgaagcagaccacatcagaccgcattcgcttggtggttcagacgacatcgacaacattcgcgtcatttgtcgtcgttgcaatcaatcgcgcggaaacggcctgaagcgaccagggcgccaacgtcagcgtccaatcaagcgcatcgagctggcccaaccggcccgcagtggggcatttcctgccccgccggcatgaatggaagggcagtgcggatggtgcggtcgggcattcgatcgtgcccggacgggtcgcccgcgacgcttctgctcggcccgctgtcgggtcgccgcgtcccggtgtgcgatcccgctggccatgaggtcccgcactgcgtgggtccgctgcgacggcaagcgccccatcaccctggctggcgctccggcctcatccacggacccgggcacatggtctggctggtcgcaggtgcgacgcgccacggccggcgatggcttcgggaccatgctcggtgacgggctggggtgctgggatctcgaccacttcgacgatcagggcgcccgggccttcatcgaccggatcgataagccgatcatcttcgccgagcggtcggtgtcggggcatggcttccacatcttcgtccggactgacgaggcccccggacgccgcaccggaaacatcgagttctactcacgccatcggttcatcagggtcacaggagaccagttcgtctgaaggtcgtgccgggtttgtgtctgatgaggagtggttgtttctcatggatgctgcggtgattcatgatgtggtgtggcgtgagggtcgcgcggatttggtggcttcgttgcgtgctcatgtgaaggcttttatgggtatgttggataggtattcggttgatgtggcgtctggtggccgtggtgggggttctgcggtagcgatgattgaccggtataggaagcgtaggggggcttgagtaggtgtctggtgttgttgggtctcaggttcctcgtcaccgggtggctgtggcgtattcggtgtctgctggcggggatgctggggagcttggtagggcttatgggttgacgcctgatccgtggcagcagcaggtgttggatgattggcttgctgtgggtggtaatggcaggcttgcttcgggtgtgtgtggggtgtttgttccgcggcagaatggcaagaatgctattttggagattgtggagttgtttaaggcgactattcagggtcgccgtattttgcatacggctcacgagttgaagtcggctcgtaaggcgtttatgcggttgcggtcgttttttgagaatgagcggcagtttcctgacttgtatcgtatggtgaagtcgattcgtgcgacgaatggccaggaggctattgtgttgcatcatccggattgtgccacgtttgagaagaagtgtggttgtccgggttggggttcggttgagtttgtggctcgtagccggggttctgctcgcgggtttacggttgatgatttggtgtgtgatgaggctcaggagttgtcggatgagcagttggaggctttgcttcctaccgtgagcgctgccccgtctggtgatcctcagcagatttttttgggtacgccgccggggccgttggctgacgggtctgtggtgttgcgtcttcgcgggcaggctttgtcgggtggtaaacggtttgcgtggacggagttttcgattcctgacgagtctgatccggatgatgtgtcgcggcagtggcggaagttggcgggtgacactaatccggcgttggggcgccgcctgaatttcgggacagtctcggatgagcatgagtcgatgtctgctgccgggtttgctcgggagcggcttggctggtgggatcgtggccagtctgcttcgtctgtgattccggcggataagtgggttcagtcggctgtggttgaggcggctctggttggcgggaaggtttttggtgtctcgttttctcgctcgggggatcgtgtcgcgttggctggtgctggtaaaacggattctggtgtgcatgttgaggttattgatggcctgtctgggacgattgttgatggtgtgggccagctggctgattggttggcgttgcgttggggtgacactgaaaaggttatggttgcagggtctggtgcggtgttgttgcagaaggctttgacggatcgtggtgttccgggtcgtggcgtgattgtggctgatactggggtgtatgtggaggcgtgtcaagccttcctggagggtgtcaggtctgggagcgtgtctcatcctcgtgccgattcgaggcgtgacatgttggatattgctgtgaggtcggctgtgcagaagaagaagggttctgcgtggggttggggttcctcgtttaaggatggttctgaggttcctttggaggctgtgtctttggcgtatcttggtgcgaagatggcgaaagcgaagcggcgtgaacggtctggtaggaagcgggtgtctgtggtatgaactcggatgagttggctctgattgagggcatgtacgatcgtattcaagggttgtcttcgtggcattgccgtattgagggctactatgagggctctaatcgggtgcgtgatttgggggttgctattccttcggagttgcagcgggtgcagacggtggtgtcatggcctgggattgcggtggatgctttggaggagcgtctggattggcttggctggactaatggtgacggctacggtttggatggtgtgtatgctgcgaatcggcttgctacggcgtcgtgtgatgttcaccttgatgcactgatttttgggttgtcgtttgtggcgatcattccccaagaggatgggtcggtgttggttcgtcctcagtcgccgaagaattgtactggccggttttctgccgatgggtcttgtttggatgctggccttgtggtgcagcagacgtgtgatcctgaggttgttgaggcggagttgttgcttcctgatgtgattgttcaggtggagcggcggggttcgcgtgagtgggttgagacgggccgtatcgagaatgtgttgggtgcggttccgttggtgcctgttgtgaatcgtcgccgtacttctaggattgatggccgttcggagattacgaggtctattagggcttacacggatgaggctgttcgcacactgttggggcagtctgtgaatcgtgatttttatgcgtatcctcagcgttgggtgactggcgtgagcgcggatgagttttcgcagccgggttgggttctgtcgatggcttctgtgtgggctgtggataaggatgatgatggtgacactccgaatgtggggtcgtttcctgtgaattctcctacaccgtattctgatcagatgcgtttgttggcgcagttgactgcgggtgaggcggctgttccggaacgctatttcgggtttatcacttctaacccgccttctggggaggctttggctgcggaggagtctcggcttgtgaagcgtgctgaacgcaggcagacgtcgtttggtcagggctggctgtcggttggtttcctggctgcccgggcgttggattcgagtgttgatgaggccgcgttttttggtgatgttggtttgcgttggcgtgatgcgtcgacgccgactcgggcggctacggctgatgctgtgacgaagcttgtgggtgctggtattttgcctgctgattctcggacggtgttggagatgttgggtttggatgatgtgcaggttgaggctgtgatgcgtcatcgtgccgagtcttcggatccgttggcggcactggctggggctatttcccgtcaaactaacgaggtttgataggcgatggcttcgggtgctgtgtcgaggcttgctgcgactgagtatcagcgtgaggctgtcaggtttgctgggaagtatgcgggctattatgccgagttgggtcgtttgtggcgtgccggcaggatgagtgacacgcagtatgtgcgtttgtgtgtggagttggagcgtgccggccatgacggttcagcagctatggcgggcaaattcgtttcagattttcgccggttgaatggtgtcgatcctggtttgatcgtgtatgacgagtttgatgctgcggcggctttggctaggtcgttttcgactatgaagattatgaatagtgacccggatagggcgaatgatacgattgatgcgatggctgcgggtgttaatcgggctgttatgaatgctggtcgtgacacggttgagtggtcggcgggtgcgcagggtaggtcgtggcgtcgggtgactgatggtgatccgtgtgctttttgtgccatgttggctacgaggtcggattatacgactaaagagcgggcgcttactactggtcatacgcggcgtcataagcgtgccggtaggcgtccgtttggttcgaagtatcatgatcattgtggttgtacggtggttgaggttgttggtccttgggaaccgaatagggctgatgccgagtatcagaggacgtatgagaaggctcgtgagtgggttgatgatcatgggttgcagcagtcgtctggcaatattttgaaggctatgcgtactgttggtggcatgagataatttgatgtggtttccggttgtgtgccgccggttatcggtgcacagggttgtctcccgcacgggggtcaacaatgttgtgttgttttccgcaaggagtgtagggttaggctatggccgatcagagtattgaggaacagaatgttgacaatgatgttgtggagtccggaaaggataacggcattgttgatacagtaaaagacgatggcgggcaggaggtagccgacaatcagttgaagaatgaaggcgagggtaaatcgccggggactgattggaaggcggaggcccgtaagtgggagtctcgtgctaaaagtaatttcgccgagttggagaagcttcgtacatcgagtgacgattctggatctactattgatgagcttcgccgcaagaatgaggaactcgaagaccggattaacgggtttgttcttgagggtgtgaagcgcgaggtggctgccgagtgtggcctgtcgggtgatgcgatcgcttttcttcacggtagcgataaggagtcgcttgccgagtctgctaaggctttgaagggtttgatcgaccatagtagtggtggtggcgcgggtgtgcgccgtcttgcggggagtgcccccgttgatgatgttaaacgacgtgagggtgtcgcgtttgtggatgctcttgtcaataattctaggagatgatttatcatggctgacgattttctttctgcagggaagcttgagcttcctggttctatgattggtgcggttcgtgaccgtgctatcgattctggtgttcttgctaaactgtcaccggagcagccgactattttcgggcctgttaagggcgccgtttttagtggtgttccgcgcgctaagattgttggcgagggcgatgttaagccttccgctagcgttgatgtttctgcgtttactgcgcagcctatcaaggttgtgactcagcagcgtgtctcggacgagtttatgtgggctgacgccgattaccgtctgggtgtgcttcaggatctgatttccccggccctgggtgcttctattggtcgcgccgttgatcttattgctttccatggtattgatcctgctacgggtaagcctgctgcggctgtcaaggtgtcgctggataagacgaataagacggttgatgccaccgattccgctacggctgatcttgttaaggctgttggtctgattgctggtgctggtttgcaggttcctaacggtgttgctttggatccggcgttctcgtttgctctgtcaactgaggtgtatccgaagggttcgccgcttgccggtcagccaatgtatcctgccgccgggttcgccggcctggataattggcgcggcctaaatgttggttcttcttcgactgtttctggtgccccggagatgtcgcctgcttctggtgttaaggctattgttggtgatttctctcgtgtccattgggggttccagcgtaacttcccgattgagctgatcgagtatggtgacccggatcagacggggcgtgacttgaagggccataatgaggttatggttcgtgccgaggctgtgctgtatgttgcgattgagtcgcttgattcgtttgctgtcgtgaaggagaaggctgccccgaagcctaatccgccggccggtaactgattcatttgttgcgataatgtttatgctgtgtgcagggggtggtgttgatgggtatcattttgaagcctgaggatattgagcctttcgccgatattcctagagagaagcttgaggcgatgattgccgatgtggaggctgtggctgtcagtgtcgccccctgtatcgctaaaccggatttcaaatatagggatgccgctaaggctattctgcgtagggctttgttgcgctggaatgatactggcgtgtcgggtcaggtgcagtatgagtctgcgggcccgtttgctcagactacacggtcgaatactcctacgaatttgttgtggccttctgagattgccgcgttgaagaagttgtgtgagggtgatagtggggctggtaaggcgttcactattacaccgaccatgaggagtagtgtgaatcattctgaggtgtgttccacggtgtggggtgagggttgctcgtgcgggtcgaatattaacggctatgctggcccgttgtgggagatatgatatgaccggttttccttacggtgaaacggttgtgatgcttcagccgactgttcgtgtcgatgatcttggtgacaaggtggaggattggtctaagcctgtcgagactgtgtaccataacgtggccatctatgcttccgtttcgcaggaggatgaggccgcggggcgtgactcggattatgagcattggacactgctgttcaagcagcctgtcaaggctgctggttatcggtgtcgttggcgtattcggggtgttgtgtgggaggctgacgggtctcctatggtgtggcatcatccgatgtctggctgggatgctggtacgcaggttaatgtgaagcgtaagaagggctgatgggttgtggcacgtgatgttgatgtgaagctgaacttgccgggtattcgtgaggtgttgaagtcttctggggtgcagggcatgttggctgagcgtggtgagcgtgtcaagcgtgcggcctcggcgaatgtgggcggtaacgcttacgatagggcccagtatcgtgccgggttgtcgtctgaggtgcaggttcaccgtgttgaggctgtggcgcgtattggcaccacctataagggtggtaaaaggattgaggctaagcatggcacgttggcgaggtcgattggggctgcgtcgtgatcgtttacggtgatcctcgaatatgggctaaacgtgtgttggcggatgatggttggctgtctgatgtaccgtgcacgggtactgtgccggatacatttgagggtgatctgatttggttggcgttggatggtggcccggagttgcatgttcgtgagcgtgtttttttgcgtgtgaatgtgttttcggatacgccggatcgtgctatgtctttggctcgccgggttgaggctgtgctggctgatggtgtggatggtgatccggtggtgttttgcaggcgttcgactgggcctgatttgctggtggatggtgcacgttttgatgtgtattcgctttttgagctgatatgtaggcctgcggagtctgaataagcttattgtttttgttttaatgtaattgtttgatatttaatgggggttgtgatggctgctacacgtaaagcgtctaatgttcgttcagcggttactggcgacgtttatattggtgacgcgcacgcgggtgattctattaagggtgtggaggcggttccttccgggcttacagctttggggtatctgtctgatgacgggtttaagattaagcctgagcgtaaaacggatgatttgaaggcttggcagaatgcggatgttgttcgcactgtggctacggagtcgtctatcgagatttctttccagctgattgagtcgaagaaggaggttatcgaactgttttggcagtcgaaggttactgccggatctgattcgggttcgttcgatatttctcctggtgccacaacaggtgttcacgccctgttgatggatattgttgatggcgatcaggttattcgctactatttccctgaggttgagctcattgatcgtgacgagattaagggcaagaatggcgaagtgtacgggtatggtgtgacgttgaaggcgtatcctgcccagattaataagactggtaatgcggtgtcgggtcgggggtggatgacggctttaaaagctgatactcctccgactcctccgccggccccggttcctccgaagcctcagccggatccgaatccgccgtccggtaactgatacacgattttaggggattgttaatagatgagtgacactggtttcacgttgaagattggtgatcgtagctgggtgttggcggatgcggaggagacggctcaggctgttcctgcccgcgttttccgtcgtgccgccaggattgcccagtcgggggagtctgcggatttcgcccaggttgaggtgatgttttctatgttggaggctgccgccccagctgacgcggtggaggccctggaggggcttcctatggttcgtgtggcggaggttttccgtgagtggatggaatacaagcctgacggtaagggtgcctcgctgggggaatagtttggctccacggcctgattgatgattatcgtggggccatcgaatacgatttccgcaccaagtttggtgtttctgtttatagtgttggtggcccgcagatgtgttggggtgaggctgtccggctggctggcgtgttgtgtaccgatacgtctagccagttggcggcccaccttaatggttggcagcgcccgtttgagtggtgcgagtgggctgtgttggacatgttggatcattacaggtctgctaatagtgaggggcagccggagcctgtggcgaggccgactgatgagcgtcgggcaaggtttacgtctgggcaggtggacgatattttggcgcgtgttcgtgccggtggcggggtgtctcgcgagattgatattatggggtgaatagtgtatgtctggtgagattgcttccgcatatgtgtcgttgtatacgaagatgcctggccttaaaagtgatgttggtaaacagttgtcgggtgttatgcctgctgaggggcagcgttcgggtagcctgtttgctaaaggcatgaagttggcgcttggtggtgcggcgatgatgggtgccatcaatgttgctaagaagggcctcaagtctatctatgatgtgactattggtggcggtattgctcgcgctatggctattgatgaggctcaggctaaactgactggtttgggtcacacgtcttctgatacgtcttcgattatgaattcggctattgaggctgtgactggtacgtcgtatgcgttgggggatgcggcgtctacggcggcggcgttgtctgcttcgggtgtgaagtctggcggtcagatgacggatgtgttgaagactgtcgcggatgtgtcttatatttcgggtaagtcgtttcaggatacgggcgctatttttacgtctgtgatggctcgcggtaagttgcagggcgatgacatgttgcagcttacgatggctggtgttcctgtgctgtctttgcttgccaggcagacgggtaaaacctcggctgaggtttcgcagatggtgtcgaaggggcagattgattttgccacgtttgcggctgcgatgaagcttggcatgggtggtgctgcgcaggcgtctggtaagacgtttgagggcgctatgaagaatgttaagggcgctttgggctatttgggtgctacggctatggcgccgtttcttaacggcctgcggcagatttttgttgcgttgaatccggttattaagtctatcacggattctgtgaagccgatgtttgctgccgtcgatgctggtatccagcggatgatgccgtctattttggcgtggattaaccgtatgccggctatgatcacgagaatgaatgcacagatgcgcgccaaggtggagcagttgaagggcatttttgcgagaatgcatttgcctgttcctaaagtgaatttgggtgccatgtttgctggcggcaccgcagtgtttggtattgttgctgcgggtgtggggaagcttgttgcagggtttgctccgttggcggttgcgttgaagaatctgttgccgtcgtttggtgctttgaggggtgccgccggggggcttggtggcgtgtttcgcgccctgggtggccctgtcgggattgtgatcggcttgtttgcggcaatgtttgccacgaacgcccagttccgtgccgctgttatgcagctggtggctgtggttggtcaggcgttgggccagattatggcagctgtgcagccgctgtttggtttggttgctggcgtggttgccaggttggcgccggtgttcggccagattatcggtatggttgctggtttggctgcccggctggtgcctgttattggtatgcttattgcccggctggttcctgttatcacccagattattggtatggtaacccaggttgctgccatgttgttgcctatgctgatgccggttattcaggctgttgttgctgtgatacggcaggttattggtgtcattatgcagttgatacctgttttgatgccggttgtgcagcagattttgggtgctgtcatgtctgttttgccgccgattgttggtttgatacggtcgctgataccggtgatcatgtcgattatgcgtgtggtggtgcaggttgttggtgctgtgctacaggtggtggcccgtattattccggttgttatgccgatttatgtttcggtgattggattcattgccaagatttatgctgcggttatcgtttttgaggctaaggttattggcgctattcttcgtactattacgtggattgtgaatcattcagtgtctggcgtgaggtctatgggcacggccatccagaatggctggaatcatattaaatcgtttacgtctgcgtttattaacggttttaagtcgatcatttctggcggcgtgaacgcggttgtggggttttttacgcggcttggtttgtcggttgcttcccatgtgaggtccggttttaacgctgcgaggggtgctgtttcttccgccatgaatgctattcggagtgttgtgtcttcggtggcgtctgctgttggcgggtttttcagttcgatggcgtctcgtgttcggaatggtgctgtgcgcgggtttaatggtgcccggagtgcggcttcttctgctatgcatgctatggggtccgctgtgtctagtggtgtgcatggtgtgctgggttttttccggaatttgcctgacaatattcggcgtgcgcttggtaatatggggtccctgttggtgtcggctggccgtgatgtggtgtccggtttaggtaatggtatcaagaatgctttgagtggcctgttggatacggtgcgtaatatgggttctcaggttgctaatgcggcgaagtcggtgttgggtattcattccccgtctcgggtgtttcgtgacgaggttggccggcaggttgttgccggtttggctgagggtattactggtaatgctggtttggcgttggatgcgatgtcgggtgtggctgggaggctgcctgatgcggttgatgcccggtttggtgtgcgatcgtctgtgggttcgtttaccccgtatggcaggtatcagcgcatgaatgataagagtgttgtggtgaatgtgaatgggcctacttatggggatcctgccgagtttgcgaagcggattgagcggcagcagcgtgacgctttgaacgcgttggcttacgtgtgattttgggggtgtggtgc

SEQ ID NO: 29 - DNA, 83 bp, synthetic construct
  source 1..83: mol_type = other DNA; note = PAC7 cos of pAN594
acaaaagggaggtatttcactaagccgtacgaggtcttgcacgggtgtgcagggggtgtgcccggtgggggttggcgggtttt

SEQ ID NO: 30 - DNA, 4670 bp, synthetic construct
  source 1..4670: mol_type = other DNA; note = operon of gp15-gp19+gp45
cgacgcggcggtctgccgacccggcaacgaccaactccccgacgggcgctgacaccggcccggcagcgtgcatgcgtgcatttccaccctcaagaaccattgactggcgacgcgcaggtgggagaattgaactgaacgctttgaacgcgttggcttacgtgtgattttgggggtgtggtgcatgtttattcctgacccgtctgatcgttctggtttgactgtgacttggtctatgttgccgttgattggtaatgatccggagcgtgtgcttcatttgacggattatacggggtcgtctccgataatgttgttgaatgattcgttgcgcggtttgggtgttcctgaggtggagcatttttctcaaactcatgttggggtgcatggctcggagtggcgcgggtttaatgtgaagcctcgcgaggtgacgctaccggtgttggtgtcgggtgttggcccggatccggtgggcggttttcgtgacggttttttgaaggcgtatgacgagttgtggtctgcttttcctcctggcgaggtgggggagttgtctgtgaagactcctgccggtcgtgagcgtgtgttgaagtgccggtttgattcggtggatgacacgtttacggtggatccggtgaacaggggttatgcgcgttatctgttgcatttgacggcttatgacccgttttggtatggggatgagcagaagtttcgtttcagtaacgctaagttgcaggattggttgggtggcggccctgtcgacggtaagggtaccgcgtttccggtggtgttgacgcctggtgttggttcgggttgggataatctgtctaataagggtgatgtgcctgcgtggcctgtgattcgtgttgaggggccgttgtcgtcgtggtctgtgcagattgatggtttgcgtgtgtcctcggattggccggtggaggagtatgattggatcactattgatacggatcctcgtaagcagtctgcgttgttggacgggtttgaggatgtgatggatcgtttgaaggagtgggagtttgcgcctatcccgcctggcggttctcggagtgtgaatattgagatggttggtttgggtgccattgttgtgtcggtgcagtacaggtttttgagggcttggtgaatagttgatggctggttttgttccgcatgtaacattgtttacaccggattatcgccgtgtggcgcctatcaatttttttgagtcgttgaagttgtcgttgaagtggaatggtttgtccactttggagttggtggtgtctggtgatcattctaggcttgacgggttgactaggccgggtgcgcggcttgtggttgattatggtggtggccagattttttctgggcctgtgcgtcgggtgcatggtgtgggtccgtggcgttcttcgcgtgtgactatcacgtgtgaggatgatattcgtctgttgtggcgtatgttgatgtggcctgtgaattatcgtcctggtatggttggtatggagtggcgtgcggatcgggattatgcccattattcgggtgcggcggagtcggtggctaagcgggtgttgggggataatgcttggcgttttccgtctggtttgtttatgaacgatgatgagagtcgtggccgctatattaaggattttcaggtgcggtttcacgtgtttgccgataagttgttgccggtgttgtcgtgggctcggatgactgtcacggtgaaccagtttgagaatgcgaagtttgatcagcgtggtttggtgtttgattgtgtgcctgctgtgacccggaaacatgtgttgactgccgagtcgggttcgattgtgtcgtgggagtatgtgcgtgacgccccgaaggcgacatctgtggtggttggtggccgtggcgagggtaaggatcggctgttttgtgaggatgttgattcggcggccgaggatgattggtttgatcgtgtcgaggtgtttaaggatgcccgtaacacggattccgagaaggtgtctctcttcgatgaggctgagcgggtgttgtccgagtcgggggctacgtcggggtttaagattgagttggctgagtcggatgtgttgcggtttggtcccggcaatctgatgcctggggatttgatctatgtggatgtgggttctgggcctattgcggagattgtgcggcagattgatgtggagtgtgtatcgcctggtgatggttggacgaaggtgactccggttgcgggggattatgaggataatccgtcggccctgttggctcgccgtgtggctggtttggctgcgggtgtgcgggatttgcaaaagttttagtaagtgattggggtttgttgtgggtattgtgtgtaaagggtttgatggtgtgttgaccgagtatgattgggctcaaatgtctggtctgatgggtaatatgccgtctgtgaaggggcctgacgattttcgtgtcggcacgacgattcagggttctacggtgttgtgtgagatcctgccggggcaggcttgggctcacggggtgatgtgcacgtcgaatagtgttgagacggtgacgggtcagcttccgggcccgggtgagactcgatacgactatgtggtgttgtctcgggattggcaggagaatacggccaagttggagattgttcccggtgggcgtgcggagcgtgccagggatgtgttgagggctgagcctggcgtgtttcatcagcagctactggcgactttggtgttgtcgtctaacgggttgcagcagcagttggataggcgtgctgtggcggctagggttgcgtttggggagtctgctgcgtgtgatcctacccctgtggagggtgaccgtgtgatggttccttcgggggctgtgtgggctaaccatgccggcgagtggatgttgttgtctcccaggattgagacgggttcgaagtcgatcatgtttggtggttctgctgtgtatgcttacacgatcccgtttgagcgccagttcagtagtccgcctgttgtggtggcgtctatggctacggcggctgggggcacggcacagattgatgtgaaagcctacaatgtgactgcccaaaattttagtttggcgtttattacgaatgatggttcgaagccgaatggtgtgcctgcggtggcgaattggattgctgtcggcgtgtgactgcacgggtgttgtggcggatggtgtgatgttggggggctgtggtgtcgtggtttactcctgcactggtggcctctatttgtaccgcgttggccacggttttgggttctgttcaggctgtcacatcccggtctaggaagcgtttacgcaggctgtcggctcaggtggatgcgatggaagagtatacgtggggtgtgcggcgcgaggtgcgaaggtttaacgccgggcttcctgatgatgtggagccgatgcatcttcctgatttgcccgagtttttgaaagatactgttgatggtggaggtgagtagggttgagggagttggaggaggagaagcggcagcgccgcaattttgagaaggcttcactggtgttgttgtttttgtcgcttgtgttgttggcggtggttgctgcgggtgctttgcgtttcggggctgtatcctctgagcgggattcggagcaggcgagggcccagtcgaatggtacggctgccaggggtttggctgcccgtgtgaagcaggcgtgtgcttcgggtggggtggagtctgtgcgtcttcaccgttctggtttgtgtgtggatgctgtgcgtgttgagcagcgtgttcagggtgtgccgggtcctgccggtgagcgcggcccgcaaggcccttcaggtcctgccggccgggatggtgttaatggttcggctgggctggttggccctgttggtccgcaaggttctccgggtttgaatggtgtgaaaggtcctgacggcttgcctggcgctaacggttcggatggccgtgatggtgttccaggtcgtgcaggtgctgacggtgtgaacggcgttgacggcgctgatggtcgggatggttctgccggtgagcgcggcccgcaaggcccttcaggtcctgccggcccgcaaggtgcacagggtgaacggggtgagcgtggtcccgccggtgcgaatggatcggatggccatgatggtaaggatgggcgctcggtggtgtctgtgtactgttccgggggccgcctggttgtgaaatatagtgacggtgtggcttccacgatatcgggttcggcggcctgccagggtgtgaaaccgtcgcctctagtgactatatcatcccacaaatagaggctcacaggggccatgggagattggggggcgtgatggcacacaccaaccgcacagccagccaagcccaccggcgctggcgggcaaggctcatcacccaagcccgacaacaaggccaaaccgaatgcccactctgcggagtcaccatcacctggaacacccacgacctgccaaccagccccgaagccgaccacatcacacccgtcagccggggaggactcaacaccctcgacaacgggcaaatcatctgcagaacatgcaacagaagcaaaggcaacagaacacaaccaaacatcaaattccaacaacaaaccacaaaaacattgattccatggtgaggatatccacgagctgcgttcggctaaacccaaaagtaaaaacccgccgaagcgggttttaacgtaaaacaggtgaaactgac

SEQ ID NO: 31 - DNA, 1910 bp, synthetic construct
  source 1..1910: mol_type = other DNA; note = pAN241 vector
caagtggcccatcgaagaggacggcaccaccatctcgccgggcaagctcaaggacgtgtccaggctgacgctcacggtgctgctgcacccctcgtgcgccatcatcgtggatccccaagattgtccggacggcggttgagcgcggcctgataggcgccgcagctcctgctcccgggccgccccggtcggcggtttactcctttcctgccggccggggcactcaagacaaccgggggccctcgcgaaattgaggggccccgcctgattgcaagggggtgcccatgaagcaacccgggccccaccaaagaatgcgggctaccttcaaggccgacaggggctggcgagtggcatgcccacggtgcgcctggcatgccaccagcacccaccttgcatggctcatggatcaggccagcacacacacctgtgcacccctgctgttgtcgcccacgccacccgacgtggagctggcaccggcaggcgacgggctgtccgtcctgtggcccgaggtggacggtgacgtgcagttcacctgcatccacaccagcaccgccacgtgcaggcaggacgcaccatgagcaccagtcgcaccggcacggccacatggttgcgccatgcagcacaggccaagcgtgaggcccaagcacgaggactcgcccgctgcccactgtgcggcgtctggatggactacgaggtcggcaagcgacccaactcggccgaagcagaccacatcagaccgcattcgcttggtggttcagacgacatcgacaacattcgcgtcatttgtcgtcgttgcaatcaatcgcgcggaaacggcctgaagcgaccagggcgccaacgtcagcgtccaatcaagcgcatcgagctggcccaaccggcccgcagtggggcatttcctgccccgccggcatgaatggaagggcagtgcggatggtgcggtcgggcattcgatcgtgcccggacgggtcgcccgcgacgcttctgctcggcccgctgtcgggtcgccgcgtcccggtgtgcgatcccgctggccatgaggtcccgcactgcgtgggtccgctgcgacggcaagcgccccatcaccctggctggcgctccggcctcatccacggacccgggcacatggtctggctggtcgcaggtgcgacgcgccacggccggcgatggcttcgggaccatgctcggtgacgggctggggtgctgggatctcgaccacttcgacgatcagggcgcccgggccttcatcgaccggatcgataagccgatcatcttcgccgagcggtcggtgtcggggcatggcttccacatcttcgtccggactgacgaggcccccggacgccgcaccggaaacatcgagttctactcacgccatcggttcatcagggtcacaggagaccagttcgtctgaagaagggggtgcgccatggctgcacaggtcagggccgtggaccccgatgagcgcccacccgcccgcaagcgggccaagaccatcacccaggccgcgaagtccggcactgaggttgaactgttggaggcactgcaggctcgcgtggcccgcgccgtgcaggaccgtgacactccgccgcgcgatctggcagcgctgacgaagcggctgatggacatcacccgggagctcgaggcggcccgggtcaaggatcaggaggcgggatctgatggtgccgtcaccgcagacgaaacatggcgaccgcaagctctctgaggtcgccaagcacctgatccttcctgaagggatcgtctcgacgggctggccggccgtgcgtgaccggtgtggcgagtggggtgtggtcttcgaccgttggcaggacggcatgggccgggtgatcctgtcgaagcgcggcagcggcctgttcgccgctggtgtgggcggggtcggcatgtcgatcccgcgccag